SEQUENCE LISTING (119 sequences)

SEQ ID NO: 1 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSWSSYLITFGQGTKVEIKRTV

SEQ ID NO: 2 - protein, 127 aa, synthetic construct
EVQLVESGGGLVQPGGSLRLSCAASGFTISSSSIHWVRQAPGKGLEWVASIYSYYGSTYYADSVKGRFTISADTSKNTAYLQMNSLRAEDTAVYYCARPVYGQLGYWTQRWSGMDYWGQGTLVTVSS

SEQ ID NO: 3 - protein, 11 aa, synthetic construct
RASQSVSSAVA

SEQ ID NO: 4 - protein, 7 aa, synthetic construct
SASSLYS

SEQ ID NO: 5 - protein, 11 aa, synthetic construct
QQYSWSSYLIT

SEQ ID NO: 6 - protein, 5 aa, synthetic construct
SSSIH

SEQ ID NO: 7 - protein, 17 aa, synthetic construct
SIYSYYGSTYYADSVKG

SEQ ID NO: 8 - protein, 18 aa, synthetic construct
PVYGQLGYWTQRWSGMDY

SEQ ID NO: 9 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSVYSPVTFGQGTKVEIKRTV

SEQ ID NO: 10 - protein, 130 aa, synthetic construct
EVQLVESGGGLVQPGGSLRLSCAASGFTISSYSIHWVRQAPGKGLEWVASISPYYGSTSYADSVKGRFTISADTSKNTAYLQMNSLRAEDTAVYYCARSWYYYEQNPYWYQKEEQALDYWGQGTLVTVSS

SEQ ID NO: 11 - protein, 9 aa, synthetic construct
QQSVYSPVT

SEQ ID NO: 12 - protein, 5 aa, synthetic construct
SYSIH

SEQ ID NO: 13 - protein, 17 aa, synthetic construct
SISPYYGSTSYADSVKG

SEQ ID NO: 14 - protein, 21 aa, synthetic construct
SWYYYEQNPYWYQKEEQALDY

SEQ ID NO: 15 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQLRHYSHLITFGQGTKVEIKRTV

SEQ ID NO: 16 - protein, 130 aa, synthetic construct
EVQLVESGGGLVQPGGSLRLSCAASGFTFSSSSIHWVRQAPGKGLEWVASISPYSGYTSYADSVKGRFTISADTSKNTAYLQMNSLRAEDTAVYYCARAYYYWSHWYMHYVSYPGGMDYWGQGTLVTVSS

SEQ ID NO: 17 - protein, 11 aa, synthetic construct
QQLRHYSHLIT

SEQ ID NO: 18 - protein, 17 aa, synthetic construct
SISPYSGYTSYADSVKG

SEQ ID NO: 19 - protein, 21 aa, synthetic construct
AYYYWSHWYMHYVSYPGGMDY

SEQ ID NO: 20 - protein, 9 aa, synthetic construct
QQSSSSLIT

SEQ ID NO: 21 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTV

SEQ ID NO: 22 - protein, 127 aa, synthetic construct
EVQLVESGGGLVQPGGSLRLSCAASGFTISSSSIHWVRQAPGKGLEWVASISSSSGSTSYADSVKGRFTISADTSKNTAYLQMNSLRAEDTAVYYCARIRHYHWYEIPYWDWAIDYWGQGTLVTVSS

SEQ ID NO: 23 - protein, 17 aa, synthetic construct
SISSSSGSTSYADSVKG

SEQ ID NO: 24 - protein, 18 aa, synthetic construct
IRHYHWYEIPYWDWAIDY

SEQ ID NO: 25 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYWGSDPITFGQGTKVEIKRTV

SEQ ID NO: 26 - protein, 130 aa, synthetic construct
EVQLVESGGGLVQPGGSLRLSCAASGFTVSSSSIHWVRQAPGKGLEWVASIYPYSGSTSYADSVKGRFTISADTSKNTAYLQMNSLRAEDTAVYYCARSLGWHRHYNYYFYFYANAIDYWGQGTLVTVSS

SEQ ID NO: 27 - protein, 10 aa, synthetic construct
QQYWGSDPIT

SEQ ID NO: 28 - protein, 17 aa, synthetic construct
SIYPYSGSTSYADSVKG

SEQ ID NO: 29 - protein, 21 aa, synthetic construct
SLGWHRHYNYYFYFYANAIDY

SEQ ID NO: 30 - protein, 17 aa, synthetic construct
SISSYSGSTSYADSVKG

SEQ ID NO: 31 - protein, 130 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSYWERYYYWYYFYQYELGLDYWGQGTLVTVSS

SEQ ID NO: 32 - protein, 129 aa, synthetic construct
EVQLVESGGGLVQPGGSLRLSCAASGFTISSSSIHWVRQAPGKGLEWVASISSYSGSTSYADSVKGRFTISADTSKNTAYLQMNSLRAEDTAVYYCARSFFKYYLWYGYHNYWSAIDYWGQGTLVTVSS

SEQ ID NO: 33 - protein, 20 aa, synthetic construct
SFFKYYLWYGYHNYWSAIDY

SEQ ID NO: 34 - protein, 11 aa, synthetic construct
QQSGYYGHLIT

SEQ ID NO: 35 - protein, 112 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSGYYGHLITFGQGTKVEIKRTV

SEQ ID NO: 36 - protein, 128 aa, synthetic construct
EVQLVESGGGLVQPGGSLRLSCAASGFTVSYSSIHWVRQAPGKGLEWVASIYSSSGYTSYADSVKGRFTISADTSKNTAYLQMNSLRAEDTAVYYCARSYYRYYLSPIYWGSWGMDYWGQGTLVTVSS

SEQ ID NO: 37 - protein, 5 aa, synthetic construct
YSSIH

SEQ ID NO: 38 - protein, 17 aa, synthetic construct
SIYSSSGYTSYADSVKG

SEQ ID NO: 39 - protein, 19 aa, synthetic construct
SYYRYYLSPIYWGSWGMDY

SEQ ID NO: 40 - protein, 10 aa, synthetic construct
QQYWHESLIT

SEQ ID NO: 41 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYWHESLITFGQGTKVEIKRTV

SEQ ID NO: 42 - protein, 130 aa, synthetic construct
EVQLVESGGGLVQPGGSLRLSCAASGFTVSSSSIHWVRQAPGKGLEWVASIYSYSGSTSYADSVKGRFTISADTSKNTAYLQMNSLRAEDTAVYYCARSFGWGQSFYYSHYFYITAFDYWGQGTLVTVSS

SEQ ID NO: 43 - protein, 17 aa, synthetic construct
SIYSYSGSTSYADSVKG

SEQ ID NO: 44 - protein, 21 aa, synthetic construct
SFGWGQSFYYSHYFYITAFDY

SEQ ID NO: 45 - protein, 21 aa, synthetic construct
SYWERYYYWYYFYQYELGLDY

SEQ ID NO: 46 - protein, 129 aa, synthetic construct
EVQLVESGGGLVQPGGSLRLSCAASGFTLSSSSIHWVRQAPGKGLEWVASIYSYYGSTYYADSVKGRFTISADTSKNTAYLQMNSLRAEDTAVYYCARYRGNMYYWESWYGSYWALDYWGQGTLVTVSS

SEQ ID NO: 47 - protein, 20 aa, synthetic construct
YRGNMYYWESWYGSYWALDY

SEQ ID NO: 48 - protein, 9 aa, synthetic construct
QQSSGYPVT

SEQ ID NO: 49 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSGYPVTFGQGTKVEIKRTV

SEQ ID NO: 50 - protein, 124 aa, synthetic construct
EVQLVESGGGLVQPGGSLRLSCAASGFTVSSSSIHWVRQAPGKGLEWVASISSYYGYTSYADSVKGRFTISADTSKNTAYLQMNSLRAEDTAVYYCARYPMFSYYYWYYGMDYWGQGTLVTVSS

SEQ ID NO: 51 - protein, 17 aa, synthetic construct
SISSYYGYTSYADSVKG

SEQ ID NO: 52 - protein, 15 aa, synthetic construct
YPMFSYYYWYYGMDY

SEQ ID NO: 53 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYWGYSLITFGQGTKVEIKRTV

SEQ ID NO: 54 - protein, 127 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARIRHYHWYEIPYWDWAIDYWGQGTLVTVSS

SEQ ID NO: 55 - protein, 10 aa, synthetic construct
QQYWGYSLIT

SEQ ID NO: 56 - protein, 127 aa, synthetic construct
EVQLVESGGGLVQPGGSLRLSCAASGFTFSSSSIHWVRQAPGKGLEWVASISPYYGSTSYADSVKGRFTISADTSKNTAYLQMNSLRAEDTAVYYCARKLEWGMFFWSPYFRALDYWGQGTLVTVSS

SEQ ID NO: 57 - protein, 18 aa, synthetic construct
KLEWGMFFWSPYFRALDY

SEQ ID NO: 58 - protein, 10 aa, synthetic construct
QQSKFSSPVT

SEQ ID NO: 59 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSKFSSPVTFGQGTKVEIKRTV

SEQ ID NO: 60 - protein, 130 aa, synthetic construct
EVQLVESGGGLVQPGGSLRLSCAASGFTFSSSSIHWVRQAPGKGLEWVASISSSYGSTSYADSVKGRFTISADTSKNTAYLQMNSLRAEDTAVYYCARGWAYSYWYYSHYYEYEVALDYWGQGTLVTVSS

SEQ ID NO: 61 - protein, 17 aa, synthetic construct
SISSSYGSTSYADSVKG

SEQ ID NO: 62 - protein, 21 aa, synthetic construct
GWAYSYWYYSHYYEYEVALDY

SEQ ID NO: 63 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSWWDPITFGQGTKVEIKRTV

SEQ ID NO: 64 - protein, 128 aa, synthetic construct
EVQLVESGGGLVQPGGSLRLSCAASGFTISSSSIHWVRQAPGKGLEWVAYIYPSSGSTSYADSVKGRFTISADTSKNTAYLQMNSLRAEDTAVYYCARPWYYSTFNKKWWDYQGLDYWGQGTLVTVSS

SEQ ID NO: 65 - protein, 9 aa, synthetic construct
QQSWWDPIT

SEQ ID NO: 66 - protein, 17 aa, synthetic construct
YIYPSSGSTSYADSVKG

SEQ ID NO: 67 - protein, 19 aa, synthetic construct
PWYYSTFNKKWWDYQGLDY

SEQ ID NO: 68 - protein, 9 aa, synthetic construct
QQDSSYPIT

SEQ ID NO: 69 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQDSSYPITFGQGTKVEIKRTV

SEQ ID NO: 70 - protein, 125 aa, synthetic construct
EVQLVESGGGLVQPGGSLRLSCAASGFTVSSSSIHWVRQAPGKGLEWVAYISSSSGSTYYADSVKGRFTISADTSKNTAYLQMNSLRAEDTAVYYCARDFYFGSYYSFGWGLDYWGQGTLVTVSS

SEQ ID NO: 71 - protein, 17 aa, synthetic construct
YISSSSGSTYYADSVKG

SEQ ID NO: 72 - protein, 16 aa, synthetic construct
DFYFGSYYSFGWGLDY

SEQ ID NO: 73 - protein, 129 aa, synthetic construct
EVQLVESGGGLVQPGGSLRLSCAASGFTFYYSSIHWVRQAPGKGLEWVASIYSYSGYTSYADSVKGRFTISADTSKNTAYLQMNSLRAEDTAVYYCARYYISGPYYAYRSYTSYAMDYWGQGTLVTVSS

SEQ ID NO: 74 - protein, 130 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSYWDMYIYWYYYVKQSRGMDYWGQGTLVTVSS

SEQ ID NO: 75 - protein, 21 aa, synthetic construct
SYWDMYIYWYYYVKQSRGMDY

SEQ ID NO: 76 - protein, 10 aa, synthetic construct
QQWWGPELVT

SEQ ID NO: 77 - protein, 111 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWWGPELVTFGQGTKVEIKRTV

SEQ ID NO: 78 - protein, 130 aa, synthetic construct
EVQLVESGGGLVQPGGSLRLSCAASGFTISSSSIHWVRQAPGKGLEWVASISSSSSYTSYADSVKGRFTISADTSKNTAYLQMNSLRAEDTAVYYCARMDISYYYYEWGFESAYYGMDYWGQGTLVTVSS

SEQ ID NO: 79 - protein, 17 aa, synthetic construct
SISSSSSYTSYADSVKG

SEQ ID NO: 80 - protein, 21 aa, synthetic construct
MDISYYYYEWGFESAYYGMDY

SEQ ID NO: 81 - protein, 128 aa, synthetic construct
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARVLLGKFWYYNYYEYSAMDYWGQGTLVTVSS

SEQ ID NO: 82 - protein, 19 aa, synthetic construct
VLLGKFWYYNYYEYSAMDY

SEQ ID NO: 83 - protein, 17 aa, synthetic construct
SIYSYSGYTSYADSVKG

SEQ ID NO: 84 - protein, 20 aa, synthetic construct
YYISGPYYAYRSYTSYAMDY

SEQ ID NO: 85 - protein, 835 aa, Homo sapiens
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECQQNPRLCKSYGTCVNTLGSYTCQCLPGFKFIPEDPKVCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 86 - protein, 742 aa, Homo sapiens
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 87 - protein, 786 aa, Homo sapiens
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECQQNPRLCKSYGTCVNTLGSYTCQCLPGFKFIPEDPKVCTDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 88 - skipped

SEQ ID NO: 89 - protein, 4 aa, synthetic construct
LLQG

SEQ ID NO: 90 - protein, 5 aa, synthetic construct
LLQGG

SEQ ID NO: 91 - protein, 6 aa, synthetic construct
LSLSQG

SEQ ID NO: 92 - protein, 8 aa, synthetic construct
GGGLLQGG

SEQ ID NO: 93 - protein, 4 aa, synthetic construct
GLLQ

SEQ ID NO: 94 - protein, 5 aa, synthetic construct
GLLQG

SEQ ID NO: 95 - protein, 7 aa, synthetic construct
GLLQGGG

SEQ ID NO: 96 - protein, 6 aa, synthetic construct
GLLQGG

SEQ ID NO: 97 - protein, 10 aa, synthetic construct
GSPLAQSHGG

SEQ ID NO: 98 - protein, 8 aa, synthetic construct
LLQLLQGA

SEQ ID NO: 99 - protein, 5 aa, synthetic construct
LLQGA

SEQ ID NO: 100 - protein, 7 aa, synthetic construct
LLQYQGA

SEQ ID NO: 101 - protein, 6 aa, synthetic construct
LLQGSG

SEQ ID NO: 102 - protein, 6 aa, synthetic construct
LLQYQG

SEQ ID NO: 103 - protein, 7 aa, synthetic construct
LLQLLQG

SEQ ID NO: 104 - protein, 5 aa, synthetic construct
SLLQG

SEQ ID NO: 105 - protein, 5 aa, synthetic construct
LLQLQ

SEQ ID NO: 106 - protein, 6 aa, synthetic construct
LLQLLQ

SEQ ID NO: 107 - protein, 5 aa, synthetic construct
LLQGR

SEQ ID NO: 108 - skipped

SEQ ID NO: 109 - protein, 130 aa, synthetic construct
EVQLVESGGGLVQPGGSLRLSCAASGFTISSYSIHWVRQAPGKGLEWVASISPYYGSTSYADSVKGRFTISADTSKNTAYLQMNSLRAEDTAVYYCARSWYYYEQEPYWYQKEEQALDYWGQGTLVTVSS

SEQ ID NO: 110 - protein, 130 aa, synthetic construct
EVQLVESGGGLVQPGGSLRLSCAASGFTISSYSIHWVRQAPGKGLEWVASISPYYGSTSYADSVKGRFTISADTSKNTAYLQMNSLRAEDTAVYYCARSWYYYEQNPYAYQKEEQALDYWGQGTLVTVSS

SEQ ID NO: 111 - protein, 130 aa, synthetic construct
EVQLVESGGGLVQPGGSLRLSCAASGFTISSYSIHWVRQAPGKGLEWVASISPYYGSTSYADSVKGRFTISADTSKNTAYLQMNSLRAEDTAVYYCARSWYYYEQNPYWAQKEEQALDYWGQGTLVTVSS

SEQ ID NO: 112 - protein, 21 aa, synthetic construct
SWYYYEQEPYWYQKEEQALDY

SEQ ID NO: 113 - protein, 21 aa, synthetic construct
SWYYYEQNPYAYQKEEQALDY

SEQ ID NO: 114 - protein, 21 aa, synthetic construct
SWYYYEQNPYWAQKEEQALDY

SEQ ID NO: 115 - protein, 6 aa, synthetic construct
HHHHHH

SEQ ID NO: 116 - DNA, 106 bp, unidentified
  source 1..106: mol_type = unassigned DNA
tgcctagttgctggggggagcaagtactcagaattcacctccaccacgtctggcactggccacaatcagacccgggccctcagggcatcagagtccggcatataga

SEQ ID NO: 117 - protein, 34 aa, unidentified
CLVAGGSKYSEFTSTTSGTGHNQTRALRASESGI

SEQ ID NO: 118 - DNA, 37 bp, synthetic construct
  source 1..37: mol_type = other DNA
tgcctagttgctggggggagcaagtacggcatataga

SEQ ID NO: 119 - protein, 11 aa, synthetic construct
CLVAGGSKYGI

(19) United States
(12) Patent Application Publication
(10) Pub. No.: US 2025/0051474 A1
(43) Pub. Date: Feb. 13, 2025

(54) ANTI-CD97 ANTIBODIES AND ANTIBODY-DRUG CONJUGATES

(71) Applicant: New York University, New York, NY (US)

(72) Inventors: Shohei KOIDE, New York, NY (US); Christopher Y. PARK, New York, NY (US); Takamitsu HATTORI, New York, NY (US); Akiko KOIDE, New York, NY (US); Dimitris PLACANTONAKIS, New York, NY (US)

(73) Assignee: New York University, New York, NY (US)

(21) Appl. No.: 18/790,680

(22) Filed: Jul. 31, 2024

Related U.S. Application Data
(60) Provisional application No. 63/530,196, filed on Aug. 1, 2023.

(51) Int. Cl. C07K 16/28; A61K 39/00; A61K 47/68; A61P 35/00

(52) U.S. Cl. CPC C07K 16/2896; A61K 47/6809; A61K 47/6889; A61P 35/00; A61K 2039/505; C07K 2317/14; C07K 2317/31; C07K 2317/55; C07K 2317/565; C07K 2317/92

(57) ABSTRACT

This application relates to isolated antibodies, or antigen-binding fragments thereof, that bind to at least one isoform of cluster of differentiation 97 (CD97), also known as adhesion G protein-coupled receptor E5 (ADGRE5), antibody-drug conjugates comprising such antibodies or antigen-binding fragments, as well as polynucleotides and vectors that encode for such antibodies or antigen-binding fragments. This application further relates to methods of producing the antibodies or antigen-binding fragments and using the antibodies or antigen-binding fragments for treatment of diseases.

### CROSS-REFERENCE TO RELATED APPLICATIONS

This patent application claims priority to U.S. Provisional Application No. 63/530,196, filed Aug. 1, 2023, the disclosure of which is herein incorporated by reference in its entirety.

### STATEMENT REGARDING FEDERALLY SPONSORED RESEARCH

This invention was made with government support under CA251669, CA016087, and NS102665 awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jul. 29, 2024, is named 243735_000384_SL.xml and is 109,321 bytes in size.

### FIELD OF THE INVENTION

This application relates to isolated antibodies, or antigen-binding fragments thereof, that bind to at least one isoform of cluster of differentiation 97 (CD97), also known as adhesion G protein-coupled receptor E5 (ADGRE5), antibody-drug conjugates comprising such antibodies or antigen-binding fragments, as well as polynucleotides and vectors that encode for such antibodies or antigen-binding fragments. This application further relates to methods of producing the antibodies or antigen-binding fragments, and using the antibodies or antigen-binding fragments for treatment of diseases.

### BACKGROUND

Glioma is the most common primary brain malignancy. In adults, two main types of glioma exist.^{1,2} The less common type is typically encountered in younger patients and is driven by a neomorphic mutation in the metabolic enzyme isocitrate dehydrogenase (IDH). The more common type, also known as glioblastoma (GBM), is observed in older patients, lacks the IDH mutation (IDH-wildtype), has an aggressive course, and represents the largest unmet need in neurooncology. The current treatment regimen for GBM involves neurosurgical resection of the tumor, followed by chemoradiotherapy. Nevertheless, these measures have done little to improve patient outcomes, with the median survival limited to about 15 months.³⁻⁵

CD97, also known as adhesion G protein-coupled receptor E5 (ADGRE5), is expressed in several cell lineages of the immune system, where it is critical for the inflammatory response,²⁰⁻²⁴ as well as in multiple liquid and solid malignancies.^{13,25-28} Among these malignancies is GBM, in which CD97 has been implicated in cellular proliferation, brain invasion, and tumor metabolism.²⁹⁻³³ However, its mechanism of action in GBM remains incompletely understood. Furthermore, little research effort has been devoted to its therapeutic targeting.

CD97 is also expressed in the vast majority of human acute myeloid leukemia (AML). AML is the most common type of acute leukemia in adults. This type of cancer usually gets worse quickly if it is not treated. The main treatment for most types of AML is chemotherapy, sometimes along with a targeted therapy drug. This might be followed by a stem cell transplant. Other drugs (besides standard chemotherapy drugs) may be used to treat people with acute promyelocytic leukemia (APL). The 5-year relative survival rate for people 20 and older with AML is 28%. For people younger than 20, the 5-year relative survival rate is 69%. Improved therapies for AML are needed.

### SUMMARY OF THE INVENTION

As specified in the Background section above, there is a need in the art for therapeutic targeting of CD97 (ADGRE5) for improved treatment of, e.g., hematologic cancers (e.g., AML) and solid cancers (e.g., glioblastoma (GBM)). The present application addresses these and other needs.

In one aspect, provided herein is an antibody, or antigen-binding fragment thereof, that binds to at least one isoform of cluster of differentiation 97 (CD97).

In some embodiments, the antibody or antigen-binding fragment comprises a heavy chain complementarity determining region 1 (CDR1), a heavy chain CDR2, a heavy chain CDR3 of a heavy chain variable region (VH) comprising an amino acid sequence of SEQ ID NO: 2, 10, 16, 22, 26, 32, 36, 42, 46, 50, 54, 56, 60, 64, 70, 74, 78, 81, 31, or 73; and/or a light chain CDR1, a light chain CDR2, and a light chain CDR3 of a light chain variable region (VL) comprising an amino acid sequence of SEQ ID NO: 1, 9, 15, 21, 25, 35, 41, 49, 59, 63, 69, 77, or 53.

In some embodiments, the antibody or antigen-binding fragment comprises a heavy chain CDR1 and a heavy chain CDR2 of a VH comprising an amino acid sequence of SEQ ID NO: 2, 10, 16, 22, 26, 32, 36, 42, 46, 50, 54, 56, 60, 64, 70, 74, 78, 81, 31, or 73; and/or a light chain CDR1, a light chain CDR2, and a light chain CDR3 of a VL comprising an amino acid sequence of SEQ ID NO: 1, 9, 15, 21, 25, 35, 41, 49, 59, 63, 69, 77, or 53, and a heavy chain CDR3 comprising N at position 100b, W at position 100e, and/or Y at position 100f (numbering according to Kabat). In some embodiments, the heavy chain CDR3 comprises N at position 100b. In some embodiments, the heavy chain CDR3 comprises W at position 100e. In some embodiments, the heavy chain CDR3 comprises Y at position 100f. In some embodiments, the heavy chain CDR3 comprises N at position 100b and W at position 100e. In some embodiments, the heavy chain CDR3 comprises N at position 100b and Y at position 100f. In some embodiments, the heavy chain CDR3 comprises W at position 100e and Y at position 100f. In some embodiments, the heavy chain CDR3 comprises N at position 100b, W at position 100e, and Y at position 100f.

In some embodiments, the antibody or antigen-binding fragment comprises
a) a heavy chain CDR1, a heavy chain CDR2, a heavy chain CDR3 of a VH comprising an amino acid sequence of SEQ ID NO: 2, and a light chain CDR1, a light chain CDR2, and a light chain CDR3 of a VL comprising an amino acid sequence of SEQ ID NO: 1;
b) a heavy chain CDR1, a heavy chain CDR2, a heavy chain CDR3 of a VH comprising an amino acid sequence of SEQ ID NO: 10, and a light chain CDR1, a light chain CDR2, and a light chain CDR3 of a VL comprising an amino acid sequence of SEQ ID NO: 9;
c) a heavy chain CDR1, a heavy chain CDR2, a heavy chain CDR3 of a VH comprising an amino acid sequence of SEQ ID NO: 16, and a light chain CDR1, a light chain CDR2, and a light chain CDR3 of a VL comprising an amino acid sequence of SEQ ID NO: 15;
d) a heavy chain CDR1, a heavy chain CDR2, a heavy chain CDR3 of a VH comprising an amino acid sequence of SEQ ID NO: 22, and a light chain CDR1, a light chain CDR2, and a light chain CDR3 of a VL comprising an amino acid sequence of SEQ ID NO: 21;
e) a heavy chain CDR1, a heavy chain CDR2, a heavy chain CDR3 of a VH comprising an amino acid sequence of SEQ ID NO: 26, and a light chain CDR1, a light chain CDR2, and a light chain CDR3 of a VL comprising an amino acid sequence of SEQ ID NO: 25;
f) a heavy chain CDR1, a heavy chain CDR2, a heavy chain CDR3 of a VH comprising an amino acid sequence of SEQ ID NO: 32, and a light chain CDR1, a light chain CDR2, and a light chain CDR3 of a VL comprising an amino acid sequence of SEQ ID NO: 21;
g) a heavy chain CDR1, a heavy chain CDR2, a heavy chain CDR3 of a VH comprising an amino acid sequence of SEQ ID NO: 36, and a light chain CDR1, a light chain CDR2, and a light chain CDR3 of a VL comprising an amino acid sequence of SEQ ID NO: 35;
h) a heavy chain CDR1, a heavy chain CDR2, a heavy chain CDR3 of a VH comprising an amino acid sequence of SEQ ID NO: 42, and a light chain CDR1, a light chain CDR2, and a light chain CDR3 of a VL comprising an amino acid sequence of SEQ ID NO: 41;
i) a heavy chain CDR1, a heavy chain CDR2, a heavy chain CDR3 of a VH comprising an amino acid sequence of SEQ ID NO: 46, and a light chain CDR1, a light chain CDR2, and a light chain CDR3 of a VL comprising an amino acid sequence of SEQ ID NO: 21;
j) a heavy chain CDR1, a heavy chain CDR2, a heavy chain CDR3 of a VH comprising an amino acid sequence of SEQ ID NO: 50, and a light chain CDR1, a light chain CDR2, and a light chain CDR3 of a VL comprising an amino acid sequence of SEQ ID NO: 49;
k) a heavy chain CDR1, a heavy chain CDR2, a heavy chain CDR3 of a VH comprising an amino acid sequence of SEQ ID NO: 54, and a light chain CDR1, a light chain CDR2, and a light chain CDR3 of a VL comprising an amino acid sequence of SEQ ID NO: 21;
1) a heavy chain CDR1, a heavy chain CDR2, a heavy chain CDR3 of a VH comprising an amino acid sequence of SEQ ID NO: 56, and a light chain CDR1, a light chain CDR2, and a light chain CDR3 of a VL comprising an amino acid sequence of SEQ ID NO: 21;
m) a heavy chain CDR1, a heavy chain CDR2, a heavy chain CDR3 of a VH comprising an amino acid sequence of SEQ ID NO: 60, and a light chain CDR1, a light chain CDR2, and a light chain CDR3 of a VL comprising an amino acid sequence of SEQ ID NO: 59;
n) a heavy chain CDR1, a heavy chain CDR2, a heavy chain CDR3 of a VH comprising an amino acid sequence of SEQ ID NO: 64, and a light chain CDR1, a light chain CDR2, and a light chain CDR3 of a VL comprising an amino acid sequence of SEQ ID NO: 63;
o) a heavy chain CDR1, a heavy chain CDR2, a heavy chain CDR3 of a VH comprising an amino acid sequence of SEQ ID NO: 70, and a light chain CDR1, a light chain CDR2, and a light chain CDR3 of a VL comprising an amino acid sequence of SEQ ID NO: 69;
p) a heavy chain CDR1, a heavy chain CDR2, a heavy chain CDR3 of a VH comprising an amino acid sequence of SEQ ID NO: 74, and a light chain CDR1, a light chain CDR2, and a light chain CDR3 of a VL comprising an amino acid sequence of SEQ ID NO: 21;
q) a heavy chain CDR1, a heavy chain CDR2, a heavy chain CDR3 of a VH comprising an amino acid sequence of SEQ ID NO: 78, and a light chain CDR1, a light chain CDR2, and a light chain CDR3 of a VL comprising an amino acid sequence of SEQ ID NO: 77;
r) a heavy chain CDR1, a heavy chain CDR2, a heavy chain CDR3 of a VH comprising an amino acid sequence of SEQ ID NO: 81, and a light chain CDR1, a light chain CDR2, and a light chain CDR3 of a VL comprising an amino acid sequence of SEQ ID NO: 21;
s) a heavy chain CDR1, a heavy chain CDR2, a heavy chain CDR3 of a VH comprising an amino acid sequence of SEQ ID NO: 31, and a light chain CDR1, a light chain CDR2, and a light chain CDR3 of a VL comprising an amino acid sequence of SEQ ID NO: 21; or
t) a heavy chain CDR1, a heavy chain CDR2, a heavy chain CDR3 of a VH comprising an amino acid sequence of SEQ ID NO: 73, and a light chain CDR1, a light chain CDR2, and a light chain CDR3 of a VL comprising an amino acid sequence of SEQ ID NO: 53.

In some embodiments, the antibody or antigen-binding fragment comprises a heavy chain CDR1 comprising the amino acid sequence of SEQ ID NO: 6, a heavy chain CDR2 comprising the amino acid sequence of SEQ ID NO: 7, and a heavy chain CDR3 comprising the amino acid sequence of SEQ ID NO: 8;
a heavy chain CDR1 comprising the amino acid sequence of SEQ ID NO: 12, a heavy chain CDR2 comprising the amino acid sequence of SEQ ID NO: 13, and a heavy chain CDR3 comprising the amino acid sequence of SEQ ID NO: 14;
a heavy chain CDR1 comprising the amino acid sequence of SEQ ID NO: 6, a heavy chain CDR2 comprising the amino acid sequence of SEQ ID NO: 18, and a heavy chain CDR3 comprising the amino acid sequence of SEQ ID NO: 19;
a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 24;
a heavy chain CDR1 comprising the amino acid sequence of SEQ ID NO: 6, a heavy chain CDR2 comprising the amino acid sequence of SEQ ID NO: 28, and a heavy chain CDR3 comprising the amino acid sequence of SEQ ID NO: 29;
a heavy chain CDR1 comprising the amino acid sequence of SEQ ID NO: 6, a heavy chain CDR2 comprising the amino acid sequence of SEQ ID NO: 30, and a heavy chain CDR3 comprising the amino acid sequence of SEQ ID NO: 33;
a heavy chain CDR1 comprising the amino acid sequence of SEQ ID NO: 37, a heavy chain CDR2 comprising the amino acid sequence of SEQ ID NO: 38, and a heavy chain CDR3 comprising the amino acid sequence of SEQ ID NO: 39;
a heavy chain CDR1 comprising the amino acid sequence of SEQ ID NO: 6, a heavy chain CDR2 comprising the amino acid sequence of SEQ ID NO: 43, and a heavy chain CDR3 comprising the amino acid sequence of SEQ ID NO: 44;
a heavy chain CDR1 comprising the amino acid sequence of SEQ ID NO: 6, a heavy chain CDR2 comprising the amino acid sequence of SEQ ID NO: 7, and a heavy chain CDR3 comprising the amino acid sequence of SEQ ID NO: 47;
a heavy chain CDR1 comprising the amino acid sequence of SEQ ID NO: 6, a heavy chain CDR2 comprising the amino acid sequence of SEQ ID NO: 51, and a heavy chain CDR3 comprising the amino acid sequence of SEQ ID NO: 52;
a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 24;
a heavy chain CDR1 comprising the amino acid sequence of SEQ ID NO: 6, a heavy chain CDR2 comprising the amino acid sequence of SEQ ID NO: 13, and a heavy chain CDR3 comprising the amino acid sequence of SEQ ID NO: 57;
a heavy chain CDR1 comprising the amino acid sequence of SEQ ID NO: 6, a heavy chain CDR2 comprising the amino acid sequence of SEQ ID NO: 61, and a heavy chain CDR3 comprising the amino acid sequence of SEQ ID NO: 62;
a heavy chain CDR1 comprising the amino acid sequence of SEQ ID NO: 6, a heavy chain CDR2 comprising the amino acid sequence of SEQ ID NO: 66, and a heavy chain CDR3 comprising the amino acid sequence of SEQ ID NO: 67;
a heavy chain CDR1 comprising the amino acid sequence of SEQ ID NO: 6, a heavy chain CDR2 comprising the amino acid sequence of SEQ ID NO: 71, and a heavy chain CDR3 comprising the amino acid sequence of SEQ ID NO: 72;
a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 75;
a heavy chain CDR1 comprising the amino acid sequence of SEQ ID NO: 6, a heavy chain CDR2 comprising the amino acid sequence of SEQ ID NO: 79, and a heavy chain CDR3 comprising the amino acid sequence of SEQ ID NO: 80;
a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 82;
a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 45; or
a heavy chain CDR1 comprising the amino acid sequence of SEQ ID NO: 37, a heavy chain CDR2 comprising the amino acid sequence of SEQ ID NO: 83, and a heavy chain CDR3 comprising the amino acid sequence of SEQ ID NO: 84.

In some embodiments, the antibody or antigen-binding fragment comprises
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 5;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 11;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 17;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 27;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 34;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 40;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 48;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 58;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 65;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 68;
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 76; or
a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 55.

In some embodiments, the antibody or antigen-binding fragment comprises
a) a heavy chain CDR1 comprising the amino acid sequence of SEQ ID NO: 6, a heavy chain CDR2 comprising the amino acid sequence of SEQ ID NO: 7, and a heavy chain CDR3 comprising the amino acid sequence of SEQ ID NO: 8, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 5;
b) a heavy chain CDR1 comprising the amino acid sequence of SEQ ID NO: 12, a heavy chain CDR2 comprising the amino acid sequence of SEQ ID NO: 13, and a heavy chain CDR3 comprising the amino acid sequence of SEQ ID NO: 14, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 11;
c) a heavy chain CDR1 comprising the amino acid sequence of SEQ ID NO: 6, a heavy chain CDR2 comprising the amino acid sequence of SEQ ID NO: 18, and a heavy chain CDR3 comprising the amino acid sequence of SEQ ID NO: 19, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 17;
d) a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 24, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
e) a heavy chain CDR1 comprising the amino acid sequence of SEQ ID NO: 6, a heavy chain CDR2 comprising the amino acid sequence of SEQ ID NO: 28, and a heavy chain CDR3 comprising the amino acid sequence of SEQ ID NO: 29, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 27;
f) a heavy chain CDR1 comprising the amino acid sequence of SEQ ID NO: 6, a heavy chain CDR2 comprising the amino acid sequence of SEQ ID NO: 30, and a heavy chain CDR3 comprising the amino acid sequence of SEQ ID NO: 33, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
g) a heavy chain CDR1 comprising the amino acid sequence of SEQ ID NO: 37, a heavy chain CDR2 comprising the amino acid sequence of SEQ ID NO: 38, and a heavy chain CDR3 comprising the amino acid sequence of SEQ ID NO: 39, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 34;
h) a heavy chain CDR1 comprising the amino acid sequence of SEQ ID NO: 6, a heavy chain CDR2 comprising the amino acid sequence of SEQ ID NO: 43, and a heavy chain CDR3 comprising the amino acid sequence of SEQ ID NO: 44, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 40;
i) a heavy chain CDR1 comprising the amino acid sequence of SEQ ID NO: 6, a heavy chain CDR2 comprising the amino acid sequence of SEQ ID NO: 7, and a heavy chain CDR3 comprising the amino acid sequence of SEQ ID NO: 47, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
j) a heavy chain CDR1 comprising the amino acid sequence of SEQ ID NO: 6, a heavy chain CDR2 comprising the amino acid sequence of SEQ ID NO: 51, and a heavy chain CDR3 comprising the amino acid sequence of SEQ ID NO: 52, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 48;
k) a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 24, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
1) a heavy chain CDR1 comprising the amino acid sequence of SEQ ID NO: 6, a heavy chain CDR2 comprising the amino acid sequence of SEQ ID NO: 13, and a heavy chain CDR3 comprising the amino acid sequence of SEQ ID NO: 57, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
m) a heavy chain CDR1 comprising the amino acid sequence of SEQ ID NO: 6, a heavy chain CDR2 comprising the amino acid sequence of SEQ ID NO: 61, and a heavy chain CDR3 comprising the amino acid sequence of SEQ ID NO: 62, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 58;
n) a heavy chain CDR1 comprising the amino acid sequence of SEQ ID NO: 6, a heavy chain CDR2 comprising the amino acid sequence of SEQ ID NO: 66, and a heavy chain CDR3 comprising the amino acid sequence of SEQ ID NO: 67, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 65;
o) a heavy chain CDR1 comprising the amino acid sequence of SEQ ID NO: 6, a heavy chain CDR2 comprising the amino acid sequence of SEQ ID NO: 71, and a heavy chain CDR3 comprising the amino acid sequence of SEQ ID NO: 72, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 68;
p) a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 75, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
q) a heavy chain CDR1 comprising the amino acid sequence of SEQ ID NO: 6, a heavy chain CDR2 comprising the amino acid sequence of SEQ ID NO: 79, and a heavy chain CDR3 comprising the amino acid sequence of SEQ ID NO: 80, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 76;
r) a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 82, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
s) a heavy chain CDR1 comprising the amino acid sequence of SEQ ID NO: 6, a heavy chain CDR2 comprising the amino acid sequence of SEQ ID NO: 23, and a heavy chain CDR3 comprising the amino acid sequence of SEQ ID NO: 45, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20; or
t) a heavy chain CDR1 comprising the amino acid sequence of SEQ ID NO: 37, a heavy chain CDR2 comprising the amino acid sequence of SEQ ID NO: 83, and a heavy chain CDR3 comprising the amino acid sequence of SEQ ID NO: 84, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 55.

In some embodiments, the antibody or antigen-binding fragment comprises a VH comprising an amino acid sequence of SEQ ID NO: 2, 10, 16, 22, 26, 32, 36, 42, 46, 50, 54, 56, 60, 64, 70, 74, 78, 81, 31, or 73; and/or a VL comprising an amino acid sequence of SEQ ID NO: 1, 9, 15, 21, 25, 35, 41, 49, 59, 63, 69, 77, or 53.

In some embodiments, the antibody or antigen-binding fragment comprises
a) a VH comprising an amino acid sequence of SEQ ID NO: 2, and a VL comprising an amino acid sequence of SEQ ID NO: 1;
b) a VH comprising an amino acid sequence of SEQ ID NO: 10, and a VL comprising an amino acid sequence of SEQ ID NO: 9;
c) a VH comprising an amino acid sequence of SEQ ID NO: 16, and a VL comprising an amino acid sequence of SEQ ID NO: 15;
d) a VH comprising an amino acid sequence of SEQ ID NO: 22, and a VL comprising an amino acid sequence of SEQ ID NO: 21;
e) a VH comprising an amino acid sequence of SEQ ID NO: 26, and a VL comprising an amino acid sequence of SEQ ID NO: 25;
f) a VH comprising an amino acid sequence of SEQ ID NO: 32, and a VL comprising an amino acid sequence of SEQ ID NO: 21;
g) a VH comprising an amino acid sequence of SEQ ID NO: 36, and a VL comprising an amino acid sequence of SEQ ID NO: 35;
h) a VH comprising an amino acid sequence of SEQ ID NO: 42, and a VL comprising an amino acid sequence of SEQ ID NO: 41;
i) a VH comprising an amino acid sequence of SEQ ID NO: 46, and a VL comprising an amino acid sequence of SEQ ID NO: 21;
j) a VH comprising an amino acid sequence of SEQ ID NO: 50, and a VL comprising an amino acid sequence of SEQ ID NO: 49;
k) a VH comprising an amino acid sequence of SEQ ID NO: 54, and a VL comprising an amino acid sequence of SEQ ID NO: 21;
l) a VH comprising an amino acid sequence of SEQ ID NO: 56, and a VL comprising an amino acid sequence of SEQ ID NO: 21;
m) a VH comprising an amino acid sequence of SEQ ID NO: 60, and a VL comprising an amino acid sequence of SEQ ID NO: 59;
n) a VH comprising an amino acid sequence of SEQ ID NO: 64, and a VL comprising an amino acid sequence of SEQ ID NO: 63;
o) a VH comprising an amino acid sequence of SEQ ID NO: 70, and a VL comprising an amino acid sequence of SEQ ID NO: 69;
p) a VH comprising an amino acid sequence of SEQ ID NO: 74, and a VL comprising an amino acid sequence of SEQ ID NO: 21;
q) a VH comprising an amino acid sequence of SEQ ID NO: 78, and a VL comprising an amino acid sequence of SEQ ID NO: 77;
r) a VH comprising an amino acid sequence of SEQ ID NO: 81, and a VL comprising an amino acid sequence of SEQ ID NO: 21;
s) a VH comprising an amino acid sequence of SEQ ID NO: 31, and a VL comprising an amino acid sequence of SEQ ID NO: 21; or
t) a VH comprising an amino acid sequence of SEQ ID NO: 73, and a VL comprising an amino acid sequence of SEQ ID NO: 53.

In some embodiments, the antibody or antigen-binding fragment comprises
i. a heavy chain CDR1, a heavy chain CDR2, a heavy chain CDR3 of a VH comprising an amino acid sequence of SEQ ID NO: 10, and a light chain CDR1, a light chain CDR2, and a light chain CDR3 of a VL comprising an amino acid sequence of SEQ ID NO: 9; or
ii. a heavy chain CDR1, a heavy chain CDR2, a heavy chain CDR3 of a VH comprising an amino acid sequence of SEQ ID NO: 64, and a light chain CDR1, a light chain CDR2, and a light chain CDR3 of a VL comprising an amino acid sequence of SEQ ID NO: 63.

In some embodiments, the antibody or antigen-binding fragment comprises
i. a heavy chain CDR1 comprising the amino acid sequence of SEQ ID NO: 12, a heavy chain CDR2 comprising the amino acid sequence of SEQ ID NO: 13, and a heavy chain CDR3 comprising the amino acid sequence of SEQ ID NO: 14, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 11; or
ii. a heavy chain CDR1 comprising the amino acid sequence of SEQ ID NO: 6, a heavy chain CDR2 comprising the amino acid sequence of SEQ ID NO: 66, and a heavy chain CDR3 comprising the amino acid sequence of SEQ ID NO: 67, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 65.

In some embodiments, the antibody or antigen-binding fragment comprises
i. a VH comprising an amino acid sequence of SEQ ID NO: 10, and a VL comprising an amino acid sequence of SEQ ID NO: 9; or
ii. a VH comprising an amino acid sequence of SEQ ID NO: 64, and a VL comprising an amino acid sequence of SEQ ID NO: 63.

In some embodiments, the antibody or antigen-binding fragment binds to isoform 1 of CD97.

In some embodiments, the antibody or antigen-binding fragment binds to isoform 2 of CD97.

In some embodiments, the antibody or antigen-binding fragment binds to isoform 3 of CD97.

In some embodiments, the antibody or antigen-binding fragment binds to both isoform 1 and isoform 2 of CD97.

In some embodiments, the antibody or antigen-binding fragment binds to isoform 1, isoform 2, and isoform 3 of CD97

In some embodiments, the antibody or antigen-binding fragment binds to all isoforms of CD97.

In another aspect, provided herein is an antibody, or antigen-binding fragment thereof, that binds to at least one isoform of cluster of differentiation 97 (CD97), wherein the antibody or antigen-binding fragment thereof binds to one or more amino acid residues of isoform 1 of CD97 selected from T351, Q376, R380, K382, K519, and T524, and any combination thereof.

In some embodiments, the antibody or antigen-binding fragment binds to the GPCR autoproteolysis-inducing (GAIN) domain in CD97.

In some embodiments, the antibody or antigen-binding fragment binds to the first EGF-like domain in CD97.

In some embodiments, the antibody or antigen-binding fragment binds to the third EGF-like domain in CD97.

In some embodiments, the antibody or antigen-binding fragment binds to isoform 1 of CD97 at a dissociation constant (K_{D}) about 1×10⁻⁷ M or less. In some embodiments, the antibody or antigen-binding fragment binds to isoform 1 of CD97 at a K_{D} of about 5×10⁻⁸ M or less. In some embodiments, the antibody or antigen-binding fragment binds to isoform 1 of CD97 at a K_{D} of about 1×10⁻⁸ M or less. In some embodiments, the antibody or antigen-binding fragment binds to isoform 1 of CD97 at a K_{D} of about 5×10⁻⁹ M or less. In some embodiments, the antibody or antigen-binding fragment binds to isoform 1 of CD97 at a dissociation constant (K_{D}) about 9.3×10⁻⁹ M.

In some embodiments, the antibody or antigen-binding fragment binds to isoform 2 of CD97 at a K_{D} about 1×10⁻⁷ M or less. In some embodiments, the antibody or antigen-binding fragment binds to isoform 2 of CD97 at a K_{D} of about 5×10⁻⁹ M or less.

In some embodiments, the K_{D} is measured using a bead-based binding assay or a cell-based assay.

In some embodiments, the antibody or antigen-binding fragment does not bind to EGF-like module-containing mucin-like hormone receptor-like 2 (EMR2). In some embodiments, the antibody or antigen-binding fragment does not bind to either isoform 1 or isoform 4 of EMR2.

In some embodiments, the antibody or antigen-binding fragment is recombinant.

In some embodiments, the antibody or antigen-binding fragment is a human antibody, a humanized antibody, a chimeric antibody, a murine antibody, a monoclonal antibody, a single chain antibody, a bispecific antibody or antigen-binding fragment thereof (e.g., bispecific T-cell engager (BiTE) or bispecific NK-cell engager), a Fab, a Fab′, a F(ab′)2, a Fv, a scFv, or a nanobody. In one embodiment, the antigen-binding fragment is a Fab.

In some embodiments, the antibody or antigen-binding fragment is an IgG antibody. In some embodiments, the antibody or antigen-binding fragment is of IgG1, IgG2, IgG3, or IgG4 subclass. In one embodiment, the antibody or antigen-binding fragment is of IgG1 subclass.

In some embodiments, the antibody or antigen-binding fragment comprises L234A, L235A, and/or P329G mutations in the IgG1 Fc region.

In some embodiments, the antibody or antigen-binding fragment further comprises a S239C mutation in the IgG1 Fc region.

In another aspect, provided herein is an antibody-drug conjugate comprising an anti-CD97 antibody or antigen-binding fragment described herein, conjugated to a heterologous moiety. In some embodiments, the heterologous moiety is a cytotoxic agent, an siRNA, a radionucleotide, a peptide, or an immune-stimulatory agent.

In some embodiments, the cytotoxic agent is a tubulin polymerization inhibitor, a DNA alkylating agent, a DNA cross-linking agent, a DNA cleaving agent, a topoisomerase I inhibitor, or a topoisomerase II inhibitor. In some embodiments, the tubulin polymerization inhibitor is auristatin E, auristatin F, monomethyl Auristatin E (MMAE), monomethyl auristatin F (MMAF), monomethyl auristatin D (MMAD), mertansine/emtansine (DM1), avtansine/soravtansine (DM4), dimethylvaline-valine-dolaisoleuine-dolaproine-phenylalanine-p-phenylenediamine (AFP), 5-benzoylvaleric acid-auristatin E ester (AEVB), AEB, a maytansinoid, or ansamitocin. In some embodiments, the DNA alkylating agent is duocarmycin. In some embodiments, the DNA cross-linking agent is pyrrolobenzodiazepines (PBD) dimer SG3199. In some embodiments, the DNA cleaving agent is calicheamicin. In some embodiments, the topoisomerase I inhibitor is SN-38, or DXd.

In another aspect, provided herein is a method for making an antibody-drug conjugate described herein. The method may comprise (a) contacting the antibody or antigen-binding fragment with the heterologous moiety under the conditions favorable for conjugation of the antibody or antigen-binding fragment to the heterologous moiety; and (b) optionally, isolating the antibody-drug conjugate produced in step (a).

In another aspect, provided herein is a polynucleotide encoding the antibody or antigen-binding fragment described herein.

In another aspect, provided herein is a vector comprising the polynucleotide described herein.

In another aspect, provided herein is a host cell expressing the antibody or antigen-binding fragment comprising the polynucleotide or the vector described herein. In some embodiments, the cell is a hybridoma. In some embodiments, the antibody or antigen-binding fragment is recombinantly produced.

In another aspect, provided herein is a method of producing the antibody or antigen-binding fragment, wherein said method comprises culturing the host cell described herein, and isolating said antibody or antigen-binding fragment.

In another aspect, provided herein is a pharmaceutical composition comprising the antibody or antigen-binding fragment, the antibody-drug conjugate, the polynucleotide, or the vector described herein, and a pharmaceutically acceptable carrier or diluent.

In another aspect, provided herein is a kit comprising (i) the antibody or antigen-binding fragment, the antibody-drug conjugate, the polynucleotide, the vector, or the pharmaceutical composition described herein, and (ii) packaging for the same.

In another aspect, provided herein is a method of killing a cell expressing at least an isoform of CD97, comprising contacting the cell with an effective amount of the antibody or antigen-binding fragment, the antibody-drug conjugate, the polynucleotide, the vector, or the pharmaceutical composition described herein. In some embodiments, the cell is a cancer cell.

In another aspect, provided herein is a method of treating or preventing a cancer associated with at least an isoform of CD97 in a subject in need thereof, comprising administering to the subject an effective amount of the antibody or antigen-binding fragment, the antibody-drug conjugate, the polynucleotide, the vector, or the pharmaceutical composition described herein. In some embodiments, the cancer is a hematologic cancer or solid cancer.

In some embodiments, the hematologic cancer is leukemia. In some embodiments, the leukemia is acute myeloid leukemia (AML), acute lymphoblastic leukemia (ALL), acute leukemia of ambiguous lineage, chronic myeloid neoplasm, non-Hodgkin lymphoma, Hodgkin lymphoma, chronic leukemia, dendritic/histiocytic neoplasm, or lymphoproliferative disorder. In some embodiments, the chronic myeloid neoplasm is myeloproliferative neoplasm or myelodysplastic neoplasm. In some embodiments, the chronic leukemia is myeloid chronic leukemia or lymphoid chronic leukemia.

In some embodiments, the solid cancer is glioma, thyroid cancer, lung cancer, colorectal cancer, head and neck cancer (e.g., head and neck squamous cell carcinoma), gastric cancer, stomach cancer, liver cancer, pancreatic cancer, renal cancer, urothelial cancer, prostate cancer, testis cancer, breast cancer, cervical cancer, endometrial cancer, ovarian cancer, gallbladder cancer, sarcoma (e.g., fibrosarcoma), or melanoma. In some embodiments, the glioma is glioblastoma (GBM).

In another aspect, provided herein is a method of treating or preventing glioma in a subject in need thereof, comprising administering to the subject an effective amount of a CD97 inhibitor. In some embodiments, the CD97 inhibitor is an antibody or an antigen-binding fragment, or a small molecule, or a combination thereof. In some embodiments, the CD97 inhibitor is the antibody or antigen-binding fragment, the antibody-drug conjugate, the polynucleotide, the vector, or the pharmaceutical composition described herein. In some embodiments, the glioma is glioblastoma (GBM).

In some embodiments, the treatment method described herein further comprises administering one or more additional therapeutic agents. In some embodiments, the one or more additional therapeutic agents comprise one or more chemotherapeutic agents, targeted therapies, stem cell transplantation radiotherapy, or a combination thereof. In some embodiments, the one or more chemotherapeutic agents comprises an anthracycline, cytarabine, all-trans-retinoic acid, arsenic trioxide, a hypomethylating agent, a BH-3 mimetic, temozolamide, hydroxyurea, 6-thioguanine, cyclophosphamide, gemtuzumab ozogamicin, midostaurin, ivosidenib, enasidenib, gilteritinib, glasdegib, quizartinib, olutasidenib, vincristine, or a combination thereof. In some embodiments, the anthracycline comprises daunorubicin and/or idarubicin. In some embodiments, the hypomethylating agent comprises azacytidine, decitabine, and/or guadecitabine.

In various embodiments of the treatment methods described herein, the subject is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic representation of the extracellular regions of CD97 and its close homolog, EMR2.

FIG. 2 shows binding analysis of anti-CD97 Fabs using purified target proteins. Binding titration of the Fab clones isolated from the first antibody discovery campaign. The K_{D} values are from the curve fitting of a 1:1 binding model. The errors shown are the standard deviation (s.d.) from the curve fitting. Data shown here are from triplicate measurements.

FIG. 3 shows epitope binning of anti-CD97 antibodies from the first discovery campaign assessed by the competitive phage ELISA. The binding of phage clones in the presence of the indicated Fab competitor is shown. A reduction in the binding signal indicates that the epitopes of a phage-displayed clone and of the Fab competitor overlap. The epitope bins are classified based on the competitive binding profile. The data shown here are triplicate.

FIG. 4 shows binding titration of the Fab clones isolated from the second antibody discovery campaign. Data are presented in the same manner as in FIG. 2.

FIGS. 5A-5B shows epitope binning of anti-CD97 antibodies from the second discovery campaign assessed by the competitive phage ELISA. The data are presented in the same manner as in FIG. 3. Data shown here are from singlicate (FIG. 5A) and triplicate (FIG. 5B) measurements.

FIG. 6 shows binding titration of anti-CD97 antibodies in the IgG1 harboring the silent Fc (LALA-PG) format to OCI-AML3 cells that express CD97. The K_{D} values are from the curve fitting of a 1:1 binding model, and the errors shown are the s.d. from the curve fitting.

FIGS. 7A-7B show epitope mapping of anti-CD97 antibodies using ExpiCHO cells expressing truncation mutants of CD97. (FIG. 7A) Staining of engineered cells with the indicated anti-CD97 antibodies is shown. The IgG concentrations used are indicated in the parentheses. Data shown here are from triplicate measurements. (FIG. 7B) Cartoon representations of the truncation mutants of CD97 and a summary of the epitopes. “+” denotes binding, and “−” denotes no binding.

FIGS. 8A-8C show data of antibody internalization assay. (FIG. 8A) The internalization assay of anti-CD97 IgG1 LALA-PG antibodies using OCI-AML3 cells. The cells were incubated with pHAb dye-conjugated antibodies at a concentration of 500 nM for 3 days. A8 showed a substantial internalization efficacy compared with the other clones. (FIG. 8B) The comparison of the internalization efficacy of A8-pHAb conjugate in the IgG1 LALA-PG and the Fab formats. The antibody concentration was 200 nM, which was sufficiently high to engage CD97 in both monovalent (Fab) and bivalent (IgG) formats of the antibody. The median fluorescence intensity was normalized to that obtained from the isotype antibody. (FIG. 8C) The dependence of A8 internalization on the CD97 expression level. The top panel shows the CD97 expression on each cell assessed by cell staining with A8 IgG1 LALA-PG. The bottom panel shows the internalization of A8 and isotype in the IgG1 LALA-PG format. The cells were incubated with pHAb dye-conjugated antibodies at a concentration of 200 nM for 24 hours.

FIGS. 9A-9B show cell viability assay data of A8-MMAF using AML cells. (FIG. 9A) Dose-response curve of A8-MMAF and isotype-MMAF against OCI-AML3 cells. Antibodies without drug conjugation were included as negative controls. A8-MMAF showed a potent cell-killing effect on OCI-AML3 cells. (FIG. 9B) The cell killing specificity of A8-MMAF. A8-MMAF inhibited cell growth only for the cells expressing a high level of CD97. Data shown here are from triplicate measurements.

FIGS. 10A-10P show that CD97 (ADGRE5) is de novo expressed in GBM. (FIG. 10A) Heatmap showing log 2 expression levels of aGPCRs and known neural markers in normal brain tissue and GBM [log₂(CPM)]. ADGRE5 is indicated by an arrow. (FIG. 10B) Heatmap showing protein expression levels of aGPCRs and known neural markers in normal brain tissue and in GBM. ADGRE5 is indicated by an arrow. “Not detected” indicates expression below the level of mass spectrometry sensitivity. Cut-off parameters for “Low”, “Medium”, and “High” expression are as previously described. (FIG. 10C) Volcano plot showing differentially expressed genes that are upregulated in a PDGC compared to human NSCs. All aGPCRs are labeled. Only ADGRE5 (circled) was significantly upregulated (p_{adj}<0.00001, log 2(fold change)>1.5). (FIG. 10D) Transcripts from bulk RNA-seq data collected from two PDGC replicates and two NSC replicates were aligned along the ADGRE5 locus. The red arrow signifies the first ADGRE5 exon. (FIG. 10E) Images of immunofluorescent and Hoechst 33342-stained paraffin-embedded specimen of human temporal lobe (normal) and GBM. MAP2A is a neuronal marker. An antibody against the CD97 extracellular domain (ECD) was used for CD97 staining. (FIG. 10F) Confocal microscopy images of a GBM specimen stained with an antibody against the CD97 ECD (red) and DAPI (blue). A merged color image is accompanied by black and white single-channel images. (FIG. 10G) CD97 surface staining via flow cytometry using an APC-conjugated antibody in proneural (n=6), classical (n=6), and mesenchymal (n=4) PDGCs, NSCs (n=6), and NHAs (n=4) (unpaired t-test; ****, p<0.0001). (FIG. 10H) Single-cell RNA/ATAC-seq data from IDH-mutant astrocytoma and IDH-wildtype GBM surgical specimens. Single cells are color-coded based on their tumor of origin. (FIG. 10I) Cluster identities were characterized and named based on their corresponding gene expression profiles. Single cells are colored based on broad cluster identities. (FIG. 10J) ADGRE5 expression in cell clusters based on integrated data. (FIG. 10K) ATAC peaks associated with the ADGRE5 locus were extracted from single-cell ATAC-seq and integrated based on the clusters in FIG. 10I. The red arrow signifies the ADGRE5 promoter. Also shown are peaks corresponding to the DDX39A promoter, which is expressed in all cell clusters. (FIG. 10L) H3K27ac ChIP-seq from the GEO shows peaks associated with the ADGRE5 locus for IDH-wildtype GBM and IDH-mutant anaplastic astrocytoma. The red arrow signifies the ADGRE5 promoter. (FIG. 10M) Schematic representation of the structure of three CD97 isoforms. EGF, epidermal growth factor-like repeat; RGD, arginylglycylaspartic acid domain; GAIN, GPCR autoproteolysis inducing domain; GPS, GPCR proteolysis site. Note that for isoforms 2 and 3, only the NTF is shown. (FIG. 10N) Bulk RNA-seq data from the GBM dataset of TCGA were used to quantify CD97 isoform expression. Blue boxes are centered around the median and encompass the two middle quartiles. (FIG. 10O) Schematic representation of the tet-inducible vector used for CD97 isoform overexpression. (FIG. 10P) Immunoblots for the CD97 ICD (equivalent to the CTF) and the ECD (equivalent to NTF). CD97 S531A is an uncleavable point mutant of isoform 1. Non-specific bands are indicated by a #sign. The same membrane was stained for CD97 ICD and then stripped for CD97 ECD staining. Error bars throughout this figure indicate SEM.

FIGS. 11A-11K show that CD97 is essential for tumor initiation in vitro and in vivo. (FIG. 11A) Schematic representations of the shRNA and CRISPR constructs used to knock down and knock out CD97, respectively. (FIG. 11B) Histograms of CD97 surface staining following knockdown or overexpression of isoform 3 of CD97 in a PDGC. (FIG. 11C) Bar graph visualizing tumorsphere formation following knockdown of CD97 and followed by rescue with a dox-inducible and a shRNA-resistant form of CD97 isoform 3 (n=5 for each PDGC; 2-way ANOVA F_{3,36}=9.795, p<0.0001; Tukey's multiple comparisons test: EV SCRsh vs. EV CD97sh: ***, p<0.001; EV CD97sh vs. OE CD97sh: *, p<0.01; OE SCRsh vs. OE CD97sh: ns, p>0.05.). (FIG. 11D) Representative wells from the tumorsphere formation assay quantified in FIG. 11C before and after dox-induced rescue. (FIG. 11E) ELDA plots for three PDGCs are displayed. (FIG. 11F) Summary plot showing calculated clonogenic frequencies for all tested PDGCs after CD97 knockdown based on ELDA assays. Paired samples are connected with a line. (n=3 per PDGC; 2-way ANOVA F_{1,28}=75.24, p<0.0001). (FIG. 11G) Graph showing compromised viability of PDGCs infected with three separate gRNAs against CD97 (orange) compared to a control gRNA against the human homologue of ROSA26 (black) in a cell competition assay (n=3 per gRNA; 2-way ANOVA F_{4,40}=10.37, p<0.001; Tukey's multiple comparisons test: ROSA26 vs. CD97: D4, D8, D12: ns, p>0.05; D16, D20: ***, p<0.001). (FIG. 11H) Bioluminescent images taken with IVIS of intracranial GBM xenografts 90 days after injection. (FIG. 11I) Immunofluorescent images of mouse brains injected with PDGCs lentivirally infected with the indicated shRNA construct, the TagRFP fluorophore, and luciferase. White arrows indicate the resulting tumor at the site of injection. (FIG. 11J) Graph depicting the average bioluminescent radiance captured by IVIS for mice harboring PDGC xenografts (n=9 mice per group; unpaired t-test; *, p<0.05). (FIG. 11K) Kaplan-Meier survival curve showing significantly increased survival of mice xenografted with a PDGC harboring CD97 shRNA (n=5 mice per group; logrank test; **, p<0.01). Error bars throughout this figure indicate SEM.

FIGS. 12A-12J show that CD97 knockdown impairs glycolytic metabolism in GBM. (FIG. 12A) Volcano plot derived from bulk RNA-seq data collected from PDGCs lentivirally infected with the scramble (SCR) or CD97 shRNA (n=3 biological replicates for each). Genes involved in canonical glycolysis and glucose transport are represented as large yellow points. Genes involved in the TCA cycle and OXPHOS (cytochrome oxidase subunits) are represented as large green points. ADGRE5 (CD97) is represented by an orange point. (FIG. 12B) The top ten pathways with the largest fold enrichment among downregulated genes using GO PANTHER pathway enrichment analysis. (FIG. 12C) Correlation matrix from bulk RNA-seq data collected from PDGCs following knockdown or overexpression of CD97 shows high correlation between ADGRE5 and glycolysis-related genes and anti-correlation with TCA cycle-related genes. HK2 (hexokinase 2) and SLC2A3 (glucose transporter 3/GLUT3) transcripts are both included because they have been implicated in Warburg metabolism. (FIG. 12D) Bar graph showing decreased lactate production after knockdown of CD97 (n=6 per PDGC; 2-way ANOVA F_{1,15}=27.24, p<0.001) and increased lactate production after CD97 overexpression in PDGCs (n=3 per PDGC; 2-way ANOVA F_{1,6}=26.07, p<0.01). (FIG. 12E) Steady-state metabolomic data reveals depletion of glycolytic metabolites after knockdown of CD97 in PDGCs [PN=proneural, n=2-3 (one replicate was removed for technical reasons); CL=classical, n=3]. (FIG. 12F) Representative Seahorse Cell Energy Phenotype graph showing OCR and ECAR changes before (baseline) and after (maximal) addition of mitochondrial stressors. (FIG. 12G and FIG. 12H) Bar graphs quantifying baseline and maximal ECAR (n=6 per PDGC; Baseline: 2-way ANOVA F_{1,10}=14.06; **, p<0.01; Maximal: 2-way ANOVA F_{1,10}=17.87, p<0.01). (FIG. 12I and FIG. 12J) Bar graphs quantifying baseline and maximal OCR (n=6 per PDGC; Baseline: 2-way ANOVA F_{1,10}=2.820, p>0.05; Maximal: 2-way ANOVA F_{1,10}=5.474; *, p<0.05). Error bars throughout this figure indicate SEM.

FIGS. 13A-13H show that CD97 activates the MAPK signaling pathway to regulate glycolysis and tumorsphere formation. (FIG. 13A) Immunoblot for phosphorylated ERK1/ERK2, total ERK1/ERK2, and GAPDH in PDGCs following CD97 knockdown. Red pixels indicate saturation. (FIG. 13B) Quantification of densitometry ratios in FIG. 13A (p-ERK/ERK: n=5-6 per PDGC; 2-way ANOVA F_{1,17}=8.407, p<0.01; ERK/GAPDH: n=5-6 per PDGC; 2-way ANOVA F_{1,17}=2.796, p>0.05). (FIG. 13C) Immunoblots for phosphorylated ERK1/ERK2, total ERK1/ERK2, and GAPDH in a PDGC following dox-induced overexpression of all three CD97 isoforms. (FIG. 13D) Quantification of densitometry ratios in FIG. 13C (all isoforms compiled for each PDGC) (p-ERK/ERK: n=4-8 per PDGC; 2-way ANOVA F_{1,21}=10.98, p<0.01; ERK/GAPDH: n=4-8; 2-way ANOVA F_{1,21}=1.554, p>0.05). (FIG. 13E) Seahorse cell energy phenotype graphs showing OCR and ECAR changes before (baseline) and after (maximal) addition of mitochondrial stressors. (FIG. 13F) Quantification of the difference in baseline OCR and ECAR between SCR and CD97 knockdown PDGCs after introduction of the MEK^{DD} mutant or an empty vector control [n=13 (all PDGCs compiled); unpaired t-test; ns, p>0.05; **, p<0.01]. (FIG. 13G) Bar graph visualizing tumorsphere formation following CD97 knockdown and introduction of the MEK^{DD} mutant (n=8-9 per PDGC; 2-way ANOVA F_{3,66}=8.707, p<0.0001; Tukey's multiple comparisons test: EV SCRsh vs. EV CD97sh: **, p<0.05; MEK^{DD} SCRsh vs. MEK^{DD} CD97sh: ns, p>0.05). (FIG. 13H) Representative wells from the assay in FIG. 13G. Error bars throughout this figure indicate SEM.

FIGS. 14A-14L show that CD97 activates the MAPK signaling cascade through recruitment of β-arrestin to its phosphorylated C-terminus and by association with THY1/CD90. (FIG. 14A) Schematic of possible CD97 signaling mechanisms tested. Listed in red are the methods for testing. (FIG. 14B) Phosphoproteomic data collected from GBM samples identify five phosphorylation sites on the CD97 cytosolic C-terminus. The heatmap depicts the percentage of GBM samples with the detected phosphorylation site. (FIG. 14C) HTRF ratios from a b-arrestin recruitment assay performed after overexpression of WT CD97 or the ΔPS mutant. Two PDGCs (n=2 for each) are compiled (n=4 per condition; unpaired t-test; ns, p>0.05; *, p<0.05; **, p<0.01). (FIG. 14D) Immunoblot for phosphorylated ERK1/ERK2 after overexpression of WT CD97 or the ΔPS mutant. (FIG. 14E) Bar graphs displaying densitometry ratios from FIG. 14D (n=7; paired t-test; ns, p>0.05; *, p<0.05). Two PDGCs (n=3-4 for each) are compiled. (FIG. 14F) Bar graphs depicting the number of tumorspheres in a tumorsphere formation assay after CD97 knockdown followed by overexpression of shRNA-resistant forms of WT CD97 or the APS mutant. The SCR shRNA groups used for normalization are not shown (n=4 per PDGC; ANOVA F_{5,12}=80.79, p<0.0001; Tukey's multiple comparisons test: EV vs. WT: ***, p<0.001; EV vs. DPS: ns, p>0.05; WT vs. DPS: *, p<0.05). (FIGS. 14G-14I) Single-cell RNA/ATAC-seq data from FIG. 10I showing expression of ADGRE5, CD55, and THY1/CD90. (FIG. 14J) A bar graph depicting the percentage of positive cells measured by flow cytometry after surface staining of PDGCs with FITC-conjugated antibodies against CD55, THY1/CD90, and an IgG control (n=3 for each PDGC; 2-way ANOVA F_{2,20}=95.11, p<0.0001; Tukey's multiple comparisons test: IgG vs. CD55: **, p<0.01; IgG vs. CD90: ****, p<0.00001; CD55 vs. CD90 ****, p<0.01). (FIG. 14K) Immunoblot for phosphorylated ERK1/ERK2 from CD97-overexpressing PDGCs plated on laminin or recombinant forms of putative ligands CD55 and THY1/CD90. (FIG. 14L) Quantification of densitometry ratios from immunoblots in FIG. 14K (n=2-4 per PDGC; Empty Vector p-ERK/ERK: 2-way ANOVA F_{2,6}=0.9450, p>0.05; Empty Vector ERK/GAPDH: 2-way ANOVA F₂,6=1.047, p>0.05; CD97 Overexpression p-ERK/ERK: 2-way ANOVA F_{2,16}=12.48, p<0.001; Tukey's multiple comparisons test: Laminin vs. CD55: ns, p<0.05; Laminin vs. CD90: ***, p<0.001; CD97 Overexpression ERK/GAPDH: 2-way ANOVA F_{2,16}=2.257, ns, p>0.05). Error bars throughout this figure indicate SEM.

FIGS. 15A-15E show that a CD97-targeting antibody-drug conjugate demonstrates enhanced killing of PDGCs compared to NSCs and NHAs. (FIG. 15A) Schematic of the MMAF-conjugated ADC binding to the CD97 receptor. (FIG. 15B) Dose-response curves generated from WST8 cell viability assays (n=3 biological replicates based on technical triplicates) after treatment of PDGCs, NHAs, and NSCs with the CD97 ADC. (FIG. 15C) Table displaying the LD₅₀ values (in nM) for the CD97 ADC on all four cell lines as calculated from dose-response curves in FIG. 15B. Curves were based on a nonlinear regression fit. (FIG. 15D) Representative images of Hoechst 33342-stained cells after six days of treatment with different concentrations of the CD97 ADC. (FIG. 15E) Quantification of Hoechst intensity in FIG. 14D. Values are normalized to the 0 nM condition. NHAs and NSCs are compiled (non-GBM; black) and the two GBM lines are compiled (GBM; red) to make differences more evident (n=1-2 for each cell line; 2-way ANOVA F_{2,8}=12.13, p<0.01; Tukey's multiple comparisons test: Non-GBM: 0 nM vs. 1.2 nM: ns, p>0.05; 0 nM vs 6 nM: *, p<0.05; GBM: 0 nM vs 1.2 nM: *, p<0.05; 0 nM vs. 6 nM: *, p<0.05). Error bars throughout this figure indicate SEM.

FIG. 16 shows a schematic outlining the proposed mechanism of action of CD97 in GBM. The interaction of CD97 with ligand CD90 leads to phosphorylation of the CD97 intracellular C-terminus and recruitment of β-arrestin. This, in turn, activates the MAPK pathway, which promotes Warburg metabolism, leading to increased proliferation, tumor initiation and migration in GBM cells.

FIGS. 17A-17E show additional expression data. (FIG. 17A) Representative histogram data from FIG. 10C, showing CD97:APC staining versus an IgG:APC control. (FIG. 17B) Dot plot displaying gene expression profiles of the single-cell clusters from FIG. 10I. The size of the circles corresponds to the percentage of cells within the cluster that express the gene. The color corresponds to the average gene expression in those cells compared to the population as a whole. (FIG. 17C) Publicly available bulk RNA-seq data from TCGA were used to investigate CD97 expression in GBM from all TCGA-defined transcriptional subtypes (classical, mesenchymal, and proneural). The neural GBM subtype is outdated and likely includes mixed samples of the other three subtypes and normal brain tissue. (FIG. 17D) Table displaying CD97 isoform (SV) 1-3 full-length and fragment sizes. (FIG. 17E) Immunoblots showing glycosylated and deglycosylated bands for the CD97 ICD (equivalent to the CTF) and the ECD (equivalent to NTF). CD97 S531A is an uncleavable point mutant of isoform 1. Endogenous CD97 expression in the PDGC is also shown (indicated by the black arrow). Lanes have been rearranged for clarity. Non-specific bands are indicated by a # sign.

FIGS. 18A-18G show that CD97 ablation impacts PDGC proliferation and viability. (FIG. 18A) Schematic of the ADGRE5 gene showing exon targeting of the shRNA and gRNA constructs used in this paper. Corresponding domains are shown beneath the gene. Note gRNA2 targets an EGF domain not found in CD97 isoform 2. (FIG. 18B) Histogram showing CD97 surface expression in PDGCs infected with the gRNA construct (mCherry+) and uninfected PDGCs (mCherry−) 20 days post-infection (dpi). Reduced CD97 surface expression is specific to PDGCs infected with a gRNA targeting the ADGRE5 gene and is not observed in those cells infected with a control gRNA targeting the human homologue of the ROSA26 locus. (FIG. 18C) Fluorescent images of DAPI-stained PDGCs in the upper (total) and lower (migrated) chamber of a transwell assay following lentiviral infection with a shRNA against CD97 or a SCR control. A bar graph quantifies the ratio of cells within the lower chamber to cells within the upper chamber [n=6 (two PDGCs compiled); paired t-test; *, p<0.05]. (FIG. 18D) A bar graph quantifying the number of tumorspheres in a tumorsphere formation assay after doxycycline-induced CD97 overexpression of isoforms 1-3. The graph is normalized to non-induced cells (n=3-4 per isoform; 2-way ANOVA F_{1,18}=1.125; ns, p>0.05). (FIG. 18E) Bar graphs quantifying the percentage of PDGCs or astrocytes within the S/G2 phase of the cell cycle following CD97 knockdown as measured by Hoechst 33342 staining. Graphs are normalized to a SCR shRNA control. Representative histograms are displayed as well. An arrow indicates the peak with cells in the S/G2 phase of the cell cycle. (GBM: n=2-3 per PDGC; 2-way ANOVA F_{1,13}=5.282, p<0.05; NHAs: n=3 unpaired t-test; ns, p>0.05). (FIG. 18F) Immunofluorescent images of Ki67-stained PDGCs following CD97 knockdown. The percentage of Ki67+ cells are quantified in the bar graph, which is normalized to the SCR shRNA control. (n=2-3 per PDGC; 2-way ANOVA F_{1,12}=47.47, p<0.0001). (FIG. 18G) The percentage of PDGCs or astrocytes that are either Annexin-V+ and/or DAPI+ after CD97 knockdown are quantified in the bar graphs, which are normalized to the SCR control. Representative dot plots are shown with quadrants labeling cells considered viable and non-viable. (GBM: n=3-4 per PDGC; 2-way ANOVA F_{1,22}=5.829, p<0.05; NHAs: n=3; unpaired t-test; ns, p>0.05). Error bars throughout this figure indicate SEM.

FIGS. 19A-19E show additional metabolic data. (FIG. 19A) Immunoblot staining against GLUT3, GAPDH, and CD97 of whole cell lysates collected from PDGCs following lentiviral transduction with a SCR shRNA, CD97 shRNA, or tet-inducible CD97 overexpression construct in the absence or presence of doxycycline. (FIG. 19B) GSEA was used to identify pathway enrichment of transcriptomic data from single replicates of three separate PDGCs following CD97 knockdown. Some of the top pathways enriched after knockdown included pathways related to cellular respiration (red). Some of the depleted pathways after CD97 knockdown included glycolytic pathways (blue). Pathways are ranked based on their normalized enrichment score (NES). (FIG. 19C) Representative GSEA plots of pathways from FIG. 19B generated using GSEA software. (FIG. 19D) A heatmap quantifying levels of heavy-carbon intermediates of glycolysis and the TCA-cycle after PDGCs were exposed to heavy-carbon glucose. The flux metabolomic assay includes three biological replicates from the same PDGC following CD97 (or SCR) knockdown. The number of heavy-carbons are listed following the metabolite. All glycolytic metabolite data was generated using PDGCs after 30 minutes of exposure. All TCA cycle metabolite data was generated using PDGCs after 120 minutes of exposure and are organized into passes based on the theoretical number of heavy-carbons incorporated from the heavy-carbon glucose. (FIG. 19E) A heatmap quantifying steady-state levels of metabolites relevant for PPP following CD97 knockdown.

FIGS. 20A-20H show additional signaling data. (FIG. 20A) An immunoblot demonstrating increased levels of ERK1/ERK2 phosphorylation following stable transfection of PDGCs with the MEK^{DD} construct. The bar graph quantifies the densitometry ratios and is normalized to the empty vector control (n=3; paired t-test; **, p<0.01). (FIG. 20B) Summary statistics for luminescent signal after PDGCs were induced for overexpression of CD97 or an empty vector control and transfected with luciferase reporters for G protein-coupled pathways (CRE, Gαₛ; SRE, Gβγ; SRF-RE, Gα_{12/13}; NFAT, Gα_{q}) (n=2-8; unpaired t-tests; ns, p>0.05). Data is normalized to uninduced controls. (FIG. 20C) Pull-down assay for GTP-bound RHOA after overexpression of WT CD97 or an EV control. A positive (+) and negative (−) control were included as described by the manufacturer. Lanes are rearranged for clarity. (FIG. 20D) Immunoblot for AKT and ERK1/ERK2 and their phosphorylated counterparts after overexpression of CD97 or an empty vector control along with the corresponding densitometry ratios displayed in the bar graph. (FIG. 20E) A heatmap showing detection of the five novel phosphorylation sites in the CPTAC dataset collected from 99 GBM samples. Values are based on a global-centered normalization. White boxes indicate no detection in that sample. (FIG. 20F) Correlation matrix summarizing the correlation between the five novel phosphorylation sites identified from the publicly available CPTAC phosphoproteomic data collected from 99 GBM samples. (FIG. 20G) Sanger sequencing result confirming removal of the DPS sequence shown in FIG. 14B. Figure discloses SEQ ID NO: 116, 117, 116, and 117-119. (FIG. 20H) Immunofluorescent staining against the CD97 N-terminus shows proper membrane localization of WT CD97 and the DPS mutant in HEK293 cells. No signal is observed upon transduction with an empty vector control. Error bars throughout the figure indicate SEM.

FIGS. 21A-21G show additional data on putative ligands. Publicly available single-cell RNA-seq data of adult and pediatric GBM from the Broad Institute Single-Cell Portal (GSE131928) were used (Neftel C. et al., An Integrative Model of Cellular States, Plasticity, and Genetics for Glioblastoma. Cell 178, 835-849.e821, doi:10.1016/j.cell.2019.06.024 (2019)). (FIG. 21A) Clusters, identifiers, and the number of cells are shown. (FIG. 21B) Transcript levels of ADGRE5 are shown. (FIG. 21C and FIG. 21D) Transcript levels of putative CD97 ligands: CD55 (FIG. 21C) and THY1/CD90 (FIG. 21D) are shown. Integrated single-cell data from IDH-wildtype GBM and IDH-mutant astrocytoma (FIG. 10I) also show ADGRE5 (FIG. 21E), CD55 (FIG. 21F), and THY1/CD90 ((FIG. 21G) expression.

FIGS. 22A-22F show additional dose-response curve data. (FIG. 22A) The human anti-CD97 antibody was conjugated to a pH sensitive dye (pHAb) and internalization was quantified via flow cytometry. A non-specific IgG isotype control was included (n=3; paired t-test; ***, p<0.001; ****, p<0.0001). (FIGS. 22B-22E) Individual dose-response curves based on WST8 viability assays in (FIG. 22B) NHAs, (FIG. 22C) proneural GBM, (FIG. 22D) NSCs, and (FIG. 22E) classical GBM, after treatment with the CD97 ADC, MMAF (impermeant cytotoxic drug) alone, or MMAE (membrane-permeant cytotoxic drug) alone (n=3 biological replicates each based on three technical triplicates). Curves were based on a nonlinear regression fit. (FIG. 22F) A table displaying the LD₅₀ values (in nM) for the CD97 ADC, MMAF, and MMAE on all four cell lines as calculated from dose-response curves in FIGS. 22B-22E. Curves were based on a nonlinear regression fit. Error bars throughout this figure indicate SEM.

FIG. 23 shows expression of CD97 RNA in various cancers based on Cancer Genome Atlas (TCGA) datasets.

FIGS. 24A-24C show that CD97 isoforms exhibit differences in supporting clonogenicity and proliferation in human AML. (FIG. 24A) Overexpression of the CD97 isoforms in HL-60 cells results in differences in clonogenicity during serial plating, with CD97 isoform 1 preferentially promoting serial replating and CD97 isoform 2 associated with increased clonogenicity in primary platings. (FIG. 24B) In contrast to CD97 isoform 1 overexpression, CRISPR-Cas9 mediated deletion of CD97 isoform 1 is associated with reduced clonogenicity. (FIG. 24C) Overexpression of a non-cleavable version of CD97 isoform 1 (Stachel mutant) enhances clonogenicity of HL-60 cells similar to CD97 isoform 1 in serial methycellulose replating assays.

FIGS. 25A-25D show that CD97 isoforms exhibit differences in supporting clonogenicity and proliferation in human AML. (FIG. 25A) Overexpression of CD97 isoform 2 in WT HL-60 is unique in its ability to promote growth. (FIG. 25B) Complementation of CD97 KO cells with each CD97 isoform partially rescues cell growth in liquid cultures, but the effect appears to be the largest with CD97 isoform 2. (FIG. 25C) In transwell migration assays, CD97 isoform 3 overexpression preferentially promotes AML cell migration. (FIG. 25D) Evaluation of CD97 cell surface expression in cells that migrated versus that did not reveals highest levels of surface CD97 in cells overexpressing CD97 isoform 3.

FIGS. 26A-26B show that overexpression of CD97 isoforms in human AML cells results in differences in disease aggressiveness in xenografts. (FIG. 26A) Flow cytometry analysis reveals similar levels of CD97 isoform overexpression in HL-60 cells xenografted into NSG mice. (FIG. 26B) Transplantation of 10⁶ HL-60 cells overexpressing each CD97 isoform results in differences in OS.

FIGS. 27A-27F depict the cryo-electron microscopy (EM) structure of A8 Fab in complex with CD97 isoform 1 and a mutation study of interface residues. (FIG. 27A and FIG. 27B) Cryo-EM structure of A8 bound to CD97 isoform 1 harboring an S531A mutation that prevents autoproteolysis. Only the variable domains of A8 and the GAIN domain of CD97 are shown for clarity. Each polypeptide and the stachel peptide segment are indicated (FIG. 27A), and the CDRs of A8 are labeled (HCDR1-3 correspond to H1-3 and LCDR1-3 correspond to L1-3) (FIG. 27B). (FIG. 27C) Close-up views of the interfaces between the A8 VH and CD97 (left) and between the A8 VL and CD97 (right). The side chains of key residues are shown and labeled. Polar interactions are marked as dashed lines. (FIG. 27D and FIG. 27E) Binding titration curves of A8 mutants against CD97 isoform 1 in a bead-based binding assay, performed in duplicate (FIG. 27D), and of A8 against CD97 GAIN mutants in a cell-based binding assay (FIG. 27E). Biotinylated CD97 isoform 1 was immobilized on beads, and A8 and its mutants in the Fab format were titrated into a solution containing beads. (FIG. 27E) CD97 GAIN mutants were transiently expressed on the surface of ExpiCHO cells, and A8 Fab was titrated into a solution containing the cells, and the bound Fab was detected using flow cytometry. The K_{D} values are from the curve fitting of a 1:1 binding model. The errors shown are the s.d. from the curve fitting. Data shown are from triplicate measurements. Mutations of residues in the binding interface observed in the structure reduced or abolished the binding of A8 to CD97, validating the cryo-EM structure of the A8-CD97 complex. (FIG. 27F) A summary of the mutation study. The A8 binding interface is outlined on the CD97 surface, and the residues mutated to Ala (left) and to Lys (right) are indicated. The darker color indicates the importance of the residues for binding (left). The side chains of key residues in HCDR3 (H3) in A8 are also displayed as a stick model (left).

FIGS. 28A-28E show optimization of ADCs and analysis of their cytotoxic effect. (FIG. 28A) Schematic representation of A8 ADCs prepared with the cysteine-based random (left) and site-specific (right) conjugation methods. The Fc region in both constructs contained L234A/L235A/P329G (LALA-PG) mutations to abolish binding to the Fc receptors. The S239C mutation was introduced for site-specific conjugation (right). (FIG. 28B) Summary of thermal stability, drug-to-antibody ratio (DAR), and conjugation method of ADCs. Data represent mean±s.d. from triplicate measurements, except that DAR for hIgG_{S239c}-MMAF is from a single measurement. The ADCs prepared with the site-specific conjugation method showed improved stability compared to those prepared with the cysteine-based random conjugation method. (FIG. 28C and FIG. 28D) Dose-response of ADCs using OCI-AML3 cells. The effect of different payloads (FIG. 28C) and antibodies (FIG. 28D) were tested, respectively. Motavizumab (mota), an anti-respiratory syncytial virus (RSV) antibody, was used as a negative control antibody. The cells were incubated with the indicated ADCs or unconjugated A8 for 4 days, and cell death was measured with Annexin V staining. The IC₅₀ values are shown. A8_{S293C}-PBD showed a potent cell-killing effect. (FIG. 28E) Specificity and toxicity assessment of A8_{S293c}-PBD. The indicated cell lines and primary cells were incubated with A8_{S293C}-PBD or unconjugated A8 for 4 days. The fold changes in cell death compared to PBS control are shown. A8_{S293C}-PBD specifically killed cells expressing high levels of CD97 and did not kill PBMC cells, indicating specific killing and suggesting low toxicity. Data shown here are from triplicate measurements. The error shows the s.d..

### DETAILED DESCRIPTION

The present disclosure describes, among other things, identification of novel mechanisms contributing to GBM tumorigenesis. In certain aspects, such novel mechanisms can involve adhesion G protein-coupled receptors (aGPCRs), which consists of 33 members.⁶ aGPCRs are characterized by large extracellular N-termini that contain both receptor-specific domains determining ligand binding and a functionally conserved GPCR autoproteolysis inducing (GAIN) domain, which catalyzes receptor cleavage at the GPCR proteolysis site (GPS).⁷⁻⁹ aGPCRs have been implicated in developmental, physiologic, and oncogenic processes.^{6,10-15} A comparison of the expression of all aGPCR members in cell types within healthy, non-neoplastic brain tissue; neural stem cells (NSCs), the putative cell-of-origin in GBM¹⁶⁻¹⁸; and patient-derived GBM cultures (PDGCs), is described herein. Transcriptomic and proteomic expression analysis of the present disclosure identified CD97 (ADGRE5) as the aGPCR with the largest differential expression profile: high expression in PDGCs derived from all transcriptional subtypes of GBM in The Cancer Genome Atlas (TCGA; proneural, classical, and mesenchymal)^{2,19}, and absence from normal brain tissue and NSCs.

CD97 is expressed in several lineages of the immune system, where it is critical for the inflammatory response,²⁰⁻²⁴ as well as in multiple liquid and solid malignancies.^{13,25-28} Among these malignancies is GBM, in which CD97 was previously implicated in cellular proliferation, brain invasion, and tumor metabolism.²⁹⁻³³ However, its mechanism of action in GBM remains incompletely understood. Furthermore, little research effort has been devoted to its therapeutic targeting. PDGCs described herein were used to demonstrate that CD97 is essential for tumor growth both in vitro and in vivo. Using transcriptomic, metabolomic, and signaling assays, the present disclosure describes the unexpected finding that CD97 helps promote glycolytic metabolism via activation of the mitogen-activated protein kinase (MAPK) signaling pathway. THY1/CD90 is also identified herein as the most likely physiologically relevant CD97 ligand in GBM and further, it is demonstrated herein that CD97 signaling depends on phosphorylation of its C-terminus and recruitment of β-arrestin. To capitalize on CD97's therapeutic potential, a CD97 antibody-drug conjugate (ADC) developed in accordance with the present disclosure, kills GBM cells, but not human astrocytes or NSCs in vitro. Collectively, these data elucidate novel receptor activation and signaling mechanisms employed by CD97 to promote tumor growth and regulate tumor metabolism in GBM, and highlight the receptor's potential as a therapeutically targetable vulnerability in GBM.

### Definitions

The term “antibody” refers to an immunoglobulin molecule capable of specific binding to a target, such as a carbohydrate, polynucleotide, lipid, polypeptide, etc., through at least one antigen recognition site, located in the variable region(s) of the immunoglobulin molecule. As used herein, the term “antibody”, e.g., anti-CD97 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-CD97 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (VL), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the “IMGT” numbering scheme, the “AHo” numbering scheme, and/or the contact definition, all of which are well known in the art. (See, e.g., Kabat, E. A., et al. (1991) Sequences of Proteins of Immunological Interest, Fifth Edition, U.S. Department of Health and Human Services, NIH Publication No, 91-3242, Chothia et al., (1989) Nature 342:877; Chothia, C. et al. (1987) J. Mol. Biol.196:901-917, Al-lazikani et al (1997) J. Molec. Biol.273:927-948; Edelman et al., Proc Natl Acad Sci USA. 1969 May; 63(1):78-85; and Almagro, J. Mol. Recognit.17:132-143 (2004); MacCallum et al., J. Mol, Biol.262:732-745 (1996), Lefranc M P et al., Dev Comp Immunol, 2003 January; 27(1):55-77; and Honegger A and Pluckthun A, J Mol Biol, 2001 Jun.8; 309(3):657-70. See also hgmp,mrc.ac.uk and bioinf.org.uklabs).

In some embodiments, the anti-CD97 antibody described herein is a full-length antibody, which contains two heavy chains and two light chains, each including a variable domain and a constant domain. Alternatively, the anti-CD97 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, CL and CH1 domains; (ii) a F(ab′)₂ fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H}, domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (se Fv). See e.g., Bird et al. (1988) Science 242:423-426; and Huston et al. (1988) Proc. Natl. Acad. Sci. USA 85:5879-5883.

Any of the antibodies described herein, e.g., anti-CD97 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

As used herein “specifically binds”, “specific binding”, “specifically recognizes” or “specifically recognition” refers to the ability of the antibodies or antigen-binding fragments of the disclosure to bind to a predetermined antigen (e.g., CD97) with a dissociation constant (K_{D}) of about 1×10⁻⁶ M or less, for example about 1×10⁻⁷ M or less, about 1×10⁻⁸ M or less, about 1×10⁻⁹ M or less, about 1×10−¹⁰ M or less, about 1×10⁻¹¹ M or less, about 1×10⁻¹² M or less, or about 1×10⁻¹³ M or less. Typically, the antibody or antigen-binding fragment binds to an antigen (e.g., CD97) with a K_{D} that is at least ten-fold less than its K_{D} for a nonspecific antigen (for example BSA or casein) as measured by e.g., surface plasmon resonance using for example a Proteon Instrument (BioRad).

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids in the polynucleotides that encode for the antibody or antigen-binding fragment described herein. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to an intended antigen (e.g., CD97) is substantially free of antibodies that specifically bind antigens other than the intended antigen.

The term “polynucleotide” as referred to herein means a polymeric form of nucleotides of at least 10 bases in length, either ribonucleotides or deoxyribonucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin or source of derivation, the “isolated polynucleotide” has one to three of the following: (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a vehicle capable of transporting a nucleic acid into a host cell. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “promoter” as used herein is defined as a DNA sequence recognized by the synthetic machinery of the cell, or introduced synthetic machinery, required to initiate the specific transcription of a polynucleotide sequence. As used herein, the term “regulatory sequence” means a nucleic acid sequence which can regulate expression of a gene product operably linked to the regulatory sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in a tissue specific manner.

The term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “percent sequence identity” means a ratio, expressed as a percent of the number of identical residues over the total number of residues compared.

Sequence identity for nucleic acid sequences may be analyzed over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters, as specified with the programs, to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters, see GCG Version 6.1. (University of Wisconsin Wis.) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J. Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, as supplied with the programs, share at least 70%, 75%, 80% or 85% sequence identity, preferably at least 90%, 91%, 92%, 93%, 94% 95%, 96%, 97%, 98% or 99% sequence identity. In certain embodiments, residue positions that are not identical differ by conservative amino acid substitutions.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative substitution or replacement, as the terms are used interchangeably herein, is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

The term “cytotoxic agent” refers to a compound that can cause harm, disturbances, or death to a cell.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “therapeutically effective” applied to dose or amount refers to that quantity of a compound or pharmaceutical composition that is sufficient to result in a desired activity upon administration to an animal in need thereof. Within the context of the present invention, the term “therapeutically effective” refers to that quantity of a compound or pharmaceutical composition that is sufficient to reduce or eliminate at least one symptom of a cancer, inflammation, or related disorder. Note that when a combination of active ingredients is administered the effective amount of the combination may or may not include amounts of each ingredient that would have been effective if administered individually.

As used herein, the phrase “pharmaceutically acceptable” refers to molecular entities and compositions that are generally regarded as physiologically tolerable.

As used herein, the term “combination” of a compound or a composition, and at least a second pharmaceutically active ingredient means at least two, but any desired combination of compound or composition can be delivered simultaneously or sequentially.

The terms “patient”, “individual”, “subject”, and “animal” are used interchangeably herein and refer to mammals, including, without limitation, human and veterinary animals (e.g., cats, dogs, cows, horses, goats, sheep, pigs, etc.) and experimental animal models. In a preferred embodiment, the subject is a human.

As used herein, the term “healthy subject” refers to a subject that is without known infections or autoimmune disorders by using conventional diagnostic methods. In certain embodiments, a healthy subject is a subject without a known first degree relative with an autoimmune disorder. In certain embodiments, a matched healthy subject is matched by age, gender, and/or ethnicity.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

### Antibodies and Antigen-Binding Fragments

The present disclosure provides isolated antibodies, or antigen-binding fragments thereof, that specifically bind to at least one isoform of cluster of differentiation 97 (CD97), also known as adhesion G protein-coupled receptor E5 (ADGRE5). In some embodiments, antibodies, or antigen-binding fragments thereof may bind to CD97 (ADGRE5) isoform 1 which has the amino acid sequence

(SEQ ID NO: 85)
MGGRVFLAFCVWLTLPGAETQDSRGCARWCPQNSSCVNATACRCNPGFSSFSEIITTPTE
TCDDINECATPSKVSCGKFSDCWNTEGSYDCVCSPGYEPVSGAKTFKNESENTCQDVDE
CQQNPRLCKSYGTCVNTLGSYTCQCLPGFKFIPEDPKVCTDVNECTSGQNPCHSSTHCL
NNVGSYQCRCRPGWQPIPGSPNGPNNTVCEDVDECSSGQHQCDSSTVCFNTVGSYSCRC
RPGWKPRHGIPNNQKDTVCEDMTFSTWTPPPGVHSQTLSRFFDKVQDLGRDSKTSSAEV
TIQNVIKLVDELMEAPGDVEALAPPVRHLIATQLLSNLEDIMRILAKSLPKGPFTYISPSNT
ELTLMIQERGDKNVTMGQSSARMKLNWAVAAGAEDPGPAVAGILSIQNMTTLLANASL
NLHSKKQAELEEIYESSIRGVQLRRLSAVNSIFLSHNNTKELNSPILFAFSHLESSDGEAGR
DPPAKDVMPGPRQELLCAFWKSDSDRGGHWATEGCQVLGSKNGSTTCQCSHLSSFAIL
MAHYDVEDWKLTLITRVGLALSLFCLLLCILTFLLVRPIQGSRTTIHLHLCICLFVGSTIFL
AGIENEGGQVGLRCRLVAGLLHYCFLAAFCWMSLEGLELYFLVVRVFQGQGLSTRWLC
LIGYGVPLLIVGVSAAIYSKGYGRPRYCWLDFEQGFLWSFLGPVTFIILCNAVIFVTTVW
KLTQKFSEINPDMKKLKKARALTITAIAQLFLLGCTWVFGLFIFDDRSLVLTYVFTILNCL
QGAFLYLLHCLLNKKVREEYRKWACLVAGGSKYSEFTSTTSGTGHNQTRALRASESGI
(UniProt KB Accession No. P48960-1).

In some embodiments, antibodies, or antigen-binding fragments thereof may bind to CD97 (ADGRE5) isoform 2 which has the amino acid sequence

(SEQ ID NO: 86)
MGGRVFLAFCVWLTLPGAETQDSRGCARWCPQNSSCVNATACRCNPGFSSFSEIITTPTE
TCDDINECATPSKVSCGKFSDCWNTEGSYDCVCSPGYEPVSGAKTFKNESENTCQDVDE
CSSGQHQCDSSTVCFNTVGSYSCRCRPGWKPRHGIPNNQKDTVCEDMTFSTWTPPPGV
HSQTLSRFFDKVQDLGRDSKTSSAEVTIQNVIKLVDELMEAPGDVEALAPPVRHLIATQL
LSNLEDIMRILAKSLPKGPFTYISPSNTELTLMIQERGDKNVTMGQSSARMKLNWAVAA
GAEDPGPAVAGILSIQNMTTLLANASLNLHSKKQAELEEIYESSIRGVQLRRLSAVNSIFL
SHNNTKELNSPILFAFSHLESSDGEAGRDPPAKDVMPGPRQELLCAFWKSDSDRGGHW
ATEGCQVLGSKNGSTTCQCSHLSSFAILMAHYDVEDWKLTLITRVGLALSLFCLLLCILT
FLLVRPIQGSRTTIHLHLCICLFVGSTIFLAGIENEGGQVGLRCRLVAGLLHYCFLAAFCW
MSLEGLELYFLVVRVFQGQGLSTRWLCLIGYGVPLLIVGVSAAIYSKGYGRPRYCWLDF
EQGFLWSFLGPVTFIILCNAVIFVTTVWKLTQKFSEINPDMKKLKKARALTITAIAQLFLL
GCTWVFGLFIFDDRSLVLTYVFTILNCLQGAFLYLLHCLLNKKVREEYRKWACLVAGGS
KYSEFTSTTSGTGHNQTRALRASESGI
(UniProt KB Accession No. P48960-2).

In some embodiments, antibodies, or antigen-binding fragments thereof may bind to CD97 (ADGRE5) isoform 3 which has the amino acid sequence

(SEQ ID NO: 87)
MGGRVFLAFCVWLTLPGAETQDSRGCARWCPQNSSCVNATACRCNPGFSSFSEIITTPTE
TCDDINECATPSKVSCGKFSDCWNTEGSYDCVCSPGYEPVSGAKTFKNESENTCQDVDE
CQQNPRLCKSYGTCVNTLGSYTCQCLPGFKFIPEDPKVCTDVDECSSGQHQCDSSTVCF
NTVGSYSCRCRPGWKPRHGIPNNQKDTVCEDMTFSTWTPPPGVHSQTLSRFFDKVQDL
GRDSKTSSAEVTIQNVIKLVDELMEAPGDVEALAPPVRHLIATQLLSNLEDIMRILAKSLP
KGPFTYISPSNTELTLMIQERGDKNVTMGQSSARMKLNWAVAAGAEDPGPAVAGILSIQ
NMTTLLANASLNLHSKKQAELEEIYESSIRGVQLRRLSAVNSIFLSHNNTKELNSPILFAF
SHLESSDGEAGRDPPAKDVMPGPRQELLCAFWKSDSDRGGHWATEGCQVLGSKNGST
TCQCSHLSSFAILMAHYDVEDWKLTLITRVGLALSLFCLLLCILTFLLVRPIQGSRTTIHLH
LCICLFVGSTIFLAGIENEGGQVGLRCRLVAGLLHYCFLAAFCWMSLEGLELYFLVVRVF
QGQGLSTRWLCLIGYGVPLLIVGVSAAIYSKGYGRPRYCWLDFEQGFLWSFLGPVTFIIL
CNAVIFVTTVWKLTQKFSEINPDMKKLKKARALTITAIAQLFLLGCTWVFGLFIFDDRSL
VLTYVFTILNCLQGAFLYLLHCLLNKKVREEYRKWACLVAGGSKYSEFTSTTSGTGHNQ
TRALRASESGI
(UniProt KB Accession No. P48960-3).

The CDR and variable region amino acid sequence identifiers of exemplary anti-CD97 antibodies of the present disclosure are shown in Table 1.

TABLE 1
Amino Acid Sequence Identifiers for CDR and
Variable Regions of Anti-CD97 Antibodies

| | VL | LCDR1 | LCDR2 | LCDR3 | VH | HCDR1 | HCDR2 | HCDR3 |
|---|---|---|---|---|---|---|---|---|
| Antibody | (SEQ | (SEQ | (SEQ | (SEQ | (SEQ | (SEQ | (SEQ | (SEQ |
| clone | ID NO) | ID NO) | ID NO) | ID NO) | ID NO) | ID NO) | ID NO) | ID NO) |
| A1 | 1 | 3 | 4 | 5 | 2 | 6 | 7 | 8 |
| A8 | 9 | 3 | 4 | 11 | 10 | 12 | 13 | 14 |
| A8 | 9 | 3 | 4 | 11 | 109 | 12 | 13 | 112 |
| N100b_{H}E | | | | | | | | |
| A8 | 9 | 3 | 4 | 11 | 110 | 12 | 13 | 113 |
| W100e_{H}A | | | | | | | | |
| A8 | 9 | 3 | 4 | 11 | 111 | 12 | 13 | 114 |
| Y100f_{H}A | | | | | | | | |
| B3 | 15 | 3 | 4 | 17 | 16 | 6 | 18 | 19 |
| B20 | 21 | 3 | 4 | 20 | 22 | 6 | 23 | 24 |
| B23 | 25 | 3 | 4 | 27 | 26 | 6 | 28 | 29 |
| B25 | 21 | 3 | 4 | 20 | 32 | 6 | 30 | 33 |
| B34 | 35 | 3 | 4 | 34 | 36 | 37 | 38 | 39 |
| C14 | 41 | 3 | 4 | 40 | 42 | 6 | 43 | 44 |
| C27 | 21 | 3 | 4 | 20 | 46 | 6 | 7 | 47 |
| D9 | 49 | 3 | 4 | 48 | 50 | 6 | 51 | 52 |
| D12 | 21 | 3 | 4 | 20 | 54 | 6 | 23 | 24 |
| D25 | 21 | 3 | 4 | 20 | 56 | 6 | 13 | 57 |
| 2A5 | 59 | 3 | 4 | 58 | 60 | 6 | 61 | 62 |
| 2V10 | 63 | 3 | 4 | 65 | 64 | 6 | 66 | 67 |
| 2W1 | 69 | 3 | 4 | 68 | 70 | 6 | 71 | 72 |
| 2X10 | 21 | 3 | 4 | 20 | 74 | 6 | 23 | 75 |
| 2X12 | 77 | 3 | 4 | 76 | 78 | 6 | 79 | 80 |
| 2Z3 | 21 | 3 | 4 | 20 | 81 | 6 | 23 | 82 |
| 2Z7 | 21 | 3 | 4 | 20 | 31 | 6 | 23 | 45 |
| 2Z11 | 53 | 3 | 4 | 55 | 73 | 37 | 83 | 84 |

In some embodiments, an antibody or antigen binding fragment described herein comprises a heavy chain complementarity determining region 1 (CDR1), a heavy chain CDR2, a heavy chain CDR3 contained within a heavy chain variable region (VH) comprising the amino acid sequence of SEQ ID NO: 2, 10, 16, 22, 26, 32, 36, 42, 46, 50, 54, 56, 60, 64, 70, 74, 78, 81, 31, 73, 109, 110, or 111. In some embodiments, an antibody or antigen binding fragment described herein comprises a VH comprising an amino acid sequence of SEQ ID NO: 2, 10, 16, 22, 26, 32, 36, 42, 46, 50, 54, 56, 60, 64, 70, 74, 78, 81, 31, 73, 109, 110, or 111, or an amino acid sequence having at least 70%, 75%, 80%, 85%, 90%, 91%, 92%, 93%, 94%, 95%, 96%, 97%, 98%, 99%, 95.5%, or 99.9% sequence identity to the amino acid sequence of SEQ ID NO: 2, 10, 16, 22, 26, 32, 36, 42, 46, 50, 54, 56, 60, 64, 70, 74, 78, 81, 31, 73, 109, 110, or 111.

In some embodiments, an antibody or antigen binding fragment described herein comprises a light chain complementarity determining region 1 (CDR1), a light chain CDR2, a light chain CDR3 contained within a light chain variable region (VL) comprising the amino acid sequence of SEQ ID NO: 1, 9, 15, 21, 25, 21, 35, 41, 21, 49, 21, 21, 59, 63, 69, 21, 77, 21, 21, or 53. In some embodiments, an antibody or antigen binding fragment described herein comprises a VL comprising an amino acid sequence of SEQ ID NO: 1, 9, 15, 21, 25, 21, 35, 41, 21, 49, 21, 21, 59, 63, 69, 21, 77, 21, 21, or 53, or an amino acid sequence having at least 70%, 75%, 80%, 85%, 90%, 91%, 92%, 93%, 94%, 95%, 96%, 97%, 98%, 99%, 95.5%, or 99.9% sequence identity to the amino acid sequence of SEQ ID NO: 1, 9, 15, 21, 25, 21, 35, 41, 21, 49, 21, 21, 59, 63, 69, 21, 77, 21, 21, or 53.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 2, 10, 16, 22, 26, 32, 36, 42, 46, 50, 54, 56, 60, 64, 70, 74, 78, 81, 31, 73, 109, 110, or 111, or a sequence having at least 70%, 75%, 80%, 85%, 90%, preferably 95% or more, such as 95%, 96%, 97%, 98%, or 99% identity thereto; and/or a light chain CDR1, a light chain CDR2, and a light chain CDR3 of a light chain variable region (VL) comprising an amino acid sequence of SEQ ID NO: 1, 9, 15, 21, 25, 21, 35, 41, 21, 49, 21, 21, 59, 63, 69, 21, 77, 21, 21, or 53, or a sequence having at least 70%, 75%, 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 2, 10, 16, 22, 26, 32, 36, 42, 46, 50, 54, 56, 60, 64, 70, 74, 78, 81, 31, or 73, or a sequence having at least 70%, 75%, 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 1, 9, 15, 21, 25, 21, 35, 41, 21, 49, 21, 21, 59, 63, 69, 21, 77, 21, 21, or 53, or a sequence having at least 70%, 75%, 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 2, 10, 16, 22, 26, 32, 36, 42, 46, 50, 54, 56, 60, 64, 70, 74, 78, 81, 31, 73, 109, 110, or 111; and a light chain CDR1, a light chain CDR2, and a light chain CDR3 of a light chain variable region (VL) comprising an amino acid sequence of SEQ ID NO: 1, 9, 15, 21, 25, 21, 35, 41, 21, 49, 21, 21, 59, 63, 69, 21, 77, 21, 21, or 53.

In some embodiments, the heavy chain CDR1 comprises the amino acid sequence of SEQ ID NO: 6, the heavy chain CDR2 comprises the amino acid sequence of SEQ ID NO: 7, and the heavy chain CDR3 comprises the amino acid sequence of SEQ ID NO: 8.

In some embodiments, the heavy chain CDR1 comprises the amino acid sequence of SEQ ID NO: 12, the heavy chain CDR2 comprises the amino acid sequence of SEQ ID NO: 13, and the heavy chain CDR3 comprises the amino acid sequence of SEQ ID NO: 14.

In some embodiments, the heavy chain CDR1 comprises the amino acid sequence of SEQ ID NO: 12, the heavy chain CDR2 comprises the amino acid sequence of SEQ ID NO: 13, and the heavy chain CDR3 comprises the amino acid sequence of SEQ ID NO: 112.

In some embodiments, the heavy chain CDR1 comprises the amino acid sequence of SEQ ID NO: 12, the heavy chain CDR2 comprises the amino acid sequence of SEQ ID NO: 13, and the heavy chain CDR3 comprises the amino acid sequence of SEQ ID NO: 113.

In some embodiments, the heavy chain CDR1 comprises the amino acid sequence of SEQ ID NO: 12, the heavy chain CDR2 comprises the amino acid sequence of SEQ ID NO: 13, and the heavy chain CDR3 comprises the amino acid sequence of SEQ ID NO: 114.

In some embodiments, the heavy chain CDR1 comprises the amino acid sequence of SEQ ID NO: 6, the heavy chain CDR2 comprises the amino acid sequence of SEQ ID NO: 18, and the heavy chain CDR3 comprises the amino acid sequence of SEQ ID NO: 19.

In some embodiments, the heavy chain CDR1 comprises the amino acid sequence of SEQ ID NO: 6, the heavy chain CDR2 comprises the amino acid sequence of SEQ ID NO: 23, and the heavy chain CDR3 comprises the amino acid sequence of SEQ ID NO: 24.

In some embodiments, the heavy chain CDR1 comprises the amino acid sequence of SEQ ID NO: 6, the heavy chain CDR2 comprises the amino acid sequence of SEQ ID NO: 28, and the heavy chain CDR3 comprises the amino acid sequence of SEQ ID NO: 29.

In some embodiments, the heavy chain CDR1 comprises the amino acid sequence of SEQ ID NO: 6, the heavy chain CDR2 comprises the amino acid sequence of SEQ ID NO: 30, and the heavy chain CDR3 comprises the amino acid sequence of SEQ ID NO: 33.

In some embodiments, the heavy chain CDR1 comprises the amino acid sequence of SEQ ID NO: 37, the heavy chain CDR2 comprises the amino acid sequence of SEQ ID NO: 38, and the heavy chain CDR3 comprises the amino acid sequence of SEQ ID NO: 39.

In some embodiments, the heavy chain CDR1 comprises the amino acid sequence of SEQ ID NO: 6, the heavy chain CDR2 comprises the amino acid sequence of SEQ ID NO: 43, and the heavy chain CDR3 comprises the amino acid sequence of SEQ ID NO: 44.

In some embodiments, the heavy chain CDR1 comprises the amino acid sequence of SEQ ID NO: 6, the heavy chain CDR2 comprises the amino acid sequence of SEQ ID NO: 7, and the heavy chain CDR3 comprises the amino acid sequence of SEQ ID NO: 47.

In some embodiments, the heavy chain CDR1 comprises the amino acid sequence of SEQ ID NO: 6, the heavy chain CDR2 comprises the amino acid sequence of SEQ ID NO: 51, and the heavy chain CDR3 comprises the amino acid sequence of SEQ ID NO: 52.

In some embodiments, the heavy chain CDR1 comprises the amino acid sequence of SEQ ID NO: 6, the heavy chain CDR2 comprises the amino acid sequence of SEQ ID NO: 23, and the heavy chain CDR3 comprises the amino acid sequence of SEQ ID NO: 24.

In some embodiments, the heavy chain CDR1 comprises the amino acid sequence of SEQ ID NO: 6, the heavy chain CDR2 comprises the amino acid sequence of SEQ ID NO: 13, and the heavy chain CDR3 comprises the amino acid sequence of SEQ ID NO: 57.

In some embodiments, the heavy chain CDR1 comprises the amino acid sequence of SEQ ID NO: 6, the heavy chain CDR2 comprises the amino acid sequence of SEQ ID NO: 61, and the heavy chain CDR3 comprises the amino acid sequence of SEQ ID NO: 62.

In some embodiments, the heavy chain CDR1 comprises the amino acid sequence of SEQ ID NO: 6, the heavy chain CDR2 comprises the amino acid sequence of SEQ ID NO: 66, and the heavy chain CDR3 comprises the amino acid sequence of SEQ ID NO: 67.

In some embodiments, the heavy chain CDR1 comprises the amino acid sequence of SEQ ID NO: 6, the heavy chain CDR2 comprises the amino acid sequence of SEQ ID NO: 71, and the heavy chain CDR3 comprises the amino acid sequence of SEQ ID NO: 72.

In some embodiments, the heavy chain CDR1 comprises the amino acid sequence of SEQ ID NO: 6, the heavy chain CDR2 comprises the amino acid sequence of SEQ ID NO: 23, and the heavy chain CDR3 comprises the amino acid sequence of SEQ ID NO: 75.

In some embodiments, the heavy chain CDR1 comprises the amino acid sequence of SEQ ID NO: 6, the heavy chain CDR2 comprises the amino acid sequence of SEQ ID NO: 79, and the heavy chain CDR3 comprises the amino acid sequence of SEQ ID NO: 80.

In some embodiments, the heavy chain CDR1 comprises the amino acid sequence of SEQ ID NO: 6, the heavy chain CDR2 comprises the amino acid sequence of SEQ ID NO: 23, and the heavy chain CDR3 comprises the amino acid sequence of SEQ ID NO: 82.

In some embodiments, the heavy chain CDR1 comprises the amino acid sequence of SEQ ID NO: 6, the heavy chain CDR2 comprises the amino acid sequence of SEQ ID NO: 23, and the heavy chain CDR3 comprises the amino acid sequence of SEQ ID NO: 45.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 5.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 11.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 17.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 20.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 27.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 34.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 40.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 48.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 58.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 65.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 68.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 76.

In some embodiments, the light chain CDR1 comprises the amino acid sequence of SEQ ID NO: 3, the light chain CDR2 comprises the amino acid sequence of SEQ ID NO: 4, and the light chain CDR3 comprises the amino acid sequence of SEQ ID NO: 55.

In some embodiments, the isolated antibody or antigen-binding fragment comprises the heavy chain CDR1 of SEQ ID NO: 6, the heavy chain CDR2 of SEQ ID NO: 7, the heavy chain CDR3 of SEQ ID NO: 8, the light chain CDR1 of SEQ ID NO: 3, the light chain CDR2 of SEQ ID NO: 4, and the light chain CDR3 of SEQ ID NO: 5.

In some embodiments, the isolated antibody or antigen-binding fragment comprises the heavy chain CDR1 of SEQ ID NO: 12, the heavy chain CDR2 of SEQ ID NO: 13, the heavy chain CDR3 of SEQ ID NO: 14, the light chain CDR1 of SEQ ID NO: 3, the light chain CDR2 of SEQ ID NO: 4, and the light chain CDR3 of SEQ ID NO: 11.

In some embodiments, the isolated antibody or antigen-binding fragment comprises the heavy chain CDR1 of SEQ ID NO: 12, the heavy chain CDR2 of SEQ ID NO: 13, the heavy chain CDR3 of SEQ ID NO: 112, the light chain CDR1 of SEQ ID NO: 3, the light chain CDR2 of SEQ ID NO: 4, and the light chain CDR3 of SEQ ID NO: 11.

In some embodiments, the isolated antibody or antigen-binding fragment comprises the heavy chain CDR1 of SEQ ID NO: 12, the heavy chain CDR2 of SEQ ID NO: 13, the heavy chain CDR3 of SEQ ID NO: 113, the light chain CDR1 of SEQ ID NO: 3, the light chain CDR2 of SEQ ID NO: 4, and the light chain CDR3 of SEQ ID NO: 11.

In some embodiments, the isolated antibody or antigen-binding fragment comprises the heavy chain CDR1 of SEQ ID NO: 12, the heavy chain CDR2 of SEQ ID NO: 13, the heavy chain CDR3 of SEQ ID NO: 114, the light chain CDR1 of SEQ ID NO: 3, the light chain CDR2 of SEQ ID NO: 4, and the light chain CDR3 of SEQ ID NO: 11.

In some embodiments, the isolated antibody or antigen-binding fragment comprises the heavy chain CDR1 of SEQ ID NO: 6, the heavy chain CDR2 of SEQ ID NO: 18, the heavy chain CDR3 of SEQ ID NO: 19, the light chain CDR1 of SEQ ID NO: 3, the light chain CDR2 of SEQ ID NO: 4, and the light chain CDR3 of SEQ ID NO: 17.

In some embodiments, the isolated antibody or antigen-binding fragment comprises the heavy chain CDR1 of SEQ ID NO: 6, the heavy chain CDR2 of SEQ ID NO: 23, the heavy chain CDR3 of SEQ ID NO: 24, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 28, the heavy chain CDR3 of SEQ ID NO: 29, the light chain CDR1 of SEQ ID NO: 3, the light chain CDR2 of SEQ ID NO: 4, and the light chain CDR3 of SEQ ID NO: 27.

In some embodiments, the isolated antibody or antigen-binding fragment comprises the heavy chain CDR1 of SEQ ID NO: 6, the heavy chain CDR2 of SEQ ID NO: 30, the heavy chain CDR3 of SEQ ID NO: 33, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 37, the heavy chain CDR2 of SEQ ID NO: 38, the heavy chain CDR3 of SEQ ID NO: 39, the light chain CDR1 of SEQ ID NO: 3, the light chain CDR2 of SEQ ID NO: 4, and the light chain CDR3 of SEQ ID NO: 34.

In some embodiments, the isolated antibody or antigen-binding fragment comprises the heavy chain CDR1 of SEQ ID NO: 6, the heavy chain CDR2 of SEQ ID NO: 43, the heavy chain CDR3 of SEQ ID NO: 44, the light chain CDR1 of SEQ ID NO: 3, the light chain CDR2 of SEQ ID NO: 4, and the light chain CDR3 of SEQ ID NO: 40.

In some embodiments, the isolated antibody or antigen-binding fragment comprises the heavy chain CDR1 of SEQ ID NO: 6, the heavy chain CDR2 of SEQ ID NO: 7, the heavy chain CDR3 of SEQ ID NO: 47, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 51, the heavy chain CDR3 of SEQ ID NO: 52, the light chain CDR1 of SEQ ID NO: 3, the light chain CDR2 of SEQ ID NO: 4, and the light chain CDR3 of SEQ ID NO: 48.

In some embodiments, the isolated antibody or antigen-binding fragment comprises the heavy chain CDR1 of SEQ ID NO: 6, the heavy chain CDR2 of SEQ ID NO: 23, the heavy chain CDR3 of SEQ ID NO: 24, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 13, the heavy chain CDR3 of SEQ ID NO: 57, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 61, the heavy chain CDR3 of SEQ ID NO: 62, the light chain CDR1 of SEQ ID NO: 3, the light chain CDR2 of SEQ ID NO: 4, and the light chain CDR3 of SEQ ID NO: 58.

In some embodiments, the isolated antibody or antigen-binding fragment comprises the heavy chain CDR1 of SEQ ID NO: 6, the heavy chain CDR2 of SEQ ID NO: 66, the heavy chain CDR3 of SEQ ID NO: 67, the light chain CDR1 of SEQ ID NO: 3, the light chain CDR2 of SEQ ID NO: 4, and the light chain CDR3 of SEQ ID NO: 65.

In some embodiments, the isolated antibody or antigen-binding fragment comprises the heavy chain CDR1 of SEQ ID NO: 6, the heavy chain CDR2 of SEQ ID NO: 71, the heavy chain CDR3 of SEQ ID NO: 72, the light chain CDR1 of SEQ ID NO: 3, the light chain CDR2 of SEQ ID NO: 4, and the light chain CDR3 of SEQ ID NO: 68.

In some embodiments, the isolated antibody or antigen-binding fragment comprises the heavy chain CDR1 of SEQ ID NO: 6, the heavy chain CDR2 of SEQ ID NO: 23, the heavy chain CDR3 of SEQ ID NO: 75, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 79, the heavy chain CDR3 of SEQ ID NO: 80, the light chain CDR1 of SEQ ID NO: 3, the light chain CDR2 of SEQ ID NO: 4, and the light chain CDR3 of SEQ ID NO: 76.

In some embodiments, the isolated antibody or antigen-binding fragment comprises the heavy chain CDR1 of SEQ ID NO: 6, the heavy chain CDR2 of SEQ ID NO: 23, the heavy chain CDR3 of SEQ ID NO: 82, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 6, the heavy chain CDR2 of SEQ ID NO: 23, the heavy chain CDR3 of SEQ ID NO: 45, the light chain CDR1 of SEQ ID NO: 3, the light chain CDR2 of SEQ ID NO: 4, and the light chain CDR3 of SEQ ID NO: 20.

In some embodiments, the isolated antibody or antigen-binding fragment comprises the heavy chain CDR1 of SEQ ID NO: 37, the heavy chain CDR2 of SEQ ID NO: 83, the heavy chain CDR3 of SEQ ID NO: 84, the light chain CDR1 of SEQ ID NO: 3, the light chain CDR2 of SEQ ID NO: 4, and the light chain CDR3 of SEQ ID NO: 55.

In some embodiments, the isolated antibody or antigen-binding fragment comprises a VH comprising an amino acid sequence of SEQ ID NO: 2, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 1, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 10, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 109, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 110, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 111, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 16, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 15, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 22, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 26, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 25, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 32, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 36, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 35, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 42, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 41, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 46, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 50, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 49, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 54, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 56, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 60, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 59, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 64, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 63, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 70, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 69, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 74, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 78, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 77, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 81, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 31, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 73, or a sequence having at least 70%, 75%, 80%, 85%, 90%, preferably 95% or more, such as 95%, 96%, 97%, 98%, or 99% identity thereto; and/or a VL comprising an amino acid sequence of SEQ ID NO: 53, or a sequence having at least 70%, 75%, 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 2; and/or a VL comprising an amino acid sequence of SEQ ID NO: 1.

In some embodiments, the isolated antibody or antigen-binding fragment comprises a VH comprising an amino acid sequence of SEQ ID NO: 10; and/or a VL comprising an amino acid sequence of SEQ ID NO: 9.

In some embodiments, the isolated antibody or antigen-binding fragment comprises a VH comprising an amino acid sequence of SEQ ID NO: 109; and/or a VL comprising an amino acid sequence of SEQ ID NO: 9.

In some embodiments, the isolated antibody or antigen-binding fragment comprises a VH comprising an amino acid sequence of SEQ ID NO: 110; and/or a VL comprising an amino acid sequence of SEQ ID NO: 9.

In some embodiments, the isolated antibody or antigen-binding fragment comprises a VH comprising an amino acid sequence of SEQ ID NO: 111; and/or a VL comprising an amino acid sequence of SEQ ID NO: 9.

In some embodiments, the isolated antibody or antigen-binding fragment comprises a VH comprising an amino acid sequence of SEQ ID NO: 16; and/or a VL comprising an amino acid sequence of SEQ ID NO: 15.

In some embodiments, the isolated antibody or antigen-binding fragment comprises a VH comprising an amino acid sequence of SEQ ID NO: 22; and/or a VL comprising an amino acid sequence of SEQ ID NO: 21.

In some embodiments, the isolated antibody or antigen-binding fragment comprises a VH comprising an amino acid sequence of SEQ ID NO: 26; and/or a VL comprising an amino acid sequence of SEQ ID NO: 25.

In some embodiments, the isolated antibody or antigen-binding fragment comprises a VH comprising an amino acid sequence of SEQ ID NO: 32; and/or a VL comprising an amino acid sequence of SEQ ID NO: 21.

In some embodiments, the isolated antibody or antigen-binding fragment comprises a VH comprising an amino acid sequence of SEQ ID NO: 36; and/or a VL comprising an amino acid sequence of SEQ ID NO: 35.

In some embodiments, the isolated antibody or antigen-binding fragment comprises a VH comprising an amino acid sequence of SEQ ID NO: 42; and/or a VL comprising an amino acid sequence of SEQ ID NO: 41.

In some embodiments, the isolated antibody or antigen-binding fragment comprises a VH comprising an amino acid sequence of SEQ ID NO: 46; and/or a VL comprising an amino acid sequence of SEQ ID NO: 21.

In some embodiments, the isolated antibody or antigen-binding fragment comprises a VH comprising an amino acid sequence of SEQ ID NO: 50; and/or a VL comprising an amino acid sequence of SEQ ID NO: 49.

In some embodiments, the isolated antibody or antigen-binding fragment comprises a VH comprising an amino acid sequence of SEQ ID NO: 54; and/or a VL comprising an amino acid sequence of SEQ ID NO: 21.

In some embodiments, the isolated antibody or antigen-binding fragment comprises a VH comprising an amino acid sequence of SEQ ID NO: 56; and/or a VL comprising an amino acid sequence of SEQ ID NO: 21.

In some embodiments, the isolated antibody or antigen-binding fragment comprises a VH comprising an amino acid sequence of SEQ ID NO: 60; and/or a VL comprising an amino acid sequence of SEQ ID NO: 59.

In some embodiments, the isolated antibody or antigen-binding fragment comprises a VH comprising an amino acid sequence of SEQ ID NO: 64; and/or a VL comprising an amino acid sequence of SEQ ID NO: 63.

In some embodiments, the isolated antibody or antigen-binding fragment comprises a VH comprising an amino acid sequence of SEQ ID NO: 70; and/or a VL comprising an amino acid sequence of SEQ ID NO: 69.

In some embodiments, the isolated antibody or antigen-binding fragment comprises a VH comprising an amino acid sequence of SEQ ID NO: 74; and/or a VL comprising an amino acid sequence of SEQ ID NO: 21.

In some embodiments, the isolated antibody or antigen-binding fragment comprises a VH comprising an amino acid sequence of SEQ ID NO: 78; and/or a VL comprising an amino acid sequence of SEQ ID NO: 77.

In some embodiments, the isolated antibody or antigen-binding fragment comprises a VH comprising an amino acid sequence of SEQ ID NO: 81; and/or a VL comprising an amino acid sequence of SEQ ID NO: 21.

In some embodiments, the isolated antibody or antigen-binding fragment comprises a VH comprising an amino acid sequence of SEQ ID NO: 31; and/or a VL comprising an amino acid sequence of SEQ ID NO: 21.

In some embodiments, the isolated antibody or antigen-binding fragment comprises a VH comprising an amino acid sequence of SEQ ID NO: 73; and/or a VL comprising an amino acid sequence of SEQ ID NO: 53.

In certain embodiments, the isolated antibody or antigen-binding fragment comprises one or more amino acid substitutions. In certain embodiments, amino acid substitutions of an antibody or portion thereof are those which: (1) reduce susceptibility to proteolysis, (2) reduce susceptibility to oxidation, (3) alter binding affinity for forming protein complexes, or (4) confer or modify other physicochemical or functional properties. For example, single or multiple amino acid substitutions (preferably conservative amino acid substitutions) may be made in the naturally occurring sequence.

A conservative amino acid substitution should not substantially change the structural characteristics of the parent sequence. Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

As used herein, the twenty naturally occurring amino acids and their abbreviations follow conventional usage. See Immunology—A Synthesis (2^{nd} Edition, E. S. Golub and D. R. Green, Eds., Sinauer Associates, Sunderland, Mass. (1991)), which is incorporated herein by reference.

In some embodiments, the antibody or antigen-binding fragment is recombinant. In certain embodiments, the antibody or antigen-binding fragment is a human antibody, a humanized antibody, a chimeric antibody, a murine antibody, a monoclonal antibody, a single chain antibody, a bispecific antibody or antigen-binding fragment thereof, a Fab, a Fab′, a F(ab′)2, a Fv, a scFv, or a nanobody.

In some embodiments, the antibodies described herein are humanized antibodies.

In some embodiments, the antibodies described herein are chimeric antibodies.

In some embodiments, the antigen-binding fragment is a Fab.

In some embodiments, the bispecific antibody or antigen-binding fragment thereof is a bispecific T-cell engager (BiTE) or bispecific NK-cell engager.

In some embodiments, the anti-CD97 antibodies disclosed herein, having the heavy chain CDRs disclosed herein, contains framework regions derived from a subclass of germline VH fragment. Such germline V_{H} regions are well known in the art. See, e.g., the IMGT database (imgt.org) or at vbase2.org/vbstat.php. Examples include the IGHV1 subfamily (e.g., IGHV1-2, IGHV1-3, IGHV1-8. IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9. IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGI-HV3-53, IGHV3-64, IGHV3-66, IGHV3-72, and IGHV3-73, IGHV3-74), the IGHV4 subfamily (e.g., IGHV44, IGHV4-28, IGHV4-31, IGHV4-34, IGHV4-39, IGHV4-59, IGI-V4-61, and IGIHV4-B), the IGHV subfamily (e.g., IGHV5-51, or IGHV6-1), and the IGHV7 subfamily (e.g., IGHV7-4-1).

Alternatively, or in addition, in some embodiments, the anti-CD97 antibody, having the light chain CDRs disclosed herein, contains framework regions derived from a germline VK fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20). and an IGKV4 framework (e.g., IGKV4-1). In other instances, the anti-CD97 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IGλ2 framework (e.g., IGλV2-8, IGλ,V2-11, IGλV2-14, IGλ2-18, IGλV2-23), an IGλ3 framework (e.g., IGλ3-1, IGλ3-9, IGλV3-10, IGλV3-12, IGλV3-16, IGλV3-19, IGλV3-21, IGλV3-25, IGλV3-27), an IGλ4 framework (e.g., IGλV4-3, IGλV4-60, GλV4-69), an IGλ5 framework (e.g., IGλV5-39, IGλV5-45), an IGλ6 framework (e.g., IGλV6-57), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλV10-54).

In some embodiments, an anti-CD97 antibody, or antigen-binding fragment thereof which binds to at least one isoform of CD97 may bind to isoform 1 of CD97. In some embodiments, the antibody or antigen-binding fragment thereof may bind to isoform 2 of CD97. In some embodiments, the antibody or antigen-binding fragment thereof may bind to isoform 3 of CD97.

In some embodiments, an anti-CD97 antibody, or antigen-binding fragment thereof, which binds to at least one isoform of CD97 may bind to both isoform 1 and isoform 2 of CD97. In some embodiments, the antibody or antigen-binding fragment thereof may bind to isoform 1, isoform 2, and isoform 3 of CD97.

In some embodiments, anti-CD97 antibodies, or antigen-binding fragments thereof, of the present disclosure bind to the same epitope as any of the exemplary antibodies set forth herein or competes against the exemplary antibody from binding CD97. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof, Overlapping epitopes include at least one common amino acid residue. An epitope can be linear, which is typically 6-15 amino acids in length. Alternatively, the epitope can be conformational. The epitope to which an antibody binds can be determined by routine technology, for example, an epitope mapping method described herein. An antibody that binds the same epitope as an exemplary antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the exemplary antibody, Whether two antibodies compete against each other from binding to a cognate antigen can be determined, e.g., by a competition assay, which is well known in the art.

In some embodiments, an anti-CD97 antibody, or antigen-binding fragment thereof, of the present disclosure binds to one or more residues in isoform 1, isoform 2, and/or isoform 3 of CD97.

In some embodiments, an anti-CD97 antibody, or antigen-binding fragment thereof, of the present disclosure binds to one or more residues in isoform 1 of CD97, and the one or more residues are selected from, e.g., T351, Q376, R380, K382, K519, and T524, and any combination thereof.

In some embodiments, the antibody, or antigen-binding fragment thereof, binds to the GPCR autoproteolysis-inducing (GAIN) domain in CD97. In some embodiments, the antibody, or antigen-binding fragment thereof, binds to the first EGF-like domain in CD97. In some embodiments, the antibody, or antigen-binding fragment thereof, binds the third EGF-like domain in CD97.

In some embodiments, an anti-CD97 antibody, or antigen-binding fragment thereof does not bind to EGF-like module-containing mucin-like hormone receptor-like 2 (EMR2). In some embodiments, an anti-CD97 antibody, or antigen-binding fragment thereof does not bind to isoform 1 of EMR2. In some embodiments, an anti-CD97 antibody, or antigen-binding fragment thereof does not bind to isoform 4 of EMR2. In some embodiments, an anti-CD97 antibody, or antigen-binding fragment thereof does not bind to either isoform 1 or isoform 4 of EMR2.

In some embodiments, an anti-CD97 antibody or antigen-binding fragment as described herein has a suitable binding affinity for the target antigen (e.g., CD97) or antigenic epitopes thereof. As used herein, “binding affinity” refers to the apparent association constant or K_{A}. The KA is the reciprocal of the dissociation constant (K)). In various embodiments, the anti-CD97 antibody, or antigen-binding fragment thereof described herein, may have a binding affinity (K_{D}) of at least about 1×10⁻⁶ M or less, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less for the target antigen or antigenic epitope. An increased binding affinity corresponds to a decreased K_{D}.

In various embodiments, the antibodies of the disclosure have the ability to bind to a predetermined antigen (e.g., CD97) with a dissociation constant (K_{D}) of 1×10⁻⁶ M or less, 5×10⁻⁷ M or less, 1×10⁻⁷ M or less, 5×10⁻⁸ M or less, 4×10⁻⁸ M or less, 3×10⁻⁸ M or less, 2×10⁻⁸ M or less, about 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, about 1×10⁻⁹ M or less, 9×10⁻¹⁰ M or less, 8×10⁻¹⁰ M or less, 7×10⁻¹⁰ M or less, 6×10⁻¹⁰ M or less, 5×10⁻¹⁰ M or less, 4×10⁻¹⁰ M or less, 3×10⁻¹⁰ M or less, 2×10⁻¹⁰ M or less, 1×10⁻¹⁰ M or less, 5×10⁻¹¹ M or less, about 1×10⁻¹¹ M or less, 5×10⁻¹² M or less, about 1×10⁻¹² M or less, 5×10⁻¹³ M or less, or about 1×10⁻¹³ M or less.

In some embodiments, an anti-CD97 antibody or antigen-binding fragment thereof described herein may bind to isoform 1 of CD97 at a dissociation constant (K_{D}) of about 1×10⁻⁶ M or less, 5×10⁻⁷ M or less, 1×10⁻⁷ M or less, 5×10⁻⁸ M or less, 4×10⁻⁸ M or less, 3×10⁻⁸ M or less, 2×10⁻⁸ M or less, about 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, about 1×10⁻⁹ M or less, 9×10⁻¹⁰ M or less, 8×10⁻¹⁰ M or less, 7×10⁻¹⁰ M or less, 6×10⁻¹⁰ M or less, 5×10⁻¹⁰ M or less, 4×10⁻¹⁰ M or less, 3×10⁻¹⁰ M or less, 2×10⁻¹⁰ M or less, 1×10⁻¹⁰ M or less, 5×10⁻¹¹ M or less, about 1×10⁻¹¹ M or less, 5×10⁻¹¹ M or less, about 1×10⁻¹² M or less, 5×10⁻¹² M or less, or about 1×10−3 M or less. As a non-limiting example, the anti-CD97 antibody, or antigen-binding fragment thereof, may bind to isoform 1 of CD97 at a dissociation constant (K_{D}) of about 1×10⁻⁷ M or less. In some embodiments, an anti-CD97 antibody or antigen-binding fragment thereof described herein may bind to isoform 1 of CD97 at a K_{D} of about 5×10⁻⁸ M or less. In some embodiments, an anti-CD97 antibody or antigen-binding fragment thereof described herein may bind to isoform 1 of CD97 at a K_{D} of about 5×10⁻⁹ M or less. In some embodiments, an anti-CD97 antibody or antigen-binding fragment thereof described herein may bind to isoform 1 of CD97 at a K_{D} of about 5×10−¹⁰ M or less.

In some embodiments, an anti-CD97 antibody or antigen-binding fragment thereof described herein may bind to isoform 1 of CD97 at a dissociation constant (K_{D}) of about 3.1×10−7 M or less. In some embodiments, an anti-CD97 antibody or antigen-binding fragment thereof described herein may bind to isoform 1 of CD97 at a dissociation constant (K_{D}) of about 1.6×10−7 M or less. In some embodiments, an anti-CD97 antibody or antigen-binding fragment thereof described herein may bind to isoform 1 of CD97 at a dissociation constant (K_{D}) of about 1.1×10−7 M or less. In some embodiments, an anti-CD97 antibody or antigen-binding fragment thereof described herein may bind to isoform 1 of CD97 at a dissociation constant (K_{D}) of about 1.6×10−8 M or less. In some embodiments, an anti-CD97 antibody or antigen-binding fragment thereof described herein may bind to isoform 1 of CD97 at a dissociation constant (K_{D}) of about 5.6×10 ⁻⁸ M or less. In some embodiments, an anti-CD97 antibody or antigen-binding fragment thereof described herein may bind to isoform 1 of CD97 at a dissociation constant (K_{D}) of about 4.5×10⁻⁸ M or less.

In some embodiments, an anti-CD97 antibody or antigen-binding fragment thereof (e.g., Fab) described herein may bind to isoform 1 of CD97 at a dissociation constant (K_{D}) of from about 2×10⁻⁹ M to about 2×10⁻⁸ M as measured using a bead-based binding assay. In some embodiments, an anti-CD97 antibody or antigen-binding fragment thereof (e.g., a Fab) described herein may bind to isoform 1 of CD97 at a dissociation constant (K_{D}) of about 2.4×10⁻⁹ M as measured using a bead-based binding assay. In some embodiments, an anti-CD97 antibody or antigen-binding fragment thereof (e.g., a Fab) described herein may bind to isoform 1 of CD97 at a dissociation constant (K_{D}) of about 9.5×10⁻⁹ M as measured using a bead-based binding assay. In some embodiments, an anti-CD97 antibody or antigen-binding fragment thereof (e.g., a Fab) described herein may bind to isoform 1 of CD97 at a dissociation constant (K_{D}) of about 1.6×10⁻⁸ M as measured using a bead-based binding assay.

In some embodiments, an anti-CD97 antibody or antigen-binding fragment thereof described herein may bind to isoform 1 of CD97 at a dissociation constant (K_{D}) of from about 2.5×10⁻⁹ to about 1.6×10⁻⁸ M as measured using a bead-based binding assay. In some embodiments, an anti-CD97 antibody or antigen-binding fragment thereof (e.g., a Fab) described herein may bind to isoform 1 of CD97 at a dissociation constant (K_{D}) of about 9.3×10⁻⁹ M as measured using a bead-based binding assay.

In some embodiments, an anti-CD97 antibody or antigen-binding fragment thereof (e.g., Fab) described herein may bind to isoform 1 of CD97 at a dissociation constant (K_{D}) of from about 3.6×10⁻⁸ M to about 5.4×10⁻⁸ M as measured using a cell-based binding assay. In some embodiments, an anti-CD97 antibody or antigen-binding fragment thereof described herein may bind to isoform 1 of CD97 at a dissociation constant (K_{D}) of about 4.5×10⁻⁸ M as measured using a cell-based binding assay.

In some embodiments, an anti-CD97 antibody or antigen-binding fragment thereof (e.g., in IgG1 format) described herein may bind to isoform 1 of CD97 at a dissociation constant (K_{D}) of about 4.5×10⁻⁹ M to about 5.3×10⁻⁹ M as measured using a cell-based binding assay.

In some embodiments, an anti-CD97 antibody or antigen-binding fragment thereof (e.g., in IgG1 format) described herein may bind to isoform 1 of CD97 at a dissociation constant (K_{D}) of from about 3.4×10⁻¹¹ M to about 2.6×10−¹⁰ M as measured using a cell-based binding assay. In some embodiments, an anti-CD97 antibody or antigen-binding fragment thereof (e.g., in IgG1 format) described herein may bind to isoform 1 of CD97 at a dissociation constant (K_{D}) of about 3×10−¹⁰ M as measured using a cell-based binding assay.

In some embodiments, an anti-CD97 antibody or antigen-binding fragment thereof described herein may bind to isoform 2 of CD97 at a dissociation constant (K_{D}) of about 1×10⁻⁶ M or less, 5×10⁻⁷ M or less, 1×10⁻⁷ M or less, 5×10⁻⁸ M or less, 4×10⁻⁸ M or less, 3×10⁻⁸ M or less, 2×10⁻⁸ M or less, about 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, about 1×10⁻⁹ M or less, 9×10⁻¹⁰ M or less, 8×10⁻¹⁰ M or less, 7×10⁻¹⁰ M or less, 6×10⁻¹⁰ M or less, 5×10⁻¹⁰ M or less, 4×10⁻¹⁰ M or less, 3×10⁻¹⁰ M or less, 2×10⁻¹⁰ M or less, 1×10⁻¹⁰ M or less, 5×10⁻¹¹ M or less, about 1×10⁻¹¹ M or less, 5×10⁻¹² M or less, about 1×10⁻¹² M or less, 5×10⁻¹³ M or less, or about 1×10⁻¹³ M or less. As a non-limiting example, the anti-CD97 antibody, or antigen-binding fragment thereof, may bind to isoform 2 of CD97 at a dissociation constant (K_{D}) of about 1×10−7 M or less. In some embodiments, an anti-CD97 antibody or antigen-binding fragment thereof described herein may bind to isoform 2 of CD97 at a K_{D} of about 5×10⁻⁹ M or less.

In some embodiments, an anti-CD97 antibody or antigen-binding fragment thereof described herein may bind to isoform 3 of CD97 at a dissociation constant (K_{D}) of about 1×10⁻⁶ M or less, 5×10⁻⁷ M or less, 1×10⁻⁷ M or less, 5×10⁻⁸ M or less, 4×10⁻⁸ M or less, 3×10⁻⁸ M or less, 2×10⁻⁸ M or less, about 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, about 1×10⁻⁹ M or less, 9×10⁻¹⁰ M or less, 8×10⁻¹⁰ M or less, 7×10⁻¹⁰ M or less, 6×10⁻¹⁰ M or less, 5×10⁻¹⁰ M or less, 4×10⁻¹⁰ M or less, 3×10⁻¹⁰ M or less, 2×10⁻¹⁰ M or less, 1×10⁻¹⁰ M or less, 5×10⁻¹¹ M or less, about 1×10⁻¹¹ M or less, 5×10⁻¹² M or less, about 1×10⁻¹² M or less, 5×10⁻¹³ M or less, or about 1×10⁻¹³ M or less. As a non-limiting example, the anti-CD97 antibody, or antigen-binding fragment thereof, may bind to isoform 3 of CD97 at a dissociation constant (K_{D}) of about 1×10⁻⁷ M or less. In some embodiments, an anti-CD97 antibody or antigen-binding fragment thereof described herein may bind to isoform 3 of CD97 at a K_{D} of about 5×10⁻⁹ M or less.

Binding affinity (or binding specificity) can be determined by a variety of methods including equilibrium dialysis, equilibrium binding, gel filtration, ELISA, biolayer interferometry (BLI), surface plasmon resonance (SPR), bead-based assay, cell-based assay, radioimmunoassay, or spectroscopy (e.g., using a fluorescence assay), In certain embodiments, a CD97 antigen, or derivative thereof, is coated onto a bead or onto the surface of an ELISA plate or other solid phase used for measurement. Examples of bead-based binding assays which may be used for measuring K_{D} of the anti-CD97 antibodies and/or antigen-binding fragments herein are described in, e.g., Nishikori et al., J Mol Biol. 2012 Dec. 14; 424(5):391-9 (PMID 23041298) and Hattori et al., J Immunol Methods. 2021 March; 490:112952 (PMID 33358997), each of which is incorporated herein by reference in its entirety and for all purposes as if fully set forth herein.

In some embodiments, a K_{D} described herein is measured using a bead-based binding assay. In some embodiments, a K_{D} described herein is measured using a cell-based binding assay.

These techniques can be used to measure the concentration of bound antibody or antigen-binding fragment as a function of target antigen concentration. Under certain conditions, the fractional concentration of bound antibody or antigen-binding fragment ([Bound]/[Total]) is generally related to the concentration of total target antigen ([Target]) by the following equation:

[Bound]/[Total]=[Target]/(K_{D}+[Target])

It is not always necessary to make an exact determination of Kr, though, since sometimes it is sufficient to obtain a quantitative measurement of affinity, e.g., determined using a method such as ELISA or FACS analysis, is proportional to K_{D}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in the in vivo setting, and an antibody with lower binding affinity is more desirable.

In some embodiments, the heavy chain of any of any of the anti-CD97 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In some embodiments, the heavy chain constant region is from an IgD, IgE, IgG, IgA, or IgM class, or sub-class thereof. In some embodiments, the heavy chain constant region is from a human IgG (a gamma heavy chain) or any IgG subfamily as described herein. In some embodiments, the heavy chain constant region is from an IgG1, IgG2, IgG3, or IgG4 subclass.

In some embodiments, the heavy chain constant region of the antibodies described herein comprise a single domain (e.g., CH1, C12, or C13) or a combination of any of the single domains, of a constant region. In some embodiments, the light chain constant region of the antibodies described herein comprises a single domain (e.g., CL), of a constant region.

In some embodiments, the anti-CD97 antibody or antigen-binding fragment described herein is of an IgG1 subclass.

In some embodiments, the anti-CD97 antibody or antigen-binding fragment described herein is of an IgG2 subclass.

In some embodiments, the anti-CD97 antibody or antigen-binding fragment described herein is of an IgG4 subclass.

In some embodiments, anti-CD97 antibodies or antigen-binding fragment described herein comprises a fragment crystallizable (Fc) region. In some embodiments, anti-CD97 antibodies or antigen-binding fragment described herein comprises a modified Fc region.

In some embodiments, an Fe region described herein is altered by replacing at least one amino acid residue with a different amino acid residue to alter the effector function(s) of the antibody. For example, one or more amino acids selected from the following positions: 234, 235, 236, 237, 297, 318, 320, 322, 330, and/or 331 may be substituted with a different amino acid residue such that the antibody has an altered affinity for an effector ligand but retains the antigen-binding ability of the parent antibody. The effector ligand to which affinity is altered can be, for example, an Fc receptor or the C1 component of complement. See, e.g., U.S. Pat. Nos. 5,624,821 and 5,648,260, both of which are incorporated herein by reference in their entirety.

In one embodiment, an. Fc region described herein comprises one or more amino acids substitutions at amino acid residues 329, 331 and 322 such that the antibody has altered C1q binding and/or reduced or abolished complement dependent cytotoxicity (CDC). See, e.g., U.S. Pat. No. 6,194,551, which is incorporated herein by reference in its entirety.

In some examples, an Fc region described herein comprises one or more amino acid residues at amino acid positions 231 and/or 239 to alter the ability of the antibody to fix complement. See, e.g., PCT Publication WO 94/29351, which is incorporated herein by reference in its entirety.

In some examples, an Fe region described herein can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include but are not limited to 236A, 239D, 239E, 268D, 267E, 268E, 268F, 324T, 332D, and 332E. Exemplary variants include but are not limited to 239D/332E, 236A/332E, 236A/239D/332E, 268F/324T, 267E/268F, 267E/324T, and 267E/268F/324T.

In some embodiments, an Fc region described herein include modifications that reduce or ablate binding to FcγR and/or complement proteins, thereby reducing or ablating Fc-mediated effector functions such as ADCC, ADCP, and CDC. Exemplary modifications include but are not limited to substitutions, insertions, and deletions at amino acid residues (EU numbering) 234, 235, 236, 237, 267, 269, 325, 328, 330, and/or 331. Exemplary substitutions include but are not limited to 234A, 235E, 236R, 237A, 267R, 269R, 325L, 328R, 330S, and 331S (e.g., 330S, and 331S). An exemplary Fc variant can comprise 236R/328R. Other modifications for reducing FcγR and complement interactions include substitutions 297A, 234A, 235A, 237A, 318A, 228P, 236E, 268Q, 309L, 330S, 331S, 2205, 226S, 229S, 2385, 233P, and 234V, as well as removal of the glycosylation at position 297 by e.g., mutational or enzymatic means or by production in organisms such as bacteria that do not glycosylate proteins.

In one embodiment, an Fc region described herein is a human IgG1.3 Fc constant region comprising L234A, L235E, and G237A substitutions. In one embodiment, an Fc region described herein is a IgG1fa.P238K (or IgG1.P238K) comprising a P238K substitution. In one embodiment, an Fc region described herein is a IgG1.lf variant comprising L234A, L235E, G237A, A330S, and P331S substitutions.

In some embodiments, an Fc region described herein is modified to enhance affinity for an inhibitory receptor FcγRIIb. Such modification can provide an Fc fusion protein with immunomodulatory activities related to FcγRIIb cells, including for example B cells and monocytes. For example, the Fc variants may provide selectively enhanced affinity to FcγRIIb relative to one or more activating receptors. Modifications that alter binding to FcγRIIb include one or more modifications at a position (EU numbering) selected from the group consisting of 234, 235, 236, 237, 239, 266, 267, 268, 325, 326, 327, 328, 330, 331, and 332. Exemplary substitutions for enhancing FcγRIIb affinity include but are not limited to 234A, 234D, 234E, 234F, 234W, 235D, 235E, 235F, 235R, 235Y, 236D, 236N, 237A, 237D, 237N, 239D, 239E, 266M, 267D, 267E, 268D, 268E, 327D, 327E, 328F, 328W, 328Y, 3305, 3315, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

Other modifications for enhancing FcγR and complement interactions include but are not limited to substitutions 298A, 333A, 334A, 326A, 2471, 339D, 339Q, 280H, 2905, 298D, 298V, 243L, 292P, 300L, 396L, 3051, and 396L. These and other modifications are reviewed in Strohl, 2009, Current Opinion in Biotechnology 20:685-691. Fc modifications that increase binding to an Fcγ receptor include amino acid modifications at any one or more of amino acid positions (EU numbering) 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region (see., Patent Publication No. WO 00/42072).

The affinities and binding properties of an Fc region for its ligand can be determined by a variety of in vitro methods (e.g., biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to fluorescent, luminescent, chromogenic, or isotopic labels.

In some embodiments, the glycosylation of an antibody is altered. Glycosylation can be altered to, for example, increase the affinity of the antibody for antigen. Such carbohydrate modifications can be accomplished by, for example, altering one or more sites of glycosylation within the antibody sequence. For example, glycosylation of the constant region on N297 can be prevented or reduced by mutating the N297 residue to another residue, e.g., N297A, or N297D and/or by mutating an adjacent amino acid, e.g., 298. Additionally, one or more amino acid substitutions can be made that result in elimination of one or more variable region framework glycosylation sites to thereby eliminate glycosylation at that site. Such aglycosylation can increase the affinity of the antibody for antigen. See, e.g., U.S. Pat. Nos. 5,714,350 and 6,350,861, both of which are incorporated herein by reference in their entirety.

In some embodiments, the antibody can be modified to increase its biological half-life. For example, the Fc region can be modified to increase its binding affinity for FcRn, by mutating one or more of following residues: 252, 254, 256, 433, 435, 436 (see, e.g., U.S. Pat. No. 6,277,375, which is incorporated herein by reference in its entirety). Specific exemplary mutations include one or more of the following: T252L, T254S, and/or T256F. In some embodiments, the antibody can be modified within the CH1 or CL region to contain a salvage receptor binding epitope taken from two loops of a CH2 domain of an Fc region of an IgG, as described in U.S. Pat. Nos. 5,869,046 and 6,121,022, each of which is incorporated herein by reference in its entirety.

Other exemplary variants that increase Fc binding to FcRn include: 250E, 250Q, 428L, 428F, 250Q/428L (Hinton et al. 2004, J. Biol. Chem. 279(8): 6213-6216; Hinton et al. 2006 Journal of Immunology 176:346-356, each of which is incorporated herein by reference in its entirety), 256A, 272A, 286A, 305A, 307A, 307Q, 311A, 312A, 376A, 378Q, 380A, 382A, 434A (Shields et al., Journal of Biological Chemistry, 2001, 276(9):6591-6604, which is incorporated herein by reference in its entirety), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524, each of which is incorporated herein by reference in its entirety). Additional exemplary variants that increase binding to FcRn and/or improve pharmacokinetic properties include mutations at positions 259, 308, 428, and 434, including for example 2591, 308F, 428L, 428M, 434S, 434H. 434F, 434Y, and 434M. Further modifications for modulating FcRn binding include those described in Yeung et al., 2010, J Immunol, 182:7663-7671, which is incorporated herein by reference in its entirety.

In some embodiments, antibodies described herein are of hybrid IgG isotypes. In some embodiments, an IgG1/IgG2 hybrid variant can be constructed by substituting IgG2 positions in the CH2 and/or CH3 region with amino acids from IgG1 at positions where the two isotypes differ. In some embodiments, a hybrid variant IgG antibody can be constructed that comprises one or more substitutions, e.g., one or more of the following amino acid substitutions: 233E, 234L, 235L, −236G (referring to an insertion of a glycine at position 236), and 327A. In some embodiments, an IgG1/IgG3 hybrid variant can be constructed by substituting IgG1 positions in the CH2 and/or CH3 region with the amino acids from IgG3 at positions where the two isotypes differ. In some embodiments, a hybrid variant IgG antibody can be constructed that comprises one or more substitutions, e.g., 274Q, 276K, 300F, 339T, 356E, 358M, 384S, 392N, 397M, 4221, 435R, and/or 436F.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006).

In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/I332E, S239D/I332E/A330L, L235V/F243L/R292P/Y300L/P396L, and M428L/N434S.

In some embodiments, an Fc region described herein may have reduced binding to FcγRs. For example, the Fc region (e.g., IgG1 Fc) with reduced FcγR binding may comprise the following three amino acid substitutions: L234A, L235E and G237A.

In some embodiments, an Fc region described herein may have reduced complement fixation. For example, the Fc region (e.g., IgG1 Fc) with reduced complement fixation may have the following two amino acid substitutions: A330S and P331S.

In some embodiments, an Fc region described herein may have essentially no effector function, i.e., it has reduced binding to FcγRs and reduced complement fixation. For example, an “effectorless” Fc region (e.g., IgG1 Fc) may comprise the following five mutations: L234A, L235E, G237A, A330S and P331S.

In some embodiments, an Fc region described herein may include mutations L234A and/or L235A (EU numbering), which can suppress FcgR binding, and/or a P329G mutation (EU numbering) to abolish complement C1q binding, e.g., to abolish all immune effector functions.

In some embodiments, an IgG4 Fe region described herein may include a S228P mutation, e.g., to stabilize stabilizes IgG4 molecules and suppress formation of half-antibodies. In some embodiments, an hIgG4 Fab described herein may comprise an exchange mutant sequence which may include a S228P mutation which can suppress Fab arm exchange.

In some embodiments, the Fc region may comprise a non-naturally occurring amino acid residue at additional and/or alternative positions as described in, e.g., PCT Patent Publications WO 00/42072; WO 01/58957; WO 02/06919; WO 04/016750; WO 04/029207; WO 04/035752; WO 04/063351; WO 04/074455; WO 04/099249; WO 05/040217; WO 05/070963; WO 05/092925 and WO 06/020114; and U.S. Pat. Nos. 5,624,821; 6,277,375; 6,737,056; 6,194,551; 7,317,091; 8,101,720; each of which is incorporated herein by reference in its entirety).

Other suitable Fc modifications are described in WO 2016/081746 or WO 2017/087678, both of which are incorporated herein by reference in their entirety.

In some embodiments, the Fe region described herein may be engineered to introduce one or more amino acids for site-specific conjugation with a heterologous moiety. In some embodiments, an IgG1 Fc has been modified to include S239C. In some embodiments, the CH1 domain of the IgG1 heavy chain constant region and/or the IgG1 light chain constant region (CL) may be engineered to introduce one or more amino acids for site-specific conjugation with a heterologous moiety (see, e.g., Junutula et al., J Immunol Methods. 2008 Mar. 20; 332(1-2):41-52), which is herein incorporated by reference in its entirety). In some embodiments, the CH1 domain of the IgG1 heavy chain constant region comprises a A121C mutation. In some embodiments, the IgG1 light chain constant region (CL) comprises a V110C mutation.

In some embodiments, a heterologous moiety is conjugated (e.g., by site-specific conjugation) to an antibody (e.g., via the Fc region) described herein by way of a glutamine residue and/or a lysine residue. In some embodiments, the glutamine residue is: (i) introduced to the N-terminus and/or C-terminus of a heavy chain of the antibody, (ii) introduced to the N-terminus and/or C-terminus of a light chain of the antibody, (iii) naturally present in a CH2 or CH3 domain of the antibody, (iv) introduced to the antibody by modifying one or more amino acids, and/or (v) Q295 or mutated from N297 to Q297 (N297Q).

In some embodiments, an antibody described herein is an aglycosylated antibody, i.e., an antibody that does not comprise a glycosylation sequence that can interfere with a transglutamination reaction, e.g., an antibody that does not comprise a saccharide group at N297 on one or more heavy chains according to the EU numbering system. In some embodiments, an antibody heavy chain of the present disclosure has an N297 mutation. In particular embodiments, an antibody heavy chain has an N297Q or an N297D mutation. The N-linked glycan at position 297 can be found as a core structure, common to all IgG found in human beings and rodents. Antibodies comprising such above-disclosed mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

### Polynucleotides and Vectors

In one aspect, the present disclosure provides one or more polynucleotides encoding an antibody or antigen-binding fragment as described herein. The isolated polynucleotides capable of encoding the variable domain segments provided herein may be included on the same, or different, vectors to produce antibodies or antigen-binding fragments.

In some embodiments, an isolated polynucleotide encoding an anti-CD97 antibody or antigen-binding fragment comprises a nucleotide sequence encoding a VH amino acid sequence of SEQ ID NO: 2, 10, 16, 22, 26, 32, 36, 42, 46, 50, 54, 56, 60, 64, 70, 74, 78, 81, 31, 73, 109, 110, or 111, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 1, 9, 15, 21, 25, 21, 35, 41, 21, 49, 21, 21, 59, 63, 69, 21, 77, 21, 21, 53, 109, 110, or 111, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 2, 10, 16, 22, 26, 32, 36, 42, 46, 50, 54, 56, 60, 64, 70, 74, 78, 81, 31, 73, 109, 110, or 111; and/or a nucleotide sequence encoding a VL amino acid sequence of SEQ ID NO: 1, 9, 15, 21, 25, 21, 35, 41, 21, 49, 21, 21, 59, 63, 69, 21, 77, 21, 21, or 53.

In some embodiments, an isolated polynucleotide encoding an anti-CD97 antibody or antigen-binding fragment comprises a nucleotide sequence encoding a VH amino acid sequence of SEQ ID NO: 2, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 1, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 2; and a nucleotide sequence encoding a VL amino acid sequence of SEQ ID NO: 1.

In some embodiments, an isolated polynucleotide encoding an anti-CD97 antibody or antigen-binding fragment comprises a nucleotide sequence encoding a VH amino acid sequence of SEQ ID NO: 10, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 10; and a nucleotide sequence encoding a VL amino acid sequence of SEQ ID NO: 9.

In some embodiments, an isolated polynucleotide encoding an anti-CD97 antibody or antigen-binding fragment comprises a nucleotide sequence encoding a VH amino acid sequence of SEQ ID NO: 109, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 109; and a nucleotide sequence encoding a VL amino acid sequence of SEQ ID NO: 9.

In some embodiments, an isolated polynucleotide encoding an anti-CD97 antibody or antigen-binding fragment comprises a nucleotide sequence encoding a VH amino acid sequence of SEQ ID NO: 110, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 110; and a nucleotide sequence encoding a VL amino acid sequence of SEQ ID NO: 9.

In some embodiments, an isolated polynucleotide encoding an anti-CD97 antibody or antigen-binding fragment comprises a nucleotide sequence encoding a VH amino acid sequence of SEQ ID NO: 111, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 9, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 111; and a nucleotide sequence encoding a VL amino acid sequence of SEQ ID NO: 9.

In some embodiments, an isolated polynucleotide encoding an anti-CD97 antibody or antigen-binding fragment comprises a nucleotide sequence encoding a VH amino acid sequence of SEQ ID NO: 16, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 15, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 16; and a nucleotide sequence encoding a VL amino acid sequence of SEQ ID NO: 15.

In some embodiments, an isolated polynucleotide encoding an anti-CD97 antibody or antigen-binding fragment comprises a nucleotide sequence encoding a VH amino acid sequence of SEQ ID NO: 22, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 22; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 26, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 25, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 26; and a nucleotide sequence encoding a VL amino acid sequence of SEQ ID NO: 25.

In some embodiments, an isolated polynucleotide encoding an anti-CD97 antibody or antigen-binding fragment comprises a nucleotide sequence encoding a VH amino acid sequence of SEQ ID NO: 32, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 32; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 36, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 35, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 36; and a nucleotide sequence encoding a VL amino acid sequence of SEQ ID NO: 35.

In some embodiments, an isolated polynucleotide encoding an anti-CD97 antibody or antigen-binding fragment comprises a nucleotide sequence encoding a VH amino acid sequence of SEQ ID NO: 42, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 41, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 42; and a nucleotide sequence encoding a VL amino acid sequence of SEQ ID NO: 41.

In some embodiments, an isolated polynucleotide encoding an anti-CD97 antibody or antigen-binding fragment comprises a nucleotide sequence encoding a VH amino acid sequence of SEQ ID NO: 46, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 46; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 50, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 49, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 50; and a nucleotide sequence encoding a VL amino acid sequence of SEQ ID NO: 49.

In some embodiments, an isolated polynucleotide encoding an anti-CD97 antibody or antigen-binding fragment comprises a nucleotide sequence encoding a VH amino acid sequence of SEQ ID NO: 54, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 54; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 56, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 56; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 60, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 59, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 60; and a nucleotide sequence encoding a VL amino acid sequence of SEQ ID NO: 59.

In some embodiments, an isolated polynucleotide encoding an anti-CD97 antibody or antigen-binding fragment comprises a nucleotide sequence encoding a VH amino acid sequence of SEQ ID NO: 64, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 63, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 64; and a nucleotide sequence encoding a VL amino acid sequence of SEQ ID NO: 63.

In some embodiments, an isolated polynucleotide encoding an anti-CD97 antibody or antigen-binding fragment comprises a nucleotide sequence encoding a VH amino acid sequence of SEQ ID NO: 70, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 69, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 70; and a nucleotide sequence encoding a VL amino acid sequence of SEQ ID NO: 69.

In some embodiments, an isolated polynucleotide encoding an anti-CD97 antibody or antigen-binding fragment comprises a nucleotide sequence encoding a VH amino acid sequence of SEQ ID NO: 74, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 74; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 78, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 77, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 78; and a nucleotide sequence encoding a VL amino acid sequence of SEQ ID NO: 77.

In some embodiments, an isolated polynucleotide encoding an anti-CD97 antibody or antigen-binding fragment comprises a nucleotide sequence encoding a VH amino acid sequence of SEQ ID NO: 81, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 81; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 31, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 21, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 31; and a nucleotide sequence encoding a VL amino acid sequence of SEQ ID NO: 21.

In some embodiments, an isolated polynucleotide encoding an anti-CD97 antibody or antigen-binding fragment comprises a nucleotide sequence encoding a VH amino acid sequence of SEQ ID NO: 73, or a sequence having at least 70%, 75%, 80%, 85%, 90%, preferably 95% or more, such as 95%, 96%, 97%, 98%, or 99% identity thereto; and/or a nucleotide sequence encoding a VL amino acid sequence of SEQ ID NO: 53, or a sequence having at least 70%, 75%, 80%, 85%, 90%, preferably 95% or more, such as 95%, 96%, 97%, 98%, or 99% identity thereto.

In some embodiments, an isolated polynucleotide encoding an anti-CD97 antibody or antigen-binding fragment comprises a nucleotide sequence encoding a VH amino acid sequence of SEQ ID NO: 73; and a nucleotide sequence encoding a VL amino acid sequence of SEQ ID NO: 53.

In some embodiments, the polynucleotide is a DNA molecule or a derivative thereof.

In some embodiments, the polynucleotide is an RNA molecule (e.g., mRNA) or a derivative thereof.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors. In some embodiments, the vector is a viral vector.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene, a puromycin resistance gene, a blasticidin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Non-limiting examples of additional vectors that can be used in accordance with the present disclosure include Moloney murine leukemia viruses (MLV), Moloney murine leukemia viruses pseudotyped with vesicular stomatitis virus G protein (MLV-VSV-G), murine stem cell viruses (MSCV), lentiviruses, lentiviruses pseudotyped with vesicular stomatitis virus G protein (LV-VSV-G), adenoviruses (e.g., Ad5, Ad41), adeno-associated viruses (such as AAV1, AAV2, AAV5, AAV8, AAV9, AAV10, AAV6), and variants and derivatives thereof. Additional suitable vectors include those described in Buckinx and Timmermans, Histochem Cell Biol (2016) 146:709-720, which is incorporated herein by reference in its entirety.

In some embodiments, bacteriophages may be engineered to incorporate a nucleotide sequence encoding an antibody described herein into the genetic material of the phage so that the antibody may be exposed on the surface of the phage.

The vectors described herein may be used to transform various cells with the genes encoding the described antibodies or antigen-binding fragments. For example, the vectors may be used to generate CD97-specific antibody or antigen-binding fragment-producing cells. Thus, in another aspect is provided host cells transformed with vectors comprising a nucleic acid sequence encoding an antibody or antigen-binding fragment thereof that specifically binds a CD97, such as the antibodies or antigen-binding fragments described and exemplified herein.

In some embodiments, a host cell comprising a polynucleotide described herein or a vector described herein can express an antibody or an antigen-binding fragment of the present disclosure. Any of a wide variety of host cells within the knowledge of one of skill in the art may be used in the practice of the present disclosure. A non-limiting example of a host cell is a hybridoma. In some embodiments, the antibody or antigen-binding fragment is recombinantly produced.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

### Antibody-Drug Conjugates

In some embodiments, the present disclosure also provides an antibody-drug conjugate comprising the antibody or antigen-binding fragment described herein conjugated to a heterologous moiety. For example, the heterologous moiety can be a cytotoxic agent, an siRNA, a radionucleotide, a peptide, or an immune-stimulatory agent.

In some embodiments, a cytotoxic agent can include, e.g., an anti-tubulin agent, a tubulin polymerization inhibitor, a DNA synthesis inhibitor, a DNA intercalating agent, a DNA alkylating agent, a DNA cross-linking agent, a DNA cleaving agent, a platinum-based agent, topoisomerase I inhibitor, topoisomerase II inhibitor, an anti-microtubule agent, an anti-mitotic agent, a taxane-related anti-neoplastic agent, an anti-metabolite, an anti-tumor plant alkaloid, and an RNA polymerase II inhibitor.

Non-limiting examples of cytotoxic agents are doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin CI, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, 11-12, ranpimase, hTNF, IL-12, ranpimase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin.

Further non-limiting examples of cytotoxic agents are antiviral drugs (e.g., abacavir, acyclovir, ampligen, cidofovir, delavirdine, didanosine, efavirenz, entecavir, fosfonet, ganciclovir, ibacitabine, immunovir, idoxuridine, inosine, lopinavir, methisazone, nexavir, nevirapine, oseltamivir, penciclovir, stavudine, trifluridine, truvada, valaciclovir, and zanamivir), daunorubicin hydrochloride, daunoriycin, rubidomycin, cerubidine, idarubicin, doxorubicin, epirubicin and morpholino derivatives, phenoxizone biscyclopeptides (e.g., dactinomycin), basic glycopeptides (e.g., bleomycin), anthraquinone glycosides (e.g., plicamycin and mithramycin), anthracenediones (e.g., mitoxantrone), azirinopyrrolo indolediones (e.g., mitomycin), macrocyclic immunosuppressants (e.g., cyclosporine, FK-506, tacrolimus, prograf, andrapamycin), navelbene, CPT-11, anastrazole, letrazole, capecitabine, reloxafine, droloxafine, allocolchicine, Halichondrin B, colchicine and colchicine derivatives, rhizoxin, thiocolchicine, trityl cysterin, vinblastine sulfate, hydroxyurea, N-methylhydrazine, epidophyllotoxin, procarbazine, mitoxantrone, leucovorin, and tegafur, combretatstatin, chalicheamicin, maytansine, DM-I, netropsin, podophyllotoxin (e.g., etoposide and teniposide), baccatin and its derivatives, anti-tubulin agents, cryptophysin, combretastatin, vincristine, vincristine sulfate, vinblastine, vindesine, vinorelbine, VP-16, camptothecin, epothilone A, epothilone B, nocodazole, colchicines, colcimid, estramustine, cemadotin, discodermolide, eleutherobin, mechlorethamine, cyclophosphamide, melphalan, carmustine, lomustine, semustine, streptozocin, chlorozotocin, uracil mustard, chlormethine, chlorambucil, pipobroman, triethylenemelamine, triethylenethiophosphoramine, busulfan, dacarbazine, temozolomide, ytarabine, cytosine arabinoside, fluorouracil, 5-fluorouracil (5-FU), floxuridine, 6-thioguanine, 6-mercaptopurine, pentostatin, methotrexate, 10-propargyl-5,8-dideazafolate, 5,8-dideazatetrahydrofolic acid, leucovorin, NCA1, auristatin, auristatin E, DNA minor groove binding agents, DNA minor groove alkylating agents, enediyne, lexitropsin, duocarmycin, taxane, puromycin, dolastatin, maytansinoid, vinca alkaloid, AFP, MMAF, MMAE, AEB, AEVB, taxoids (e.g., paclitaxel and paclitaxel derivatives (TAXOL®, Bristol-Myers Squibb Oncology, Princeton, N.J.), ABRAXANE® (American Pharmaceutical Partners, Schaumberg, Ill.), as well as docetaxel and docetaxel derivatives), CC-1065, SN-38, topotecan, morpholino-doxorubicin, rhizoxin, cyanomorpholino-doxorubicin, dolastatin-10, echinomycin, fludarabine phosphate, pentostatine, gemcitabine, Ara-C, deoxycoformycin, mitomycins such as mitomycin-C, L-asparaginase, azathioprine, brequinar, antibiotics (e.g., anthracycline, gentamicin, cefalotin, vancomycin, telavancin, daptomycin, azithromycin, erythromycin, rocithromycin, furazolidone, amoxicillin, ampicillin, carbenicillin, flucloxacillin, methicillin, penicillin, ciprofloxacin, moxifloxacin, ofloxacin, doxycycline, minocycline, oxytetracycline, tetracycline, streptomycin, rifabutin, ethambutol, and rifaximin), enediyne antibiotics (e.g., calicheamicin, calicheamicin gammalI and calicheamicin omegaIl, and dynemicin, including dynemicin A),. “Taxanes” include paclitaxel, as well as any active taxane derivative or pro-drug. Chemotherapeutic agents such as erlotinib (TARCEVA®, Genentech/OSI Pharm.), bortezomib (VELCADE®, Millenium Pharm.), fulvestrant (FASLODEX®, AstraZeneca), sunitinib (Sutent®, Pfizer), letrozole (FEMARA®, Novartis), imatinib mesylate (GLEEVEC®, Novartis), PTK787/ZK 222584 (Novartis), oxaliplatin (Eloxatin®, Sanofi), leucovorin, lapatinib (TYKERB®, GSK572016, GlaxoSmithKline), lonafarnib (SCH 66336), sorafenib (BAY43-9006, Bayer Labs.), and gefitinib (IRESSA®, AstraZeneca), AG1478, AG1571 (SU 5271; Sugen), alkylating agents such as thiotepa and cyclosphosphamide (CYTOXAN®); alkyl sulfonates such as busulfan, improsulfan and piposulfan; antifolate antineoplastic such as pemetrexed (ALIMTA® Eli Lilly); aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (such as bullatacin and bullatacinone); a camptothecin (including the synthetic analogue topotecan); bryostatin; callystatin; CC-1065 (including its synthetic analogues adozelesin, carzelesin and bizelesin); cryptophycins (such as cryptophycin 1 and cryptophycin 8); dolastatin; duocarmycin (including its synthetic analogues KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE™ Cremophor-free, albumin, nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); mitoxantrone; NAVELBINE® vinorelbine; novantrone; teniposide; edatrexate; daunomycin; aminopterin; xeloda; ibandronate; topoisomerase inhibitor RFS 2000; difluoromethylornithine (DMFO); retinoids such as retinoic acid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; nitrosoureas such as carmustine, chlorozotocin, fotemustine, lomustine, nimustine, andranimnustine; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (ADRIAMYCIN®) (including morpholino-doxorubicin, cyanomorpholino-doxorubicin, 2-pyrrolino-doxorubicin and deoxydoxorubicin), epirubicin, esorubicin, idarubicin, marcellomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; and pharmaceutically acceptable salts, esters, acids, prodrugs, or derivatives of any of the above.

In some embodiments, the cytotoxic agent may be selected from monomethyl auristatin E (MMAE), azonafide, α-amanitin, duocarmycin TM, pyrrolobenzodiazepine (PBD), PNU-159682, and pharmaceutically acceptable salts, esters, and analogs thereof.

In some embodiments, a tubulin polymerization inhibitor is auristatin E, auristatin F, monomethyl Auristatin E (MMAE), monomethyl auristatin F (MMAF), monomethyl auristatin D (MMAD), mertansine/emtansine (DM1), avtansine/soravtansine (DM4), dimethylvaline-valine-dolaisoleuine-dolaproine-phenylalanine-p-phenylenediamine (AFP), 5-benzoylvaleric acid-auristatin E ester (AEVB), AEB, a maytansinoid, or ansamitocin.

In some embodiments, the DNA alkylating agent is duocarmycin.

In some embodiments, the DNA cross-linking agent is pyrrolobenzodiazepines (PBD) dimer SG3199.

In some embodiments, the DNA cleaving agent is calicheamicin.

In some embodiments, the topoisomerase I inhibitor is SN-38 or DXd.

In some embodiments, a heterologous moiety of the present disclosure is an oligonucleotide (e.g., siRNA or antisense oligonucleotide). In some embodiments, an oligonucleotide is described herein may be used, e.g., to silence CD97.

Non-limiting examples of immune-stimulatory agents include 4-1BB, 4-1BBL, a C-C motif chemokine Ligand 13 (CXCL13), a C-C motif chemokine ligand 19 (CCL19), a C-C motif chemokine ligand 21 (CCL21), a C-C motif chemokine ligand 4 (CCL4), a soluble lymphotoxin (sLT), a type I interferon, a type III interferon, adenosine deaminase 1 and 2, an agonist of CD40, an antagonist of TGF beta, an interferon gamma (IFNg), and flt3 ligand, CD40 ligand (CD40L), GITR, GITR ligand, ICOS, ICOS ligand, IL-12, IL-15, IL-18, IL-2, IL-21, IL-24, interferon alpha 1 (IFNa1), interferon alpha 4 (IFNa4), interferon gamma, macrophage inflammatory protein-1 alpha (MIP-1 alpha), measles P protein (measles P; MeV.P), OX40 ligand, OX40 or flt3, p14 fusion-associated small transmembrane (FAST) (P14-FAST), TNF alpha, and GM-CSF.

In some embodiments, the immune-stimulatory agent is an immune checkpoint inhibitor. Exemplary immune checkpoint inhibitors include, but are not limited to, a programmed cell death protein 1 (PD-1) inhibitor, a programmed death-ligand 1 (PD-L1) inhibitor, Lymphocyte Activation Gene 3 (LAG-3 or CD223) inhibitor, a cytotoxic T-lymphocyte-associated antigen 4 (CTLA-4) inhibitor, a Cluster of Differentiation 47 (CD47) inhibitor, a T cell immunoglobulin and mucin domain-containing protein 3 (TIM-3) inhibitor, a B7 homolog 3 protein (B7-H3 or CD276) inhibitor, a B7-H4 inhibitor, a V-domain Ig suppressor of T cell activation (VISTA or PD-1H) inhibitor, a T cell immunoreceptor with Ig and ITIM Domains (TIGIT) inhibitor, a signal regulatory protein alpha (SIRPA) inhibitor, a signaling lymphocytic activation molecule family members (SLAMF) inhibitor, a poliovirus receptor-related immunoglobulin domain-containing protein (PVRIG or CD112R) inhibitor, an adenosine A2A receptor (A2aR) inhibitor, an adenosine A2b receptor (A2bR) inhibitor, a G protein-coupled receptor 171 (GPR171) inhibitor, an insulin like growth factor binding protein 7 (IGFBP7) inhibitor, a Cluster of Differentiation 93 (CD93) inhibitor, a CD96 inhibitor, a CD226 inhibitor, a natural killer group protein 2A (NKG2A) inhibitor, a natural killer group protein 2D (NKG2D) inhibitor, a killer cell lectin like receptor G1 (KLRG1) inhibitor, a human endogenous retrovirus-H long terminal repeat-associating protein 2 (HHLA2) inhibitor, a killer cell immunoglobulin like receptor, three Ig domains and long cytoplasmic tail 3 (KIR3DL3) inhibitor, a sialic acid-binding immunoglobulin-like lectin (Sigelac)-15 inhibitor, a Cluster of Differentiation 24 (CD24) inhibitor, a sialic acid-binding immunoglobulin-like lectin (Sigelac)-10 inhibitor, a P-selectin glycoprotein ligand-1 (PSGL-1) inhibitor, a V-set and Ig domain-containing protein 3 (VSIG3 also BT-IgSF and IGSF11) inhibitor, a leucine rich repeats and immunoglobulin like domains 1 (LRIG1) inhibitor, a fibrinogen-like protein 1 (FGL1) inhibitor, a B and T lymphocyte attenuator (BTLA) inhibitor, a Leukocyte associated immunoglobulin like receptor 1 (LAIR-1) inhibitor, a Cluster of Differentiation 160 (CD160) inhibitor, a leukocyte immunoglobulin-like receptor subfamily B member 2 (LILRB2) inhibitor, a leukocyte immunoglobulin-like receptor (LILRB4) inhibitor, and any combinations thereof.

In some embodiments, the immune checkpoint inhibitor is a PD-1 inhibitor. Exemplary PD-1 inhibitors include, but are not limited to, nivolumab, pembrolizumab, BAT1308, acrixolimab, balstilimab, budigalimab, cadonilimab, camrelizumab, cemiplimab, cetrelimab, danvilostomig, dostarlimab, eciskafusp alfa, enlonstobart, ezabenlimab, fanastomig, fidasimtamab, finotonlimab, geptanolimab, iparomlimab, ivonescimab, izuralimab, lipustobart, lodapolimab, lomvastomig, lorigerlimab, nofazinlimab, ociperlimab, penpulimab, peresolimab, pidilizumab, pimivalimab, pradusinstobart, prolgolimab, pucotenlimab, reozalimab, retifanlimab, rilvegostomig, rosnilimab, rulonilimab, sabestomig, sasanlimab, serplulimab, sintilimab, spartalizumab, tebotelimab, tiragolumab, tislelizumab, tobemstomig, toripalimab, volrustomig, vudalimab, zeluvalimab, zimberelimab, a variant or any combinations thereof. In some embodiments, the immune checkpoint inhibitor is a PD-L1 inhibitor. Exemplary PD-L1 inhibitors include, but are not limited to, durvalumab, avelumab, atezolizumab, bintrafusp alfa, a variant or any combinations thereof.

In some embodiments, the immune checkpoint inhibitor is a LAG-3 inhibitor. Exemplary LAG-3 inhibitors include, but are not limited to, relatlimab (BMS-986016), ABL501, CB213, EMB-02, favezelimab (MK-420/22D2), fianlimab (REGN3767), FS118, GSK2831781 (IMP731), IBI323, ieramilimab (LAG525/IMP701/BAP050), miptenalimab (BI-754111/496G6), pavunalimab (XmAb841), Sym022, tebotelimab (MGD013), tobemstomig (RG-6139/RO-7247669), TSR-033, tuparstobart (INCAGNO2385), BGA-1953, or a variant or any combinations thereof.

In some embodiments, the immune checkpoint inhibitor is a CTLA-4 inhibitor. Exemplary CTLA-4 inhibitors include, but are not limited to, ipilimumab, tremelimumab, XmAb20717, ONC-392, XmAb22841, BMS-986249, ADG116, ATOR-1015, ADG126, YH001, botensilimab, HBM4003, lorigerlimab, SI-B003, AK104, KN046, quavonlimab, BNT316/ONC-392 (gotistobart), porustobart (HBM4003), or a variant or combination thereof.

In some embodiments, the immune checkpoint inhibitor is a TIGIT inhibitor. Exemplary TIGIT inhibitors include, but are not limited to, BMS-986207, ociperlimab, BGB-A1217, tiragolumab, domvanalimab, ASP8374, vibostolimab, IBI-939, etigilimab, COM902, M6223, EOS884448, BAT6021, HLX301, or a variant or any combinations thereof.

In some embodiments, the immune checkpoint inhibitor is a SLAMF inhibitor. In some embodiments, the SLAMF inhibitor is elotuzumab, or a variant thereof. In some embodiments, the immune checkpoint inhibitor is a PVRIG inhibitor. In some embodiments, the PVRIG inhibitor is COM701, JS009, or a variant thereof. In some embodiments, the immune checkpoint inhibitor is a CD96 inhibitor. In some embodiments, the CD96 inhibitor is GSK6097608, or a variant thereof.

In some embodiments, the immune-stimulatory agent is a 4-1BB agonist, a OX40 agonist, a CD40 agonist, a CD30 agonist, a GITR agonist, an ICOS agonist, a CD27 agonist, a CD28 agonist, a CD28H/TMIGD2 agonist, a NCR3 agonist, a NCR1 agonist, a NCR2 agonist, a 4-1BB agonist, a DR3 agonist, a CD226 agonist, a CRTAM agonist, a HVEM agonist, a TNFR1 agonist, a TNFR2 agonist, a CD2 agonist, a CD7 agonist, a STING agonist, a TLR4 agonist, a TLR7 agonist, a TLR8 agonist, or a TLR9 agonist, or any combinations thereof.

In some embodiments, the immune-stimulatory agent is a STING agonist, a TLR7 agonist, a TLR8 agonist, or a TLR9 agonist, or any combination thereof, as described in e.g., Tsuchikama et al., Nat Rev Clin Oncol. 2024 March; 21(3):203-223., which is herein incorporated by reference in its entirety.

In some embodiments, the number of heterologous moieties conjugated to an antibody or antigen-binding fragment described herein forms a ratio. In some embodiments, the ratio is referred to as a DAR (drug-to-antibody) ratio, in which the drug as referred to herein is a heterologous moiety described herein (e.g., a cytotoxic agent, an siRNA, a radionucleotide, a peptide, or an immune-stimulatory agent). In some embodiments, the DAR ratio of the heterologous moiety to the antibody or antigen-binding fragment is about 1, 2, 3, 4, 5, 6, 7, 8, 9, 10, 11, 12, 13, 14, 15, 16, 20, 24, 30, 36 or greater. In some embodiments, the DAR ratio of the heterologous moiety to the antibody or antigen-binding fragment is about 1 or greater. In some embodiments, the DAR ratio of the heterologous moiety to the antibody or antigen-binding fragment is about 2 or greater. In some embodiments, the DAR ratio of the heterologous moiety to the antibody or antigen-binding fragment is about 3 or greater. In some embodiments, the DAR ratio of the heterologous moiety to the antibody or antigen-binding fragment is about 4 or greater. In some embodiments, the DAR ratio of the heterologous moiety to the antibody or antigen-binding fragment is about 5 or greater. In some embodiments, the DAR ratio of the heterologous moiety to the antibody or antigen-binding fragment is about 6 or greater. In some embodiments, the DAR ratio of the heterologous moiety to the antibody or antigen-binding fragment is about 7 or greater. In some embodiments, the DAR ratio of the heterologous moiety to the antibody or antigen-binding fragment is about 8 or greater. In some embodiments, the DAR ratio of the heterologous moiety to the antibody or antigen-binding fragment is about 9 or greater. In some embodiments, the DAR ratio of the heterologous moiety to the antibody or antigen-binding fragment is about 10 or greater. In some embodiments, the DAR ratio of the heterologous moiety to the antibody or antigen-binding fragment is about 11 or greater. In some embodiments, the DAR ratio of the heterologous moiety to the antibody or antigen-binding fragment is about 12 or greater. In some embodiments, the DAR ratio of the heterologous moiety to the antibody or antigen-binding fragment is about 16 or greater. In some embodiments, the DAR ratio of the heterologous moiety to the antibody or antigen-binding fragment is about 20 or greater. In some embodiments, the DAR ratio of the heterologous moiety to the antibody or antigen-binding fragment is about 24 or greater.

In some embodiments, the DAR ratio of the heterologous moiety to the antibody or antigen-binding fragment is about 1, 2, 3, 4, 5, 6, 7, 8, 9, 10, 11, 12, 13, 14, 15, 16, 20, 24, 30, or 36. In some embodiments, the DAR ratio of the heterologous moiety to the antibody or antigen-binding fragment is about 1. In some embodiments, the DAR ratio of the heterologous moiety to the antibody or antigen-binding fragment is about 2. In some embodiments, the DAR ratio of the heterologous moiety to the antibody or antigen-binding fragment is about 3. In some embodiments, the DAR ratio of the heterologous moiety to the antibody or antigen-binding fragment is about 4. In some embodiments, the DAR ratio of the heterologous moiety to the antibody or antigen-binding fragment is about 5. In some embodiments, the DAR ratio of the heterologous moiety to the antibody or antigen-binding fragment is about 6. In some embodiments, the DAR ratio of the heterologous moiety to the antibody or antigen-binding fragment is about 7. In some embodiments, the DAR ratio of the heterologous moiety to the antibody or antigen-binding fragment is about 8. In some embodiments, the DAR ratio of the heterologous moiety to the antibody or antigen-binding fragment is about 9. In some embodiments, the DAR ratio of the heterologous moiety to the antibody or antigen-binding fragment is about 10. In some embodiments, the DAR ratio of the heterologous moiety to the antibody or antigen-binding fragment is about 11. In some embodiments, the DAR ratio of the heterologous moiety to the antibody or antigen-binding fragment is about 12. In some embodiments, the DAR ratio of the heterologous moiety to the antibody or antigen-binding fragment is about 13. In some embodiments, the DAR ratio of the heterologous moiety to the antibody or antigen-binding fragment is about 14. In some embodiments, the DAR ratio of the heterologous moiety to the antibody or antigen-binding fragment is about 15. In some embodiments, the DAR ratio of the heterologous moiety to the antibody or antigen-binding fragment is about 16.

Methods for making the antibody-drug conjugates disclosed herein is also contemplated as within the scope of this disclosure. In one aspect, provided herein is a method for making an antibody-drug conjugate described herein comprising
(a) contacting the antibody or antigen-binding fragment with the heterologous moiety under the conditions favorable for conjugation of the antibody or antigen-binding fragment to the heterologous moiety; and
(b) optionally, isolating the antibody-drug conjugate produced in step (a).

In order to facilitate the coupling between the heterologous moiety and the antibody, it is possible to directly conjugate the two agents, or to introduce a spacer molecule between them. Suitable spacers include poly(alkylene) glycols such as polyethylene glycol, and peptide linkers. Many suitable coupling techniques are well known in the art. Suitable agents allowing covalent, electrostatic or noncovalent binding of the moiety to the antibody include benzoquinone, carbodiimide and more particularly EDC (1-ethyl-3-[3-dimethyl-aminopropyl]-carbodiimide hydrochloride), dimaleimide, dithiobis-nitrobenzoic acid (DTNB), N-succinimidyl S-acetyl thio-acetate (SATA), the bridging agents having one or more phenylazide groups reacting with the ultraviolets (U.V.) and preferably N-[-4-(azidosalicylamino)butyl]-3′-(2′-pyridyldithio)-propionamide (APDP), N-succinimid-yl 3-(2-25 pyridyldithio)propionate (SPDP), 6-hydrazino-nicotinamide (HYNIC). Another form of coupling, especially for the radioelements, includes the use of a bifunctional ion chelator. For example, chelates derived from EDTA or DTPA which have been developed for binding metals, especially radioactive metals, and immunoglobulins. Thus, DTPA and its derivatives can be substituted by different groups on the carbon chain in order to increase the stability and the rigidity of the ligand-metal complex, as is well known in the art.

In some embodiments, conjugation of a heterologous moiety to an antibody or antigen-binding fragment described herein is carried out via the covalent-nature binding, or binding by the use of adapter molecules or linkers.

Covalent binding requires prior activation of the heterologous moieties. In some embodiments, covalent strategies occur via carbodiimide chemistry, maleimide chemistry or “click chemistry”, as discussed in detail below.

In some embodiments, a heterologous moiety described herein (e.g., a cytotoxic agent) is conjugated to an anti-CD97 antibody or antigen-binding fragment. In some embodiments, a heterologous moiety described herein (e.g., a cytotoxic agent) is conjugated to an anti-CD97 antibody or antigen-binding fragment directly. In some embodiments, a heterologous moiety described herein (e.g., a cytotoxic agent) is conjugated to an anti-CD97 antibody or antigen-binding fragment via a linker covalently connecting the anti-CD97 antibody or antigen-binding fragment with the heterologous moiety.

In some embodiments, the heterologous moiety described herein (e.g., a cytotoxic agent) is conjugated to the anti-CD97 antibody or antigen-binding fragment by a chemical ligation process. In some embodiments, the heterologous moiety described herein (e.g., a cytotoxic agent) is conjugated to the anti-CD97 antibody or antigen-binding fragment by a native ligation. In some embodiments, the conjugation is as described in e.g., Dawson, et al. Science 1994, 266, 776-779; Dawson, et al. J. Am. Chem. Soc. 1997, 119, 4325-4329; Hackeng, et al. Proc. Natl. Acad. Sci. USA 1999, 96, 10068-10073; or Wu, et al. Angew. Chem. Int. Ed. 2006, 45, 4116-4125, each of which is herein incorporated by reference in its entirety. In some embodiments, the conjugation is as described in U.S. Pat. No. 8,936,910, which is herein incorporated by reference in its entirety. In some embodiments, the heterologous moiety described herein (e.g., a cytotoxic agent) is conjugated to the anti-CD97 antibody or antigen-binding fragment either site-specifically or non-specifically via native ligation chemistry.

In some embodiments, the heterologous moiety described herein (e.g., a cytotoxic agent) is conjugated to the anti-CD97 antibody or antigen-binding fragment by a site-directed method utilizing an enzyme-catalyzed process. In some embodiments, the site-directed method utilizes SMARTag™ technology (Catalent, Inc.). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized heterologous moiety described herein (e.g., a cytotoxic agent) via hydrazino-Pictet-Spengler (HIPS) ligation. (see Wu et al., PNAS 106(9): 3000-3005 (2009); Agarwal, et al., PNAS 110(1): 46-51 (2013), each of which is herein incorporated by reference in its entirety).

In some embodiments, the heterologous moiety described herein (e.g., a cytotoxic agent) is conjugated to the anti-CD97 antibody or antigen-binding fragment by a site-directed method utilizing a “traceless” coupling technology (Philochem). In some embodiments, the “traceless” coupling technology utilizes an N-terminal 1,2-aminothiol group on the anti-CD97 antibody or antigen-binding fragment which is then conjugated with a heterologous moiety described herein (e.g., a cytotoxic agent) containing an aldehyde group. (see Casi et al., JACS 134(13): 5887-5892 (2012), which is herein incorporated by reference in its entirety).

In some embodiments, the heterologous moiety described herein (e.g., a cytotoxic agent) is conjugated to the anti-CD97 antibody or antigen-binding fragment by a site-directed method utilizing an unnatural amino acid incorporated into the anti-CD97 antibody or antigen-binding fragment. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., PNAS 109(40): 16101-16106 (2012), which is herein incorporated by reference in its entirety).

In some embodiments, the enzyme-catalyzed process comprises transglutaminase (TG), e.g., microbial transglutaminase (mTG). In some cases, the heterologous moiety described herein (e.g., a cytotoxic agent) is conjugated to the anti-CD97 antibody or antigen-binding fragment utilizing a microbial transglutaminase-catalyzed process. In some embodiments, mTG catalyzes the formation of a covalent bond between the amide side chain of a glutamine within the recognition sequence and a primary amine of a functionalized heterologous moiety described herein (e.g., a cytotoxic agent). In some embodiments, mTG is produced from Streptomyces mobarensis. (see Strop et al., Chemistry and Biology 20(2) 161-167 (2013), which is herein incorporated by reference in its entirety).

In some embodiments, a sequence of amino acids comprising an acceptor glutamine residue are incorporated into (e.g., appended to) a polypeptide sequence, under suitable conditions, for recognition by a TG. This sequence leads to cross-linking by the TG through a reaction between an amino acid side chain within the sequence of amino acids and a reaction partner. The recognition tag may be a peptide sequence that is not naturally present in the polypeptide comprising the TG recognition tag. In some embodiments, the TG recognition tag comprises at least one Gln.

In some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gln, Ile, Met, Pro, Thr, Lys, or Trp or nonconventional amino acid). In some embodiments, the acyl donor glutamine-containing tag comprises an amino acid sequence selected from the group consisting of LLQ, LLQG (SEQ ID NO: 89), LLQGG (SEQ ID NO: 90), LSLSQG (SEQ ID NO: 91), GGGLLQGG (SEQ ID NO: 92), GLLQ (SEQ ID NO: 93), GLLQG (SEQ ID NO: 94), GLLQGGG (SEQ ID NO: 95), GLLQGG (SEQ ID NO: 96), GSPLAQSHGG (SEQ ID NO: 97), LLQLLQGA (SEQ ID NO: 98), LLQGA (SEQ ID NO: 99), LLQYQGA (SEQ ID NO: 100), LLQGSG (SEQ ID NO: 101), LLQYQG (SEQ ID NO: 102), LLQLLQG (SEQ ID NO: 103), SLLQG (SEQ ID NO: 104), LLQLQ (SEQ ID NO: 105), LLQLLQ (SEQ ID NO: 106), and LLQGR (SEQ ID NO: 107). See, e.g., PCT Publication No. WO2012/059882, which is herein incorporated by reference in its entirety. In some embodiments, the acyl donor glutamine-containing tag is present at the N-terminus of the antibody or antigen-binding fragment. In some embodiments, the acyl donor glutamine-containing tag is present at the C-terminus of the antibody or antigen-binding fragment. In some embodiments, the acyl donor glutamine-containing tag is present both at the N-terminus and the C-terminus of the antibody or antigen-binding fragment.

In some embodiments, the heterologous moiety described herein (e.g., a cytotoxic agent) is conjugated to the anti-CD97 antibody or antigen-binding fragment by a method which utilizes a sequence-specific transpeptidase (see, e.g., PCT Publication No. WO2014/140317, which is herein incorporated by reference in its entirety). Other conjugation methods include those described in U.S. Patent Publication Nos. 2015/0105539 and 2015/0105540, each of which is herein incorporated by reference in its entirety.

In some embodiments, the heterologous moiety described herein (e.g., a cytotoxic agent) is conjugated to the anti-CD97 antibody or antigen-binding fragment utilizing Azide-Alkyne Cycloaddition (CuAAC) click chemistry. Azides and alkynes can undergo catalyst free [3+2]cycloaddition by a using the reaction of activated alkynes with azides. Such catalyst-free [3+2]cycloaddition can be used in the methods described herein to conjugate an anti-CD97 antibody or antigen-binding fragment and the heterologous moiety described herein (e.g., a cytotoxic agent). Alkynes can be activated by ring strain such as, by way of example only, eight-membered ring structures, or nine-membered, appending electron-withdrawing groups to such alkyne rings, or alkynes can be activated by the addition of a Lewis acid such as, by way of example only, Au(I) or Au(III).

Alkynes activated by ring strain have been described and used in “copperless” [3+2]cycloaddition. Non-limiting examples include cyclooctynes and difluorocyclooctynes (Agard et al., J. Am. Chem. Soc., 126 (46):15046-15047 (2004)), dibenzocyclooctynes (PCT International Publication No. WO 2009/067663 A1 (2009)), aza-dibenzocyclooctynes (Debets et al., Chem. Comm., 46:97-99 (2010)), and cyclononynes (Dommerholt et al., Angew. Chem. 122:9612-9615 (2010)). In some embodiments, a tetrazine (Tzn)-activated anti-CD97 antibody or antigen-binding fragment may be cross-linked to a trans-cyclooctene (TCO)-activated heterologous moiety described herein (e.g., a cytotoxic agent). In some embodiments, a TCO-activated anti-CD97 antibody or antigen-binding fragment may be crosslinked to a Tzn-activated heterologous moiety described herein (e.g., a cytotoxic agent).

Complexes described herein may comprise a linker that connects an antibody or antigen-binding fragment to a heterologous moiety (e.g., a cytotoxic agent). A linker comprises at least one covalent bond. In some embodiments, a linker may be a single bond, e.g., a disulfide bond or disulfide bridge, that connects an antibody or antigen-binding fragment to a heterologous moiety (e.g., a cytotoxic agent). However, in some embodiments, a linker may connect an antibody or antigen-binding fragment to a heterologous moiety (e.g., a cytotoxic agent) through multiple covalent bonds. A linker is generally stable in vitro and in vivo, and may be stable in certain cellular environments. Additionally, generally a linker does not negatively impact the functional properties of either the antibody or antigen-binding fragment or the heterologous moiety (e.g., a cytotoxic agent).

A precursor to a linker typically will contain two different reactive species that allow for attachment to both the antibody or antigen-binding fragment and a heterologous moiety (e.g., a cytotoxic agent. In some embodiments, the two different reactive species may be a nucleophile and/or an electrophile. In some embodiments, a linker is connected to an antibody or antigen-binding fragment via conjugation to a lysine residue or a cysteine residue of the antibody or antigen-binding fragment. In some embodiments, a linker is connected to a cysteine residue of an antibody or antigen-binding fragment via a maleimide-containing linker, wherein optionally the maleimide-containing linker comprises a maleimidocaproyl or maleimidomethyl cyclohexane-1-carboxylate group. In some embodiments, a linker is connected to a cysteine residue of an antibody or antigen-binding fragment or thiol functionalized heterologous moiety via a 3-arylpropionitrile functional group. In some embodiments, a linker is connected to an antibody or antigen-binding fragment and/or a heterologous moiety (e.g., a cytotoxic agent) via an amide bond, a hydrazide, a triazole, a thioether or a disulfide bond.

In some embodiments, a linker described herein is a cleavable linker or a non-cleavable linker. In some embodiments, the linker is a cleavable linker. In other embodiments, the linker is a non-cleavable linker.

A cleavable linker may be a protease-sensitive linker, a pH-sensitive linker, or a glutathione-sensitive linker. These linkers are generally cleavable only intracellularly and are preferably stable in extracellular environments.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g. a cysteine residue.

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyneazide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a heterologous moiety comprising a (G), sequence (see, e.g., Proft T. Sortase-mediated protein ligation: an emerging biotechnology tool for protein modification and immobilization. Biotechnol Lett. 2010, 32(1):1-10).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species, an optionally substituted sulfur species, or a poly(alkylene oxide), e.g. polyethylene oxide or polypropylene oxide.

In some cases, the linker is a non-polymeric linker. A non-polymeric linker refers to a linker that does not contain a repeating unit of monomers generated by a polymerization process. Exemplary non-polymeric linkers include, but are not limited to, C1-C30 alkyl group (e.g., a C5, C4, C 3, C 2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C1-C30 alkyl group (e.g., a C5, C4, C 3, C 2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups. In some cases, the non-polymeric linker does not encompass a polyalkylene oxide (e.g., PEG). In some cases, the non-polymeric linker does not encompass a PEG.

In some embodiments, the linker comprises a homobifunctional linker. Exemplary homobifunctional linkers include, but are not limited to, bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-113-(4-azidosalicylamido)ethyl]disulfide (BASED), organoazide, organoalkyne, Lomant's reagent dithiobis (succinimidylpropionate) DSP, 3′3′-dithiobis(sulfosuccinimidyl proprionate (DTSSP), disuccinimidyl suberate (DSS), formaldehyde, glutaraldehyde, 1,4-butanediol diglycidyl ether, adipic acid dihydrazide, carbohydrazide, o-toluidine, 3,3′-dimethylbenzidine, benzidine, a,a′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Non-limiting examples of heterobifunctional linker include carbonyl-reactive and sulfhydrylreactive cross-linkers such as 4-(4-N-maleimidophenyl) butyric acid hydrazide (MPBH), 4-(N-maleimidomethyl) cyclohexane-1-carboxyl-hydrazide-8 (M2C2H), 3-(2-pyridyldithio)propionyl hydrazide (PDPH), amine-reactive and sulfhydryl cross-linkers such as N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LCsPDP), succinimidyloxycarbonyl-a-methyl-a-(2-pyridyldithio) toluene (sMPT), sulfosuccinimidyl-6-[a-methyl-a-(2-pyridyldithio)toluamido]hexanoate (sulfo-LC-sMPT), succinimidyl-4-(N-maleimidomethyl) cyclohexane-1-car-boxylate (sMCC), sulfosuccinimidyl-4-(N-maleimidomethyl) cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MB s), N-succinimidyl (4-iodoacteyl)aminobenzoate (sIAB), sulfosuccinimidyl (4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(y-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino) hexanoyl)amino]hexanoate (sIAXX), succinimidyl 4-(((iodoacetyl) amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyll,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido2′-nitrophenylamino)hexanoate (sANPAH), sulfo succinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl) butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), carboxylate-reactive and photoreactive cross-linkers such as 4-(p-azidosalicylamido) butylamine (AsBA), and arginine-reactive and photoreactive cross-linkers such as p-azidophenyl glyoxal (APG); sulfhydryl-reactive and photoreactive crosslinkers such as 1-(p-Azidosalicylamido)-4-(iodoacetamido) butane (AsIB), N-[4-(p-azidosalicylamido)butyl]-3′-(2′-pyridyldithio)propionamide (APDP), benzophenone-4-iodoacetamide, benzophenone-4-maleimide carbonylreactive and photoreactive cross-linkers such as p-azidobenzoyl hydrazide (ABH).

In some embodiments, the linker comprises a reactive functional group. In some cases, the reactive functional group comprises a nucleophilic group that is reactive to an electrophilic group present on an anti-CD97 antibody or antigen-binding fragment. Exemplary electrophilic groups include carbonyl groups such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (me). In some cases, the linker comprises maleimidocaproyl (me). In some cases, the linker is maleimidocaproyl (me). In other embodiments, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some embodiments, the self-stabilizing maleimide is a maleimide group described in Lyon, et al., Nat. Biotechnol. 32(10):1059-1062 (2014). In some embodiments, the linker comprises a self-stabilizing maleimide. In some embodiments, the linker is a self-stabilizing maleimide.

In some embodiments, the linker is a traceless linker or a linker in which after cleavage does not leave behind a linker moiety (e.g., an atom or a linker group) to an anti-CD97 antibody or antigen-binding fragment or a polynucleotide B. Exemplary traceless linkers include, but are not limited to, aryl-triazene linkers, germanium linkers, silicium linkers, sulfur linkers, selenium linkers, nitrogen linkers, phosphorus linkers, boron linkers, chromium linkers, or phenylhydrazide linker. In some embodiments, the linker is a traceless linker described in Blaney, et al., Chem. Rev. 102: 2607-2024 (2002) or U.S. Pat. No. 6,821,783.

In some embodiments, the linker is a linker described in U.S. Pat. Nos. 6,884,869; 7,498,298; 8,288,352; 8,609,105; or 8,697,688; U.S. Patent Publication Nos. US2014/0127239; US2013/028919; US2014/286970; US2013/0309256; US2015/037360; and US2014/0294851; or International Application Publication Nos. WO2015/057699; WO2014/080251; WO2014/197854; WO2014/145090; WO2014/177042, WO2022/015656, each of which is herein incorporated by reference in its entirety.

In some embodiments, a linker is absent. In some cases, a linker is a non-polymeric linker. In some cases, a linker is a polymeric linker.

In some embodiments, the linker comprises an alkyl group. In some embodiments, the linker comprises a C1-C30 alkyl group, or a C1-C24 alkyl group, or a C1-C20 alkyl group, or a C1-C16 alkyl group, or a C1-C12 alkyl group, or a C1-C10 alkyl group, or a C1-C8 alkyl group, or a C1-C6 alkyl group, or a C1-C4 alkyl group. In some cases, a linker is a C1-C6 alkyl group, such as for example, a C 3, C 4, C 3, C 2, or C1 alkyl group. In some cases, the C1-C6 alkyl group is an unsubstituted C1-C6 alkyl group. As used in the context of a linker, alkyl means a saturated straight or branched hydrocarbon radical containing up to six carbon atoms. In some embodiments, the linker comprises a homobifunctional linker or a heterobifunctional linker described herein.

In some cases, a linker is an oligomeric or a polymeric linker. In some embodiments, a linker is a natural or synthetic oligomer or polymer, consisting of branched or unbranched monomers, and/or cross-linked network of monomers in two or three dimensions. In some embodiments, the linker comprises a polysaccharide, lignin, rubber, or polyalkylen oxide (e.g., polyethylene glycol).

In some embodiments, polymeric linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. In some embodiments, the linker comprises polyalkylene oxide. In some embodiments, the linker comprises PEG. In some embodiments, the linker comprises polyethylene imide (PEI) or hydroxy ethyl starch (HES).

In some embodiments, the linker comprises a polyalkylene oxide (e.g., PEG) comprising discrete ethylene oxide units. In some cases, the linker comprises between about 2 and about 48 ethylene oxide units. In some cases, the polymer linker comprises about 2, about 3, about 4, about 5, about 6, about 7, about 8, about 9, about 10, about 11, about 12, about 13, about 14, about 15, about 16, about 17, about 18, about 19, about 20, about 24, about 30, about 36, about 42, or about 48 ethylene oxide units.

In some embodiments, the anti-CD97 antibody or antigen-binding fragment is conjugated to the heterologous moiety described herein (e.g., a cytotoxic agent) using a protamine linker (see, e.g., U.S. Patent Application Publication Nos. US2002/0132990, US2004/0023902, US2007/012152, and US2010/0209440, each of which is herein incorporated by reference in its entirety). In some embodiments, a protamine linker encompassed for use in the antibody-drug conjugates described herein comprises a sequence disclosed in US 2010/0209440, which is herein incorporated by reference in its entirety.

Acid cleavable linkers can also be used in the antibody-drug conjugates described herein and include, but are not limited to, bismaleimideothoxy propane, adipic acid dihydrazide linkers and acid labile transferrin conjugates that contain a sufficient portion of transferrin to permit entry into the intracellular transferrin cycling pathway. Conjugates linked via acid cleavable linkers should be preferentially cleaved in acidic intracellular compartments, such as the endosome.

Photocleavable linkers can also be used with in the antibody-drug conjugates described herein. Photocleavable linkers are cleaved upon exposure to light, thereby releasing the targeted agent upon exposure to light. Example photocleavable linkers may comprise nitrobenzyl group as a photocleavable protective group for cysteine; water soluble photocleavable copolymers, including hydroxypropylmethacrylamide copolymer, glycine copolymer, fluorescein copolymer and methylrhodamine copolymer; and nitrobenzyloxy carbonyl chloride cross linking reagents that produce photocleavable linkages. Such linkers are particularly useful in treating dermatological or ophthalmic conditions. In addition, other tissues, such as blood vessels that can be exposed to light using fiber-optics during angioplasty in the prevention or treatment of restenosis may benefit from the use of photocleavable linkers. After administration of the conjugate, the body part is exposed to light, resulting in release of the targeted moiety from the conjugate. Heat sensitive linkers would also have similar applicability.

In some embodiments, antibodies can be conjugated or recombinantly fused to a diagnostic, detectable or therapeutic agent or any other molecule. The conjugated or recombinantly fused antibodies can be useful, e.g., for monitoring or prognosing the onset, development, progression and/or severity of a disease as part of a clinical testing procedure, such as determining the efficacy of a particular therapy. Such diagnosis and detection can be accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹¹³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁷Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Encompassed herein are antibodies recombinantly fused or chemically conjugated (including both covalent and non-covalent conjugations) to a heterologous protein or polypeptide (or fragment thereof, preferably to a polypeptide of about 10, about 20, about 30, about 40, about 50, about 60, about 70, about 80, about 90 or about 100 amino acids) to generate fusion proteins. In particular, provided herein are fusion proteins comprising an antigen-binding fragment of a monoclonal antibody (e.g., a Fab fragment, Fd fragment, Fv fragment, F(ab)2 fragment, a VH domain, a VH CDR, a VL domain or a VL CDR) and a heterologous protein, polypeptide, or peptide. In a specific embodiment, the heterologous protein, polypeptide, or peptide that the antibody is fused to is useful for targeting the antibody to a particular cell type.

In one embodiment, a fusion protein provided herein comprises an anti-CD97 antibody described herein, or an antigen-binding fragment thereof, and a heterologous polypeptide. In another embodiment, a fusion protein provided herein comprises one, two, or more VH domains having the amino acid sequence of any one of the VH domains of an anti-CD97 antibody described herein or one or more VL domains having the amino acid sequence of any one of the VL domains of an anti-CD97 antibody described herein and a heterologous polypeptide. In another embodiment, a fusion protein provided herein comprises one, two, or more VH CDRs having the amino acid sequence of any one of the VH CDRs of an anti-CD97 antibody described herein and a heterologous polypeptide. In another embodiment, a fusion protein comprises one, two, or more VL CDRs having the amino acid sequence of any one of the VL CDRs of an anti-CD97 antibody described herein and a heterologous polypeptide. In another embodiment, a fusion protein provided herein comprises at least one VH domain and at least one VL domain of an anti-CD97 antibody described herein and a heterologous polypeptide. In yet another embodiment, a fusion protein provided herein comprises at least one VH CDR and at least one VL CDR of an anti-CD97 antibody described herein and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein.

Moreover, antibodies can be fused to marker sequences, such as a peptide to facilitate purification. In preferred embodiments, the marker amino acid sequence is a hexa-histidine peptide (SEQ ID NO: 115) (i.e., His-tag), such as the tag provided in a pQE vector (QIAGEN, Inc.), among others, many of which are commercially available. As described in Gentz et al., 1989, Proc. Natl. Acad. Sci. USA 86:821-824, for instance, hexa-histidine (SEQ ID NO: 115) provides for convenient purification of the fusion protein. Other peptide tags useful for purification include, but are not limited to, the hemagglutinin (“HA”) tag, which corresponds to an epitope derived from the Influenza hemagglutinin protein (Wilson et al., 1984, Cell 37:767), and the “flag” tag.

Methods for fusing or conjugating therapeutic moieties (including polypeptides) to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; C U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992; which are incorporated herein by reference in their entireties.

In particular, fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the monoclonal antibodies described herein or generated in accordance with the methods provided herein (e.g., antibodies with higher affinities and lower dissociation rates). See, e.g., U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding a monoclonal antibody described herein or generated in accordance with the methods provided herein may be recombined with one or more components, motifs, sections, parts, domains, fragments, etc. of one or more heterologous molecules.

An antibody can also be conjugated to a second antibody to form an antibody hetero-conjugate as described in U.S. Pat. No. 4,676,980, which is incorporated herein by reference in its entirety.

An antibody can also linked directly or indirectly to one or more antibodies to produce bispecific/multispecific antibodies.

An antibody can also be attached to solid supports, which are particularly useful for immunoassays or purification of an antigen. Such solid supports include, but are not limited to, glass, cellulose, polyacrylamide, nylon, polystyrene, polyvinyl chloride or polypropylene.

In some embodiments, one or more heterologous moieties is conjugated to an anti-CD97 antibody or antigen-binding fragment. In some embodiments, about 1, 2, 3, 4, 5, 6, 7, 8, 9, 10, 11, 12, 13, 14, 15, 16, 20, 24, 30, 36 or more heterologous moieties are conjugated to one anti-CD97 antibody or antigen-binding fragment. In some embodiments, 1 heterologous moiety is conjugated to one anti-CD97 antibody or antigen-binding fragment. In some embodiments, 2 heterologous moieties are conjugated to one anti-CD97 antibody or antigen-binding fragment. In some embodiments, 3 heterologous moieties are conjugated to one anti-CD97 antibody or antigen-binding fragment. In some embodiments, 4 heterologous moieties are conjugated to one anti-CD97 antibody or antigen-binding fragment. In some embodiments, 5 heterologous moieties are conjugated to one anti-CD97 antibody or antigen-binding fragment. In some embodiments, 6 heterologous moieties are conjugated to one anti-CD97 antibody or antigen-binding fragment. In some embodiments, 7 heterologous moieties are conjugated to one anti-CD97 antibody or antigen-binding fragment. In some embodiments, 8 heterologous moieties are conjugated to one anti-CD97 antibody or antigen-binding fragment. In some embodiments, 9 heterologous moieties are conjugated to one anti-CD97 antibody or antigen-binding fragment. In some embodiments, 10 or more heterologous moieties are conjugated to one anti-CD97 antibody or antigen-binding fragment.

### Preparation of Antibodies or Antigen-Binding Fragments

Antibodies or antigen-binding fragments capable of binding at least one isoform of CD97 as described herein can be made by any method known in the art, including but not limited to, recombinant technology.

For example, nucleic acids encoding the heavy and light chain of an anti-CD97 antibody as described herein can be cloned into one expression vector, each nucleotide sequence being in operable linkage to a suitable promoter. In one example, each of the nucleotide sequences encoding the heavy chain and light chain is in operable linkage to a distinct promoter.

Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-CD97 antibody, as also described herein. The recombinant expression vector can be introduced into a suitable host cell by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be co-incubated under suitable conditions allowing for the formation of the antibody.

In one example, two recombinant expression vectors are provided, one encoding the heavy chain of the anti-CD97 antibody and the other encoding the light chain of the anti-CD97 antibody. Both of the two recombinant expression vectors can be introduced into a suitable host cell by a conventional method, e.g., calcium phosphate-mediated transfection. Alternatively, each of the expression vectors can be introduced into suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-CD97 antibody as described herein, vectors (e.g., expression vectors) containing such; and host cells comprising the vectors are within the scope of the present disclosure.

In some embodiments, the antibody or antigen-binding fragment described herein are isolated from an animal immunized with a CD97-containing antigen, or genetically modified to produce the antibody or antigen-binding fragment. The animal may be genetically modified with the polynucleotide or the vector described herein.

In some embodiments, the animal is a dairy animal, such as, but not limited to, a goat, a cow, a buffalo, a sheep, or a camel. In such cases, the antibody or antigen-binding fragment may be isolated from milk produced by the dairy animal.

Anti-CD97 antibodies prepared as described herein can be characterized using methods known in the art, whereby reduction, amelioration, or neutralization of biological activity associated with the target CD97 is detected and/or measured. For example, in some embodiments, an ELISA-type assay is suitable for qualitative or quantitative measurement of binding of the antibody to the target CD97.

### Pharmaceutical Compositions

Any of the antibodies or antigen-binding fragments, antibody-drug conjugates, polynucleotides, or vectors described herein can be present in a pharmaceutical composition (such as a formulation) that can includes other agents, excipients, or stabilizers. In various embodiments, a pharmaceutical composition described herein may comprise (i) an antibody or antigen-binding fragment described herein, (ii) an antibody-drug conjugate described herein, (iii) a polynucleotide described herein, and/or (iv) a recombinant vector described herein, and a pharmaceutically acceptable carrier or adjuvant.

It is understood that the compounds of the present disclosure can uses amino acids independently selected from L and D forms (e.g., the peptide may contain two serine residues, each serine residue having the same or opposite absolute stereochemistry), etc., are intended for the use of both L- and D-form amino acids.

Accordingly, the compounds of the present disclosure also include substantially pure stereoisomeric form of the specific compound with respect to the asymmetric center of the amino acid residue, for example about 90% de, such as greater than about 95% to 97% de, or 99% de. For larger compounds, as well as mixtures thereof (such as racemic mixtures). Such diastereomers may be prepared, for example, by asymmetric synthesis using chiral intermediates, or the mixture may be divided by conventional methods, such as chromatography or the use of dividing agents.

If the compounds of the disclosure require purification, chromatographic techniques such as high-performance liquid chromatography (HPLC) and reverse phase HPLC can be used. Peptides may be characterized by mass spectrometry and/or other suitable methods.

If the compound contains one or more functional groups that can be protonated or deprotonated (e.g., at physiological pH), the compound can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

In some embodiments, it is envisioned that two or more combinations of the compounds of the disclosure will be administered to the subject. It is believed that the compound (s) may also be administered in combination with one or more additional therapeutic agents. This combination can allow separate, continuous or simultaneous administration with the other active ingredients of the above compounds. This combination may be provided in the form of a pharmaceutical composition.

Combination agents can be administered, for example, simultaneously or staggered in time (i.e., at different times and at equal or different time intervals for any part of a kit). The ratio of the total amount of combination agents administered in a combination can vary, e.g., to address the needs of a subpopulation of patients to be treated or the needs of a single patient, and different needs are the age of the patient, it can be due to gender, weight, etc.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

In some embodiments, the composition further comprises a targeting agent or a carrier that promotes the delivery of the inhibitors of endocytosis to an area affected by the chronic pain. Exemplary carriers include liposomes, micelles, nanodisperse albumin and its modifications, polymer nanoparticles, dendrimers, inorganic nanoparticles of different compositions.

The appropriate formulation for the compound of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The formulation may also include suitable excipients, such as antioxidants. Examples of antioxidants include phenolic compounds such as BHT or Vitamin E, reducing agents such as methionine or sulfites, and metal chelating agents such as EDTA.

The compounds or pharmaceutically acceptable salts thereof described herein can be prepared in parenteral dosage forms such as those suitable for, e.g., intravascular (intravenous or intraarterial), intraperitoneal, intratumoral, intraventricular, intrapleural or intramuscular administration delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol and). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. Suitable pharmaceutical forms for injection can be delivered by any suitable route, including intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. A dosage unit form may contain a predetermined amount of active substance calculated to produce a desired therapeutic effect described herein. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 g to about 2000 mg. Expressed in proportion, the active compound may be present in a carrier of about 0.25 g to about 1000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the inhibitor of endocytosis. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the inhibitor of endocytosis with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Kits

The present disclosure further provides a kit which may contain any of various compositions of the present disclosure, including antibodies or antigen-binding fragments thereof, antibody-drug conjugates, polynucleotides, or vectors of the disclosure.

In one aspect, a kit may comprise (i) an antibody-drug conjugate described herein, a polynucleotide described herein, and/or a vector described herein, and (ii) packaging for the same.

In some embodiments, a kit can comprise: (a) a container that contains a pharmaceutical composition described herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, a kit may further comprise, one or more of (i) a diluent, (ii) a buffer, (iii) a filter (iv) a syringe, and/or (v) a needle.

In some embodiments, the components of the kit may be provided in one or more liquid solutions. A liquid solutions described herein may be an aqueous solution such as a sterile aqueous solution. The components of the kit may also be provided as solids, which may be converted into liquids such as by addition of suitable solvents, which may be provided in another distinct container.

In some a pharmaceutical composition described herein may be lyophilized.

In some embodiments, kits may comprise a lyophilized formulation described herein in a suitable container and instructions for its reconstitution and/or use. Non-limiting examples of suitable containers include, e.g., syringes (such as dual chamber syringes), vials (such as dual chamber vials), bottles, and test tubes. In various embodiments, a container may be a multi-use container. The container may be formed from a variety of materials such as plastic or glass. The kit and/or container may contain instructions upon or accompanying the container which can denote directions for reconstitution of, e.g., a lyophilized formulation and/or use of the kit. In some embodiments, a label may denote that the lyophilized formulation is to be reconstituted to an appropriate concentration. The label may denote that the formulation is useful or intended for any route of administration disclosed herein.

The container containing the formulation may be a multi-use vial, which may allow for repeat administrations (e.g., from 2-6 administrations) of a reconstituted formulation. The kit may further comprise a second container comprising a suitable diluent (e.g., sodium bicarbonate solution).

Upon mixing of the diluent and a lyophilized formulation, a final concentration in the reconstituted formulation can reached. The kit may further include other materials desirable from a commercial and/or user perspective, including, e.g., other filters, needles, syringes, buffers, diluents, and/or package inserts which may comprise, e.g., instructions for use.

Kits may contain a single container that contains the formulation of the pharmaceutical composition with or without other components (e.g., other compounds or pharmaceutical compositions of such other compounds) or may have a separate container for each component.

Kits may include a formulation of the disclosure packaged for use in combination with the co-administration of a second compound (such as adjuvants (e.g., GM-CSF, a natural product, a hormone or antagonist, an anti-angiogenesis agent or inhibitor, an apoptosis-inducing agent or a chelator a chemotherapeutic agent) or a pharmaceutical composition thereof. The components of the kit may pre-mixed and/or pre-complexed or each component of the kit may be in a separate distinct container prior to administration to a patient.

In some embodiments, the container of a therapeutic kit may be a vial, flask, test tube, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiments, a kit may contain an apparatus (e.g., syringes, one or more needles, pipettes, eye droppers, etc.) which may permit administration of agents of the disclosure which are components of the kit.

### Methods of Use

The pharmaceutical compositions comprising, e.g., antibodies and antigen-binding fragments, antibody-drug conjugates, polynucleotides, and/or vectors of the present disclosure, and e.g., a pharmaceutically acceptable carrier and/or diluent disclosed herein, may be used for various therapeutic applications (in vivo and ex vivo) and as research tools.

In one aspect, the present disclosure provides a method of killing a cell expressing at least an isoform of CD97, the method comprising contacting the cell with an effective amount of an antibody or antigen-binding fragment described herein, an antibody-drug conjugate described herein, a polynucleotide described herein, a vector described herein, or a pharmaceutical composition described herein.

In some embodiments, the cell is a cancer cell.

In some embodiments, the cell expresses at least isoform 1 of CD97. In some embodiments, the cell expresses at least isoform 2 of CD97. In some embodiments, the cell expresses at least isoform 3 of CD97.

In some embodiments, the cell expresses isoform 1, isoform 2 and isoform 3 of CD97.

In some embodiments, the cell predominantly expresses isoform 3 of CD97.

In another aspect, the present disclosure provides a method of treating or preventing a cancer associated with at least an isoform of CD97 in a subject in need thereof, the method comprising administering to the subject an effective amount of an antibody or antigen-binding fragment described herein, an antibody-drug conjugate described herein, a polynucleotide described herein, a vector described herein, or a pharmaceutical composition described herein.

In some embodiments, the cancer is a hematologic cancer or solid cancer.

In some embodiments, the hematologic cancer is leukemia. Non-limiting examples of leukemia are acute myeloid leukemia (AML), acute lymphoblastic leukemia (ALL), acute leukemias of ambiguous lineage, chronic myeloid neoplasms (e.g., myeloproliferative or myelodysplastic neoplasms), non-Hodgkin and Hodgkin lymphomas, chronic leukemias (both myeloid and lymphoid), dendritic/histiocytic neoplasms, and other lymphoproliferative disorders.

A solid cancer may include, without limitation, glioma, thyroid cancer, lung cancer, colorectal cancer, head and neck cancer (e.g., head and neck squamous cell carcinoma), gastric cancer, stomach cancer, liver cancer, pancreatic cancer, renal cancer, urothelial cancer, prostate cancer, testis cancer, breast cancer, cervical cancer, endometrial cancer, ovarian cancer, gallbladder cancer, sarcoma (e.g., fibrosarcoma), or melanoma.

In some embodiments, the glioma is glioblastoma (GBM) or other forms of glioma.

In another aspect, the present disclosure provides a method of treating or preventing glioma in a subject in need thereof, the method comprising administering to the subject an effective amount of a CD97 inhibitor.

In some embodiments, the CD97 inhibitor is an antibody or an antigen-binding fragment or a small molecule, or a combination thereof.

In some embodiments, the CD97 inhibitor is an antibody or antigen-binding fragment described herein, an antibody-drug conjugate described herein, a polynucleotide described herein, or a vector described herein.

In some embodiments, the method further comprises administering one or more additional therapeutic agents. Non-limiting examples of additional therapeutic agents include chemotherapeutic agents, targeted therapies, stem cell transplantation radiotherapy, or a combination thereof.

In some embodiments, the one or more chemotherapeutic agents comprises an anthracycline, cytarabine, all-trans-retinoic acid, arsenic trioxide, a hypomethylating agent, a BH-3 mimetic (e.g. venetoclax), temozolamide, hydroxyurea, 6-thioguanine, cyclophosphamide, gemtuzumab ozogamicin, midostaurin, ivosidenib, enasidenib, gilteritinib, glasdegib, quizartinib, olutasidenib, vincristine, or a combination thereof.

In some embodiments, the anthracycline comprises daunorubicin and/or idarubicin.

In some embodiments, the hypomethylating agent comprises azacytidine, decitabine and/or guadecitabine.

In some embodiments, the glioma is glioblastoma (GBM).

In some embodiments, the subject is human.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Antibodies and Antibody-Drug Conjugates Targeting the Extracellular Regions of Human CD97

CD97 has been identified as an AML antigen, expressed in the vast majority of human AMLs. CD97 can be an excellent therapeutic target in AML: 1) CD97 is one of the most commonly expressed AML antigens; 2) CD97 regulates blast growth, survival, and differentiation; 3) CD97 regulates leukemia stem cell (LSC) function, as demonstrated in serial transplantation experiments of primary AML; and, 4) CD97 is not required for hematopoietic stem cell (HSC) function, suggesting low toxicity of CD97-targeting therapeutics. Highlighting its clinical importance, CD97 mRNA expression is an independent predictor of disease-free and overall survival in AML.

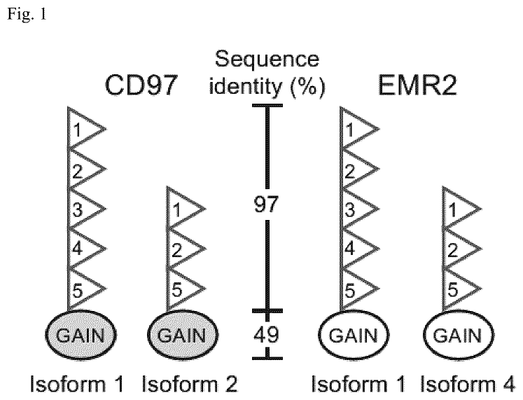

CD97 is an adhesion class G-protein coupled receptor (aGPCR) characterized by a large, extracellular ligand-binding domain and a GPCR-Autoproteolysis-INducing (GAIN) domain that can induce signals. Isoforms of CD97 produced by alternative splicing differ in the composition of the ligand-binding domain. These isoforms differ by the inclusion and exclusion of a total of five EGF-like domains, EGF1-EGF5, encoded by the CD97 gene in the extracellular region (FIG. 1) (2).

### Materials and Methods

### Bead-Based Binding Assay of Fab Clones

Binding titration of the Fab clones was performed by a bead binding assay, as described previously (7,8). Briefly, biotinylated antigens were immobilized on Dynabeads M-280 streptavidin (Thermo Fisher). The biotinylated Fab clones were titrated into a solution containing the antigen-coated beads. After incubation, the beads were washed three times and incubated with anti-human F(ab′)2-Alexa Fluor 647 (Jackson ImmunoResearch). The beads were further washed three times and analyzed on a HyperCyt (Intellicyt).

Competitive phage ELISA

Competitive phage ELISA was performed as described previously (3,5) with modifications. Briefly, the wells of the 96-well ELISA plate (Thermo Fisher) were coated with neutravidin (Thermo Fisher), and blocked with 0.5% (w/v) BSA in TBS. Biotinylated antigens were added to the wells, and the plate was washed with TBST (TBS containing 0.1% (v/v) Tween20) three times using the plate washer (BioTek). Purified anti-CD97 Fabs were first added to the plates and incubated for 30 min. The diluted culture supernatants containing phage were then transferred into the wells. After washing the wells with TBS-T three times, anti-M13HRP (Sino Biological) was added to the wells. One-step ultra TMB-ELISA solution (Thermo Fisher) was used as a substrate, then the reaction was stopped by adding 2 M sulfuric acid. The absorbance at 450 nm was measured using a BioTek Epoch plate reader (BioTek).

### Cell-Based Binding Assay of Anti-CD97 Antibodies

The cell-based binding assay was performed as described previously (9). Briefly, the cells were washed and suspended in PBS-BSA (PBS containing 1% (w/v) BSA (Gemini Bio)). The IgG1 LALA-PG antibodies were mixed with the cells and incubated at 4° C. for 30 min. After incubation, the cells were washed three times and incubated with anti-human F(ab′)2-Alexa Fluor 647 (Jackson ImmunoResearch). The cells were further washed three times and analyzed on a HyperCyt (Intellicyt).

For the epitope mapping assay and the CD97 mutational study, ExpiCHO cells (Thermo Fisher) were transfected with the vectors encoding truncation mutants of CD97 or CD97 GAIN mutants according to the manufacturer's protocol. After induction, the cells were stained with anti-CD97 IgG1 LALA-PG antibodies for the epitope mapping assay and the A8 Fab antibody for the mutational study in the presence of anti-V5 tag antibody (Sigma). After 30 min incubation at 4° C., the cells were washed three times and incubated with anti-human F(ab′)2-Alexa Fluor 488 (Jackson ImmunoResearch) and anti-rabbit IgG DyLight 650 (ThermoFisher). The cells were further washed three times and analyzed on a HyperCyt or iQue (Sartorius). The cell population expressing CD97 was first gated based on anti-V5 tag antibody staining, then the binding of anti-CD97 antibodies to the gated population was analyzed.

### Antibody Internalization Assay

Anti-CD97 antibodies were conjugated with the pHAb amine-reactive dye (Promega) according to the manufacturer's protocol. Dye-conjugated antibodies were purified using the Capturem Protein G miniprep columns (Takara). The dye-conjugated antibodies were then mixed with the cells and incubated at 37° C. with 5% CO₂. The cells were harvested and analyzed by flow cytometry to determine internalization. The fluorescence from pHAb dye was detected using the PE channel.

### Generation of Anti-CD97 Antibody-Drug Conjugate (ADC) and Cell Viability Assay

The drug conjugation reaction was performed as described previously (10). Briefly, the interchain disulfide bonds of A8 IgG1 LALA-PG were cleaved with DTT. A 9.5-fold molar excess of MC-Val-Cit-PAB-MMAF (BOC Science) was added to the reduced antibody. After 1 hour of incubation, the reaction was quenched by adding excess cysteine. The excess drug and cysteine were removed with the Zeba spin desalting column (Thermo Fisher). For the site-specific conjugation, a 10-fold molar excess of MC-Val-Cit-PAB-MMAF (BOC Science) or a 7.5-fold molar excess of MC-Val-Ala-PBD (MedchemExpress) was added to the antibody harboring a S239C mutation. After overnight incubation at room temperature, the excess drug was removed either by size-exclusion chromatography or buffer exchange using Amicon concentrators (Millipore).

For the cell viability assay, the cells were mixed with ADC (20,000 cells/well), and incubated at 37° C. with 5% CO₂ for 3 days. After incubation, cell viability was assessed by PrestoBlue assay (Thermo Fisher), following the manufacturer's protocol. The fluorescence was measured using a FlexStation 3 multi-mode microplate reader (Molecular Devices). Dose-response curves and respective ECso were generated using Prism software (GraphPad). For the cell-killing assay, a 96-well plate was coated with 0.1% poly-D-lysine. The cells were mixed with a diluted Incucyte® Annexin V Green Dye (Sartorius) and ADC (13,500 cells/well), and incubated in an Incucyte live-cell analysis system (Sartorius) at 37° C. with 5% CO₂ for 4 days. The percentage of dead cells was analyzed using Incucyte Software (Sartorious).

### Crvo-EM Grid Preparation

A8 Fab-CD97 isoform 1 complex at a concentration of 5 mg/ml was frozen in R0.6/1 gold foil on gold 300 mesh Cryo-EM grids (QUANTIFOIL) as follows. Cryo-EM grids were first hydrophilized using a PELCO easiGlow™ Glow Discharge Cleaning System (Ted Pella). (1H, 1H, 2H, 2H-Perfluorooctyl)-P-D-Maltopyranoside (Anatrace) was added to samples at a final concentration of 0.7 mM. The sample (3 μl) was loaded onto the hydrophilized grids and frozen in liquid ethane using a FEI Vitrobot™ Mark IV (Thermo Scientific).

### Cryo-EM Data Collection and Processing

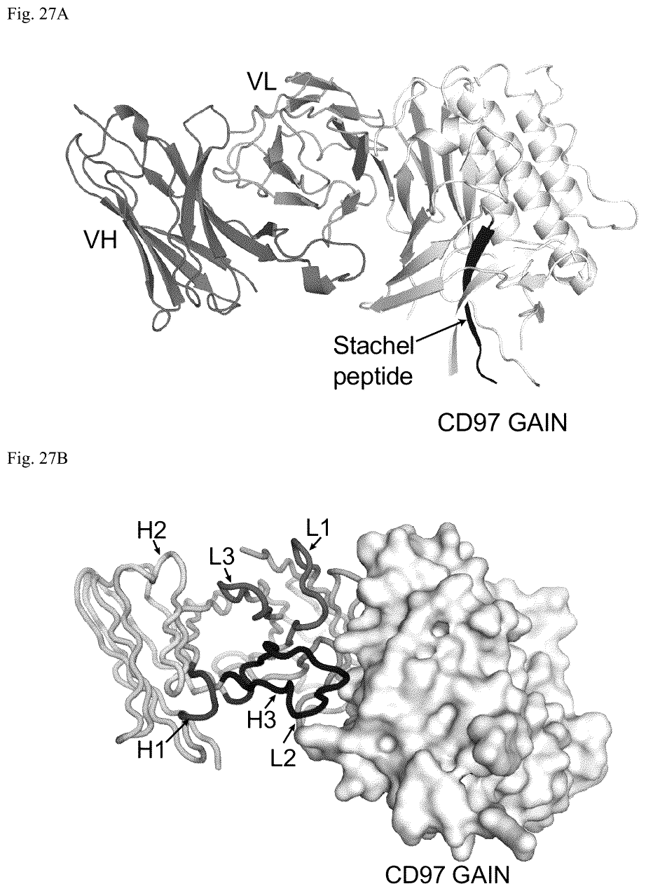

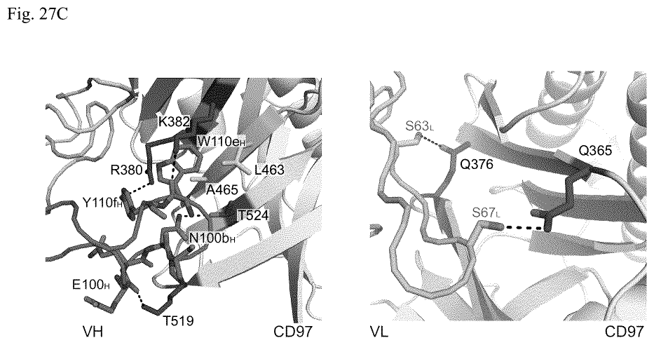

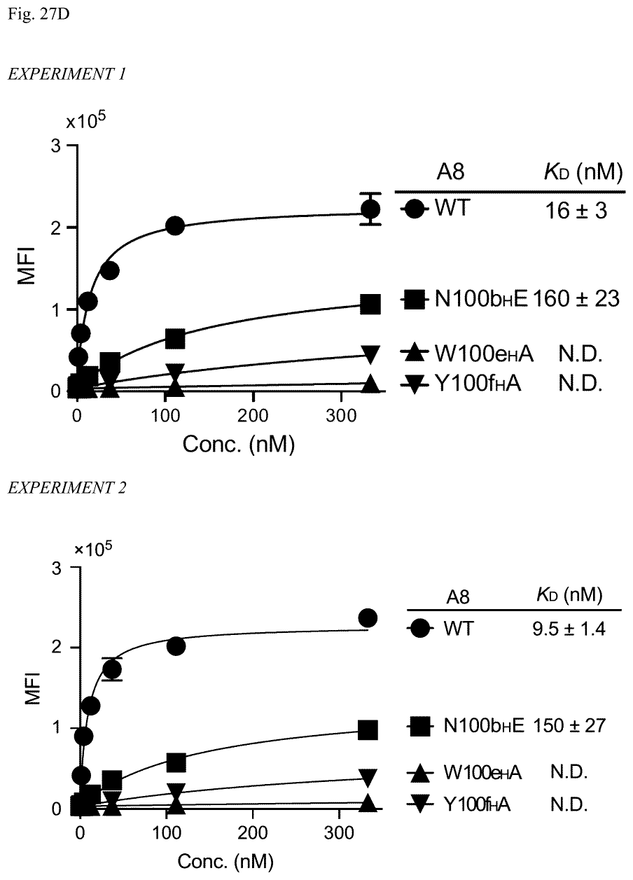

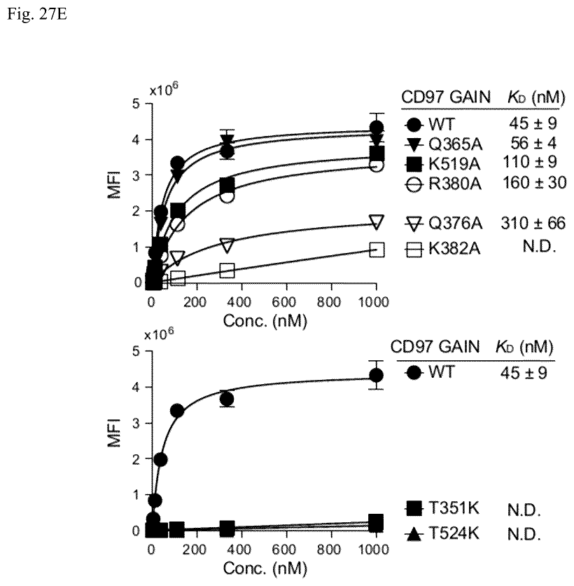

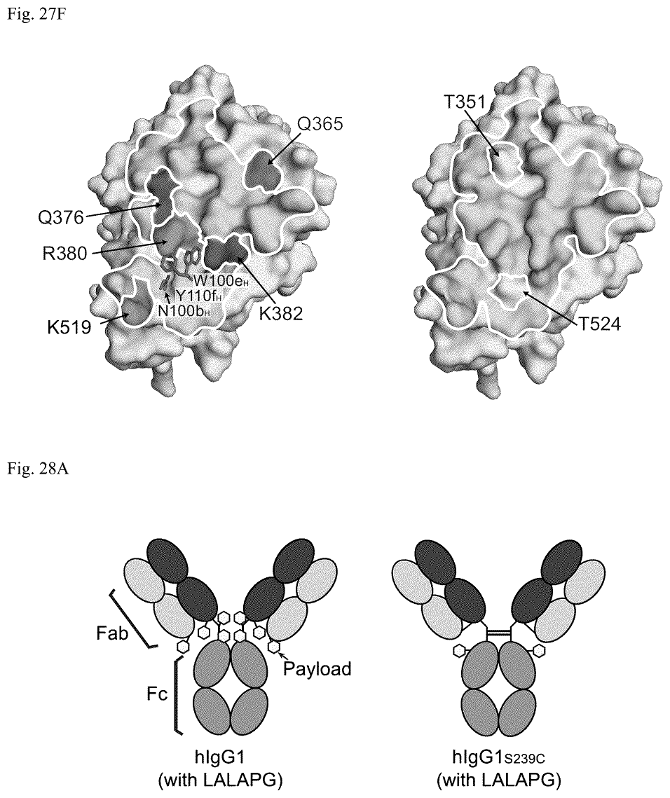

Cryo-EM data were collected using a Titan-Krios 300 kV TEM (Thermo Fisher scientific) equipped with a Gatan K3 camera. The 6,186 images were collected with a 105,000× magnification (final pixel size of images: 0.825 Å). Collected data were processed using the CryoSPARC™ software platform. Resulting maps were used to build structure models using Coot and PHENIX software. The computed structural model AF-P48960-F1 for CD97 isoform 1 and PDBID 6AZ2 for A8 Fab were used as starting models. Further refinement was performed iteratively using real-space refinement in PHENIX software and manual refinement with Coot software (FIGS. 27A-27F).

### MALDI-TOF Mass Spectrometry to Assess Drug-to-Antibody Ratio (DAR)

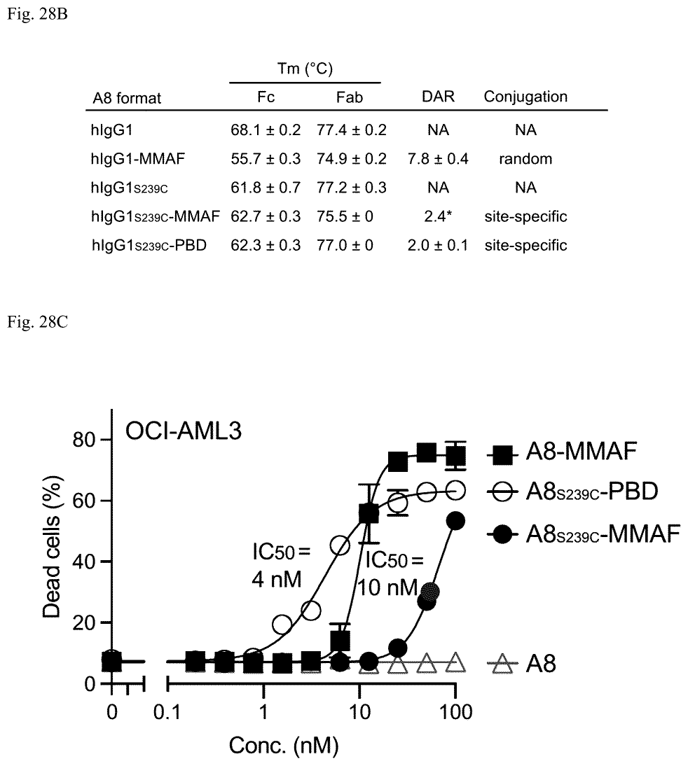

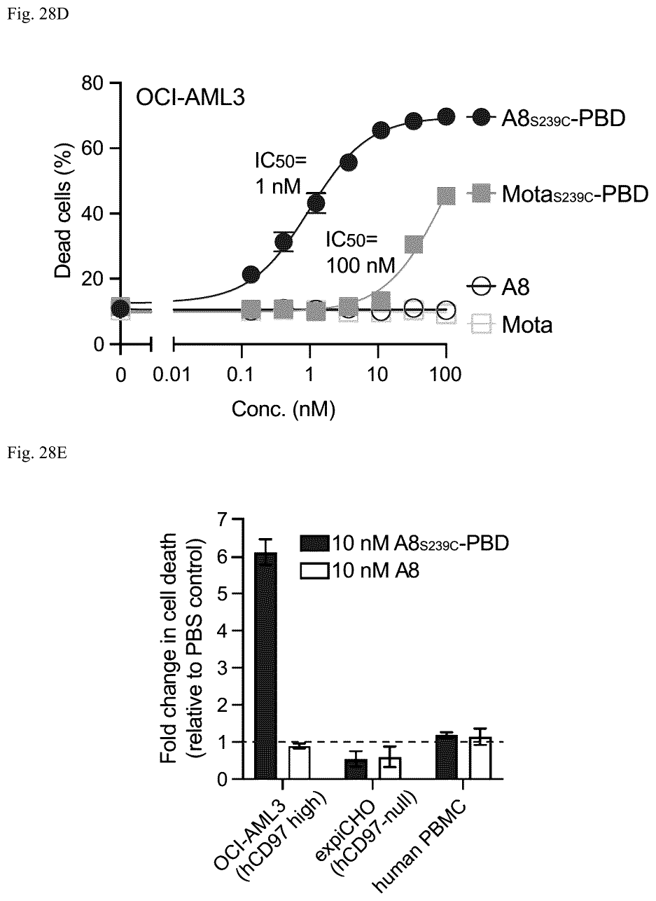

The protein samples (1 μl each) were mixed with 10 μl of matrix solution (10 mg/mL sinapinic acid in 50% acetonitrile, 0.05% TFA). Three microliters of the mixture were spotted on the target plate. The samples were analyzed using an autoflex maX mass spectrometer (Bruker). DARs of ADCs are calculated using the difference in mass between the ADC and the unconjugated antibody (FIGS. 28A-28B).

### Thermal Shift Assay

The protein samples (5 μL each) were mixed with 20 μL of a 1:50 dilution of 5,000× SYPRO-Orange (Thermo) and transferred into a 384-well PCR plate (Dot Scientific). The thermal shift assay was conducted using CFX384 Touch™ Real-Time PCR Detection System (BioRad). The derivative melt curves and Tm values were generated and determined using CFX Maestro software (BioRad) (FIGS. 28A-28B).

### Results

### Expression and Purification of CD97 Extracellular Regions

The genes encoding the ectodomains of CD97 (isoforms 1 and 2) and EMR2 (isoforms 1 and 4) (FIG. 1) were synthesized (Integrated DNA Technologies) and cloned into the mammalian expression vector pBCAG. The expi293F cells (Thermo Fisher) were transfected with the vectors using the ExpiFectamine 293 transfection kit (Thermo Fisher) according to the manufacturer's protocol. The cells were incubated at 37° C. with 8% CO₂. The proteins were purified from the cell culture supernatant by immobilized metal affinity chromatography (IMAC). The purified proteins were biotinylated in vitro using the BirA enzyme in the presence of 0.5 mM Biotin and 10 mM ATP. After the removal of the BirA enzyme by IMAC, the proteins were further purified by size exclusion chromatography using a Superdex 200 10/300 Increase (Cytiva). The purity of the proteins was confirmed by SDS-PAGE.

### Antibody Discovery

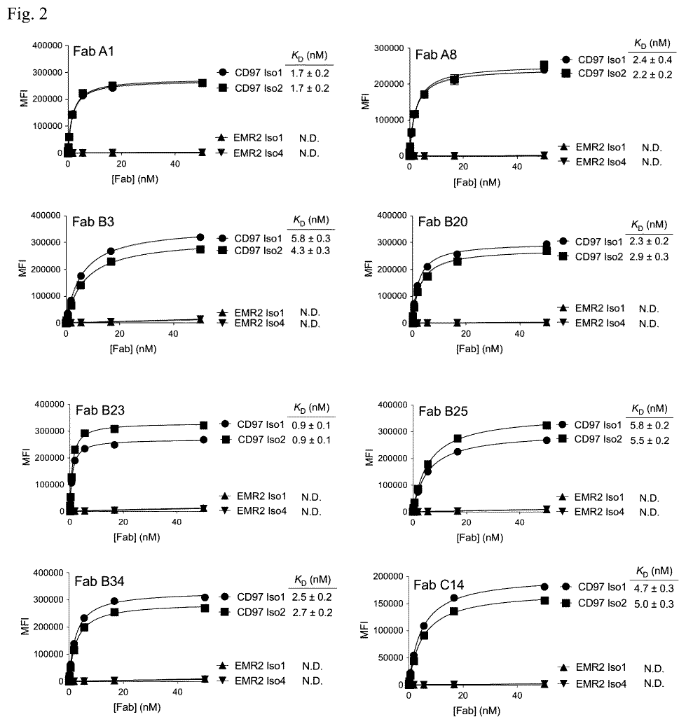

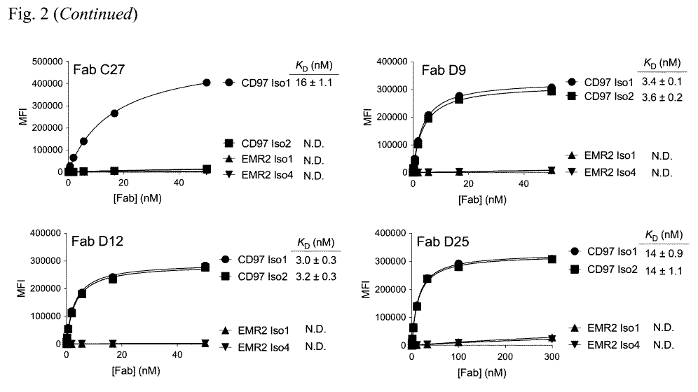

The sorting of a synthetic human antibody (sAb) library was performed as described previously (3,4). For the first antibody discovery campaign, a synthetic human antibody library was sorted using CD97 antigens at concentrations of 100 nM (the first round), 100 nM (the second round), 50 nM (the third round) and 20 nM (the fourth round). The identified antibody clones were first produced as Fab with a C-terminal biotinylation tag (Avi-tag), as described previously (3,4). Many clones that bound to CD97 with high affinity were identified (FIG. 2). Most of these clones bound to CD97 isoforms 1 and 2 but not to EMR2, suggesting their high selectivity. Clone C27 bound only to CD97 isoform 1, indicating the feasibility of developing isoform-selective antibodies against CD97.

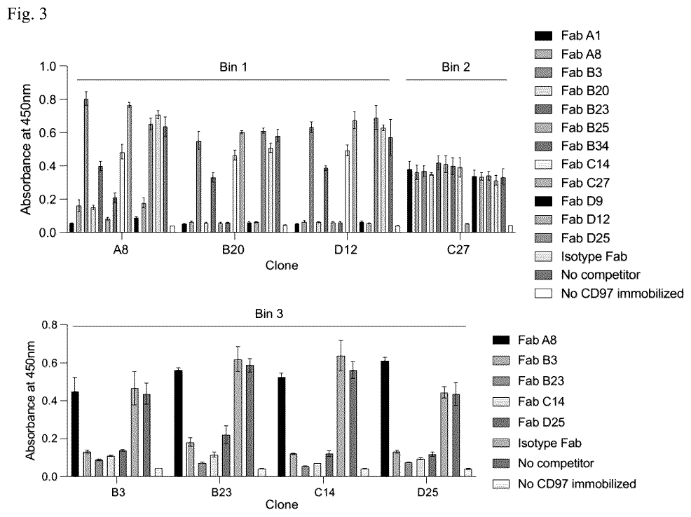

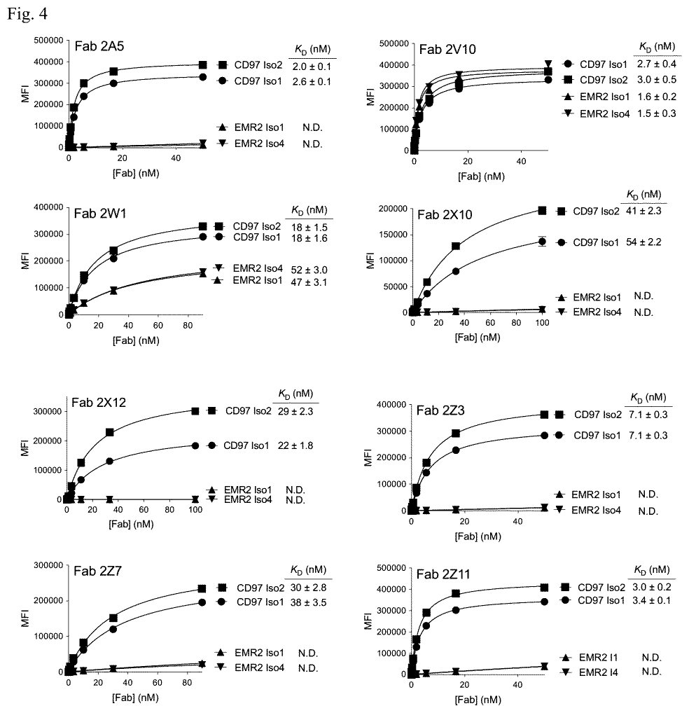

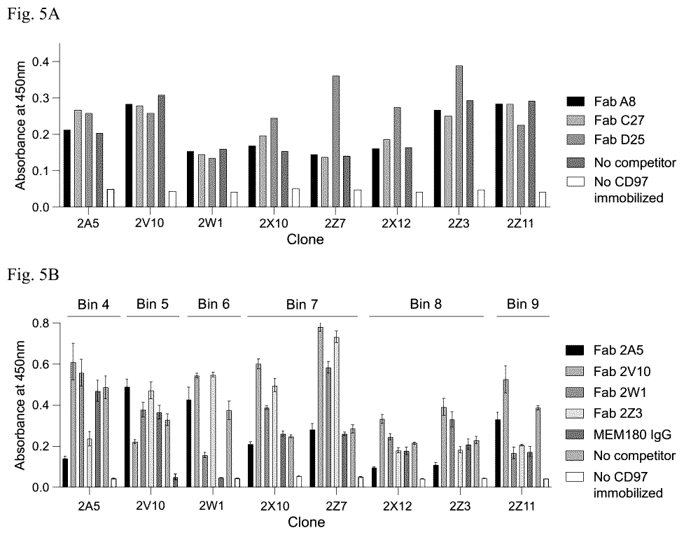

Epitope binning of these antibodies was performed by the competitive phage ELISA (3,5) using purified antibodies as competitors (FIG. 3). Judging from the patterns of competition, these antibodies were classified into three distinct epitope bins.

To identify additional antibodies that bind to epitopes outside the three epitopes identified above, the second antibody discovery campaign was performed. This time, the synthetic human antibody library was sorted by using purified CD97 proteins or OCI-AML3 cells in the presence of a mixture of Fabs identified from the first campaign in order to enrich clones that bind to novel epitopes. Additional antibody clones that showed high affinity were identified (FIG. 4). These new clones showed more diverse specificity profiles in terms of binding to CD97 isoforms and to EMR2 than the antibodies from the first campaign.

The epitopes of the new antibodies were assessed using competitive phage ELISA, first using antibodies from the first campaign as competitors. None of the competitors inhibited the binding of the new clones (FIG. 5A), indicating that the new clones indeed bound to distinct epitopes from those of the first set of antibodies. Additional competitive ELISA identified six additional epitope bins.

Altogether, many potent antibodies targeting a total of nine epitopes in the CD97 extracellular region were developed.

### Antibody Characterization Using CD97 on Cells

Next, representative antibodies were produced in the human IgG1 format with the LALA-PG mutations that abrogate Fc receptor binding (6). The genes encoding the VH domains of the antibodies were cloned into a modified version of pFUSEss-CHIg-hG1 (InvivoGen) harboring the LALA-PG (L234A, L235A, P329G) mutations, and the genes encoding the VL domains into pFUSEss-CLIg-hk (InvivoGen). These IgG1 proteins were produced using the ExpiCHO expression system according to the manufacturer's protocol (Thermo Fisher), and purified with Protein A affinity chromatography.

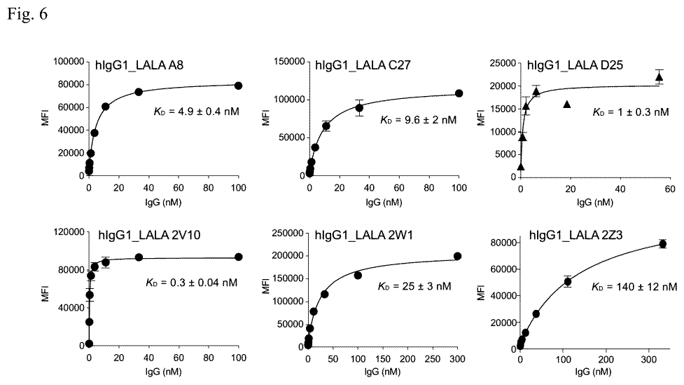

These antibodies potently bound to OCI-AML3 cells, suggesting that they recognize endogenous CD97 on the cell surface (FIG. 6).

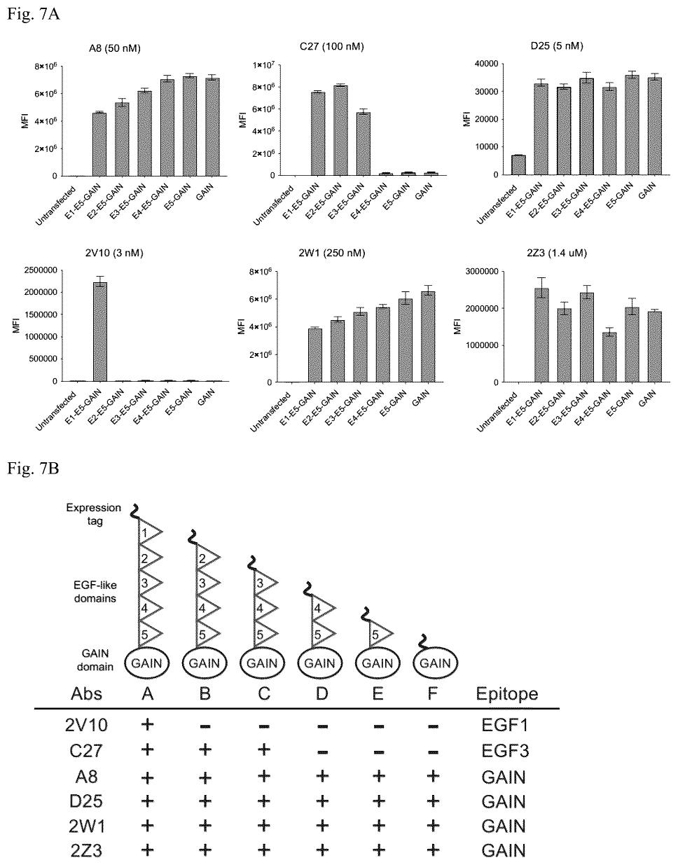

To further define the epitopes of these antibodies, truncation mutants of CD97 were engineered in which the EGF-like domains were systematically deleted from the N-terminus (FIG. 7), and antibody binding to cells expressing these variants was tested. The results showed that two antibodies, 2V10 and C27 bound to EGF1 and EGF3, respectively, and the other antibodies tested bound to the GAIN domain.

### Antibody Internalization

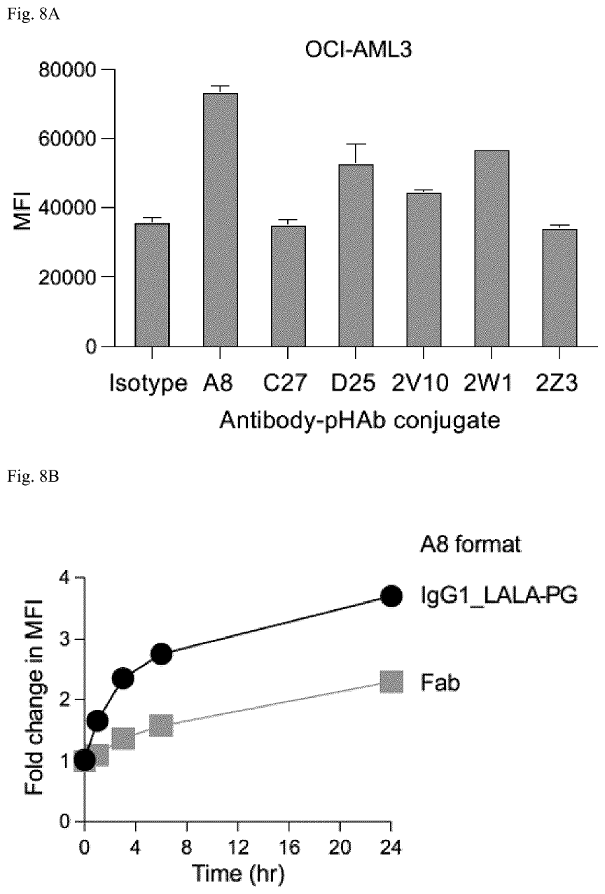

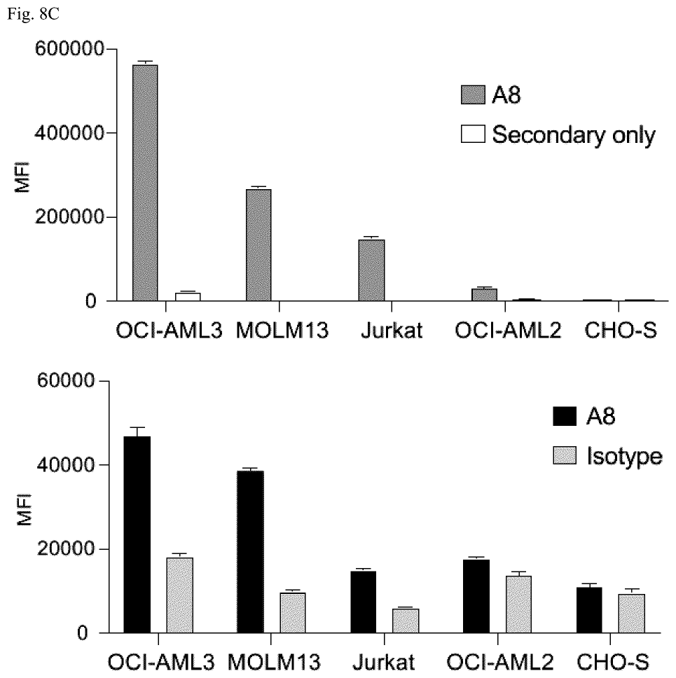

To identify antibodies that internalize into CD97-expressing cells, representative antibodies were conjugated with the pHAb dye and an internalization assay was performed. The pHAb dye is pH sensitive and has low or no fluorescence at a pH higher than 7. Upon internalization and acidification in endosomes and lysosomes, the dye becomes fluorescent. The antibodies showed varied degrees of fluorescence enhancement upon their addition to OCI-AML3 cells (FIG. 8A). Clone A8 showed the highest internalization among the tested antibodies. Its internalization efficiency was substantially higher in the IgG1 LALA-PG format than in the Fab format, suggesting the importance of the bivalent format for facilitating internalization.

A8 internalization was then tested using a panel of cell lines with different levels of CD97 on the surface (FIG. 8C). The internalization efficacy correlated with the expression level of CD97, further supporting that the internalization of A8 conjugate is mediated by CD97.

Selective Killing of AML Cells with Antibody-Drug Conjugate

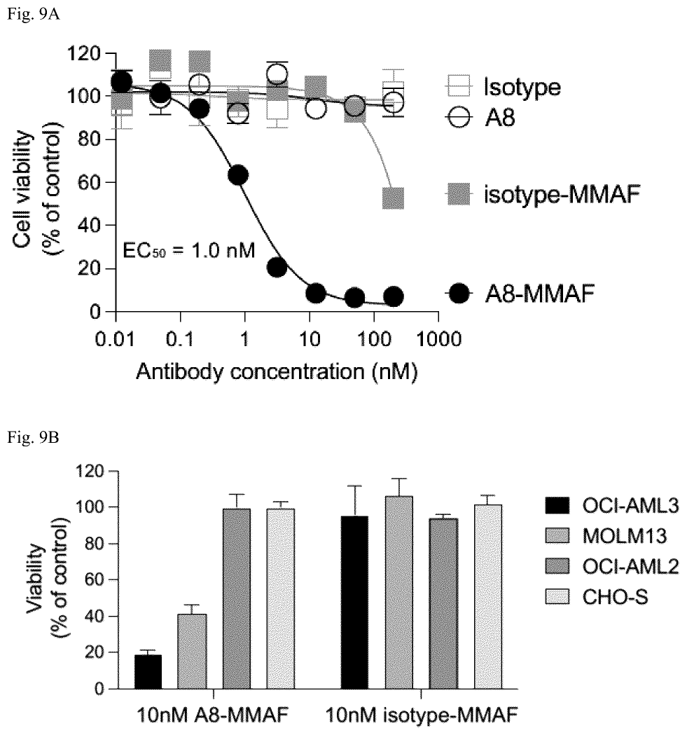

The A8 antibody conjugated with a cell-impermeable drug, MMAF, was prepared. This antibody-drug conjugate potently killed OCI-AML3 cells in a dose-dependent manner, whereas isotype-MMAF was >100 times less effective and unconjugated A8 had no efficacy (FIG. 9A). The cytotoxic efficacy of A8-MMAF correlated with the cell-surface CD97 level (FIG. 9B and FIG. 8C top), indicating that A8-MMAF is a CD97-selective cytotoxic agent. These results strongly suggest the feasibility of selective killing of cells, particularly cancer cells, that have high levels of CD97 on the surface.

Determination of the cryo-EM structure of A8 Fab in complex with CD97 isoform 1 and a mutation study of interface residues

The cryo-EM structure of A8 Fab in complex with CD97 isoform 1 harboring an S531A mutation for prevention of autoproteolysis was determined (FIGS. 27A-27B). Cryo-EM grids were prepared and the data were collected and processed as described herein. Resulting maps were used to build structural models which were further iteratively refined. The interfaces between the V_{H} and V_{L} of the A8 Fab and CD97 isoform 1, including side chains of key residues and polar interactions, are shown in FIG. 27C. Binding titration curves of wild type A8 and N109E (A8 N100bHE), W112A (A8 W100eHA) and Y113A A8 (A8 Y100fHA) mutants against CD97 isoform 1 were determined using a bead-based binding assay described herein (FIG. 27D). Binding titration curves of A8 against CD97 GAIN mutants (Q365A, K519A, R380A, Q376A, K382A, T351K and T524K) were determined using a cell-based binding assay described herein (FIG. 27E). K_{D} values were determined from the curve fitting of a 1:1 binding model. Mutations of residues in the binding interface observed in the structure reduced or abolished the binding of A8 to CD97, validating the cryo-EM structure of the A8-CD97 complex. A summary of the mutation study is depicted in FIG. 27F. The mutations in N100bH, W100eH and Y100fH in CDRH3 residues of A8 substantially reduced or abolished binding to CD97, indicating those residues are essential for A8-CD97 interaction. The Ala mutations in Q376 and K382 positions in the CD97 GAIN domain substantially reduced binding to A8, indicating those resides are essential for A8-CD97 interaction. The Lys mutations in T351 and T524 in the CD97 GAIN domain completely abolished binding to CD97, indicating these residues are closely involved in A8-CD97 interaction.

### A8 Antibody-Drug Conjugate (ADC) Optimization and Assessment of Cytotoxicity.

A8 ADCs were prepared using cysteine-based random or site-specific conjugation methods (FIGS. 28A-28B). The Fc region in both constructs contained L234A/L235A/P329G (LALA-PG) mutations to abolish Fc receptor binding. A S239C mutation was included for site-specific conjugation. A summary of thermal stability, drug-to-antibody ratio (DAR), and conjugation methods of the A8 ADCs is shown in FIG. 28B. The specific A8 formats tested were: hIgG1, hIgG1-MMAF (cysteine-based random conjugation), hIgG1_{S239c}, hIgG1_{S239c}-MMAF (site-specific conjugation), and hIgG1_{S239c}-PBD (site-specific conjugation). The A8 ADCs prepared with the site-specific conjugation method demonstrated improved stability as compared to those prepared with the cysteine-based random conjugation method. Dose-response curves of A8 ADCs tested using OCI-AML3 cells are shown in FIGS. 28C-28D. Motavizumab (mota), an anti-RSV antibody, was used as a negative control antibody. The cells were incubated with the A8 ADCs or unconjugated A8 for 4 days, and cell death was measured with Annexin V staining. A8_{S293C}-PBD showed a potent cell-killing effect.

For assessment of specificity and toxicity assessment of A8_{S293C}-PBD, cell lines OCI-AML3 (hCD97 high) and ExpiCHO (hCD97-null), and primary cell human peripheral blood mononuclear cells (PBMCs) were incubated with A8_{S293C}-PBD or unconjugated A8 for 4 days. A8_{S293C}-PBD specifically killed cells expressing high levels of CD97 and did not kill PBMC cells, indicating specific killing and suggesting low toxicity (FIG. 28E).

### Amino Acid Sequences of Exemplary Antibody Clones Binding to CD97: CDR Residues (Kabat Scheme) in Bold

Antibody Clone A1
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQYSWSSYLITFGQGTKVEIKRTV (SEQ ID NO: 1)
LCDR1: RASQSVSSAVA (SEQ ID NO: 3)
LCDR2: SASSLYS (SEQ ID NO: 4)
LCDR3: QQYSWSSYLIT (SEQ ID NO: 5)
V_{H}: EVQLVESGGGLVQPGGSLRLSCAASGFTISSSSIHWVRQAPGKGLEWVASIYSYYGSTYYADSVKGRF
TISADTSKNTAYLQMNSLRAEDTAVYYCARPVYGQLGYWTQRWSGMDYWGQGTLVTVSS
(SEQ ID NO: 2)
HCDR1: SSSIH (SEQ ID NO: 6)
HCDR2: SIYSYYGSTYYADSVKG (SEQ ID NO: 7)
HCDR3: PVYGQLGYWTQRWSGMDY (SEQ ID NO: 8)
Antibody Clone A8
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSVYSPVTFGQGTKVEIKRTV (SEQ ID NO: 9)
LCDR1: RASQSVSSAVA (SEQ ID NO: 3)
LCDR2: SASSLYS (SEQ ID NO: 4)
LCDR3: QQSVYSPVT (SEQ ID NO: 11)
V_{H}: EVQLVESGGGLVQPGGSLRLSCAASGFTISSYSIHWVRQAPGKGLEWVASISPYYGSTSYADSVKGRF
TISADTSKNTAYLQMNSLRAEDTAVYYCARSWYYYEQNPYWYQKEEQALDYWGQGTLVTVSS
(SEQ ID NO: 10)
HCDR1: SYSIH (SEQ ID NO: 12)
HCDR2: SISPYYGSTSYADSVKG (SEQ ID NO: 13)
HCDR3: SWYYYEQNPYWYQKEEQALDY (SEQ ID NO: 14)
Antibody Clone B3
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQLRHYSHLITFGQGTKVEIKRTV (SEQ ID NO: 15)
LCDR1: RASQSVSSAVA (SEQ ID NO: 3)
LCDR2: SASSLYS (SEQ ID NO: 4)
LCDR3: QQLRHYSHLIT (SEQ ID NO: 17)
V_{H}: EVQLVESGGGLVQPGGSLRLSCAASGFTFSSSSIHWVRQAPGKGLEWVASISPYSGYTSYADSVKGRF
TISADTSKNTAYLQMNSLRAEDTAVYYCARAYYYWSHWYMHYVSYPGGMDYWGQGTLVTVSS
(SEQ ID NO: 16)
HCDR1: SSSIH (SEQ ID NO: 6)
HCDR2: SISPYSGYTSYADSVKG (SEQ ID NO: 18)
HCDR3: AYYYWSHWYMHYVSYPGGMDY (SEQ ID NO: 19)
Antibody Clone B20
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTISSSSIHWVRQAPGKGLEWVASISSSSGSTSYADSVKGRF
TISADTSKNTAYLQMNSLRAEDTAVYYCARIRHYHWYEIPYWDWAIDYWGQGTLVTVSS
(SEQ ID NO: 22)
HCDR1: SSSIH (SEQ ID NO: 6)
HCDR2: SISSSSGSTSYADSVKG (SEQ ID NO: 23)
HCDR3: IRHYHWYEIPYWDWAIDY (SEQ ID NO: 24)
Antibody Clone B23
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQYWGSDPITFGQGTKVEIKRTV (SEQ ID NO: 25)
LCDR1: RASQSVSSAVA (SEQ ID NO: 3)
LCDR2: SASSLYS (SEQ ID NO: 4)
LCDR3: QQYWGSDPIT (SEQ ID NO: 27)
V_{H}: EVQLVESGGGLVQPGGSLRLSCAASGFTVSSSSIHWVRQAPGKGLEWVASIYPYSGSTSYADSVKGRF
TISADTSKNTAYLQMNSLRAEDTAVYYCARSLGWHRHYNYYFYFYANAIDYWGQGTLVTVSS
(SEQ ID NO: 26)
HCDR1: SSSIH (SEQ ID NO: 6)
HCDR2: SIYPYSGSTSYADSVKG (SEQ ID NO: 28)
HCDR3: SLGWHRHYNYYFYFYANAIDY (SEQ ID NO: 29)
Antibody Clone B25
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTISSSSIHWVRQAPGKGLEWVASISSYSGSTSYADSVKGRF
TISADTSKNTAYLQMNSLRAEDTAVYYCARSFFKYYLWYGYHNYWSAIDYWGQGTLVTVSS (SEQ ID
NO: 32)
HCDR1: SSSIH (SEQ ID NO: 6)
HCDR2: SISSYSGSTSYADSVKG (SEQ ID NO: 30)
HCDR3: SFFKYYLWYGYHNYWSAIDY (SEQ ID NO: 33)
Antibody Clone B34
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSGYYGHLITFGQGTKVEIKRTV (SEQ ID NO: 35)
LCDR1: RASQSVSSAVA (SEQ ID NO: 3)
LCDR2: SASSLYS (SEQ ID NO: 4)
LCDR3: QQSGYYGHLIT (SEQ ID NO: 34)
V_{H}: EVQLVESGGGLVQPGGSLRLSCAASGFTVSYSSIHWVRQAPGKGLEWVASIYSSSGYTSYADSVKGRF
TISADTSKNTAYLQMNSLRAEDTAVYYCARSYYRYYLSPIYWGSWGMDYWGQGTLVTVSS (SEQ ID
NO: 36)
HCDR1: YSSIH (SEQ ID NO: 37)
HCDR2: SIYSSSGYTSYADSVKG (SEQ ID NO: 38)
HCDR3: SYYRYYLSPIYWGSWGMDY (SEQ ID NO: 39)
Antibody Clone C14
V_{L}: DIQMTQSPSSLSASVGDRVTITCRASQSVSSAVAWYQQKPGKAPKLLIYSASSLYSGVPSRESGSRSG
TDFTLTISSLQPEDFATYYCQQYWHESLITFGQGTKVEIKRTV (SEQ ID NO: 41)
LCDR1: RASQSVSSAVA (SEQ ID NO: 3)
LCDR2: SASSLYS (SEQ ID NO: 4)
LCDR3: QQYWHESLIT (SEQ ID NO: 40)
V_{H}: EVQLVESGGGLVQPGGSLRLSCAASGFTVSSSSIHWVRQAPGKGLEWVASIYSYSGSTSYADSVKGRF
TISADTSKNTAYLQMNSLRAEDTAVYYCARSFGWGQSFYYSHYFYITAFDYWGQGTLVTVSS
(SEQ ID NO: 42)
HCDR1: SSSIH (SEQ ID NO: 6)
HCDR2: SIYSYSGSTSYADSVKG (SEQ ID NO: 43)
HCDR3: SFGWGQSFYYSHYFYITAFDY (SEQ ID NO: 44)
Antibody Clone C27
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTLSSSSIHWVRQAPGKGLEWVASIYSYYGSTYYADSVKGRF
TISADTSKNTAYLQMNSLRAEDTAVYYCARYRGNMYYWESWYGSYWALDYWGQGTLVTVSS (SEQ ID
NO: 46)
HCDR1: SSSIH (SEQ ID NO: 6)
HCDR2: SIYSYYGSTYYADSVKG (SEQ ID NO: 7)
HCDR3: YRGNMYYWESWYGSYWALDY (SEQ ID NO: 47)
Antibody Clone D9
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSGYPVTFGQGTKVEIKRTV (SEQ ID NO: 49)
LCDR1: RASQSVSSAVA (SEQ ID NO: 3)
LCDR2: SASSLYS (SEQ ID NO: 4)
LCDR3: QQSSGYPVT (SEQ ID NO: 48)
V_{H}: EVQLVESGGGLVQPGGSLRLSCAASGFTVSSSSIHWVRQAPGKGLEWVASISSYYGYTSYADSVKGRF
TISADTSKNTAYLQMNSLRAEDTAVYYCARYPMFSYYYWYYGMDYWGQGTLVTVSS (SEQ ID NO:
50)
HCDR1: SSSIH (SEQ ID NO: 6)
HCDR2: SISSYYGYTSYADSVKG (SEQ ID NO: 51)
HCDR3: YPMFSYYYWYYGMDY (SEQ ID NO: 52)
Antibody Clone D12
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTFSSSSIHWVRQAPGKGLEWVASISSSSGSTSYADSVKGRF
TISADTSKNTAYLQMNSLRAEDTAVYYCARIRHYHWYEIPYWDWAIDYWGQGTLVTVSS
(SEQ ID NO: 54)
HCDR1: SSSIH (SEQ ID NO: 6)
HCDR2: SISSSSGSTSYADSVKG (SEQ ID NO: 23)
HCDR3: IRHYHWYEIPYWDWAIDY (SEQ ID NO: 24)
Antibody Clone D25
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTFSSSSIHWVRQAPGKGLEWVASISPYYGSTSYADSVKGRF
TISADTSKNTAYLQMNSLRAEDTAVYYCARKLEWGMFFWSPYFRALDYWGQGTLVTVSS
(SEQ ID NO: 56)
HCDR1: SSSIH (SEQ ID NO: 6)
HCDR2: SISPYYGSTSYADSVKG (SEQ ID NO: 13)
HCDR3: KLEWGMFFWSPYFRALDY (SEQ ID NO: 57)
Antibody Clone 2A5
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSKESSPVTFGQGTKVEIKRTV (SEQ ID NO: 59)
LCDR1: RASQSVSSAVA (SEQ ID NO: 3)
LCDR2: SASSLYS (SEQ ID NO: 4)
LCDR3: QQSKESSPVT (SEQ ID NO: 58)
V_{H}: EVQLVESGGGLVQPGGSLRLSCAASGFTFSSSSIHWVRQAPGKGLEWVASISSSYGSTSYADSVKGRF
TISADTSKNTAYLQMNSLRAEDTAVYYCARGWAYSYWYYSHYYEYEVALDYWGQGTLVTVSS
(SEQ ID NO: 60)
HCDR1: SSSIH (SEQ ID NO: 6)
HCDR2: SISSSYGSTSYADSVKG (SEQ ID NO: 61)
HCDR3: GWAYSYWYYSHYYEYEVALDY (SEQ ID NO: 62)
Antibody Clone 2V10
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSWWDPITFGQGTKVEIKRTV (SEQ ID NO: 63)
LCDR1: RASQSVSSAVA (SEQ ID NO: 3)
LCDR2: SASSLYS (SEQ ID NO: 4)
LCDR3: QQSWWDPIT (SEQ ID NO: 65)
V_{H}: EVQLVESGGGLVQPGGSLRLSCAASGFTISSSSIHWVRQAPGKGLEWVAYIYPSSGSTSYADSVKGRF
TISADTSKNTAYLQMNSLRAEDTAVYYCARPWYYSTENKKWWDYQGLDYWGQGTLVTVSS (SEQ ID
NO: 64)
HCDR1: SSSIH (SEQ ID NO: 6)
HCDR2: YIYPSSGSTSYADSVKG (SEQ ID NO: 66)
HCDR3: PWYYSTENKKWWDYQGLDY (SEQ ID NO: 67)
Antibody Clone 2W1
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQDSSYPITFGQGTKVEIKRTV (SEQ ID NO: 69)
LCDR1: RASQSVSSAVA (SEQ ID NO: 3)
LCDR2: SASSLYS (SEQ ID NO: 4)
LCDR3: QQDSSYPIT (SEQ ID NO: 68)
V_{H}: EVQLVESGGGLVQPGGSLRLSCAASGFTVSSSSIHWVRQAPGKGLEWVAYISSSSGSTYYADSVKGRF
TISADTSKNTAYLQMNSLRAEDTAVYYCARDFYFGSYYSFGWGLDYWGQGTLVTVSS (SEQ ID NO:
70)
HCDR1: SSSIH (SEQ ID NO: 6)
HCDR2: YISSSSGSTYYADSVKG (SEQ ID NO: 71)
HCDR3: DFYFGSYYSFGWGLDY (SEQ ID NO: 72)
Antibody Clone 2X10
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTFSSSSIHWVRQAPGKGLEWVASISSSSGSTSYADSVKGRF
TISADTSKNTAYLQMNSLRAEDTAVYYCARSYWDMYIYWYYYVKQSRGMDYWGQGTLVTVSS
(SEQ ID NO: 74)
HCDR1: SSSIH (SEQ ID NO: 6)
HCDR2: SISSSSGSTSYADSVKG (SEQ ID NO: 23)
HCDR3: SYWDMYIYWYYYVKQSRGMDY (SEQ ID NO: 75)
Antibody Clone 2X12
VL: DIQMTQSPSSLSASVGDRVTITCRASQSVSSAVAWYQQKPGKAPKLLIYSASSLYSGVPSRFSGSRSG
TDFTLTISSLQPEDFATYYCQQWWGPELVTFGQGTKVEIKRTV (SEQ ID NO: 77)
LCDR1: RASQSVSSAVA (SEQ ID NO: 3)
LCDR2: SASSLYS (SEQ ID NO: 4)
LCDR3: QQWWGPELVT (SEQ ID NO: 76)
V_{H}: EVQLVESGGGLVQPGGSLRLSCAASGFTISSSSIHWVRQAPGKGLEWVASISSSSSYTSYADSVKGRF
TISADTSKNTAYLQMNSLRAEDTAVYYCARMDISYYYYEWGFESAYYGMDYWGQGTLVTVSS
(SEQ ID NO: 78)
HCDR1: SSSIH (SEQ ID NO: 6)
HCDR2: SISSSSSYTSYADSVKG (SEQ ID NO: 79)
HCDR3: MDISYYYYEWGFESAYYGMDY (SEQ ID NO: 80)
Antibody Clone 2Z3
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTFSSSSIHWVRQAPGKGLEWVASISSSSGSTSYADSVKGRF
TISADTSKNTAYLQMNSLRAEDTAVYYCARVLLGKFWYYNYYEYSAMDYWGQGTLVTVSS (SEQ ID
NO: 81)
HCDR1: SSSIH (SEQ ID NO: 6)
HCDR2: SISSSSGSTSYADSVKG (SEQ ID NO: 23)
HCDR3: VLLGKFWYYNYYEYSAMDY (SEQ ID NO: 82)
Antibody Clone 2Z7
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV (SEQ ID NO: 21)
LCDR1: RASQSVSSAVA (SEQ ID NO: 3)
LCDR2: SASSLYS (SEQ ID NO: 4)
LCDR3: QQSSSSLIT (SEQ ID NO: 20)
V_{H}: EVQLVESGGGLVQPGGSLRLSCAASGFTFSSSSIHWVRQAPGKGLEWVASISSSSGSTSYADSVKGRF
TISADTSKNTAYLQMNSLRAEDTAVYYCARSYWERYYYWYYFYQYELGLDYWGQGTLVTVSS
(SEQ ID NO: 31)
HCDR1: SSSIH (SEQ ID NO: 6)
HCDR2: SISSSSGSTSYADSVKG (SEQ ID NO: 23)
HCDR3: SYWERYYYWYYFYQYELGLDY (SEQ ID NO: 45)
Antibody Clone 2Z11
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQYWGYSLITFGQGTKVEIKRTV (SEQ ID NO: 53)
LCDR1: RASQSVSSAVA (SEQ ID NO: 3)
LCDR2: SASSLYS (SEQ ID NO: 4)
LCDR3: QQYWGYSLIT (SEQ ID NO: 55)
V_{H}: EVQLVESGGGLVQPGGSLRLSCAASGFTFYYSSIHWVRQAPGKGLEWVASIYSYSGYTSYADSVKGRF
TISADTSKNTAYLQMNSLRAEDTAVYYCARYYISGPYYAYRSYTSYAMDYWGQGTLVTVSS (SEQ ID
NO: 73)
HCDR1: YSSIH (SEQ ID NO: 37)
HCDR2: SIYSYSGYTSYADSVKG (SEQ ID NO: 83)
HCDR3: YYISGPYYAYRSYTSYAMDY (SEQ ID NO: 84)
Antibody Clone A8 N100b_{H}E
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSVYSPVTFGQGTKVEIKRTV (SEQ ID NO: 9)
LCDR1: RASQSVSSAVA (SEQ ID NO: 3)
LCDR2: SASSLYS (SEQ ID NO: 4)
LCDR3: QQSVYSPVT (SEQ ID NO: 11)
V_{H}: EVQLVESGGGLVQPGGSLRLSCAASGFTISSYSIHWVRQAPGKGLEWVASISPYYGSTSYADSVKGRF
TISADTSKNTAYLQMNSLRAEDTAVYYCARSWYYYEQEPYWYQKEEQALDYWGQGTLVTVSS
(SEQ ID NO: 109)
HCDR1: SYSIH (SEQ ID NO: 12)
HCDR2: SISPYYGSTSYADSVKG (SEQ ID NO: 13)
HCDR3: SWYYYEQEPYWYQKEEQALDY (SEQ ID NO: 112)
N100b_{H}E mutation relative to the amino acid sequence of Antibody Clone A8 shown in
underlined text above.
Antibody Clone A8 W100e_{H}A
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSVYSPVTFGQGTKVEIKRTV (SEQ ID NO: 9)
LCDR1: RASQSVSSAVA (SEQ ID NO: 3)
LCDR2: SASSLYS (SEQ ID NO: 4)
LCDR3: QQSVYSPVT (SEQ ID NO: 11)
V_{H}: EVQLVESGGGLVQPGGSLRLSCAASGFTISSYSIHWVRQAPGKGLEWVASISPYYGSTSYADSVKGRF
TISADTSKNTAYLQMNSLRAEDTAVYYCARSWYYYEQNPYAYQKEEQALDYWGQGTLVTVSS
(SEQ ID NO: 110)
HCDR1: SYSIH (SEQ ID NO: 12)
HCDR2: SISPYYGSTSYADSVKG (SEQ ID NO: 13)
HCDR3: SWYYYEQNPYAYQKEEQALDY (SEQ ID NO: 113)
W100eA mutation relative to the amino acid sequence of Antibody Clone A8 shown in
underlined text above.
Antibody Clone A8 Y100f_{H}A
V_{L}: DIQMTQSPSSLSASVGDRVTITCRASQSVSSAVAWYQQKPGKAPKLLIYSASSLYSGVPSRFSGSRSG
TDFTLTISSLQPEDFATYYCQQSVYSPVTFGQGTKVEIKRTV (SEQ ID NO: 9)
LCDR1: RASQSVSSAVA (SEQ ID NO: 3)
LCDR2: SASSLYS (SEQ ID NO: 4)
LCDR3: QQSVYSPVT (SEQ ID NO: 11)
V_{H}: EVQLVESGGGLVQPGGSLRLSCAASGFTISSYSIHWVRQAPGKGLEWVASISPYYGSTSYADSVKGRF
TISADTSKNTAYLQMNSLRAEDTAVYYCARSWYYYEQNPYWAQKEEQALDYWGQGTLVTVSS
(SEQ ID NO: 111)
HCDR1: SYSIH (SEQ ID NO: 12)
HCDR2: SISPYYGSTSYADSVKG (SEQ ID NO: 13)
HCDR3: SWYYYEQNPYWAQKEEQALDY (SEQ ID NO: 114)

Y100fA mutation relative to the amino acid sequence of Antibody Clone A8 shown in underlined text above.

### REFERENCES

1. Martin G H, Roy N, Chakraborty S, Desrichard A, Chung S S, Woolthuis C M, Hu W, Berezniuk I, Garrett-Bakelman F E, Hamann J, Devlin S M, Chan T A, Park C Y. CD97 is a critical regulator of acute myeloid leukemia stem cell function. J Exp Med. 2019; 216(10):2362-77. Epub 20190801. doi: 10.1084/jem.20190598. PubMed PMID: 31371381; PMCID: PMC6781010.
2. Abbott R J, Spendlove I, Roversi P, Fitzgibbon H, Knott V, Teriete P, McDonnell J M, Handford P A, Lea S M. Structural and functional characterization of a novel T cell receptor co-regulatory protein complex, CD97-CD55. J Biol Chem. 2007; 282(30):22023-32. Epub 2007/04/24. doi: 10.1074/jbc.M702588200. PubMed PMID: 17449467.
3. Miller K R, Koide A, Leung B, Fitzsimmons J, Yoder B, Yuan H, Jay M, Sidhu S S, Koide S, Collins E J. T cell receptor-like recognition of tumor in vivo by synthetic antibody fragment. PloS one. 2012; 7(8):e43746. Epub 2012/08/24. doi: 10.1371/journal.pone.0043746. PubMed PMID: 22916301; PMCID: 3423377.
4. Oury J, Zhang W, Leloup N, Koide A, Corrado A D, Ketavarapu G, Hattori T, Koide S, Burden S J. Mechanism of disease and therapeutic rescue of Dok7 congenital myasthenia. Nature. 2021; 595(7867):404-8. Epub 2021/06/25. doi: 10.1038/s41586-021-03672-3. PubMed PMID: 34163073; PMCID: PMC8277574.
5. Sidhu S S, Lowman H B, Cunningham B C, Wells J A. Phage display for selection of novel binding peptides. Methods Enzymol. 2000; 328:333-63. PubMed PMID: 11075354.
6. Lo M, Kim H S, Tong R K, Bainbridge T W, Vernes J M, Zhang Y, Lin Y L, Chung S, Dennis M S, Zuchero Y J, Watts R J, Couch J A, Meng Y G, Atwal J K, Brezski R J, Spiess C, Ernst J A. Effector-attenuating Substitutions That Maintain Antibody Stability and Reduce Toxicity in Mice. J Biol Chem. 2017; 292(9):3900-8. Epub 2017/01/13. doi: 10.1074/jbc.M116.767749. PubMed PMID: 28077575; PMCID: PMC5339770.
7. Nishikori S, Hattori T, Fuchs S M, Yasui N, Wojcik J, Koide A, Strahl B D, Koide S. Broad ranges of affinity and specificity of anti-histone antibodies revealed by a quantitative Peptide immunoprecipitation assay. J Mol Biol. 2012; 424(5):391-9. Epub 2012/10/09. doi: 10.1016/j.jmb.2012.09.022. PubMed PMID: 23041298; PMCID: 3502729.
8. Hattori T, Lai D, Dementieva I S, Montano S P, Kurosawa K, Zheng Y, Akin L R, Swist-Rosowska K M, Grzybowski A T, Koide A, Krajewski K, Strahl B D, Kelleher N L, Ruthenburg A J, Koide S. Antigen clasping by two antigen-binding sites of an exceptionally specific antibody for histone methylation. Proc Natl Acad Sci USA. 2016; 113(8):2092-7. doi: 10.1073/pnas.1522691113. PubMed PMID: 26862167; PMCID: 4776465.
9. Hattori T, Koide A, Noval M G, Panchenko T, Romero L A, Teng K W, Tada T, Landau N R, Stapleford K A, Koide S. The ACE2-binding Interface of SARS-CoV-2 Spike Inherently Deflects Immune Recognition. J Mol Biol. 2021; 433(3):166748. Epub 20201211. doi: 10.1016/j.jmb.2020.166748. PubMed PMID: 33310017; PMCID: PMC7833242.
10. Doronina S O, Mendelsohn B A, Bovee T D, Cerveny C G, Alley S C, Meyer D L, Oflazoglu E, Toki B E, Sanderson R J, Zabinski R F, Wahl A F, Senter P D. Enhanced activity of monomethylauristatin F through monoclonal antibody delivery: effects of linker technology on efficacy and toxicity. Bioconjug Chem. 2006; 17(1):114-24. doi: 10.1021/bc0502917. PubMed PMID: 16417259.

### Example 2. The Expression Profile and Tumorigenic Mechanisms of CD97 (ADGRE5) in Glioblastoma Render it a Targetable Vulnerability

Glioblastoma (GBM) is the most common and aggressive primary brain malignancy. Adhesion G protein-coupled receptors (aGPCRs) have attracted interest for their functional role in gliomagenesis and their potential as treatment targets. To identify therapeutically targetable opportunities among aGPCR family members in unbiased fashion, expression levels of all aGPCRs in GBM and non-neoplastic brain tissue were analyzed. Using bulk and single cell transcriptomic and proteomic data, it was show herein that CD97 (ADGRE5), an aGPCR previously implicated in GBM pathogenesis, is the most promising aGPCR target in GBM, by virtue of its abundance in all GBM tumors and its de novo expression profile in GBM compared to normal brain tissue and neural progenitors. CD97 knockdown or knockout significantly reduced the tumor initiation capacity of patient-derived GBM cultures (PDGC) in vitro and in vivo. Transcriptomic and metabolomic data from PDGCs suggest that CD97 promotes glycolytic metabolism. The oncogenic and metabolic effects of CD97 are mediated by the MAPK pathway. Activation of MAPK signaling depends on phosphorylation of the cytosolic C-terminus of CD97 and recruitment of β-arrestin. Using single-cell RNA-sequencing and biochemical assays, it was demonstrated herein that THY1/CD90 is the most likely CD97 ligand in GBM. Lastly, it was shown herein that targeting of PDGCs with an anti-CD97 antibody-drug conjugate in vitro selectively kills tumor cells but not human astrocytes or neural stem cells. The present study identified CD97 as an important regulator of tumor metabolism in GBM, elucidated mechanisms of receptor activation and signaling, and provided strong scientific rationale for developing biologics to target it for therapeutic purposes.

Glioma is the most common primary brain malignancy. In adults, two main types of glioma exist. The less common type is typically encountered in younger patients and is driven by a neomorphic mutation in the metabolic enzyme isocitrate dehydrogenase (IDH). The more common type, also known as glioblastoma (GBM), is observed in older patients, lacks the IDH mutation (IDH-wildtype), has an aggressive course, and represents the largest unmet need in neuro-oncology. The current treatment regimen for GBM involves neurosurgical resection of the tumor, followed by chemoradiotherapy. Nevertheless, these measures have done little to improve patient outcomes, with the median survival limited to about 15 months.³⁻⁵ It is clear that in order to improve GBM treatment, new targetable vulnerabilities of tumor cells and their microenvironment should be identified.

In the effort to identify novel mechanisms contributing to GBM tumorigenesis, the present inventors became interested in the adhesion family of G protein-coupled receptors (aGPCRs), which consists of 33 members.⁶ Adhesion GPCRs are characterized by large extracellular N-termini that contain both receptor-specific domains determining ligand binding and a functionally conserved GPCR autoproteolysis inducing (GAIN) domain, which catalyzes receptor cleavage at the GPCR proteolysis site (GPS).⁷⁻⁹ Emerging evidence has implicated several aGPCRs in developmental, physiologic, and oncogenic processes.^{6,10-15} This prompted the present inventors to compare the expression of all aGPCR members in cell types within healthy, non-neoplastic brain tissue; neural stem cells (NSCs), the putative cell-of-origin in GBM¹⁶⁻¹⁸; and patient-derived GBM cultures (PDGCs). The present transcriptomic and proteomic expression analysis identified CD97 (ADGRE5) as the aGPCR with the largest differential expression profile: high expression in PDGCs derived from all transcriptional subtypes of GBM in The Cancer Genome Atlas (TCGA; proneural, classical, and mesenchymal)^{2,19}, and absence from normal brain tissue and NSCs.

CD97 is expressed in several lineages of the immune system, where it is critical for the inflammatory response,²⁰⁻²⁴ as well as in multiple liquid and solid malignancies.^{13,25-28} Among these malignancies is GBM, in which CD97 was previously implicated in cellular proliferation, brain invasion, and tumor metabolism.²⁹⁻³³ However, its mechanism of action in GBM remains incompletely understood. Furthermore, little research effort has been devoted to its therapeutic targeting. Here, PDGCs were used to demonstrate that CD97 is essential for tumor growth both in vitro and in vivo. Using transcriptomic, metabolomic, and signaling assays, it was found herein that CD97 helps promote glycolytic metabolism via activation of the mitogen-activated protein kinase (MAPK) signaling pathway. THY1/CD90 was also identified as the most likely physiologically relevant CD97 ligand in GBM. It was also demonstrated that CD97 signaling depends on phosphorylation of its C-terminus and recruitment of β-arrestin. To capitalize on CD97's therapeutic potential, it was shown that a CD97 antibody-drug conjugate (ADC) that had been developed in-house specifically kills GBM cells, but not human astrocytes or NSCs in vitro. Collectively, these data elucidate novel receptor activation and signaling mechanisms employed by CD97 to promote tumor growth and regulate tumor metabolism in GBM, and highlight the receptor's potential as a therapeutically targetable vulnerability in GBM.

### Materials and Methods

### Cell Culture

PDGCs were established and maintained as previously described.^{11,37-85} In brief, fresh operative specimens were obtained from patients undergoing surgery for resection of GBM after informed consent (NYU IRB study 12-01130). Specimens were mechanically minced using surgical blades followed by enzymatic dissociation using Accutase (Cat #AT104, Innovative Cell Technologies). Cells were either long-term maintained in spheroid suspension cultures (tumorspheres) on untreated cell culture dishes or grown as attached cultures on dishes pretreated with poly-L-ornithine (PLO; Cat #P4957, Sigma) and laminin (Cat #23017015, Thermo Fisher). The GBM growth medium consisted of Neurobasal medium (Cat #21103049, Gibco) supplemented with N2 (Cat #17-502-049, Gibco), B27 (Cat #12587010, Gibco), nonessential amino acids (Cat #11140050, Gibco), and GlutaMax (Cat #35050061, Gibco). Twenty ng/ml recombinant basic Fibroblast Growth Factor (bFGF; Cat #233-FB-O1M, R&D) and 20 ng/ml Epidermal Growth Factor (EGF; Cat #236-EG-01M, R&D) were added to the medium every other day. Parental tumors of these patient-derived cultures underwent DNA methylation, mutational and copy number variation profiling using previously described assays.³⁷ All tumors used for PDGCs had a wildtype IDH genetic background.

Human embryonic stem cell (WA09) derived NSCs were established and maintained as previously described in detail.¹⁶ HEK293T (Cat #632180, Takara) cells were cultured in Dulbecco's modified Eagle's medium (DMEM; Cat #11965-118, Gibco) supplemented with 10% fetal bovine serum (FBS; Cat #PS-FB2, Peak Serum) and sodium pyruvate (Cat #11360070, Gibco). NHAs (Cat #CC-2565, Lonza) were cultured in PLO/laminin-coated tissue culture plates in DMEM media with 10% FBS and N2 supplement. All cells were cultured in humidified cell culture incubators at 37° C. balanced with 5% CO₂ and at 21% O₂.

### Flow Cytometry Surface Staining

Cells were enzymatically dissociated using Accutase and 5×10⁵ cells were pelleted at 1000 rpm for 5 minutes. Cells were washed in Dulbecco's phosphate-buffered saline (DPBS; Cat #14190-144, Gibco) before resuspension in fluorescence-activated cell sorting (FACS) buffer [0.5% BSA (Cat #A3733-100G, Sigma) in DPBS with 2 mM EDTA (Cat #AM9260G, Thermo)] with diluted primary antibody for 30 minutes at 4° C. Cells were washed in FACS buffer before a final resuspension in FACS buffer and subsequent transfer to a 5 mL round bottom polysterene test tube through a cell strainer (Cat #352235, Falcon). Samples were run on a BD LSRFortessa (BD Biosciences).

### Immunofluorescent Staining of Tissue

GBM tumors were collected as surgical specimens and normal brain tissue was collected from patients post mortem. Tissues were washed for paraffin-embedding and sectioning by the Center for Biospecimen Research & Development (CBRD) core at NYU. Slides were deparaffinized and rehydrated by submersion into xylene (15 minutes) (Cat #1330-20-7, Crystalgen), 100% ethanol (3 minutes) (Cat #UN1170, Fisher bioreagents), 95% ethanol (3 minutes) (Cat #E7148-500ML, Sigma-Aldrich), and 70% ethanol (5 minutes) (Cat #2401, Decon Laboratories). The slides were washed three times in DPBS with 0.1% Tween-20 (PBS-T; Cat #H5151, Promega). Antigen retrieval was performed by submerging the slides in citrate buffer [2.94 g sodium citrate tribasic dihydrate (Cat #S4641-500G, Sigma-Aldrich), 0.5 mL Tween-20, and 1000 mL ddH₂O] and microwaving for 10 minutes at 800 W followed by 10 minutes at 200 W. The slides were then allowed to cool slowly at room temperature and were washed three times with PBS-T. Slides were blocked for 30 minutes using 50 μL normal goat serum (Cat #ab7481, Abcam) in 1 mL DPBS. Slides were then stained with primary antibody solution at 4° C. overnight. Slides were then washed three times with PBS-T and stained with secondary antibody for one hour at room temperature in the dark. Slides were washed, stained with 1:2000 Hoechst 33342 dye (Cat #H3570, Life Technologies) or 100× DAPI (Cat #D8417-1MG, Sigma) in DPBS, covered in ProLong gold antifade reagent (Cat #P36934, Thermo), and mounted with a coverslip (Cat #10813, Ibidi) for imaging.

### Plasmids and Molecular Cloning

All overexpression plasmids were based on the lentiviral vector pCW57-RFP-P2A-MCS (Plasmid #78933, Addgene). DPS CD97 was generated using primers against the CD97 isoform 3 expression vector and was assembled using Gibson Assembly. Short hairpin RNA design and cloning into the pRSI9-U6-(sh)-UbiC-TagRFP-2A-Puro (Plasmid #28289, Addgene) shRNA expression vector have been described previously.²⁶ Guide RNAs were designed using Benchling, selected based on off-target scores, and cloned into the LentiCRISPRV2-mCherry (Plasmid #99154, Addgene) backbone. The MEK^{DD} construct was in a pBabe-neomycin (Plasmid #1767, Addgene) expression vector.

### Lentiviral Infection and Transfection

Cells were transduced using lentivirus as described previously.⁸⁶ In short, lentivirus was produced by co-transfecting HEK293T cells with expression plasmids of interest and packaging plasmids psPax2 and pMD2.G. Lentivirus was collected from the cell culture supernatant 24, 48, and 72 hours after transfection and concentrated using the Lenti-X concentrator (Cat #631231, Contech Takara). For lentiviral transduction, GBM, or NHA medium was supplemented with 4 mg/ml protamine sulfate and cells were treated with viral particles at a multiplicity of infection (MOI) of three. Infected cells were isolated by FACS with the SH800Z sorter (Sony Biotechnology) or by puromycin selection (5 mg/mL). Doxycycline induction was done by adding 1 mg/mL doxycycline to the medium. HEK293T cells were transfected with plasmid DNA using Lipofectamine 2000 (Cat #11668-019, Invitrogen), following the manufacturer's protocol.

### Immunoblot Staining and Sample Deglycosylation

Cell medium was aspirated and cells were washed with DPBS (Cat #14190-250, Gibco). RIPA buffer (Cat #89901, Thermo) with Protease/Phosphatase inhibitor (Cat #88669, Fisher) was added directly to cells, which were incubated on ice for 15 minutes. A cell scraper was used to collect the whole cell lysate, which was transferred to a 1.5 mL tube (Cat #C2170, Thomas Scientific). Samples were sonicated (ON: 15s, OFF: 60s, 8 cycles, High setting, 4° C.) using a Bioruptor300 (Diagrenode). Samples were spun at 15000 rcf for 10 minutes at 4° C. and the supernatant was transferred to a fresh tube. If samples were deglycosylated, the Protein Deglycosylation Mix II (Cat #P6044, NEB) was used according to the manufacturer's protocol and samples were incubated at 25° C. for 30 minutes and at 37° C. for 16 hours. Bovine serum albumin (BSA) protein standards and a detergent compatible (DC) Protein Assay Kit (Cat #5000112, BioRad) were used to measure protein concentrations of the whole cell lysates according to the manufacturer's protocol. Up to 40 μg whole cell lysate was mixed in 4× Laemmli Buffer (Cat #1610747, BioRad) and run in a 12% Mini-PROTEAN TGX Precast Protein Gel (Cat #4561044, BioRad) for 1 hour at 120 V. Protein gels were then transferred to a PVDF membrane (Cat #1620177, BioRad) for 2 hours at 90 V at 4° C. Membranes were blocked [2% BSA in tris-buffered saline with 0.1% Tween-20 (TBS-T)] for 1 hour at room temperature. Membranes were stained with primary antibody overnight at 4° C. Membranes were then washed three times in TBS-T for 5 minutes before incubation with a secondary antibody for 1 hour at room temperature in the dark. The membrane was finally washed three times in TBS-T for 5 minutes and imaged using an iBrightFL1000 (Invitrogen). Bands for non-phosphorylated species were imaged using fluorescent secondary antibodies and by measuring fluorescent signal. Bands for phosphorylated species were imaged using an HRP-conjugated antibody and by measuring chemiluminescent signal using appropriate West Pico PLUS chemiluminescent substrates (Cat #34577, Thermo) according to the manufacturer's protocol.

### Hoechst Staining

Cells were resuspended (7 days post infection) in DPBS with 1:1000 Hoechst 33342 dye and incubated at 37° C. for 30 minutes. Cells were spun down and resuspended in FACS buffer and transferred to tubes for flow cytometry.

### Annexin-V Staining

One million cells were pelleted (7 days post infection) and washed once in DPBS and then washed once in 1× Binding Buffer (Cat #BMS500BB, eBioscience) in ddH₂O. Cells were resuspended in 100 mL 1× Binding Buffer with 5 ml Annexin-V eFluor450 (Cat #88-8006-74, eBioscience) and were incubated for 15 minutes in the dark at room temperature. Three hundred mL 1× Binding Buffer was added to the cells and they were spun down and resuspended in 200 mL 1× Binding Buffer with 100× DAPI solution. After a 10-minute incubation in the dark at room temperature, cells were resuspended in FACS buffer and transferred to a tube for flow cytometry. Cells that were incubated at 56° C. for 20 minutes were used a positive control for non-viable cells. Cells that were either Annexin-V positive (PE-Cy7) and/or DAPI (Pacific Blue) positive were considered non-viable.

### Transwell Migration Assay

PDGCs were dissociated using Accutase and counted (4 days post infection). The cells were resuspended to a concentration of 50,000 cells/100 mL in serum-free Neurobasal media. Meanwhile a 1:30 Matrigel (Cat #354277, Thermo) to Neurobasal medium solution was made and added to the top well of a transwell permeable support 24-well plate (Cat #3422, costar) and incubated at room temperature for two hours. The Matrigel solution was aspirated and the well was washed once with DPBS. One hundred mL of cells were added to the top well. The bottom well was filled with 600 mL of Neurobasal medium with 10% FBS. The plate was incubated at 37° C. for 24 hours. After 24 hours, the media was aspirated and replaced with medium containing 1:1000 Hoechst 33342 dye and was incubated for 10 minutes at 37° C. The media was then aspirated and replaced with DPBS. Three fluorescent images were taken of the cells on the membrane (total cells). The cells in the upper portion of the well were gently removed using a cotton swab. Three fluorescent images were taken of the remaining cells in the lower portion of the well (migrated cells). The fluorescent intensity was measured and the fraction of migrated cells to total cells was calculated.

### Tumorsphere Formation Assay

Cells were enzymatically dissociated using Accutase and counted using Countess II (Invitrogen). Cells were spun down at 1000 rpm for 5 minutes and brought to a concentration of 100-500 cells/50 μL. One row (12 wells) of a 96-well plate (Cat #7200656, Fisher) were filled with 50 p L of the cell suspension. Cells were fed every two days with EGF and bFGF supplements and were given 14 days to form tumorspheres. Ninety-six-well plates were then scanned using an Evos Cell Imaging System (Evos) and tumorspheres were counted using ImageJ software.

### Limiting Dilution Assays

Short hairpin RNA transduced PDGCs were dissociated using Accutase and counted. Cells were plated in 24 wells of a 96-well plate at four separate concentrations: 1000 cells/well, 300 cells/well, 30 cell/well, and 3 cells/well. Cells were then fed every two days and given 14 days to grow. The number of completely empty wells were recorded. Clonogenic frequencies were calculated using ELDA software (bioinf.wehi.edu.au/software/elda/). For plotting, the log₁₀ of each frequency was taken.

### Cell Competition Assay

GBM cells were lentivirally infected with a LentiCRISPRv2 gRNA:Cas9:mCherry construct with an MOI of 3. Cells were then enzymatically dissociated using Accutase into single cells and counted. 100,000 infected, mCherry+ cells were mixed with 100,000 uninfected cells. Flow cytometry [BD LSRFortessa (BD Biosciences)] was used to measure the initial proportion of mCherry+ cells. Every four days, tumorspheres were enzymatically dissociated, the proportion of mCherry+ cells was measured via flow cytometry, and 100,000 cells were replated. This was done until day 20.

### Luciferase Assay

PDGCs lentivirally infected with a dox-inducible CD97-overexpression or empty vector were transfected with luciferase signaling-reporter plasmids: cAMP response element-Luciferase (Cat #E8471, Promega), SRE-Luciferase (Cat #E1340, Promega), SRF-RE-Luciferase (Cat #E1350, Promega), and NFAT-RE-Luciferase (Cat #E8481, Promega). Twenty-four hours after transfection, cells were reseeded in black 96-well plates at a density of 75,000 cells per well with medium containing doxycycline (or dox-free medium). Forty-eight hours after transfection, cells were lysed and luciferase activity was detected using the Bright-Glo Luciferase assay system (Cat #E2650, CisBio) and a BioTek Synergy H1 microplate reader according to the manufacturer's protocol.

### In Vivo GBM Xenografts

Mice were housed within NYU Langone Medical Center's Animal Facilities. All animal procedures were performed according to an IACUC-approved protocol. Orthotopic intracranial xenografts have been described in detail previously.⁸⁷ In short, equal numbers of male and female immunodeficient NSG (NOD.Cg-Prkdc^{scid} Il2rg^{tm1Wjl}/SzJ) mice (6-8 weeks of age) were anesthetized with intraperitoneal injection of ketamine/xylazine (10 mg/kg and 100 mg/kg, respectively). A midline skin incision was made and a small hole was drilled through the skull 2 mm off the midline and 2 mm anterior to the coronal suture. Mice were then stereotactically injected with 2.5×10¹ GBM cells lentivirally infected with a luciferase-containing plasmid. The skin incision was sutured and animals were closely monitored during the recovery period. Mice were weighed every two weeks and sacrificed once they had lost 20% of their maximum body weight or according to ethical guidelines.

### IVIS Imaging of In Vivo GBM Xenografts

In vivo GBM xenografts were monitored using an IVIS Lumina XR (PerkinElmer) as described previously.⁸⁸ First, mice were weighed and injected intraperitoneally with 10 μL/g body weight Luciferin substrate solution [D-Luciferin Potassium Salt (LUCK-300, Gold Biotechnology) diluted in DPBS to a final concentration of 20 mg/mL]. Mice were anesthetized using isoflurane and inserted into the IVIS imaging system. Thirteen minutes after Luciferin injection, mice were imaged at a 150 second exposure time. Living Image software (PerkinElmer) was used to quantify the “Radiance (Photons)” within the selected ROI.

### Bulk RNA-Sequencing

Three replicates of a PDGC (knockdown, scrambled, overexpression, and empty vector) and one replicate each of three PDGCs (knockdown and scrambled) were plated adherently in a 6-well plate. Two replicates of NSCs were also plated adherently on a 6-well plate. RNA was extracted using a RNeasy Mini Kit (Cat #74104, Qiagen) according to the manufacturer's protocol. Samples were submitted to the NYU Langone Genome Technology Core (GTC) for sequencing on an Illumina NovaSeq 6000 flow cell. FastQ files were aligned to the reference genome (hgl9) using the splice aware aligner STAR (v2.5.0c), and the read counts were generated. DESeq2 was used for normalization, differential gene expression analysis, and to generate volcano plots. Log-processed counts per million (CPM) values were used to generate a heatmap of aGPCR expression in GBM cells using Seq-N-Slide (rna-star-groups-dge) software. Peaks were visualized at the ADGRE5 locus (chrl9:14488844-14534630; hgl9 reference genome) using the WashU Epigenome Browser (epigenomegateway.wustl.edu/browser/) and Integrative Genomics Viewer (IGV).

### Single-Cell 10× Multiome (ATAC+RNA) Sequencing

Fresh IDH-mutant astrocytoma and IDH-wildtype GBM surgical specimens were immediately flash frozen in liquid nitrogen. Tissue was homogenized using a pestle (Cat #12-141-367; Thermo). The sample was then processed as described in the “10× Genomics Nuclei Isolation from Complex Tissues for Single Cell Multiome ATAC+GEX Sequencing Demonstrated Protocol CG000375 Rev B” and nuclei were processed for sequencing on an Illumina NovaSeq 6000 flow cell. Data were processed, normalized, integrated for the two specimens, and clustered using Seurat and Signac packages based on RNA and ATAC peak data in R. Only cells with 500-5000 features and with a percentage of mitochondrial genes less than 15% were included in the analysis. Clusters were identified based on the expression of relevant markers (neuronal, oligodendrocytic, astrocytic, and immune) and based on upregulated enriched pathways determined by GO. Oligodendrocytic and neuroglial clusters were compiled together as “Normal brain cells”. Immune populations from the two datasets were clustered together as “Immune cells”. IDH-mutant astrocytoma and IDH-wildtype GBM clusters were identified by their high astrocytic expression profile along with high expression of other oncogenic markers (EGFR, TIMP3, SOX2, TUBA1A, MYC, HIF1A, FN1, HK2, TMOD1, and IGFBP5). ATAC-seq data was visualized at the ADGRE5 locus (chrl9:14380000-14420000; hg38 reference genome) for the clusters using the Signac package in R.

### Pathway Enrichment Analysis and Correlation Matrix Analysis

Differentially expressed downregulated genes were plugged into GO PANTHER software (pantherdb.org/). The top 10 depleted pathways upon CD97 knockdown were plotted based on log₁₀(fold enrichment) values. GSEA software (gsea-msigdb.org/gsea/index.jsp) was used to generate GSEA plots from gene count data. DGCA in R was used to generate a correlation matrix based on RNA-seq data after CD97 knockdown or overexpression in PDGC replicates. Genes designated as glycolytic stem from GO:0061621 along with select glucose transporters. Genes designated as TCA-cycle/OXPHOS include those from GO:0006099 and all cytochrome oxidase subunits. Positive integers designate positive correlation in gene expression patterns, while negative integers designate negative correlation in gene expression patterns.

### Steady-State Metabolomics

Three replicates of two PDGCs (knockdown and scrambled) were plated non-adherently on 6-well plates. After two days, all cells were collected into a 1.5 mL tube and spun at 3000 rcf for 1 minute at 4° C. The supernatant was aspirated and cells were washed in 1 mL of DPBS and immediately pelleted again. Cell pellets were flash-frozen and submitted to the Metabolomics Laboratory for hybrid metabolomics analysis using liquid chromatography/mass spectrometry (LC-MS/MS). A more detailed description of the methods performed for metabolite extraction, LC-MS/MS hybrid metabolomics, and relative metabolite quantification is provided in “Steady-state and flux metabolomics” section below. One replicate was excluded for technical reasons.

### Flux Metabolomics

Three replicates of a PDGC were plated on 6-well plates (knockdown and scrambled). Cells were given fresh Neurobasal-A medium, no D-glucose, no sodium pyruvate (Cat #A2477501, Thermo) with 25 mM [U-¹³C6]-glucose (Cat #389374-10.00G, Sigma). Triplicates were collected and spun at 3000 rcf for 1 minute at 4° C. at three separate timepoints (5, 30, and 120 minutes). Cell pellets were washed with 1 mL DPBS and flash-frozen. Tubes were submitted to the Metabolomics Laboratory for hybrid metabolomics analysis using LC-MS/MS. A more detailed description of the methods performed for metabolite extraction, LC-MS/MS with hybrid metabolomics, and relative quantification is provided in “Steady-state and flux metabolomics section” below.

### Lactate Assay

Lentivirally infected GBM cells were plated on PLO/laminin treated 6-well plates in 1 mL of GBM medium and given 24 hours to grow. A lactate assay cocktail was made [50% Glycine/Hydrazine 0.6M solution (Cat #G5418, Sigma), 1% 240 mM NAD+(Cat #N0632, Sigma), 2 μL/mL Lactate Dehydrogenase (5U/uL) (Cat #10127230001, Roche), 49% sterile ddH₂O]. A series of lactate standards ranging from 10 mM to 0 mM were made (Cat #AC18987-0050, Fisher). Two hundred μL of the lactate cocktail was added to the wells of a 96-well plate. Five μL of each standard or sample was added in triplicates to the wells containing the lactate cocktail. The solution was mixed and incubated for 1 hour at 37° C. The solution was mixed again and the absorbance at 340 nm was read using a Synergy H1 Plate Reader (BioTek).

### Seahorse Cell Energy Phenotype Assay

Seahorse XFe24 Analyzer (Agilent) and the Seahorse XF Cell Energy Phenotype Test Kit (Cat #103325-100, Agilent) were used to perform assays according to the manufacturer's protocol. In brief, a Seahorse cell culture plate was treated with PLO/laminin and 10,000 dissociated GBM cells were plated per well, with five technical replicates per condition accompanied with a blank control well. Cells were incubated overnight at 37° C. in a CO₂ incubator to allow for attachment. Meanwhile, a Seahorse Utility plate/sensor cartridge was hydrated and calibrated using Calibration buffer (Cat #100840-000, Agilent) in a non-CO₂ incubator at 37° C. overnight. Cell medium was aspirated and replaced with 500 μL Assay medium [Agilent Base Medium (Cat #102353-100, Agilent), 1 mM pyruvate (Cat #103578-100, Agilent), 2 mM glutamine (Cat #103579-100, Agilent), 10 mM glucose (Cat #103577-100, Agilent)] and was incubated for 1 hour in a non-CO₂ incubator at 37° C. The mitochondrial stressors carbonyl cyanide p-(tri-fluoromethoxy)phenyl-hydrazone (FCCP) and oligomycin were prepared to a final molar concentration of 1.25 μM and 10 μM, respectively, and were added to Port A of the sensor cartridge. The Seahorse cell plate and sensor cartridge were run on a Seahorse XF Cell Energy Phenotype Assay program using Agilent Wave software. Both OCR and ECAR were measured at three baseline timepoints and six timepoints after the addition of the mitochondrial stressors.

### MEK Mutant Transfection and Selection

GBM cells were transfected with the neomycin-resistant MEK^{DD} construct or a neomycin-resistant empty vector control using Lipofectamine Stem Transfection Reagent (Cat #STEM00008, Thermo) overnight. Cells were treated for three days with media containing 1 mg/mL G418 sulfate (Cat #61-234-RG, Corning) dissolved in DPBS. Cells were given 7-14 days to expand in low dose (100 μg/mL) G418 medium before they were used for specified experiments.

### RHOA-Pulldown Assay

The RhoA Pull-down Activation Assay Biochem Kit (Cat #BK036, Cytoskeleton) was ordered and the manufacturer's protocol was followed. Five hundred mg of cell lysate was used and 15 mL beads.

### HTRF-Based β-Arrestin Recruitment Assay

GBM cells overexpressing wildtype CD97, the APS mutant, or an empty vector control were plated (50,000 cells/well) on a PLO/laminin coated white 96-well plate. A homogenous time-resolved fluorescence (HTRF)-based 0-arrestin2 recruitment kit was used and followed according to the manufacturer's protocol (Cat #62BDBAR2PEB, cisbio). HTRF values were measured using a FlexStation 3 Multi-Mode Microplate Reader (Molecular Devices).

### Ligand-Based Immunoblots

Six-well plates were coated with PLO/laminin. Wells were then additionally coated with recombinant human CD55 (Cat #2009-CD-050, R&D) or recombinant human THY1/CD90 (Cat #16897-HCCH, Sino Biological) at a concentration of 24 μg/mL overnight. GBM cells overexpressing CD97 or an empty vector control were seeded (˜1×10⁶ cells/well) on the plate. Whole cell lysates were collected for immunoblotting 24 hours after seeding.

### Generation of Antibody-Drug Conjugate (ADC) Against CD97

The gene encoding the ectodomain of CD97 isoform 1 was synthesized (Integrated DNA Technologies) and cloned into the mammalian expression vector pBCAG.⁸⁹⁻⁹⁰ The expi293F cells (Thermo Fisher) were transfected with the vector using the ExpiFectamine 293 Transfection Kit (Thermo Fisher) according to the manufacturer's protocol. The CD97 protein was purified from the cell culture supernatant by immobilized metal affinity chromatography (IMAC) followed by size exclusion chromatography. The sorting of a synthetic human antibody (sAb) library and identification of anti-CD97 sAb by phage ELISA were performed essentially as described previously.^{91,92} The gene encoding the VH domain of the antibody was cloned into a modified version of pFUSEss-CHIg-hG1 (InvivoGen) harboring the LALA-PG (L234A, L235A, P329G) mutations that abrogate Fc receptor binding,⁹³ and the gene encoding the VL domain into pFUSEss-CLIg-hk (InvivoGen). The antibody in this IgG1-LALA-PG format was produced using the ExpiCHO expression system according to the manufacturer's protocol (Thermo Fisher), and purified with Protein A affinity chromatography. The drug conjugation reaction was performed as described previously.⁹⁴ Briefly, the interchain disulfide bonds of the antibody were cleaved with DTT. A 9.5-fold molar excess of MC-Val-Cit-PAB-MMAF (BOC Science) was added to the reduced antibody, and after 1 hour of incubation, the reaction was quenched by adding excess cysteine. The excess drug and cysteine were removed with the Zeba spin desalting column (Thermo Fisher). Approximately 6-7 drug molecules were conjugated per antibody.

### Antibody Internalization Assay

Two PDGCs were seeded in 96-well plates in triplicates. Antibodies were conjugated with the pHAb amine reactive dye (Cat #G9841, Promega) according to the manufacturer's protocol. Dye-conjugated antibodies were purified using the Capturem Protein G miniprep columns (Cat #635725, Takara). Purified antibodies were dialyzed into DPBS overnight. The concentration of both the anti-CD97 antibody and the isotype control used for the assay was 30 microgram/ml and the cells were incubated for 20 hours. The cells were then harvested and analyzed by flow cytometry to determine internalization. The pH sensitive pHAb dye that is conjugated to each antibody has low or no fluorescence (PE channel) at pH>7. Upon internalization, the dye becomes fluorescent at an acidic pH present in early endosomes and lysosomes. The increase in mean fluorescence intensity was measured via the PE channel.

### Antibody-Drug Conjugate Dose-Response Curves

Ninety-six-well plates were coated with PLO/laminin. 104 cells were plated per well (100 μL per well; each condition in triplicates) and allowed to adhere overnight. Serial dilutions of ADC, MMAF, and MMAE were prepared in appropriate cell medium to give the following final concentrations: 150 nM, 30 nM, 6 nM, 1.2 nM, 240 μM, 50 μM, 10 μM, 3 μM, 1.4 μM, and 0. Ten μL from dilutions were added to wells. On the seventh day, cells were stained using 1:2000 Hoechst 33342 dye (Cat #H3570, Life technologies) and representative images were taken. Ten μL of WST8 (Cat #ab228554, Abcam) was added to each well and the absorbance at 460 nm was measured using a Synergy H1 Plate Reader (BioTek) after a 3-hour incubation at 37° C. Dose response curves and respective LD₅₀ curves were generated using GraphPad Prism software (version 8.4.3). Curves were based on a nonlinear regression fit.

### Databases Used and Data Availability

The Allen Brain Map Human M1 10× Transcriptomics Explorer database was used to visualize expression levels (counts per million; CPM) in normal human brain cells. Proteomic data for normal brain was collected and annotated as described in Perna et al.³⁴ Publicly available H3K27ac ChIP-seq from the Gene Expression Omnibus (GEO) was displayed via the Integrative Genomics Viewer (IGV) (software.broadinstitute.org/software/igv/). Peaks associated around the ADGRE5 locus (chrl9:14488844-14534630; hgl9 reference genome) are shown for all specimens [IDH-wildtype GBM: GSM1824800, GSM1824811; IDH-mutant anaplastic astrocytoma: GSM1824813, GSM1824808 (anaplastic astrocytoma)]. GBM phosphoproteomic and proteomic data were from the GBM datasets of the Clinical Proteomic Tumor Analysis Consortium (CPTAC) included in Wang et al. analyzing multi-omic profiles from 99 treatment-naïve GBM specimens.³⁵ Publicly available single-cell RNA-seq data of adult and pediatric glioblastoma from the Broad Institute Single Cell Portal (GSE131928) were used to look at expression of ADGRE5 and its putative ligands THY1/CD90 and CD55. Bulk RNA-sequencing and single cell RNA/ATAC-sequencing data have been deposited at GEO under GSE230393 and GSE230389 accession numbers, respectively.

### Statistical Analysis

All experiments were performed in biological replicates of at least three repeats unless otherwise specified. Statistical analysis was performed using GraphPad Prism (version 8.4.3). Summary statistics are represented as mean±standard error of the mean (SEM) unless otherwise indicated. Statistical significance was calculated using either Students t-test, logrank test (for Kaplan-Meier survival curves), 1-way analysis of variance (ANOVA), or 2-way ANOVA, with Tukey's or Sidak's post hoc test for multiple comparisons. P values <0.05 were considered statistically significant (*, p<0.05; **, p<0.01; ***, p<0.001; ****, p<0.0001).

### Steady-State and Flux Metabolomics

Extraction of metabolites from Plasma—Prior to extraction, samples were moved from −80° C. storage to wet ice and thawed. Extraction buffer, consisting of 80% methanol (Fisher Scientific) and 500 nM metabolomics amino acid mix standard (Cambridge Isotope Laboratories), was prepared and placed on dry ice. Samples were extracted by mixing 50 μL of sample with 950 μL of extraction buffer in 2.0 mL screw cap vials containing ˜100 μL of disruption beads (Research Products International). Each sample was homogenized for 10 cycles on a bead blaster homogenizer (Benchmark Scientific). Cycling consisted of a 30 second homogenization time at 6 m/s followed by a 30 second pause. Samples were subsequently spun at 21,000 g for 3 minutes at 4° C. A set volume of each (450 μL) was transferred to a 1.5 mL tube and dried down by speedvac (Thermo Fisher). Samples were reconstituted in 50 μL of Optima LC/MS grade water (Fisher Scientific). Samples were sonicated for 2 minutes, then spun at 21,000 g for 3 minutes at 4° C. 20 μL were transferred to LC vials containing glass inserts for analysis. The remaining sample was placed in −80° C. for long term storage.

LC-MS/MS with the hybrid metabolomics method—Samples were subjected to an LC-MS analysis to detect and quantify known peaks. A metabolite extraction was carried out on each sample based on a previously described method (Jones, D. R., et al., A nano ultra-performance liquid chromatography-high resolution mass spectrometry approach for global metabolomic profiling and case study on drug-resistant multiple myeloma. Anal Chem 86, 3667-3675, doi:10.1021/ac500476a (2014)). The LC column was a Millipore™ ZIC-pHILIC (2.1×150 mm, 5 m) coupled to a Dionex Ultimate 3000™ system and the column oven temperature was set to 25° C. for the gradient elution. A flow rate of 100 L/min was used with the following buffers; A) 10 mM ammonium carbonate in water, pH 9.0, and B) neat acetonitrile. The gradient profile was as follows; 80-20% B (0-30 minutes), 20-80% B (30-31 minutes), 80-80% B (31-42 minutes). Injection volume was set to 2 L for all analyses (42 minutes total run time per injection). MS analyses were carried out by coupling the LC system to a Thermo Q Exactive HF™ mass spectrometer operating in heated electrospray ionization mode (HESI). Method duration was 30 minutes with a polarity switching data-dependent Top 5 method for both positive and negative modes. Spray voltage for both positive and negative modes was 3.5 kV and capillary temperature was set to 320° C. with a sheath gas rate of 35, aux gas of 10, and max spray current of 100 μA. The full MS scan for both polarities utilized 120,000 resolution with an AGC target of 3e6 and a maximum IT of 100 ms, and the scan range was from 67-1000 m/z. Tandem MS spectra for both positive and negative mode used a resolution of 15,000, AGC target of le5, maximum IT of 50 ms, isolation window of 0.4 m/z, isolation offset of 0.1 m/z, fixed first mass of 50 m/z, and 3-way multiplexed normalized collision energies (nCE) of 10, 35, 80. The minimum AGC target was le4 with an intensity threshold of 2e5. All data were acquired in profile mode.

Relative quantification of metabolites—The resulting Thermo™ RAW files were converted to mzXML format using ReAdW.exe version 4.3.1 to enable peak detection and quantification. The centroided data were searched using an in-house python script Mighty_skeleton version 0.0.2 and peak heights were extracted from the mzXML files based on a previously established library of metabolite retention times and accurate masses adapted from the Whitehead Institute (Chen, W. W., et al., Absolute Quantification of Matrix Metabolites Reveals the Dynamics of Mitochondrial Metabolism. Cell 166, 1324-1337.e1311, doi:10.1016/j.cell.2016.07.040 (2016)), and verified with authentic standards and/or high-resolution MS/MS spectral manually curated against the NIST14MS/MS (Sim6n-Manso, Y. et al., Metabolite profiling of a NIST Standard Reference Material for human plasma (SRM 1950): GC-MS, LC-MS, NMR, and clinical laboratory analyses, libraries, and web-based resources. Anal Chem 85, 11725-11731, doi:10.1021/ac402503m (2013)) and METLIN (2017) (Smith, C. A. et al., METLIN: a metabolite mass spectral database. Ther Drug Monit 27, 747-751, doi:10.1097/01.ftd.0000179845.53213.39 (2005)) tandem mass spectral libraries. Metabolite peaks were extracted based on the theoretical m/z of the expected ion type e.g., [M+H]+, with a ±5 part-per-million (ppm) tolerance, and a ±7.5 second peak apex retention time tolerance within an initial retention time search window of ±0.5 minutes across the study samples. The resulting data matrix of metabolite intensities for all samples and blank controls was processed with an in-house statistical pipeline Metabolyze version 1.0 and final peak detection was calculated based on a signal to noise ratio (S/N) of 3× compared to blank controls, with a floor of 10,000 (arbitrary units). For samples where the peak intensity was lower than the blank threshold, metabolites were annotated as not detected, and the threshold value was imputed for any statistical comparisons to enable an estimate of the fold change as applicable. The resulting blank corrected data matrix was then used for all group-wise comparisons, and t-tests were performed with the Python SciPy (1.1.0) (Jones, E., Oliphant, T. & Peterson, P. SciPy: Open Source Scientific Tools for Python. (2001)) library to test for differences and generate statistics for downstream analyses. Any metabolite with p-value <0.05 was considered significantly regulated (up or down).

### Results

### CD97 is De Novo Expressed in GBM Relative to Normal Brain

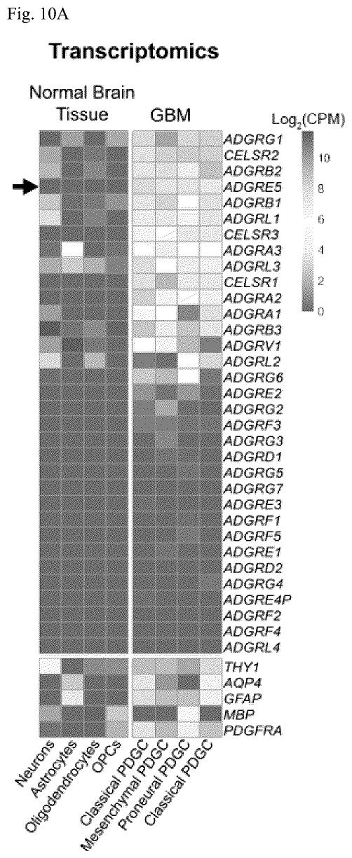

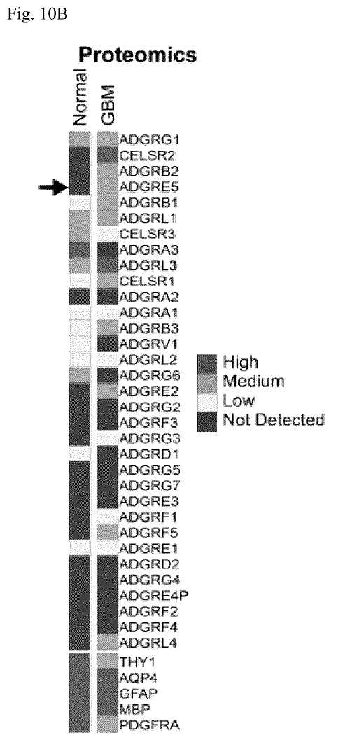

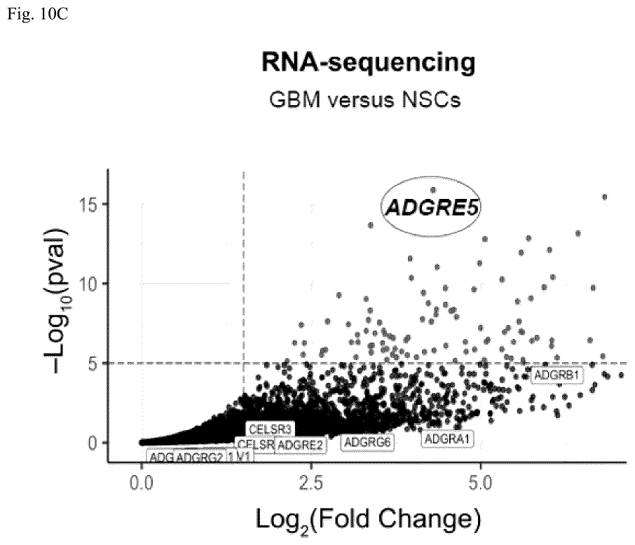

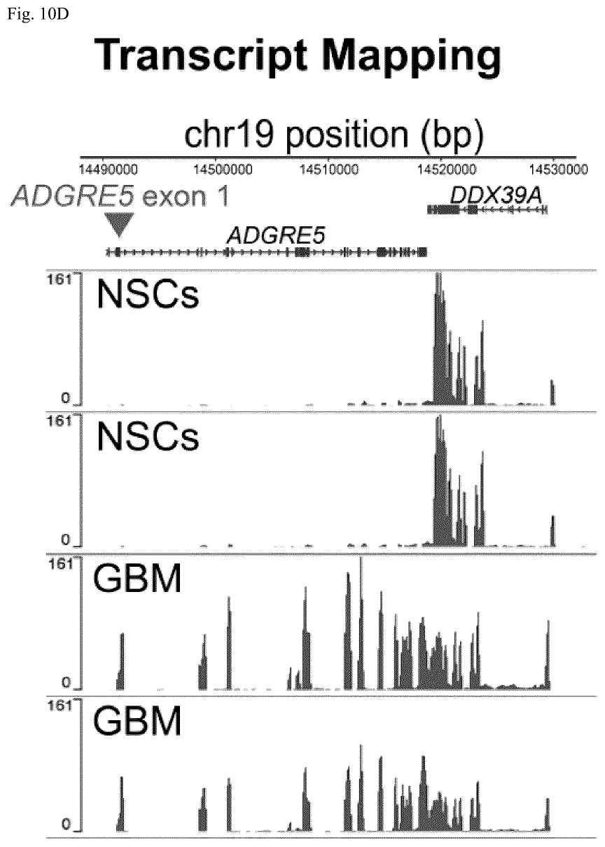

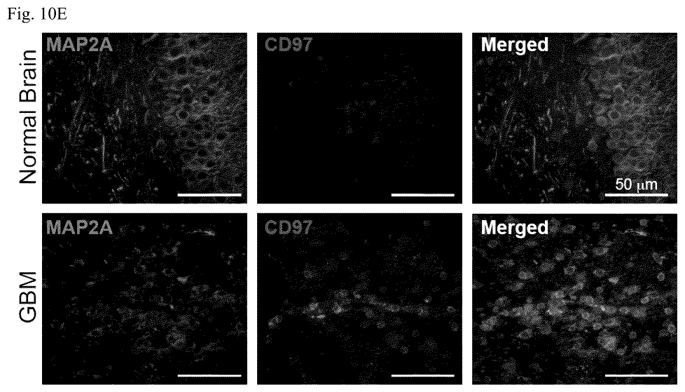

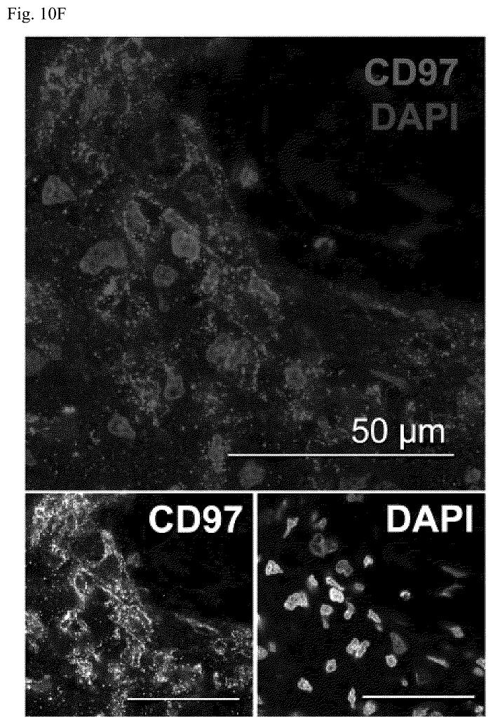

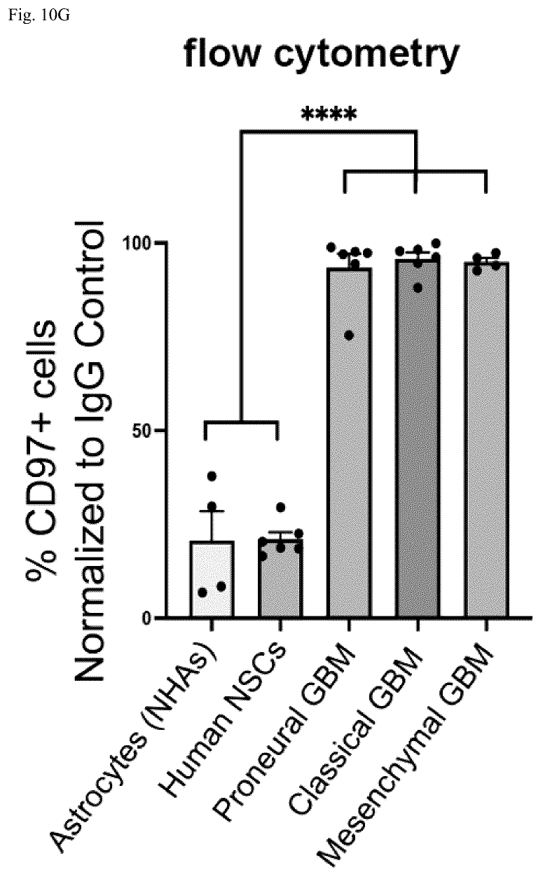

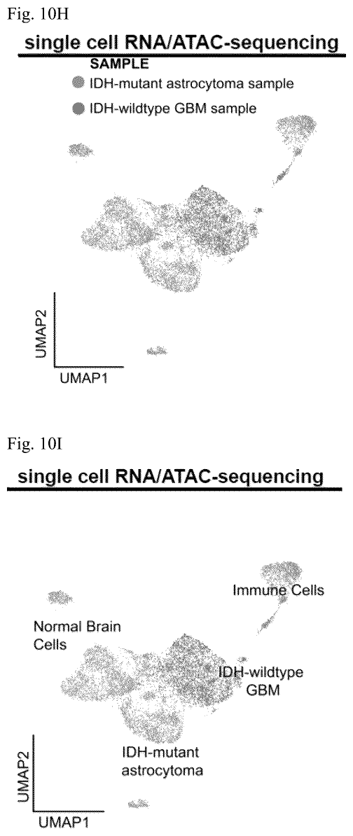

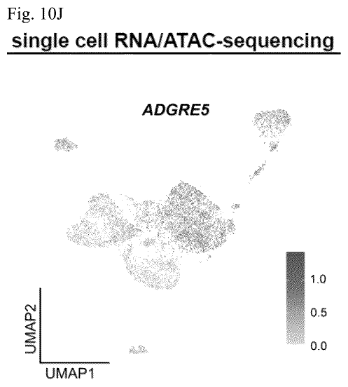

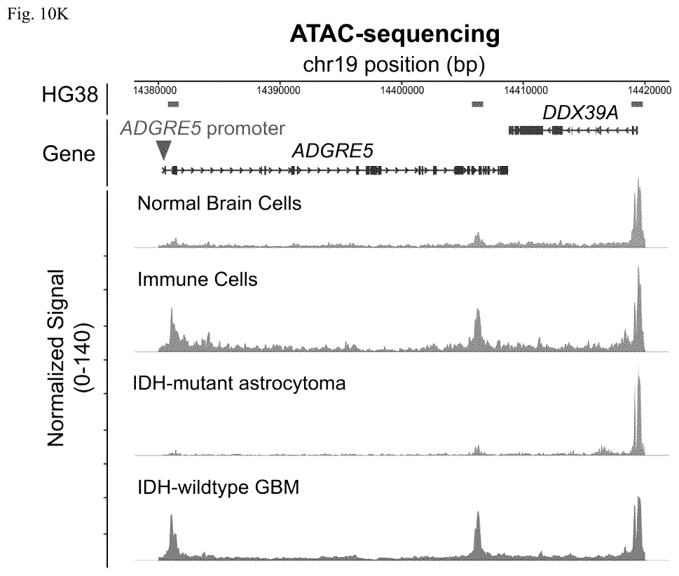

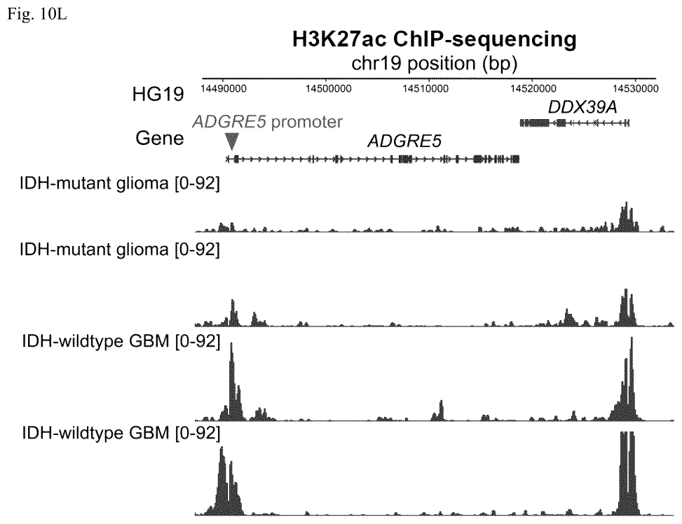

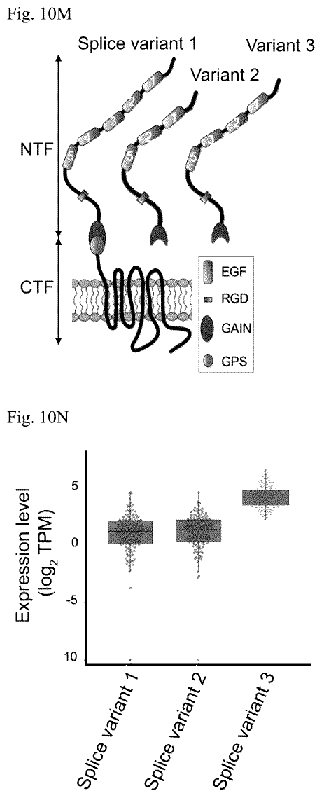

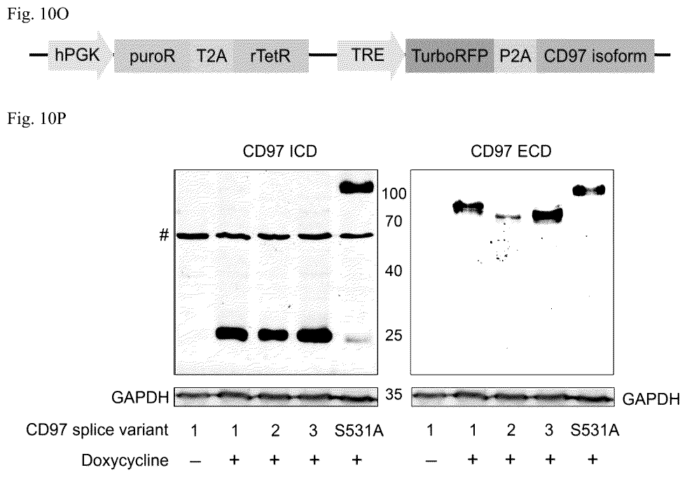

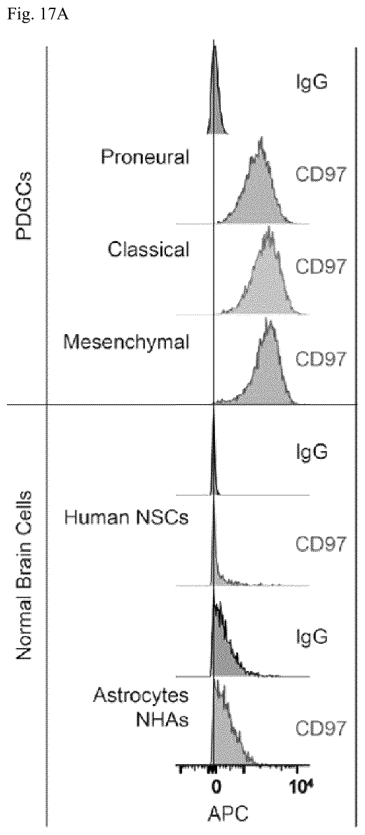

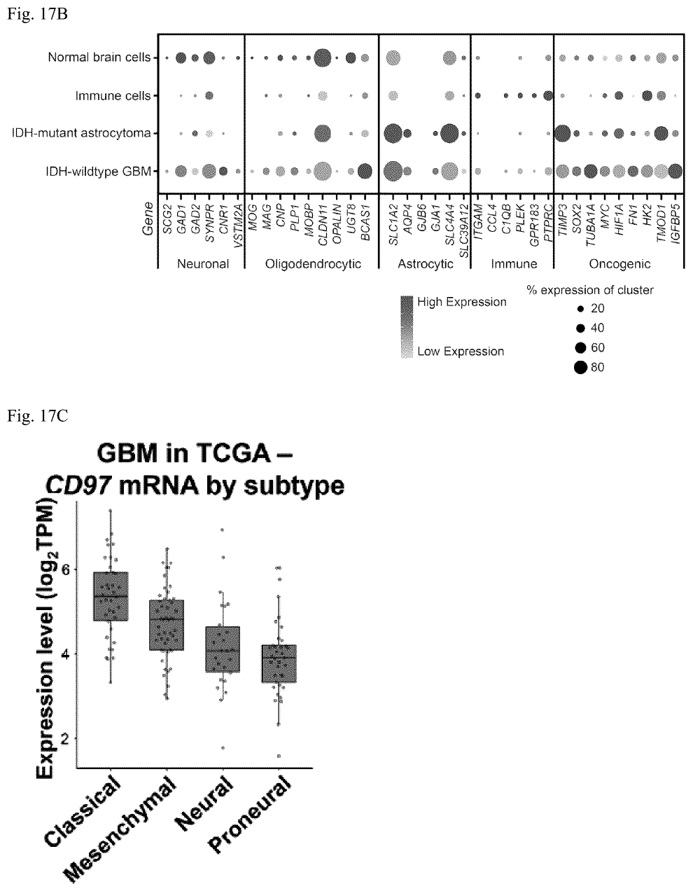

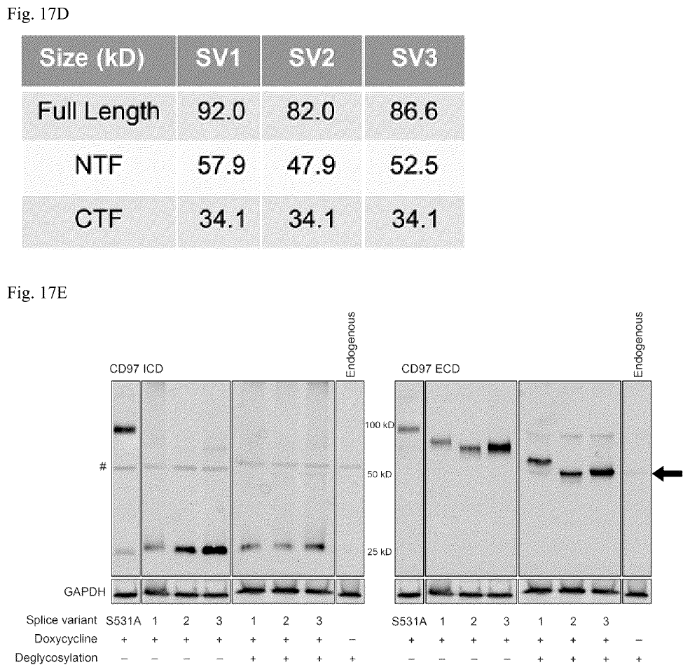

To compare the expression patterns of aGPCRs in GBM relative to normal, non-neoplastic brain tissue, a combined transcriptomic and proteomic approach was undertaken. First, the mRNA expression levels of all 33 aGPCRs were analyzed in normal brain tissue using the Allen Brain Map database, and in PDGCs collected in-house (FIG. 10A). The transcriptional data suggested upregulated expression of several aGPCRs in GBM compared to normal brain cell types. Second, proteomic data from normal brain³⁴ and GBM (CPTAC—Clinical Proteomic Tumor Analysis Consortium)³⁵ were interrogated, and confirmed de novo expression at the protein level for some aGPCRs was confirmed (FIG. 10B). Third, the differential gene expression of aGPCRs between human NSCs, the putative cell-of-origin in GBM,¹⁸ and PDGCs was compared using bulk RNA-sequencing (RNA-seq) data (FIGS. 10C, 10D). Collectively, it was found herein that ADGRE5 (CD97) fulfilled criteria for the single most differentially expressed aGPCR in GBM compared to healthy brain: it was one of the most upregulated aGPCRs in PDGCs and was detected in all 99 GBM specimens from the CPTAC database³⁵; it was absent from non-neoplastic brain tissue at the transcriptomic and proteomic level; and the statistical significance of its transcriptional upregulation in PDGCs compared to NSCs was the highest genome-wide. To confirm these findings, immunofluorescent staining of normal temporal lobe tissue sections and several (n=10) GBM tissue sections was performed and it was confirmed that CD97 was uniquely expressed in GBM and not in normal brain (FIG. 10E). Confocal microscopy confirmed plasma membrane localization of the receptor in tumor cells within GBM biospecimens (FIG. 10F). Using flow cytometry, robust surface expression of CD97 was found in all tested PDGCs (representing all three TCGA-defined transcriptional subtypes of GBM: proneural, classical, and mesenchymal^{2,19}), and low expression in non-neoplastic human astrocytes (NHAs) and NSCs (FIG. 10G; FIG. 17A). These data suggest that CD97 is de novo expressed in GBM relative to non-neoplastic brain tissue and that it is ubiquitously expressed in all GBM tumors.

It was then tested whether ADGRE5 expression differs between IDH-wildtype GBM and low-grade IDH-mutant gliomas using single-cell RNA/ATAC (Assay for Transposase-Accessible Chromatin)-seq data collected from an IDH-mutant WHO (World Health Organization) grade II astrocytoma and an IDH-wildtype GBM (FIG. 10H). After identifying cell clusters based on single-cell RNA-seq gene expression (FIG. 10I; FIG. 17B) via Seurat, it was found that ADGRE5 expression was highest in IDH-wildtype GBM tumor cells and immune cells, but nearly absent in IDH-mutant low-grade astrocytoma cells (FIG. 10J). Analysis of single-cell ATAC-seq data using Signac demonstrated open, accessible chromatin at the ADGRE5 locus only in GBM and immune cell clusters, but not IDH-mutant astrocytoma cells and normal brain lineages (FIG. 10K). Similarly, chromatin immunoprecipitation (ChIP)-seq for H3K27 acetylation, an activating chromatin modification, obtained from publicly available Gene Expression Omnibus (GEO) datasets³⁶, showed increased activation around the ADGRE5 promoter in IDH-wildtype GBM but not in IDH-mutant gliomas (FIG. 10L). Together, these findings suggest that CD97 expression is abundant in IDH-wildtype GBM but only limited in low-grade IDH-mutant astrocytoma.

Alternative splicing of ADGRE5 mRNA generates three distinct isoforms that differ in the number of epidermal growth factor (EGF)-like repeats in the N-terminal extracellular domain (ECD) of the receptor (FIG. 10M).²s Analysis of TCGA RNA-seq data indicated that isoform 3, with four EGF repeats in the N-terminus, was the predominant variant in GBM (FIG. 10N). CD97 transcript was found in all transcriptional subtypes of GBM in TCGA (FIG. 17C). The extent of autoproteolytic cleavage for each aGPCR sometimes varies depending on the cellular context and cell type.³⁷ To test whether CD97 undergoes cleavage at the GPS site in GBM, all three isoforms of CD97 and a non-cleavable point mutant of CD97 (CD97 S531A)³⁸ were expressed exogenously in PDGCs (FIG. 10O). Immunoblotting against the ECD and the intracellular domain (ICD) of CD97 revealed that the vast majority of the receptor undergoes cleavage in PDGCs to generate a membrane-bound C-terminal fragment (CTF) and an extracellular N-terminal fragment (NTF), regardless of the isoform that is overexpressed (FIG. 10P). To resolve discrepancies in the expected and observed molecular weights of the NTF (FIG. 17D), whole cell lysates collected from a PDGC endogenously expressing CD97 or exogenously overexpressing the CD97 isoform were deglycosylated before immunoblot staining. The deglycosylation resulted in band shifts to the expected molecular weights (FIG. 17E), suggesting that the CD97 NTF was glycosylated. Interestingly, the band corresponding to endogenously-expressed deglycosylated CD97 NTF appeared to match the molecular weight of the NTF of exogenously-expressed CD97 isoform 3 (FIG. 17E). This finding supported the transcriptomic TCGA data indicating that the predominant species of CD97 in GBM is isoform 3 (with four extracellular EGF repeats) and suggested that CD97 undergoes autoproteolytic cleavage in GBM.

### CD97 Loss Compromises GBM Growth

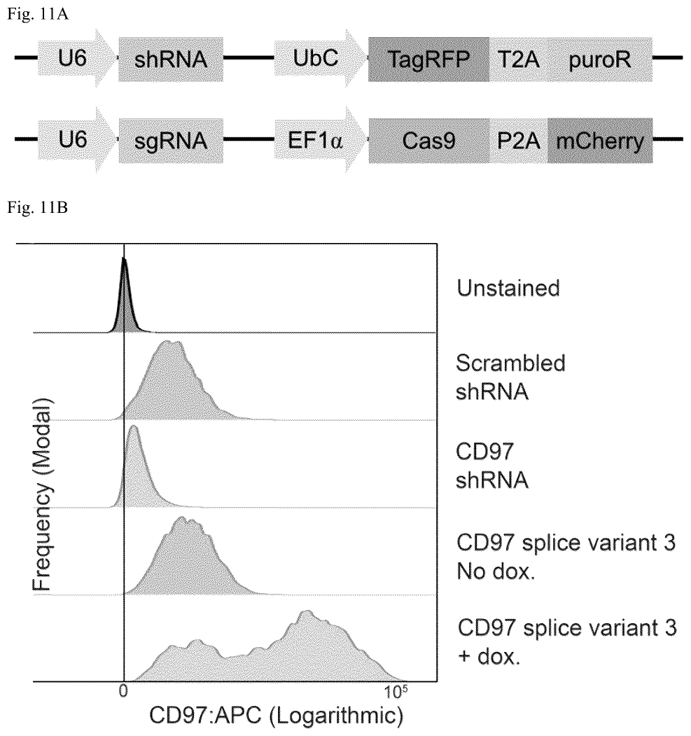

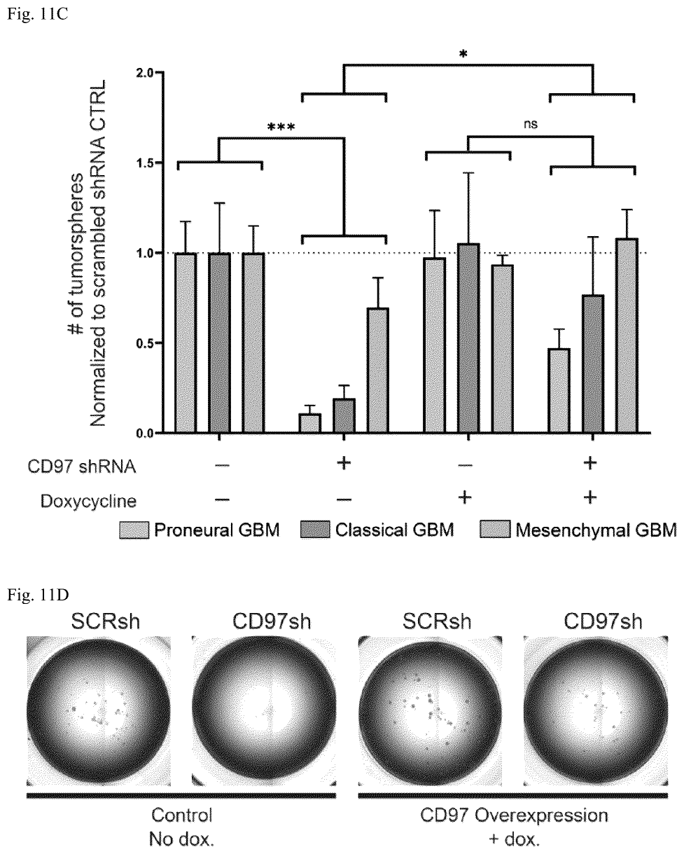

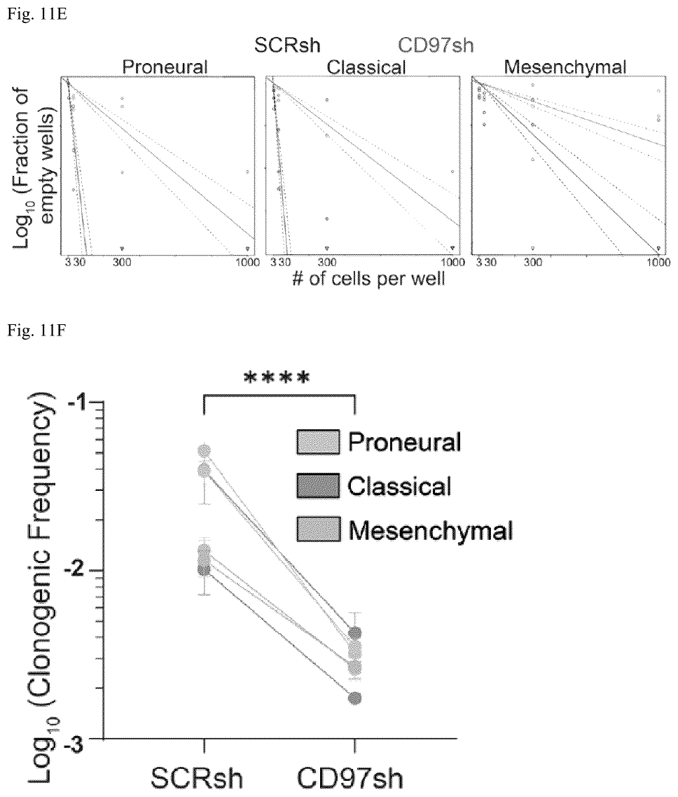

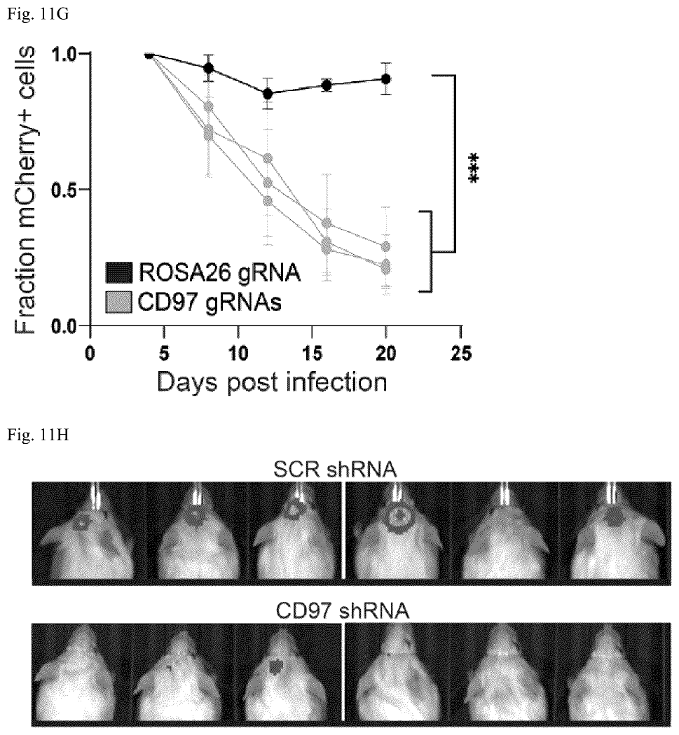

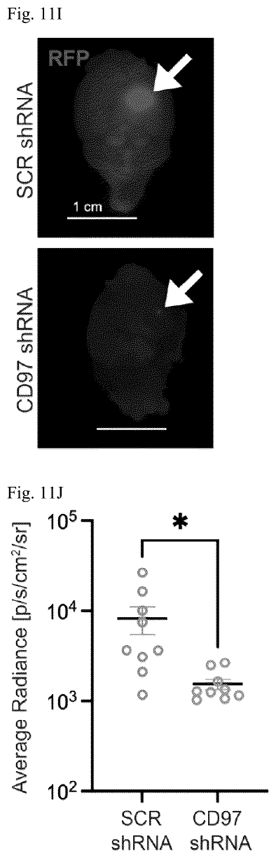

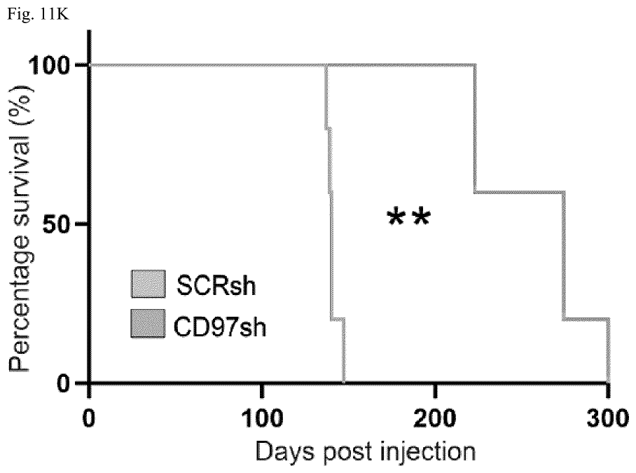

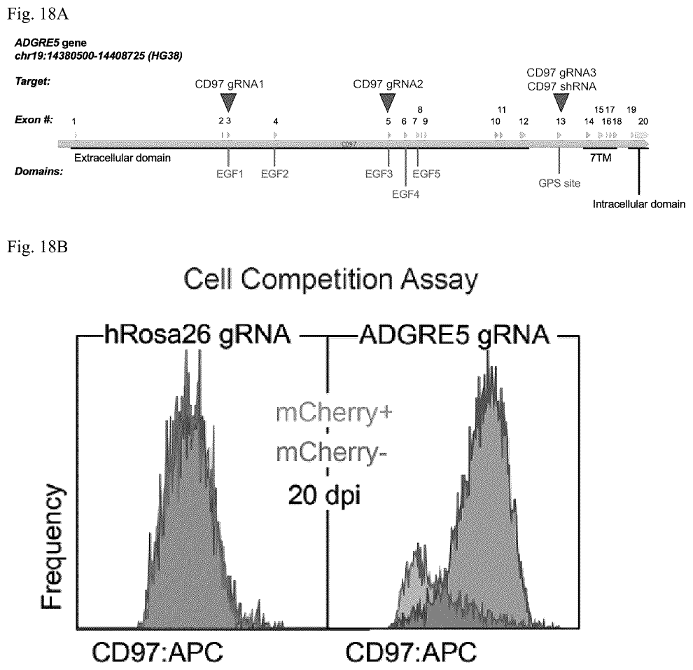

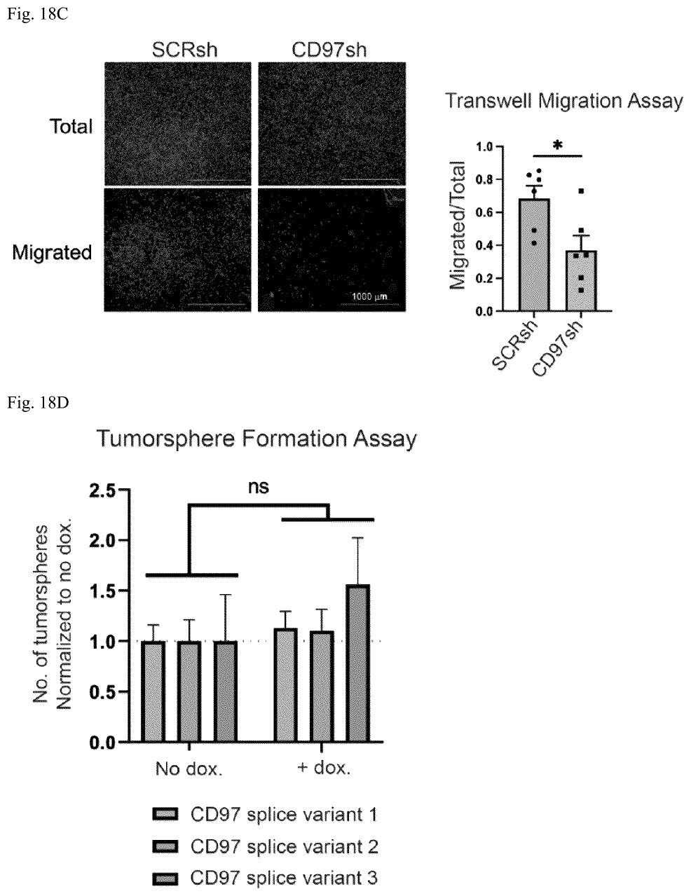

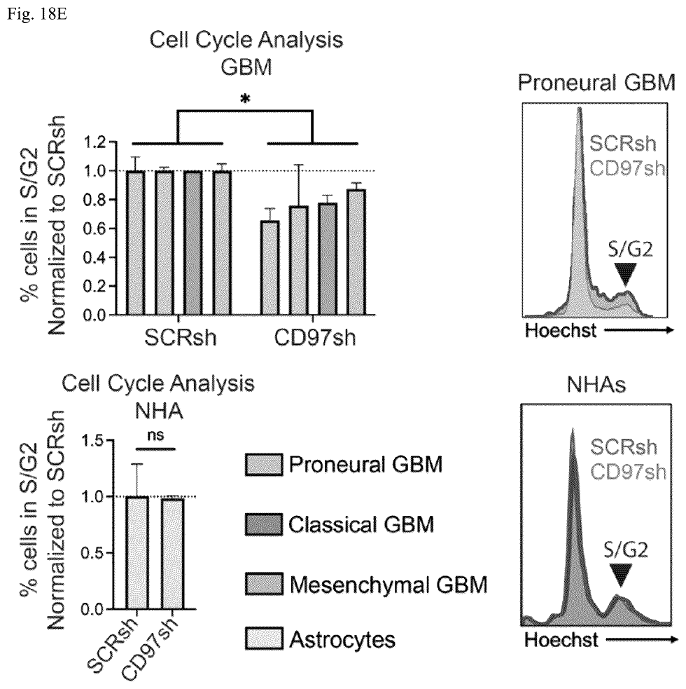

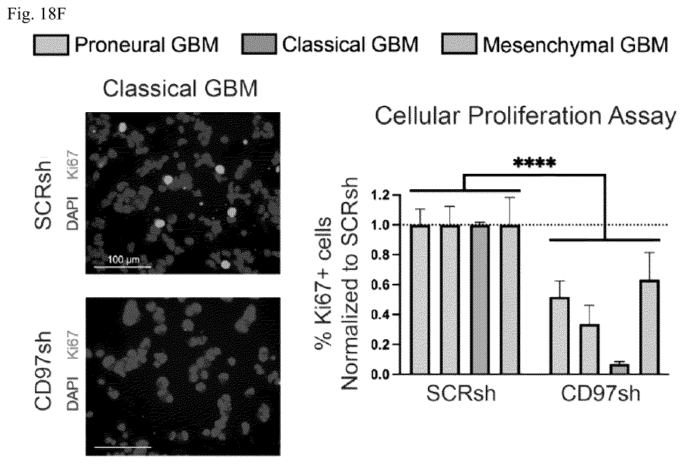

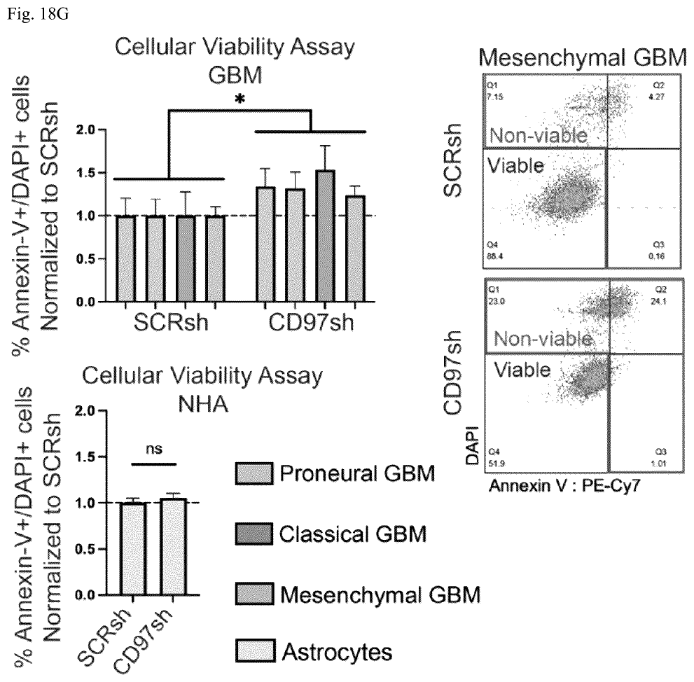

To further investigate the role of CD97 in GBM, short hairpin RNAs (shRNAs) and guide RNAs (gRNAs) were established targeting all isoforms of CD97 (FIG. 11A; FIG. 18A). Flow cytometric surface staining confirmed loss of CD97 upon shRNA-mediated knockdown (FIG. 11B) and CRISPR-Cas9-mediated knockout (FIG. 18B). Since CD97 is implicated in GBM cell migration^{29,32,33}, a transwell migration assay was performed which confirmed significant reduction in PDGC migration after lentiviral infection with the CD97 shRNA compared to a scrambled (SCR) shRNA control (FIG. 18C). Importantly, knockdown of CD97 produced a substantial growth defect in the PDGCs in vitro. This was quantified using tumorsphere formation assays, which revealed a significant loss of tumorsphere formation after CD97 knockdown (FIGS. 11C, 11D). Overexpression of a doxycycline (dox)-inducible shRNA-resistant form of CD97 cDNA alone did not increase tumorsphere formation in PDGCs (FIG. 18D), but rescued the impairment in tumorsphere formation following CD97 knockdown (FIGS. 11C, 11D), suggesting specificity of the phenotype observed with CD97 shRNA. The effects of CD97 knockdown on PDGCs were also observed in extreme limiting dilution assays (FIGS. 11E, 11F), suggesting a diminished clonogenic frequency and impaired GBM stem cell self-renewal in the absence of CD97. Significant effects of CD97 were also observed on cellular proliferation using cell cycle analysis (FIG. 18E) and Ki67 immunofluorescent staining (FIG. 18F), as well as on cellular viability using flow cytometry for Annexin-V and DAPI (4′,6-diamidino-2-phenylindole) staining (FIG. 18G). In contrast, CD97 knockdown in NHAs did not compromise their proliferation or their viability (FIGS. 18E, 18G). CRISPR-Cas9 knockout of CD97 phenocopied the shRNA knockdown, and significantly hindered PDGC growth and viability relative to a gRNA directed at the safe harbor human ROSA26 locus in a cell competition assay (FIG. 11G). To determine whether CD97 knockdown also impaired tumor formation in vivo, intracranial xenograft assays were performed in immunodeficient NSG mice. Implantation with PDGCs following CD97 knockdown resulted in significantly less tumor growth (FIGS. 11H-11J) and significantly prolonged mouse survival (FIG. 11K). These findings demonstrate that CD97 loss compromises PDGC proliferation and viability and reduces tumor initiation in vitro and in vivo.

### CD97 Knockdown Impairs Glycolytic Metabolism

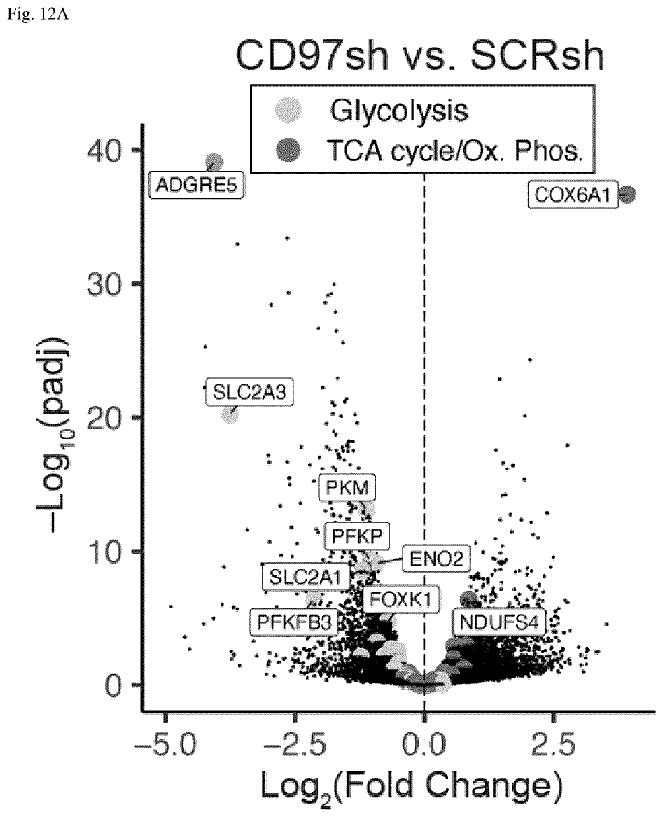

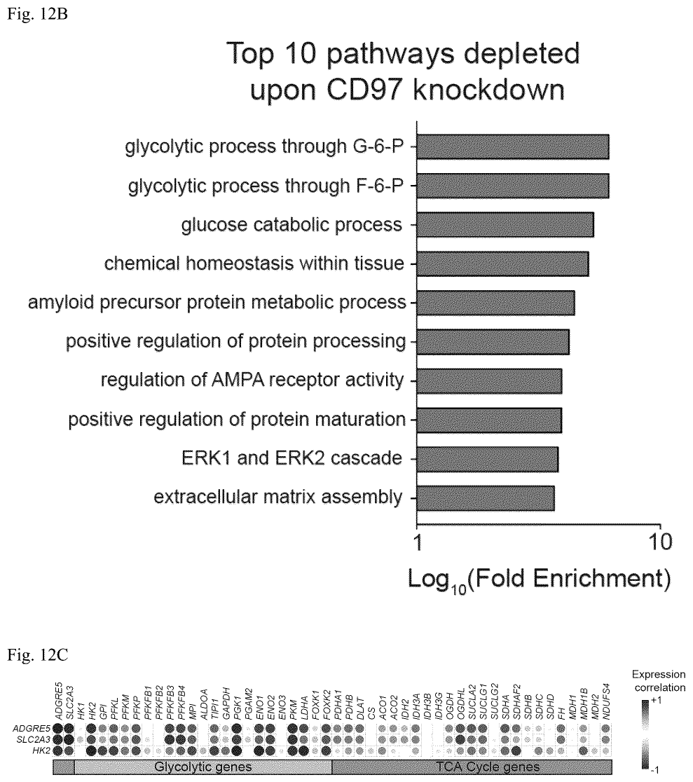

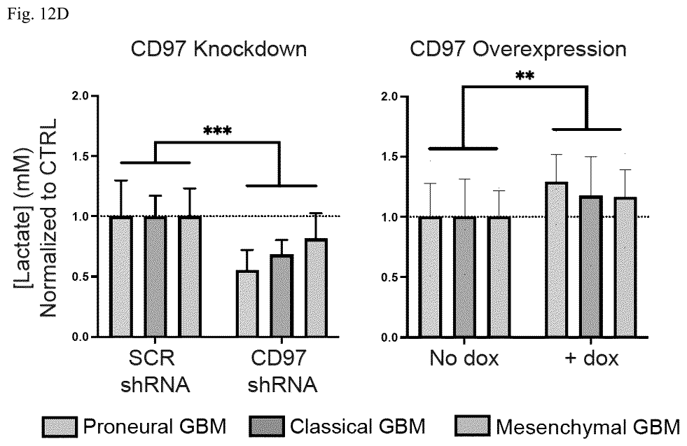

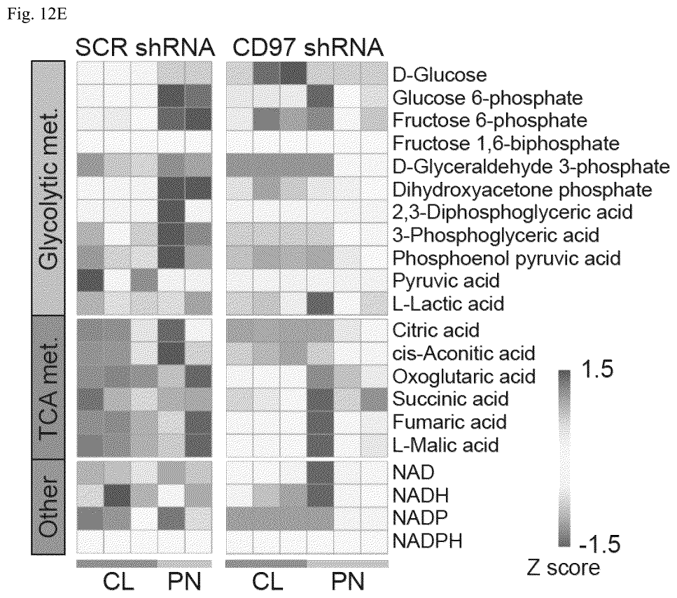

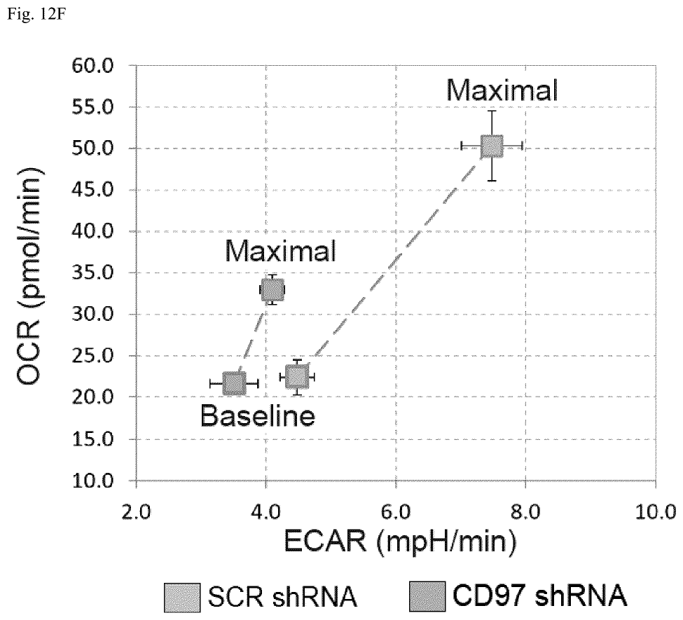

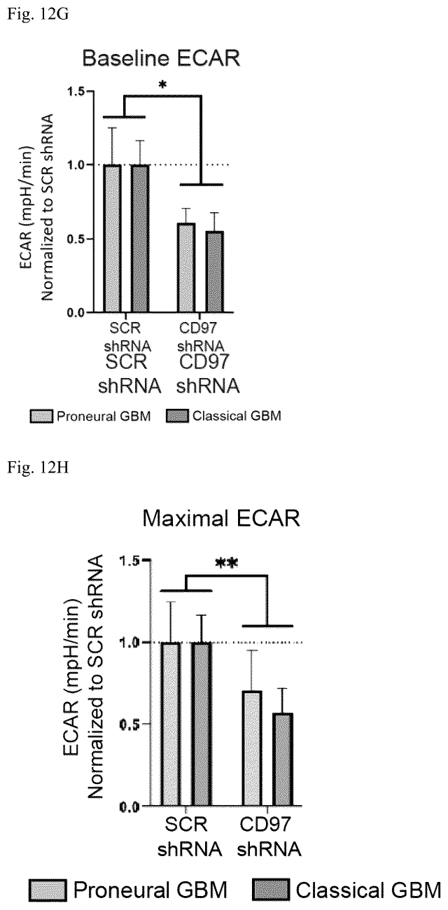

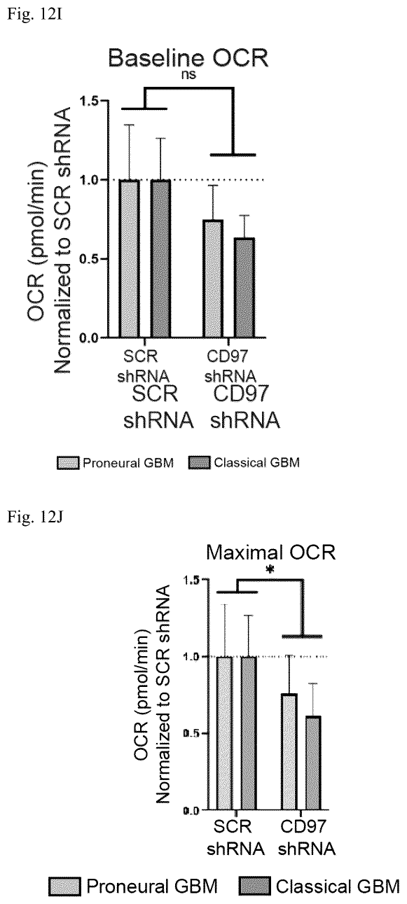

To elucidate mechanisms underlying the actions of CD97 in PDGCs, bulk RNA-seq was performed following knockdown of the receptor in one of the PDGCs. In order to capture early consequences of CD97 knockdown before any effects on cellular viability, cells were collected four days after infection with the lentiviral shRNA vector. Over 3000 genes were differentially expressed between the CD97 knockdown and the SCR shRNA control PDGCs (FIG. 12A). Among the most downregulated genes was ADGRE5, confirming a robust knockdown of the receptor (FIG. 12A). Gene Ontology (GO) PANTHER pathway analysis to detect gene sets that were depleted upon CD97 knockdown identified glycolytic metabolism as the most impacted biological process (FIG. 12B). This was noteworthy, because GBM tumor cells exhibit increased reliance on glycolytic metabolism, a phenomenon known as the Warburg effect, compared to normal brain cells.³⁹⁻⁴² This led to hypothesize that CD97 plays a role in promoting glycolysis in GBM.

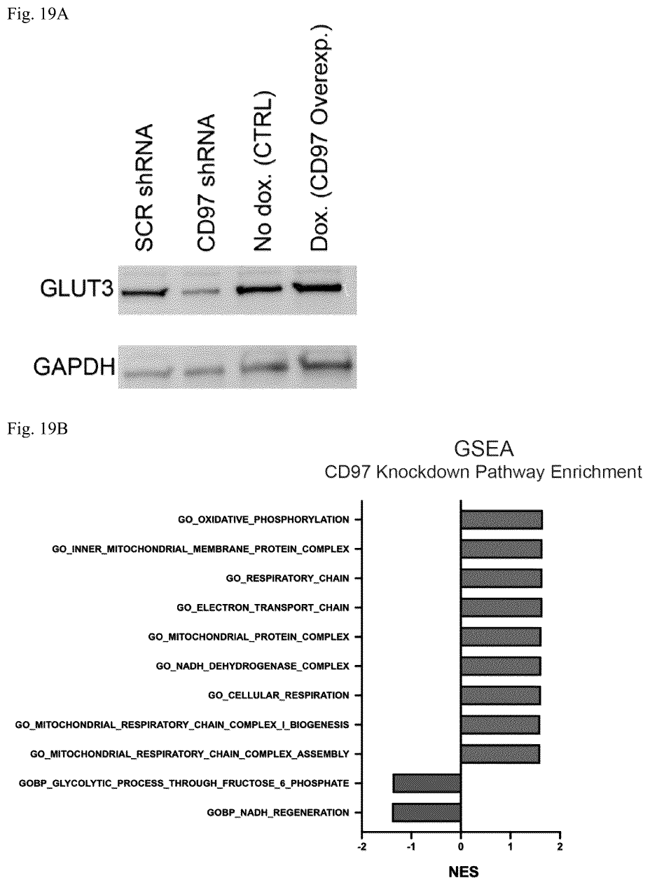

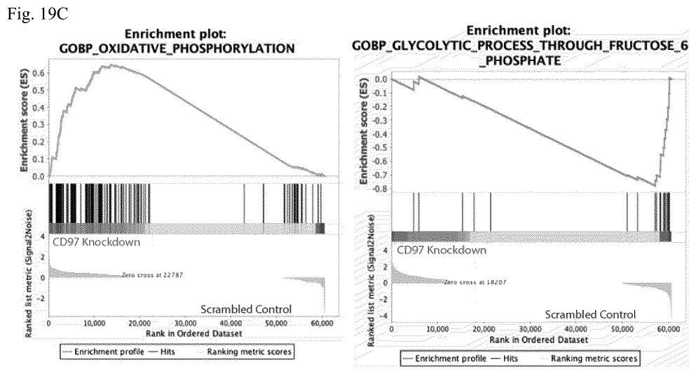

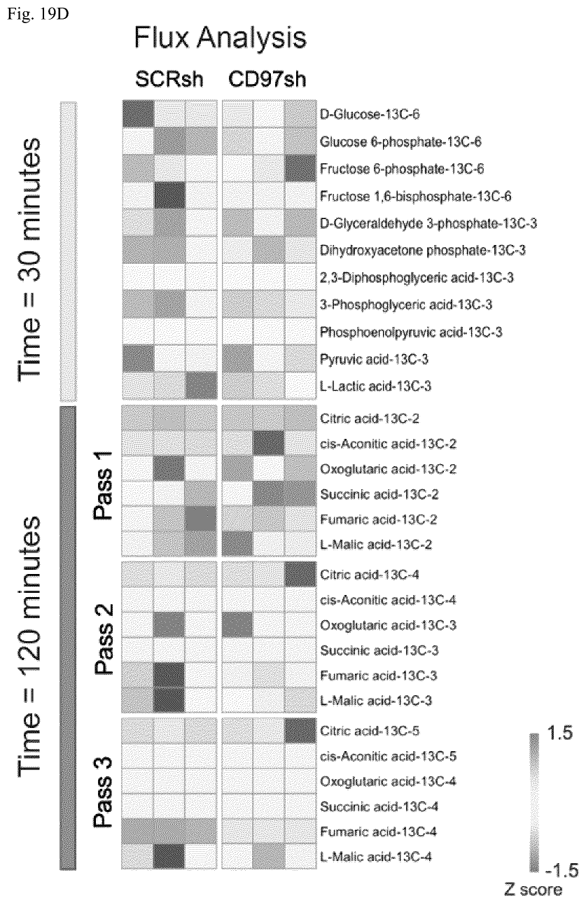

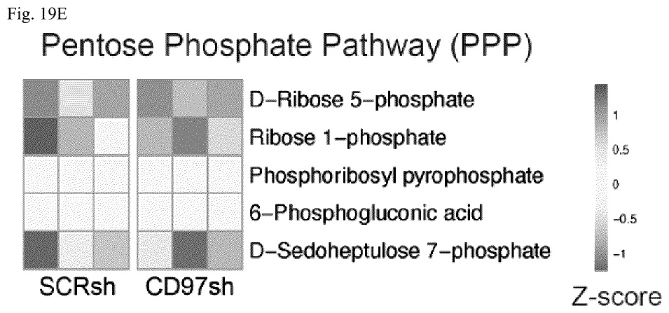

Indeed, when the differential gene expression data were revisited, it was found that the majority of genes involved in glycolysis and glucose transport were downregulated upon CD97 knockdown, while the opposite was observed for genes involved in the TCA cycle and in OXPHOS (FIG. 12A). In order to further visualize this, Differential Gene Correlation Analysis (DGCA) of transcriptomic data from a PDGC was performed following both overexpression or knockdown of CD97 (FIG. 12C). This analysis also revealed that ADGRE5 expression levels positively correlated with many genes relevant to glycolysis and negatively correlated with genes relevant to the TCA cycle (FIG. 12C). A reduction in the glucose transporter GLUT3 (SLC2A3) after CD97 knockdown at the protein level was also confirmed via immunoblot staining (FIG. 19A). Of note, several glycolytic enzymes and glucose transporters have been previously implicated in GBM pathogenesis⁴²⁻⁴⁵ and metabolic reprogramming.⁴⁶⁻⁴⁸ In order to ensure that the effects of CD97 knockdown on the transcription of metabolic genes were generalizable, Gene Set Enrichment Analysis (GSEA) was performed on RNA-seq data collected from three other PDGCs following CD97 knockdown. Here, a depletion of glycolytic processes and an enrichment of OXPHOS-related transcripts following CD97 knockdown was also observed (FIGS. 19B, 19C).

Based on the transcriptomic data, it was decided to investigate the metabolic consequences of CD97 perturbation in the PDGCs. Since Warburg metabolism is characterized by glucose catabolism biased toward lactate generation rather than pyruvate conversion to acetyl-CoA for utilization in the TCA cycle, lactate levels were measured in the culture medium after shRNA-mediated silencing or overexpression of CD97 in the PDGCs (FIG. 12D). Indeed, CD97 knockdown resulted in reduced levels of extracellular lactate, while levels increased upon CD97 overexpression. Both steady-state and flux metabolomic analysis were performed next in order to gain further insight on the regulation of enzymatic steps of glucose metabolism (FIGS. 12E, 19D). Steady-state metabolomic data showed a depletion of glycolytic metabolites following CD97 knockdown. Given that glycolytic intermediates feed into both the TCA cycle and the pentose-phosphate pathway (PPP), which is essential for nucleic acid synthesis and redox homeostasis, it is no surprise that these metabolites were also depleted after knockdown (FIGS. 12E, 19E).⁴⁹

Metabolic flux data generated using [U-¹³C₆]-glucose also revealed reduced levels of heavy carbon incorporation into glycolytic metabolites, while the effects on TCA cycle metabolites were less evident (FIG. 19D). Collectively, the transcriptomic and metabolomic data suggest that CD97 knockdown leads to a global reduction in glucose catabolism, which is likely to account for the reduced proliferation and viability of GBM cells. It was postulated herein that the upregulation of transcripts encoding enzymes of the TCA cycle and OXPHOS reflects inadequate compensation aimed to meet energy demands impacted by reduced glycolysis.

To further test the hypothesis that CD97 promotes glycolytic metabolism at a functional level, the rates of glycolysis and mitochondrial respiration were measured using a Seahorse XF Cell Energy Phenotype assay. This assay measures the extracellular acidification rate (ECAR), largely accounted for by lactate production, and the oxygen consumption rate (OCR) before and after the addition of mitochondrial stressors (oligomycin and FCCP). PDGCs following CD97 knockdown exhibited a loss of the ability to respond to mitochondrial stressors (FIG. 12F). Closer investigation revealed a significantly decreased ECAR both at baseline and after the addition of mitochondrial stressors (maximal ECAR) (FIGS. 12G, 12H). Though the OCR levels were also decreased following CD97 knockdown, these effects were not as striking as those with ECAR and did not reach statistical significance at baseline (FIGS. 121, 12J). Together, these data suggest that CD97 promotes glycolytic metabolism in GBM.

### CD97 Activates the MAPK/ERK Signaling Pathway

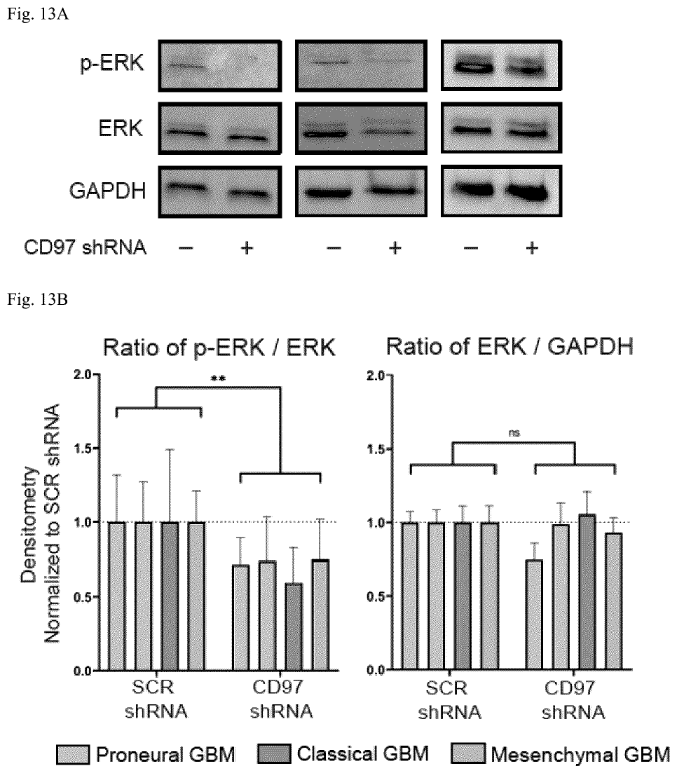

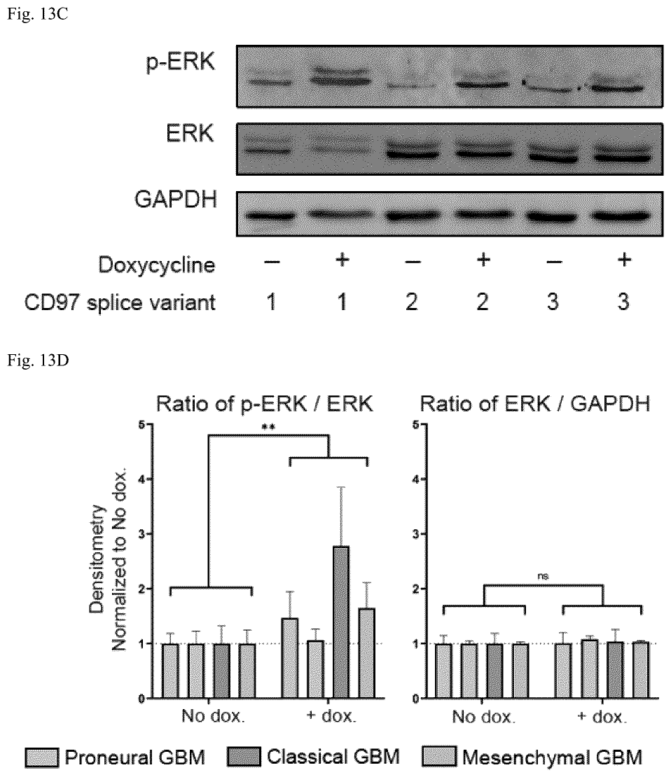

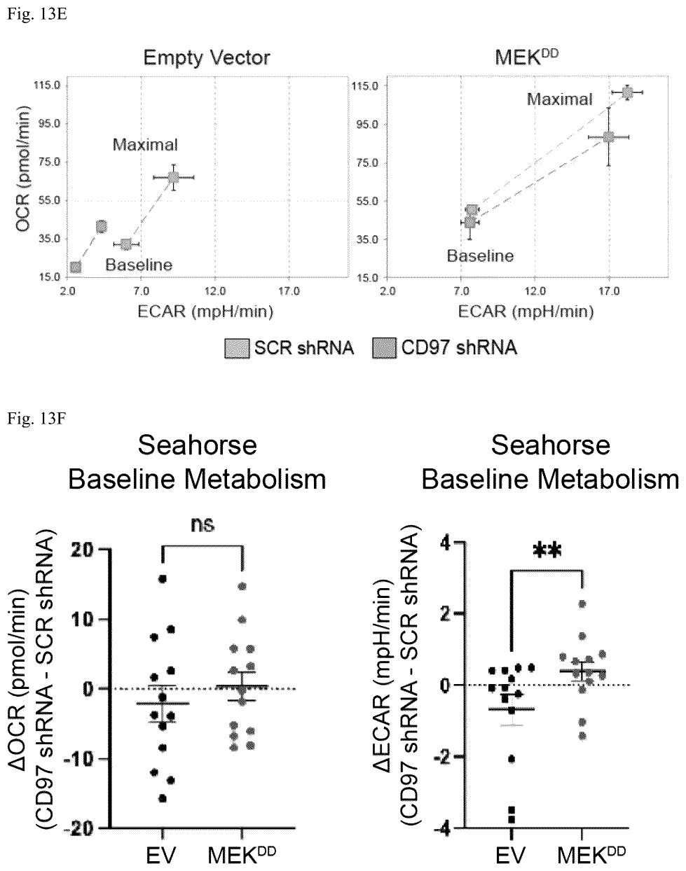

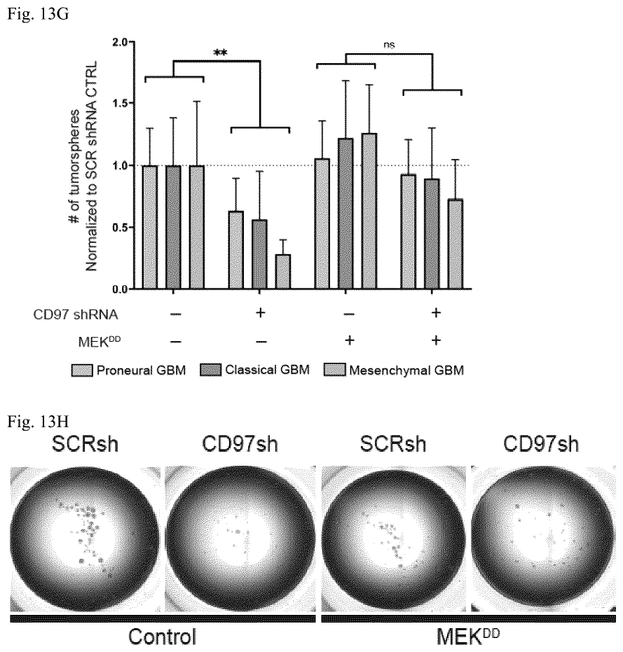

In an effort to identify signaling pathways activated by CD97 in GBM, the GO PANTHER pathway analysis of RNA-seq data shown previously was revisited (FIG. 12B). Here, transcripts involved in the ERK1/ERK2 signaling cascade were depleted upon CD97 knockdown in the present PDGCs. To confirm whether the MAPK pathway was activated by CD97 in the present PDGCs, immunoblot staining was performed against phosphorylated ERK1/ERK2 following CD97 knockdown or overexpression. CD97 knockdown significantly decreased levels of phosphorylated ERK1/ERK2 (FIGS. 13A, 13B). In contrast, overexpression of all CD97 isoforms led to significant increases in phosphorylated ERK1/ERK2 in most of the present PDGCs (FIGS. 13C, 13D). These results suggested that CD97 activates the MAPK signaling pathway.

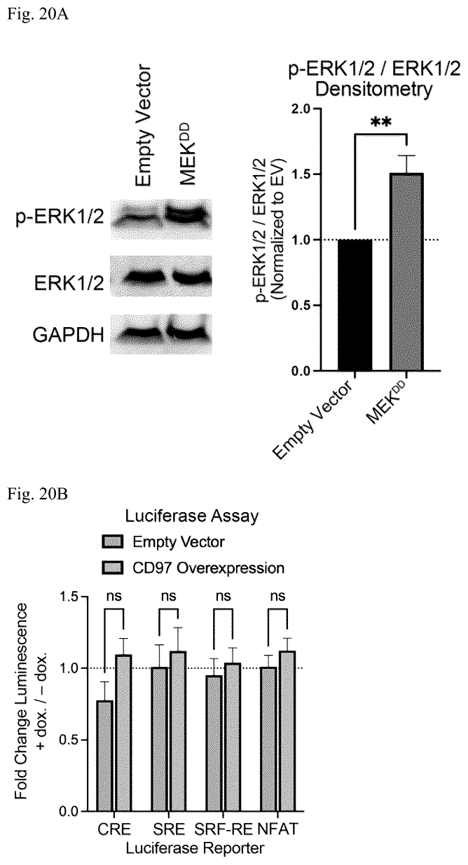

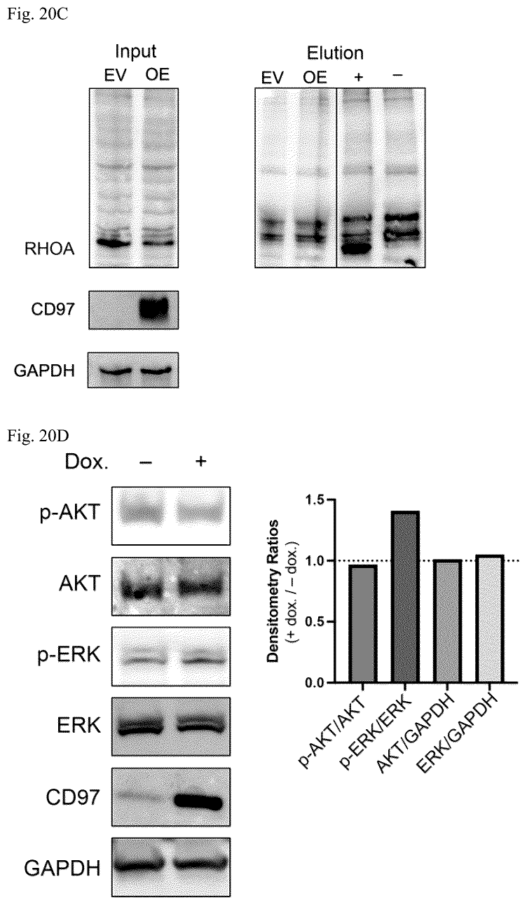

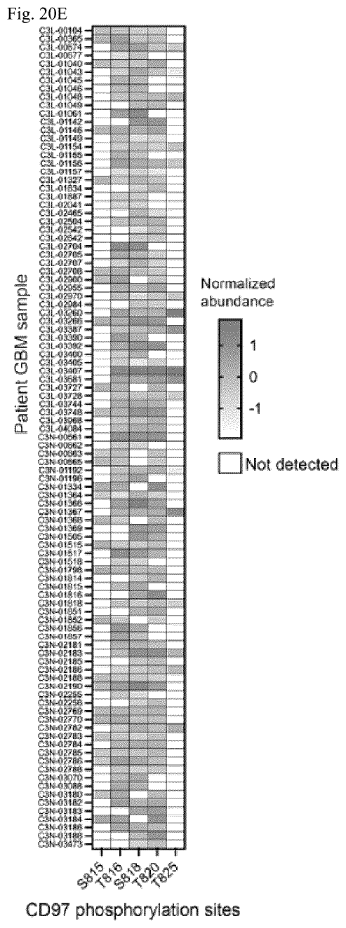

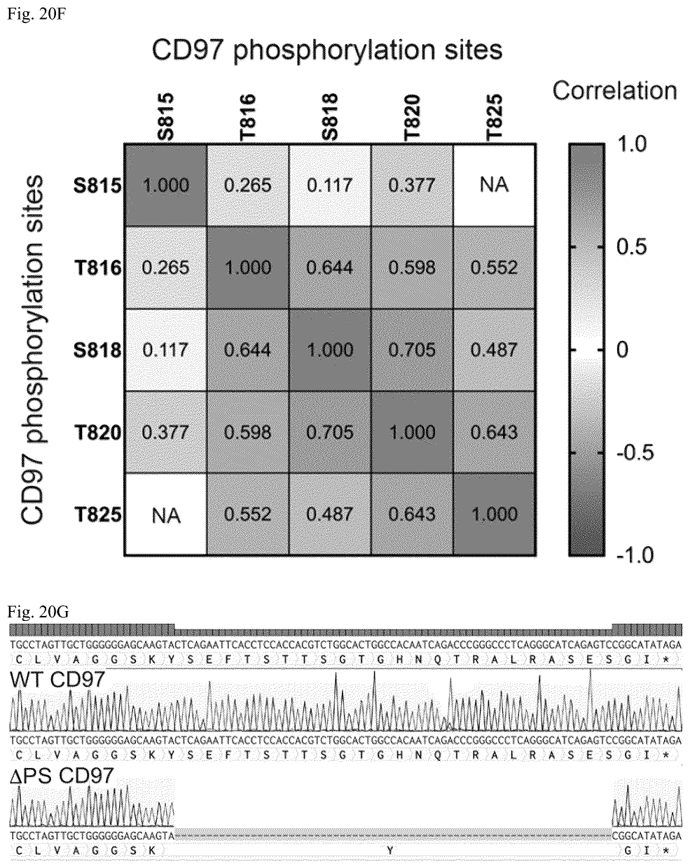

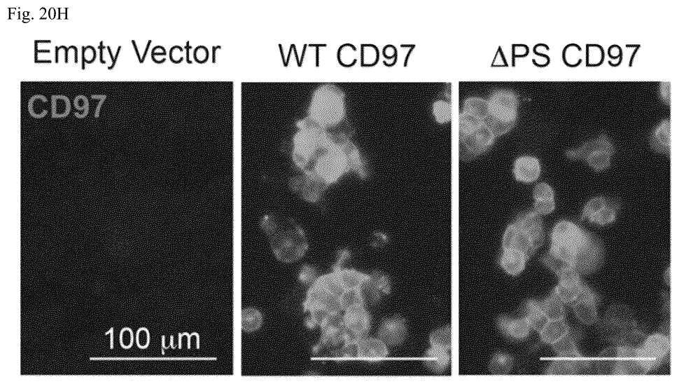

To test whether activation of the MAPK signaling pathway rescues the metabolic and growth phenotypes observed after CD97 knockdown, transfected PDGCs were stably transfected with a phosphomimetic MEK mutant (MEK S218D,S222D; denoted as MEK^{DD} onward), which was expected to constitutively phosphorylate and activate ERK1/ERK2.⁵⁰ It was confirmed that the MEK^{DD} mutant increased ERK1/ERK2 activation by performing an immunoblot against phosphorylated ERK (FIG. 20A). Transfection of PDGCs with the MEK^{DD} mutant restored their metabolic profile in Seahorse Cell Energy Phenotype assays following CD97 knockdown (FIG. 13E). Of particular interest was the finding that MEK^{DD} rescued the CD97 knockdown-imparted defect in ECAR, but not OCR (FIG. 13F), suggesting the CD97-MAPK signaling axis not only promotes glycolysis but also biases conversion of pyruvate to lactate rather than to acetyl-CoA. Equally importantly, the MEK^{DD} construct also rescued tumorsphere formation after knockdown of CD97 in PDGCs (FIGS. 13G, 13H). Together, these data suggest that CD97 activates MAPK signaling to promote glycolytic metabolism, thus enabling tumor growth in GBM.

### CD97 Activates the MAPK Signaling Cascade Through Phosphorylation of its C-Terminus and Recruitment of β-Arrestin

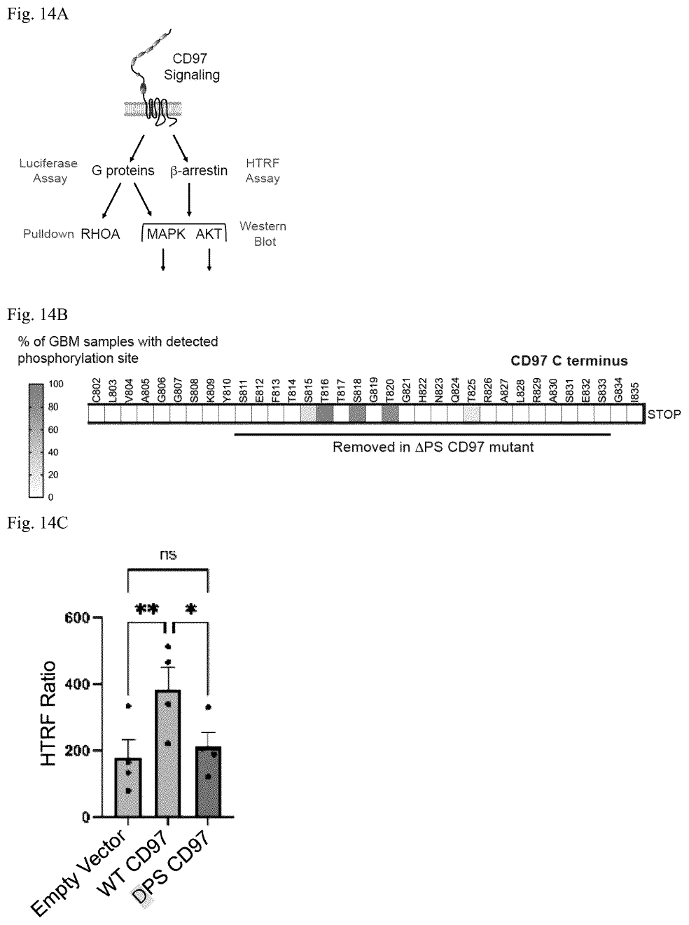

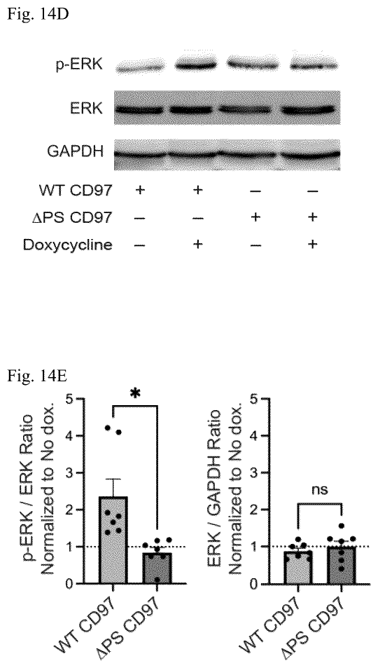

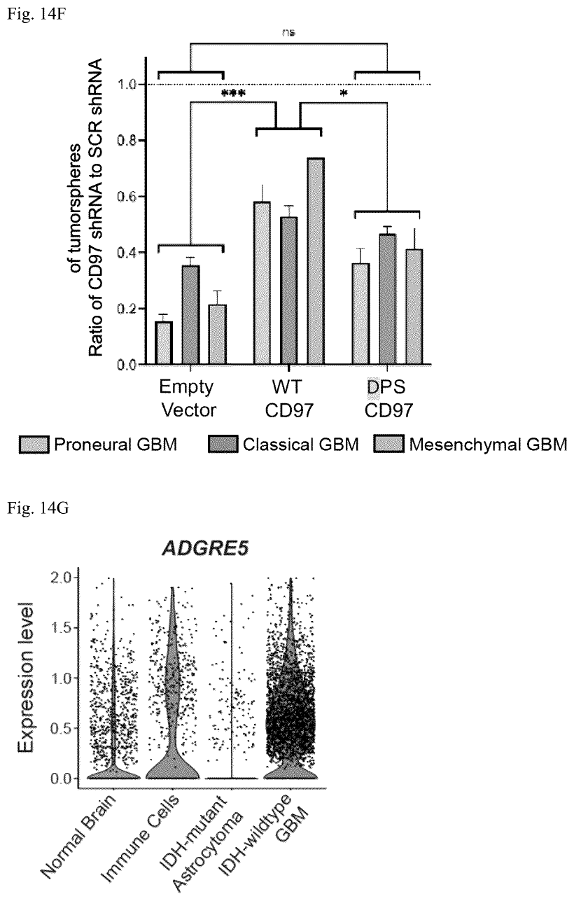

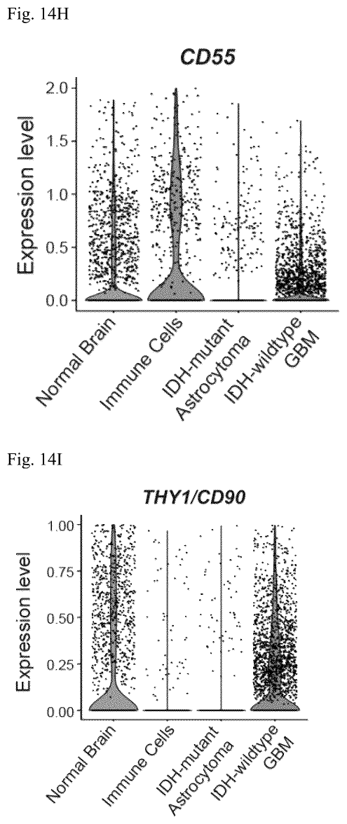

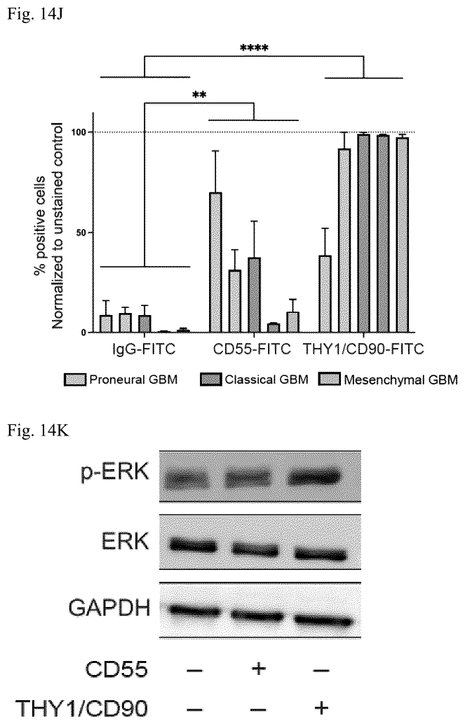

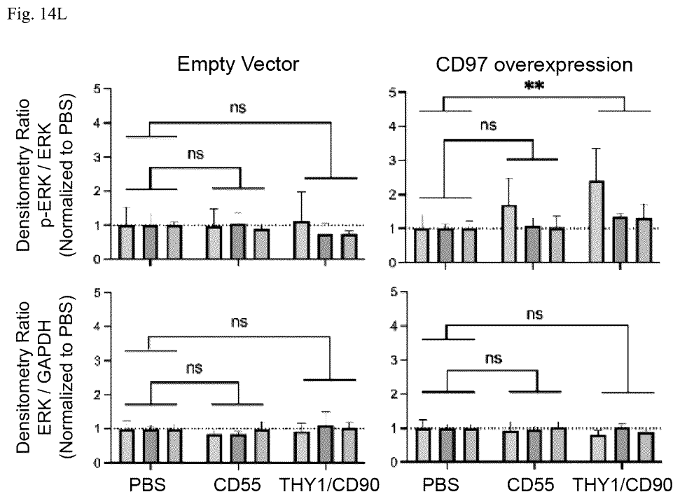

In addition to the MAPK signaling pathway, past research on CD97 has identified other signaling mechanisms, including G protein-mediated Gαᵢ (reduces cyclic-AMP) and Gα_{12/13} (activates RHOA) activation, and PI3K/AKT pathway activation (FIG. 14A).^{27,31,51,52} it was investigated whether CD97 influenced these other signaling pathways in the present PDGCs. CD97 overexpression in the present PDGCs did not alter canonical G protein-mediated signaling as measured by luciferase reporter assays (FIG. 20B). In addition, CD97 overexpression did not alter Gα_{12/13}-mediated activation of RHOA in pulldown assays specific to GTP-bound RHOA (FIG. 20C). Finally, phosphorylation of AKT was unchanged by perturbations in CD97 levels (FIG. 20D). This led to the exploration of alternative GPCR signaling mechanisms.

Many GPCRs are phosphorylated by GPCR kinases (GRKs), a modification which leads to recruitment of β-arrestin.⁵³ β-arrestin acts as a scaffold enabling recruitment of components of the MAPK pathway to GPCRs, thereby facilitating subsequent activation of this signaling pathway⁵⁴⁻⁵⁵, in addition to its well-described roles in GPCR desensitization and internalization.⁶³ To determine whether CD97 undergoes phosphorylation in GBM, phosphoproteomic data from GBM were analyzed in the CPTAC database.³⁵ Indeed, five novel serine/threonine phosphorylation sites were identified within the cytosolic C-terminus of CD97 (FIG. 14B). Three of these phosphorylation sites (T816, S818, and T820) were found in the vast majority of the GBM samples tested and also demonstrated a strong positive correlation in their abundance to one another (FIGS. 20E, 20F). This finding prompted the present inventors to test whether CD97 phosphorylation recruits β-arrestin as a potential mechanism for MAPK pathway activation. A CD97 isoform 3 truncation mutant (denoted as APS CD97 onward) was generated lacking amino acids 762-784, which include the five novel and one previously described⁵² phosphorylated residues (FIG. 20G). Localization of the APS CD97 mutant to the cell membrane was verified via immunofluorescent staining of the CD97 N-terminus after construct overexpression in HEK293 cells (FIG. 20H). When a fluorescence resonance energy transfer (FRET)-based β-arrestin recruitment assay was performed in GBM cells, enhanced signal upon wildtype (WT) CD97 overexpression was observed, an effect which was no longer observed after overexpression of the APS CD97 construct (FIG. 14C). Furthermore, overexpression of the APS CD97 mutant was not able to induce phosphorylation of ERK1/ERK2 (FIGS. 14D, 14E) and failed to rescue tumorsphere formation in PDGCs following CD97 knockdown, as measured by a tumorsphere formation assay (FIG. 14F). These data suggest that the CD97-MAPK signaling axis requires phosphorylation of the cytosolic C-terminus of CD97 and recruitment of β-arrestin.

### THY1/CD90 is the Most Likely CD97 Ligand in GBM

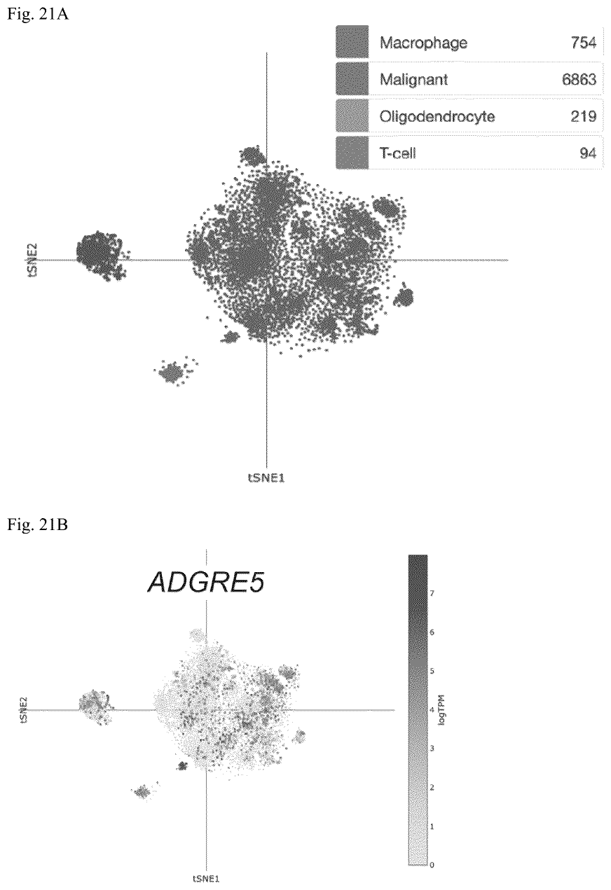

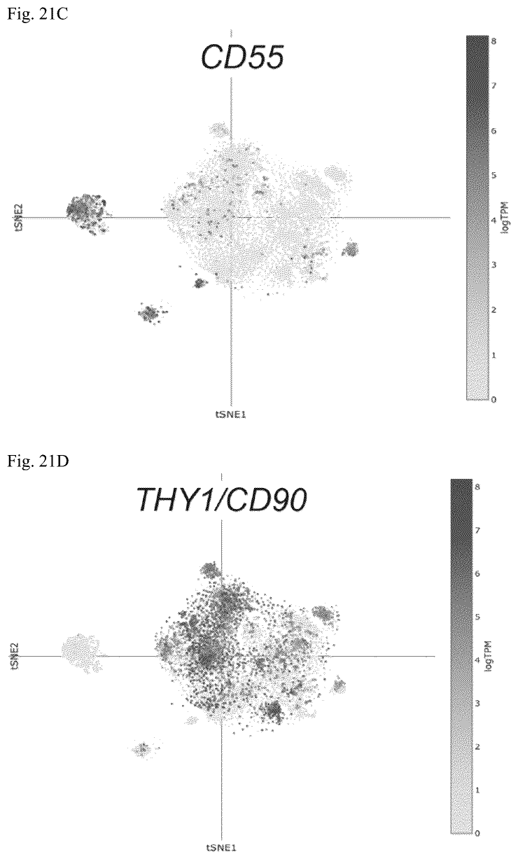

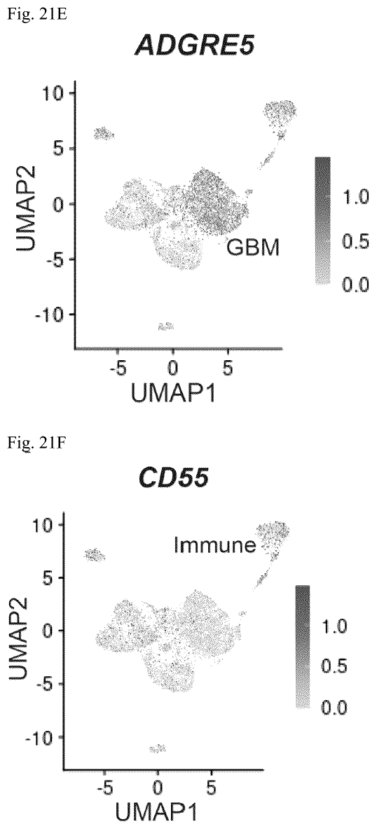

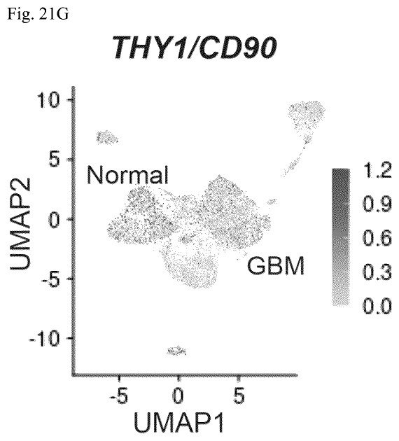

CD97 is known to interact with several ligands, including CD55, a glycosylphosphatidylinositol (GPI)-linked membrane-tethered protein that regulates the complement cascade in immune cells, and THY1/CD90, another GPI-linked protein found on neurons, immune cells, and activated endothelium.⁵⁶⁻⁵⁸ To gain insight into the expression profile of putative CD97 ligands in GBM of the present disclosure, as well as publicly available single-cell RNA-seq data in GBM were investigated (FIGS. 14G-14I, 21A-21F).⁵⁸⁻⁵⁹ While CD55 expression was largely confined to immune cells, THY1/CD90 was found in tumor cells, as well as non-neoplastic brain cell lineages (FIGS. 14G-14I). That THY1/CD90 is abundantly expressed in GBM cells, as well as neurons, was corroborated by the present initial investigation of transcript levels in the PDGCs and brain cell types (FIG. 10A). Furthermore, surface staining of PDGCs for CD55 and THY1/CD90 in flow cytometry assays revealed varied expression of CD55, but consistently high levels of THY1/CD90 (FIG. 14J).

Based on these findings, it was then tested whether further enhance CD97-activated MAPK signaling could be found in PDGCs by exposure to recombinant forms of CD55 and THY1/CD90. It was found that plating CD97-overexpressing PDGCs on wells coated with recombinant THY1/CD90 resulted in increased levels of phosphorylated-ERK1/ERK2 (FIG. 14K), an effect that was not observed to the same extent in cells transduced with an empty vector (EV) control (FIG. 14L). Though CD55 also led to some increases in phosphorylated-ERK1/ERK2, the effect was limited and did not reach statistical significance. Collectively, these data suggest that THY1/CD90 is the most likely activating ligand of CD97 in GBM.

### A CD97-Targeting Antibody-Drug Conjugate (ADC) Selectively Kills PDGCs but not Astrocytes or NSCs

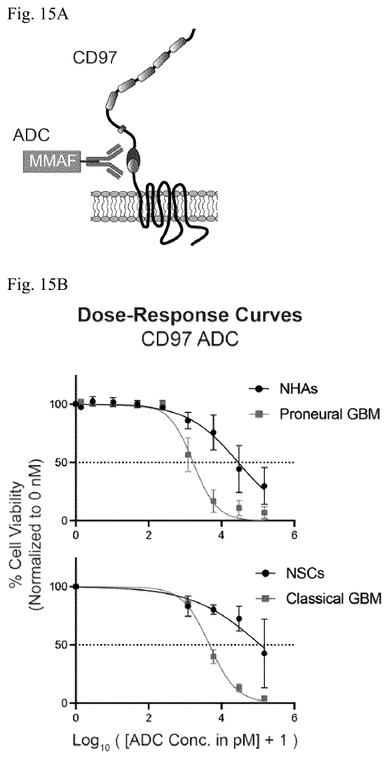

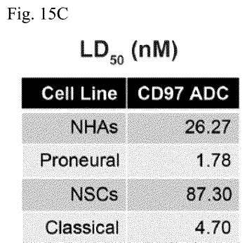

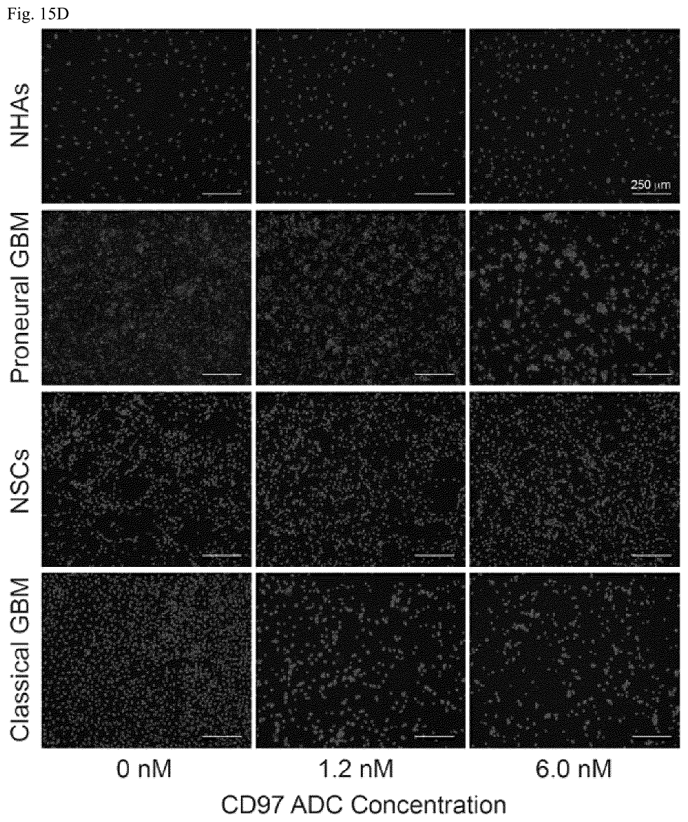

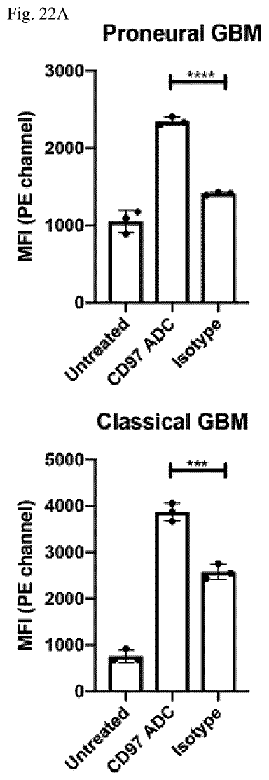

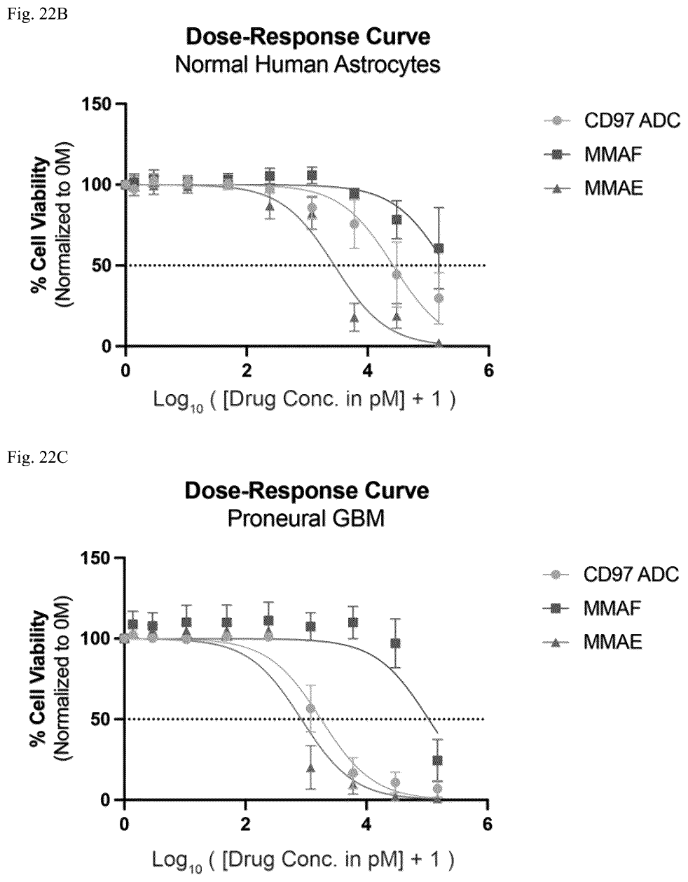

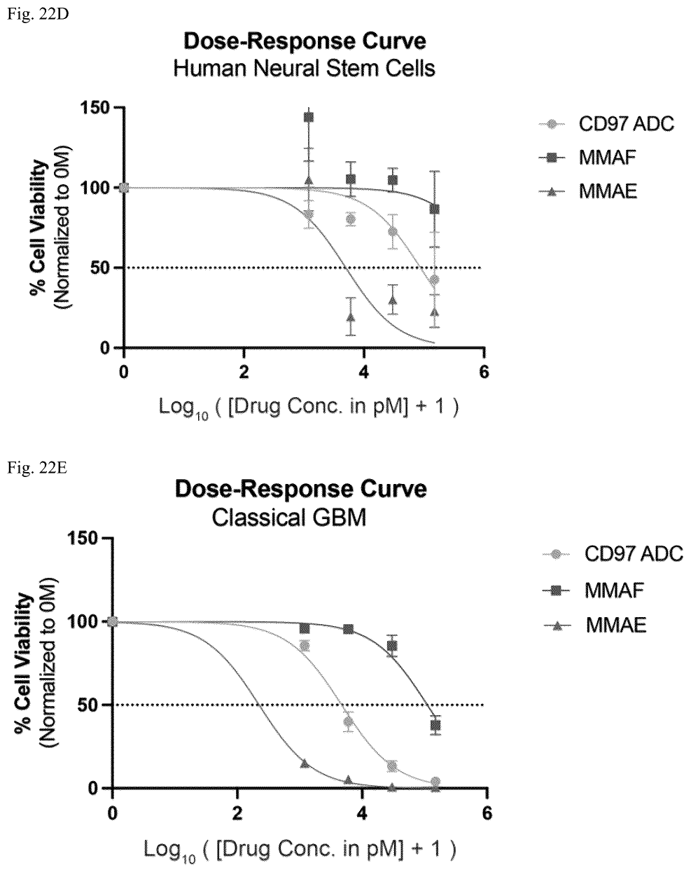

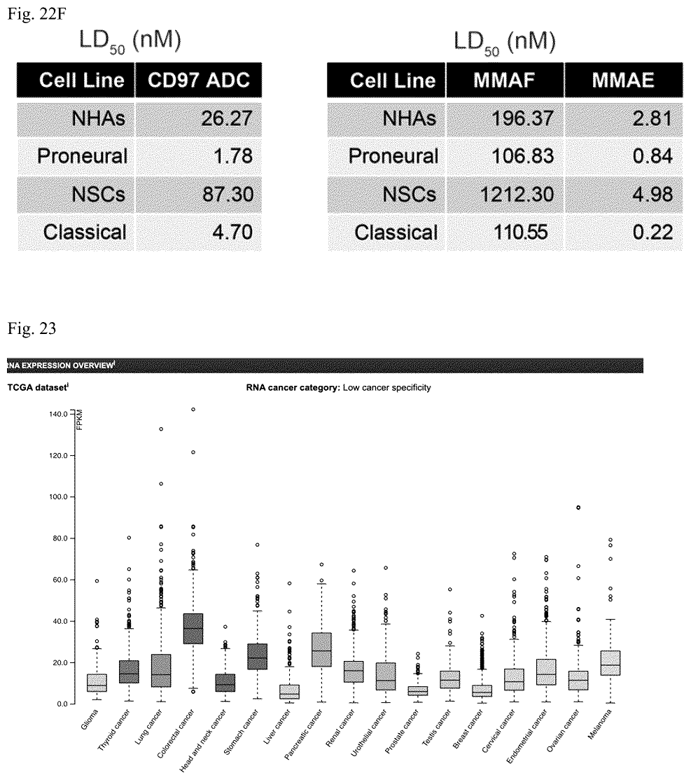

Since CD97 exhibits high expression in GBM and is absent from normal brain tissue, it was tested whether CD97 could be targeted therapeutically. A human CD97-specific antibody was generated by performing in vitro selection of a human synthetic antibody library (FIG. 15A). It was found that the anti-CD97 antibody exhibited significantly increased internalization into PDGCs when compared to an IgG1 LALA isotype control that does not bind to any known target (FIG. 22A). This antibody was then conjugated to monomethyl auristatin F (MMAF), a membrane-impermeant anti-tubulin agent blocking cytoskeletal polymerization and thereby cytotoxic to dividing cells⁶⁰, to generate an anti-CD97 antibody-drug conjugate (ADC). A lower lethal dose (LD₅₀) was observed in PDGCs compared to normal brain cells (NHAs and NSCs) in WST8 viability assays (FIGS. 15B, 15C, 22B-22E). Since the WST8 reagent relies on the presence of cellular metabolites, the assay's range depends on the metabolic activity of the cells, which varies considerably among PDGCs, NSCs, and relatively non-proliferative NHAs. Therefore, cells were also stained with Hoechst 33342 dye after treatment and representative images were taken to obtain cell counts (FIGS. 15D, 15E). From these images, the specificity of the CD97 ADC was even more evident, as only GBM cells decreased in number, while NSC and NHA numbers remained stable. Together, these findings suggest that the de novo expression of CD97 in GBM relative to healthy brain can be exploited therapeutically through the use of ADCs.

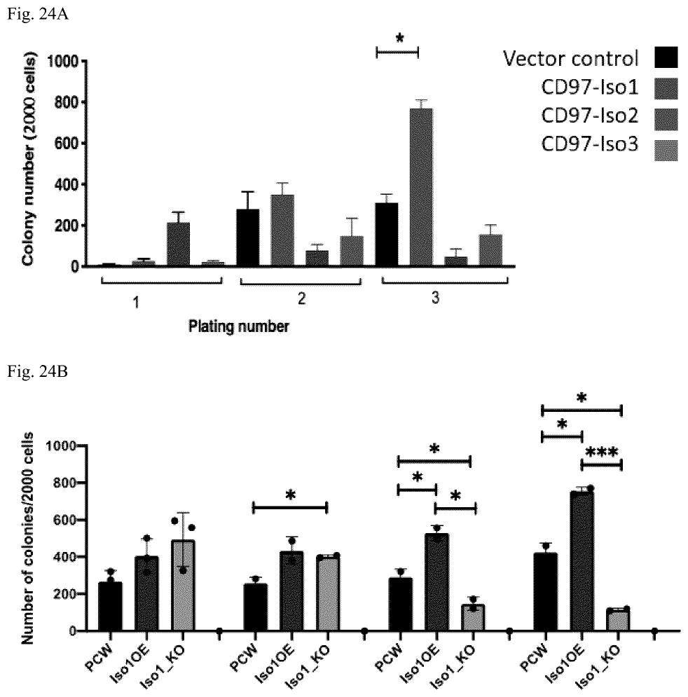

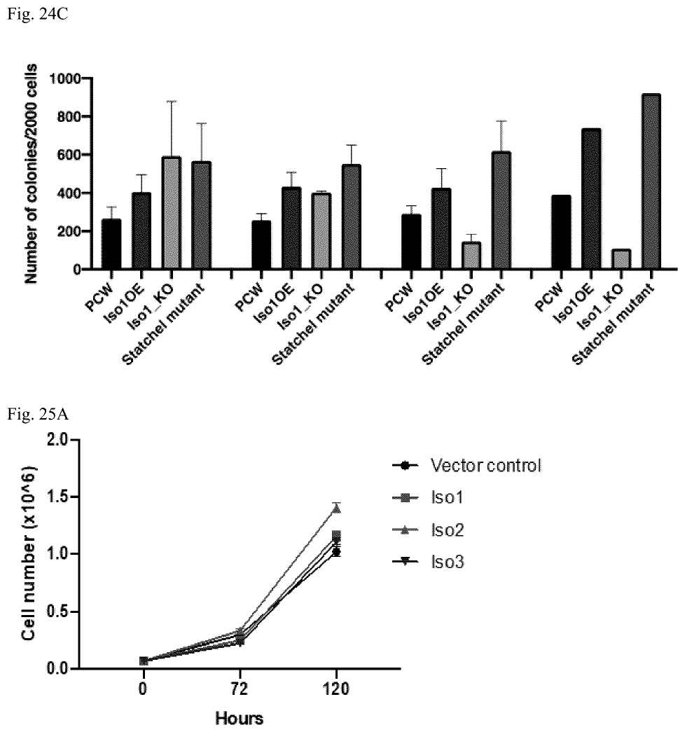

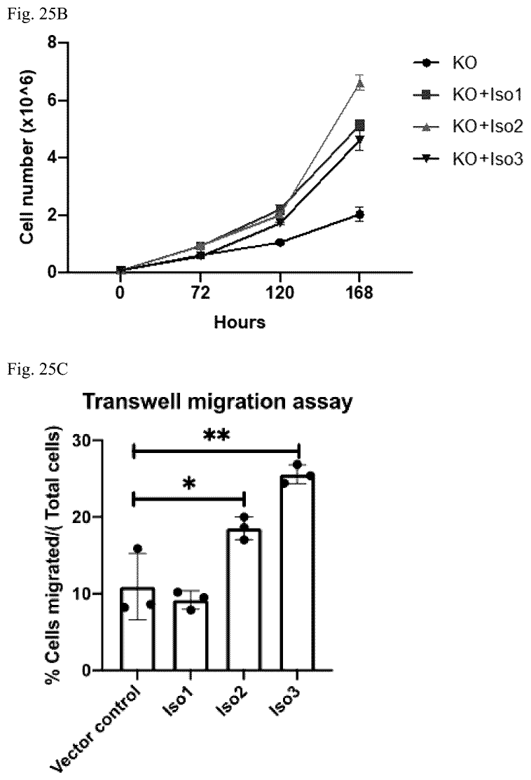

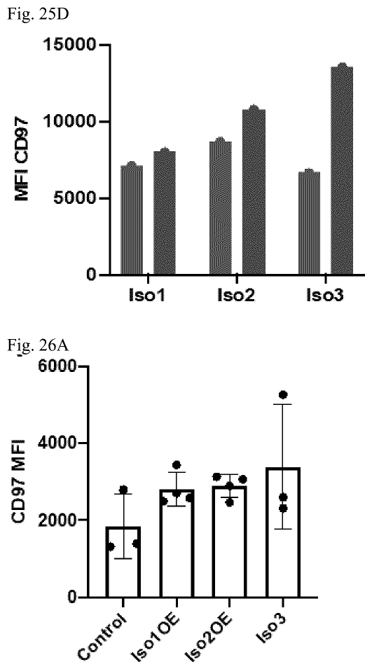

Given the variable expression of CD97 isoforms in human AML, it was tested herein whether the various isoforms support different biologic functions relevant to human AML biology. Overexpression of the CD97 isoforms in HL-60 cells resulted in differences in clonogenicity during serial plating in methylcellulose, with CD97 isoform 1 preferentially promoting serial replating and CD97 isoform 2 associated with increased clonogenicity in primary platings (FIG. 24A). In contrast to CD97 isoform 1, CRISPR-Cas9 mediated deletion of CD97 isoform 1 was associated with reduced clonogenicity (FIG. 24B). Overexpression of a non-cleavable version of CD97isol (stachel mutant) enhanced clonogenicity of HL-60 cells similar to CD97 isoform 1 in serial methycellulose replating assays (FIG. 24C). Overexpression of CD97 isoform 2 in WT HL-60 was unique in its ability to promote growth (FIG. 25A). Complementation of CD97 KO cells with each CD97 isoform partially rescued cell growth in liquid cultures, but the effect appeared to be the largest with CD97 isoform 2 (FIG. 25B). In transwell migration assays, CD97 isoform 3 overexpression preferentially promoted AML cell migration (FIG. 25C). Evaluation of CD97 cell surface expression in cells that migrated versus that did not, revealed highest levels of surface CD97 in cells overexpressing CD97 isoform 3 (FIG. 25D). Collectively, these data indicate that the various CD97 isoforms exhibit differences in supporting clonogenicity and proliferation, with CD97 isoform 1 likely promoting self-renewal, CD97 isoform 2 promoting proliferation, and CD97 isoform 3 promoting cell migration.

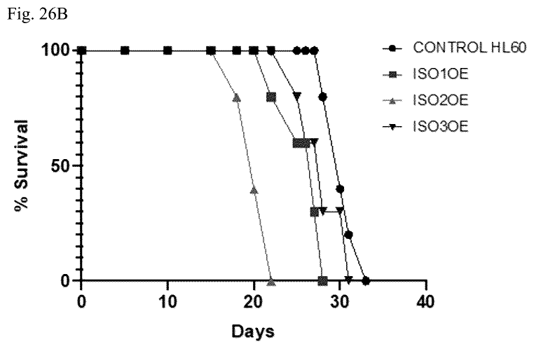

To test these potential functional differences in vivo, xenograft studies of HL-60 cells overexpressing each of the CD97 isoforms were performed in immunodeficient mice and assessed survival. After confirming similar levels of CD97 isoform overexpression in HL-60 cells by flow cytometry (FIG. 26A), equal numbers of cells were transplanted into NSG mice. Transplantation of 10⁶ HL-60 cells overexpressing each CD97 isoform resulted in differences in OS (FIG. 26B). These data indicate that CD97 isoforms exert differential effects on disease aggressiveness in xenografts, with CD97 isoform 2 associated with the most aggressive disease.

### DISCUSSION

GBM tumors resist multimodal treatment regimens, all but guaranteeing disease recurrence in patients. Identifying new targets for GBM is essential for ameliorating therapy, an already difficult task given GBM's extensive intertumoral and intratumoral heterogeneity.^{3-5,2,19} Adhesion GPCRs are an understudied group of transmembrane receptors with emerging roles in oncology¹⁰⁻¹² that show promise as therapeutic targets.⁶⁻⁹ Here, the aGPCR CD97 (ADGRE5) was identified as a ubiquitously expressed target in GBM, regardless of TCGA transcriptional subtype, thereby overcoming issues of intertumoral heterogeneity. Equally importantly, CD97 is absent from normal non-neoplastic brain tissue, further increasing its potential as a therapeutic target for GBM. In fact, the transcriptional upregulation of CD97 mRNA in GBM relative to NSCs, the putative cell of origin in glioma, was associated with the highest statistical significance genome-wide (FIG. 10). Consistent with this expression profile, PDGCs treated with CD97 ADC of the present disclosure demonstrated a significantly greater cell death than human astrocytes and NSCs, which lack CD97 expression (FIG. 15). Collectively, the data of the present disclosure suggest that CD97 in GBM is, by virtue of its expression profile, the most appealing treatment target among the aGPCRs, and possibly one of the most targetable cell surface proteins genome-wide. Furthermore, its essential tumorigenic functions in GBM suggest therapeutic targeting cannot only be accomplished via ADC platforms, but that neutralizing antibodies may also be beneficial. A crucial next step in investigating the therapeutic benefits of the CD97 ADC of the present disclosure would be to treat orthotopic tumor xenografts in vivo with the ADC compound. The treatment schedule and dosage, along with the method of drug administration, whether it be systemic or intracranial/intratumoral, would require optimization. The endogenous expression of CD97 by immune cells and other extracranial tissues warrants concern for peripheral toxicity if the ADC were to be administered systemically in patients. CD97 neutralization with a systemically administered monoclonal antibody, for example, has been found to reduce granulocyte infiltration to sites of inflammation and relieve arthritic activity in mice²⁴, corroborating the concern for possible extracranial toxicity of systemically administered CD97 biologics. In order to avoid targeting immune cells or other CD97-expressing tissues, delivery of antibody therapy within the tumor or the brain may be preferred. Novel approaches are being developed to carry out such localized treatments.⁶¹ Future studies would also be needed in order to test the impact of this CD97-targeting ADC on other CD97-expressing malignancies.

Historically, CD97 was first discovered as a leukocyte receptor and characterized in the context of the immune system.^{20-22,62} Since then, however, CD97 has been implicated in multiple malignancies, including hematologic (leukemia) and solid (esophageal, stomach, colorectal, hepatocellular, pancreatic, thyroid, prostate, ovarian, breast) tumors.²⁵⁻²⁸ CD97 expression in GBM was first demonstrated in 2012, when it was found to confer a migratory phenotype.³³ More recent work has also implicated CD97 in cellular proliferation, GBM stem cell self-renewal, and tumor metabolism,²⁹⁻³² but the underlying mechanisms have not been elucidated. Here, a methodical approach was taken to characterize the oncogenic function of CD97 in GBM using PDGCs from all TCGA-defined transcriptional subtypes (proneural, classical, mesenchymal) and advanced genetic, transcriptomic, metabolomic, cellular, and biochemical approaches. The detailed present study not only described phenotypic contributions of CD97 to cellular proliferation, tumor initiation, GBM stem cell self-renewal, metabolism, and migration, but also presented detailed molecular mechanisms accounting for these phenotypes.

Tumor metabolism, which relies both on the availability of nutrients and their utilization by tumor cells, is a critical determinant of oncogenesis at the interface of tumor cell-intrinsic biology and the microenvironment. GBM, like many other malignancies, manifests a dependency on glycolytic metabolism, rather than tricarboxylic acid (TCA) cycle and oxidative phosphorylation (OXPHOS). The predilection for glycolysis, which occurs in both normoxic conditions and in the severely hypoxic niches of GBM, is known as Warburg metabolism.³⁹⁻⁴¹⁻⁶³ This metabolic adaptation is thought to allow generation of sufficient consumable energy in the form of ATP, but also enable carbon skeleton conservation that is essential for biomass generation in enlarging tumors.⁶⁴ The mechanisms that regulate GBM's dependency on glycolysis remain incompletely understood. The data of the present disclosure suggest an important role for CD97 in the regulation of GBM cellular metabolism, and glycolysis in particular. ADGRE5 mRNA transcript levels show a striking positive correlation with transcripts encoding most glycolytic enzymes, and a negative correlation with the expression of most TCA cycle enzymes. Using steady-state metabolomics, it was found that CD97 knockdown resulted in a profound depletion of the majority of glycolytic metabolites, and also metabolites within the TCA cycle. Since glycolytic products feed into TCA cycle and mitochondrial metabolism via the conversion of pyruvate to acetyl-CoA, it is not surprising that reduced levels of glycolytic metabolites resulted in reduced levels of TCA cycle intermediates. Mechanistically, it was theorized that CD97 signaling promotes transcription of glycolytic transcripts, including LDHA (lactate dehydrogenase A) (FIG. 12C), which catalyzes conversion of pyruvate to lactate at the expense of the pyruvate to acetyl-CoA reaction that feeds into the TCA cycle. It was postulated that the upregulation of certain TCA cycle and OXPHOS transcripts upon CD97 knockdown signifies a compensatory, but ultimately inadequate, adaptation by cells in order to increase ATP production and meet cellular energy needs. More investigation is needed to determine the full mechanism whereby CD97 is impacting rates of glycolysis beyond effects on transcription rates of relevant genes.

The synthesis of the present experimental data led to formulate the hypothesis that CD97's effects on GBM cellular metabolism and tumor growth are driven by its activation of the MAPK/ERK pathway.³¹⁻⁵¹ This pathway has been implicated in a slew of cellular functions, including cellular metabolism, proliferation, and migration.⁶⁵ From the metabolism point of view, MAPK signaling modulates several enzymes involved in glycolytic and TCA cycle/OXPHOS pathways.^{45,66,67} Mutations that lead to overactivation of the MAPK pathway, such as in RAS or RAF, are often observed in malignancies and have been linked to their metabolic reliance on glycolysis and the PPP.⁴⁹⁻⁶⁸ Pancreatic tumors driven by the oncogenic KRAS G12D mutation, for example, exhibit overactivation of the MAPK pathway, leading to redirection of glycolytic intermediates into the PPP and overall metabolic reprogramming.⁴⁹ Determining the exact mechanism by which CD97-activated MAPK signaling influences cell metabolism and other cell functions is a crucial next step of investigation prompted by the present study. For example, whether CD97-MAPK signaling leads to post-translational modifications, such as phosphorylation, of relevant metabolic enzymes remains to be seen.

The impact of CD97 on GBM biology mirrors some of its actions in physiological processes. Within the immune system, CD97 has been shown to be critical for the inflammatory response by mediating cellular adhesion and migration.^{20-22,62} For example, CD97-expressing immune cells have been shown to bind to THY1/CD90-expressing activated endothelial cells⁵⁸, which may represent an important step in leukocyte recruitment and diapedesis at inflamed tissues. The present demonstration of CD97-mediated regulation of cellular metabolism does not necessarily exclude these previously-established functions in inflammation and cellular migration. In fact, it is well-documented that inflammation induces a metabolic shift in immune cells, leading to a greater reliance on glycolysis.⁶⁹ Furthermore, CD97 is expressed in macrophages, neutrophils, and T cells, and is upregulated upon their activation during the immune response, an event accompanied by an increased metabolic reliance on glycolysis.^{20,21,48,62,70-73} The link between metabolism and cell motility is thoroughly documented, but their interplay remains complex.74-7 Migratory cells, for example, have been shown to increase glucose uptake by upregulating expression of glucose transporters. This enables localized bursts in glycolysis at sites undergoing cellular contraction needed for cell motility.^{74,76} In this way, CD97's role in cellular metabolism may complement its previously established role in cellular migration. It is likely that CD97 plays similar functions in activated immune cells and in cancer cells, both of which share predilections for glycolytic metabolism and exhibit extended capacities of migration and proliferation.

Past studies on CD97 have suggested dependencies on multiple signaling mechanisms. A study in prostate cancer cells found that CD97 coupled with Gα_{12/13}, ultimately activating the RHOA pathway and promoting cellular migration.²⁷ Another study in human retinal pigment epithelium found that overexpression of CD97 activates MAPK signaling, as measured by increased serum response element (SRE)-driven luciferase expression in a luciferase-reporter system.⁷⁸ Interestingly, this finding was not observed herein using an SRE-driven luciferase assay in the present PDGCs, nor any other G protein-coupling of CD97 was detected in GBM cells using other luciferase-driven reporter systems (FIG. 20B). It was previously found that CD97 activated both MAPK and AKT signaling in leukemic stem cells.²⁶ In the present PDGCs, increased levels of phosphorylated ERK1/ERK2were only observed, but not phosphorylated AKT (FIG. 20D). A study in colorectal cancer cells demonstrated that CD97 underwent phosphorylation at its C-terminus, which ultimately regulated proper cytoskeletal organization and adhesion.⁵² In a different study, GRK6 was shown to phosphorylate CD97, leading to receptor internalization.⁷⁹ The present analysis of phosphoproteomic data from GBM biospecimens identified robust phosphorylation of novel serine/threonine residues at the cytosolic C-terminus of CD97, a finding that prompted us to investigate the involvement of β-arrestin in CD97-MAPK signaling. β-arrestin is known to act as a scaffold for MAPK components, thereby facilitating pathway activation.⁵⁴ Indeed, β-arrestin recruitment to the phosphorylated C-terminus of CD97 was shown herein. In order to test the role of the phosphorylated C-terminus in CD97 signaling, a DPS CD97 mutant was generated lacking the novel phosphorylated serine/threonine residues and found that the truncation abolished β-arrestin recruitment and MAPK activation.

Another important aspect of the present study was the characterization of CD97-ligand interactions of relevance in GBM. CD97 has a number of known extracellular ligands and binding partners, including the GPI-linked proteins CD55 and THY1/CD90.⁵⁸⁻⁵⁹ In contrast to CD55, THY1/CD90, a known neuronal marker, exhibited abundant expression in GBM cells (FIGS. 10A, 10B, 14I, 14J, 21D). When CD97-overexpressing PDGCs were plated on recombinant THY1/CD90, a significantly more pronounced increase in MAPK signaling relative to plating on CD55 was observed (FIGS. 14K, 14L). Together these data suggest that CD97-activated MAPK signaling most likely relies on the activating ligand THY1/CD90.⁵⁷ Previous studies have implicated THY1/CD90 in GBM pathogenesis and other malignancies.^{80,81} THY1/CD90 is known to form complexes with integrins, which have also been described as CD97 binding partners.⁸² Further insight into the binding interaction between CD97, THY1/CD90, and other putative ligands would help elucidate how crucial this binding is to the phosphorylation of the CD97 C-terminus, recruitment of β-arrestin, and activation of the MAPK signaling pathway. Additionally, the impact of autoproteolytic cleavage, which was observed for both endogenous and exogenous CD97 in PDGCs, on CD97 signaling is a future research aim.

Gliomas are broadly categorized based on their expression of a neomorphic mutation in the IDH enzyme.⁵ Wildtype IDH is found in both the cytoplasm and the mitochondria, where it catalyzes the decarboxylation of isocitrate to α-ketoglutarate (α-KG). The mutant form of IDH, however, generates the oncometabolite 2-hydroxyglutarate (2-HG). In response to 2-HG accumulation, IDH-mutant cells increase levels of OXPHOS and reduce lactate production by silencing LDHA expression.⁸³⁻⁸⁴ This metabolic reprogramming in IDH-mutant glioma lies in stark contrast with that which occurs in IDH-wildtype GBM, where cells are characterized by increased Warburg metabolism. The present single-cell multiomic and ChIP-seq analyses found that ADGRE5 gene expression was robust in IDH-wildtype GBM, but quite limited in low-grade IDH-mutant glioma (FIG. 10I). This finding correlates with the metabolic profiles of the two glioma types and is consistent with the present hypothesis that CD97 promotes Warburg metabolism.

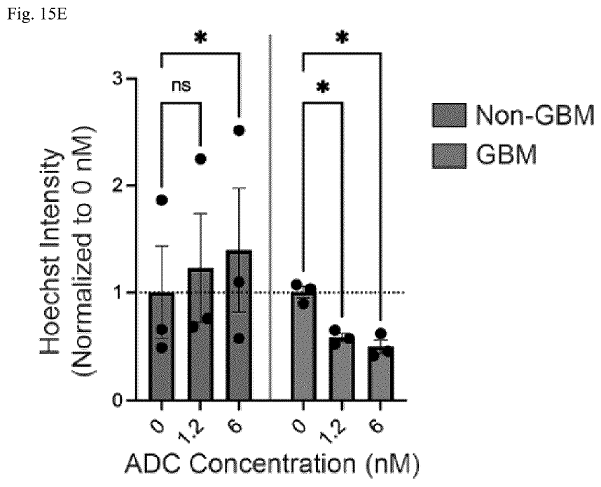

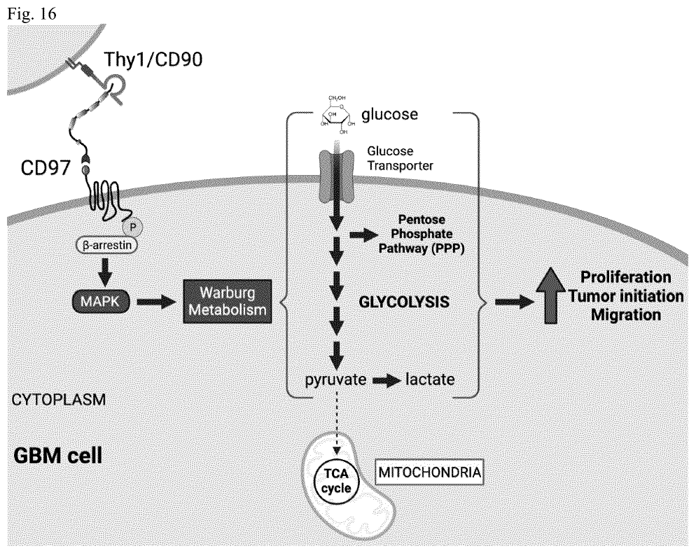

In summary, the present study identified CD97 as the aGPCR with the widest differential expression pattern in IDH-wildtype GBM versus normal brain tissue and NSCs, the putative cell-of-origin. It was also shown that CD97 functions to promote Warburg metabolism via a signaling mechanism that includes phosphorylation of the receptor's cytosolic C-terminus, recruitment of 0-arrestin, and activation of MAPK signaling (FIG. 16). Finally, a novel CD97-ADC was used to selectively target GBM cells. Overall, the present studies reveal novel insights regarding the role of CD97 in GBM, suggesting that CD97 represents an appealing therapeutic vulnerability in GBM and that its targeting will be impactful in future clinical trials.

### REFERENCES

1. Ceccarelli, M. et al. Molecular Profiling Reveals Biologically Discrete Subsets and Pathways of Progression in Diffuse Glioma. Cell 164, 550-563 (2016). doi.org:10.1016/j.cell.2015.12.028.
2. Verhaak, R. G. et al. Integrated genomic analysis identifies clinically relevant subtypes of glioblastoma characterized by abnormalities in PDGFRA, IDH1, EGFR, and NFl. Cancer Cell 17, 98-110 (2010). doi.org:10.1016/j.ccr.2009.12.020.
3. Ostrom, Q. T. et al. CBTRUS statistical report: Primary brain and central nervous system tumors diagnosed in the United States in 2006-2010. Neuro Oncol 15 Suppl 2, iil-56 (2013). doi.org:10.1093/neuonc/not151.
4. Stupp, R. et al. Radiotherapy plus concomitant and adjuvant temozolomide for glioblastoma. N Engl J Med 352, 987-996 (2005). doi.org:10.1056/NEJMoa043330
5. Louis, D. N. et al. The 2021 WHO Classification of Tumors of the Central Nervous System: a summary. Neuro Oncol 23, 1231-1251 (2021). doi.org:10.1093/neuonc/noab106.
6. Stephan, G., Ravn-Boess, N. & Placantonakis, D. G. Adhesion G protein-coupled receptors in glioblastoma. Neurooncol Adv 3, vdab046 (2021). doi.org:10.1093/noajnl/vdab046.
7. Krishnan, A., Nijmeijer, S., de Graaf, C. & Schioth, H. B. Classification, Nomenclature, and Structural Aspects of Adhesion GPCRs. Handb Exp Pharmacol 234, 15-41 (2016). doi.org:10.1007/978-3-319-41523-9_2.
8. Hamann, J. et al. International Union of Basic and Clinical Pharmacology. XCIV. Adhesion G protein-coupled receptors. Pharmacol Rev 67, 338-367 (2015). doi.org:10.1124/pr.114.009647.
9. Liebscher, I. et al. A tethered agonist within the ectodomain activates the adhesion G protein-coupled receptors GPR126 and GPR133. Cell Rep 9, 2018-2026 (2014). doi.org:10.1016/j.celrep.2014.11.036.
10. Zhang, S. et al. GPR56 Drives Colorectal Tumor Growth and Promotes Drug Resistance through Upregulation of MDR1 Expression via a RhoA-Mediated Mechanism. Mol Cancer Res 17, 2196-2207 (2019). doi.org:10.1158/1541-7786.Mcr-19-0436.
11. Bayin, N. S. et al. GPR133 (ADGRD1), an adhesion G-protein-coupled receptor, is necessary for glioblastoma growth. Oncogenesis 5, e263 (2016). doi.org:10.1038/oncsis.2016.63.
12. Cui, H. et al. GPR126 regulates colorectal cancer cell proliferation by mediating HDAC2 and GLI2 expression. Cancer Sci 112, 1798-1810 (2021). doi.org:10.1111/cas.14868.
13. Aust, G. et al. CD97: a dedifferentiation marker in human thyroid carcinomas. Cancer Res 57, 1798-1806 (1997).
14. Ackerman, S. D., Garcia, C., Piao, X., Gutmann, D. H. & Monk, K. R. The adhesion GPCR Gpr56 regulates oligodendrocyte development via interactions with Ga12/13 and RhoA. Nat Commun 6, 6122 (2015). doi.org:10.1038/ncomms7122.
15. Bianchi, E. et al. Control of oviductal fluid flow by the G-protein coupled receptor Adgrdl is essential for murine embryo transit. Nat Commun 12, 1251 (2021). doi.org:10.1038/s41467-021-21512-w.
16. Modrek, A. S. et al. Low-Grade Astrocytoma Mutations in IDH1, P53, and ATRX Cooperate to Block Differentiation of Human Neural Stem Cells via Repression of SOX2. Cell Rep 21, 1267-1280 (2017). doi.org:10.1016/j.celrep.2017.10.009.
17. Modrek, A. S., Bayin, N. S. & Placantonakis, D. G. Brain stem cells as the cell of origin in glioma. World J Stem Cells 6, 43-52 (2014). doi.org:10.4252/wjsc.v6.il.43.
18. Lee, J. H. et al. Human glioblastoma arises from subventricular zone cells with low-level driver mutations. Nature 560, 243-247 (2018). doi.org:10.1038/s41586-018-0389-3.
19. Brennan, C. W. et al. The somatic genomic landscape of glioblastoma. Cell 155, 462-477 (2013). doi.org:10.1016/j.cell.2013.09.034.
20. Jaspars, L. H., Vos, W., Aust, G., Van Lier, R. A. & Hamann, J. Tissue distribution of the human CD97 EGF-TM7 receptor. Tissue Antigens 57, 325-331 (2001). doi.org:10.1034/j.1399-0039.2001.057004325.x.
21. Hamann, J. et al. CD97 in leukocyte trafficking. Adv Exp Med Biol 706, 128-137 (2010). doi.org:10.1007/978-1-4419-7913-1_11.
22. Spendlove, I. & Sutavani, R. The role of CD97 in regulating adaptive T-cell responses. Adv Exp Med Biol 706, 138-148 (2010). doi.org:10.1007/978-1-4419-7913-1_12.
23. Leemans, J. C. et al. The epidermal growth factor-seven transmembrane (EGF-TM7) receptor CD97 is required for neutrophil migration and host defense. J Immunol 172, 1125-1131 (2004). doi.org:10.4049/jimmunol.172.2.1125.
24. Kop, E. N. et al. CD97 neutralisation increases resistance to collagen-induced arthritis in mice. Arthritis Res Ther 8, R155 (2006). doi.org:10.1186/ar2049.
25. Aust, G., Zheng, L. & Quaas, M. To Detach, Migrate, Adhere, and Metastasize: CD97/ADGRE5 in Cancer. Cells 11 (2022). doi.org:10.3390/cells11091538.
26. Martin, G. H. et al. CD97 is a critical regulator of acute myeloid leukemia stem cell function. J Exp Med 216, 2362-2377 (2019). doi.org:10.1084/jem.20190598.
27. Ward, Y. et al. LPA receptor heterodimerizes with CD97 to amplify LPA-initiated RHO-dependent signaling and invasion in prostate cancer cells. Cancer Res 71, 7301-7311 (2011). doi.org:10.1158/0008-5472.Can-11-2381.
28. Aust, G. et al. CD97, but not its closely related EGF-TM7 family member EMR2, is expressed on gastric, pancreatic, and esophageal carcinomas. Am J Clin Pathol 118, 699-707 (2002). doi.org:10.1309/a6ab-vf3f-7m88-c0ej.
29. Safaee, M. et al. Overexpression of CD97 confers an invasive phenotype in glioblastoma cells and is associated with decreased survival of glioblastoma patients. PLoS One 8, e62765 (2013). doi.org:10.1371/journal.pone.0062765.
30. Safaee, M. et al. Proportional upregulation of CD97 isoforms in glioblastoma and glioblastoma-derived brain tumor initiating cells. PLoS One 10, e0111532 (2015). doi.org:10.1371/journal.pone.0111532.
31. Safaee, M. M. et al. CD97 is associated with mitogenic pathway activation, metabolic reprogramming, and immune microenvironment changes in glioblastoma. Sci Rep 12, 1464 (2022). doi.org:10.1038/s41598-022-05259-y.
32. Eichberg, D. G., Slepak, T. I., Pascoini, A. L., Komotar, R. J. & Ivan, M. E. Genetic manipulation of adhesion GPCR CD97/ADGRE5 modulates invasion in patient-derived glioma stem cells. J Neurooncol 153, 383-391 (2021). doi.org:10.1007/s11060-021-03778-8.
33. Chidambaram, A., Fillmore, H. L., Van Meter, T. E., Dumur, C. I. & Broaddus, W. C. Novel report of expression and function of CD97 in malignant gliomas: correlation with Wilms tumor 1 expression and glioma cell invasiveness. J Neurosurg 116, 843-853 (2012). doi.org:10.3171/2011.11.Jns111455.
34. Perna, F. et al. Integrating Proteomics and Transcriptomics for Systematic Combinatorial Chimeric Antigen Receptor Therapy of AML. Cancer Cell 32, 506-519.e505 (2017). doi.org:10.1016/j.ccell.2017.09.004.
35. Wang, L. B. et al. Proteogenomic and metabolomic characterization of human glioblastoma. Cancer Cell 39, 509-528.e520 (2021). doi.org:10.1016/j.ccell.2021.01.006.
36. Flavahan, W. A. et al. Insulator dysfunction and oncogene activation in IDH mutant gliomas. Nature 529, 110−114 (2016). doi.org:10.1038/naturel6490.
37. Frenster, J. D. et al. Functional impact of intramolecular cleavage and dissociation of adhesion G protein-coupled receptor GPR133 (ADGRD1) on canonical signaling. J Biol Chem 296, 100798 (2021). doi.org:10.1016/j.jbc.2021.100798.
38. Hsiao, C. C. et al. Site-specific N-glycosylation regulates the GPS auto-proteolysis of CD97. FEBS Lett 583, 3285-3290 (2009). doi.org:10.1016/j.febslet.2009.09.001.
39. Kathagen-Buhmann, A. et al. Glycolysis and the pentose phosphate pathway are differentially associated with the dichotomous regulation of glioblastoma cell migration versus proliferation. Neuro Oncol 18, 1219-1229 (2016). doi.org:10.1093/neuonc/now024.
40. Stanke, K. M., Wilson, C. & Kidambi, S. High Expression of Glycolytic Genes in Clinical Glioblastoma Patients Correlates With Lower Survival. Front Mol Biosci 8, 752404 (2021). doi.org:10.3389/fmolb.2021.752404.
41. Warburg, O., Wind, F. & Negelein, E. THE METABOLISM O F TUMORS I N THE BODY. J Gen Physiol 8, 519-530 (1927). doi.org:10.1085/jgp.8.6.519.
42. Wolf, A. et al. Hexokinase 2 is a key mediator of aerobic glycolysis and promotes tumor growth in human glioblastoma multiforme. J Exp Med 208, 313-326 (2011). doi.org:10.1084/jem.20101470.
43. Libby, C. J. et al. A role for GLUT3 in glioblastoma cell invasion that is not recapitulated by GLUT1. Cell Adh Migr 15, 101-115 (2021). doi.org:10.1080/19336918.2021.1903684.
44. Phillips, E. et al. PFKFB4 interacts with FBXO28 to promote HIF-la signaling in glioblastoma. Oncogenesis 11, 57 (2022). doi.org:10.1038/s41389-022-00433-3.
45. Yang, W. et al. ERK1/2-dependent phosphorylation and nuclear translocation of PKM2 promotes the Warburg effect. Nat Cell Biol 14, 1295-1304 (2012). doi.org:10.1038/ncb2629.
46. Hu, Y. et al. Dual roles of hexokinase 2 in shaping microglial function by gating glycolytic flux and mitochondrial activity. Nat Metab 4, 1756-1774 (2022). doi.org:10.1038/s42255-022-00707-5.
47. DeWaal, D. et al. Hexokinase-2 depletion inhibits glycolysis and induces oxidative phosphorylation in hepatocellular carcinoma and sensitizes to metformin. Nat Commun 9, 446 (2018). doi.org:10.1038/s41467-017-02733-4.
48. Freemerman, A. J. et al. Metabolic reprogramming of macrophages: glucose transporter 1 (GLUT1)-mediated glucose metabolism drives a proinflammatory phenotype. J Biol Chem 289, 7884-7896 (2014). doi.org:10.1074/jbc.M113.522037.
49. Ying, H. et al. Oncogenic Kras maintains pancreatic tumors through regulation of anabolic glucose metabolism. Cell 149, 656-670 (2012). doi.org:10.1016/j.cell.2012.01.058.
50. Gopalbhai, K. et al. Negative regulation of MAPKK by phosphorylation of a conserved serine residue equivalent to Ser212 of MEK1. J Biol Chem 278, 8118-8125 (2003). doi.org:10.1074/jbc.M211870200.
51. Li, C. et al. CD97 promotes gastric cancer cell proliferation and invasion through exosome-mediated MAPK signaling pathway. World J Gastroenterol 21, 6215-6228 (2015). doi.org:10.3748/wjg.v21.i20.6215.
52. Hilbig, D. et al. Mechano-Dependent Phosphorylation of the PDZ-Binding Motif of CD97/ADGRE5 Modulates Cellular Detachment. Cell Rep 24, 1986-1995 (2018). doi.org:10.1016/j.celrep.2018.07.071.
53. Drube, J. et al. GPCR kinase knockout cells reveal the impact of individual GRKs on arrestin binding and GPCR regulation. Nat Commun 13, 540 (2022). doi.org:10.1038/s41467-022-28152-8.
54. McDonald, P. H. et al. Beta-arrestin 2: a receptor-regulated MAPK scaffold for the activation of JNK3. Science 290, 1574-1577 (2000). doi.org:10.1126/science.290.5496.1574.
55. Laporte, S. A. et al. The beta2-adrenergic receptor/betaarrestin complex recruits the clathrin adaptor AP-2 during endocytosis. Proc Natl Acad Sci USA 96, 3712-3717 (1999). doi.org:10.1073/pnas.96.7.3712.
56. Miwa, T. et al. Characterization of glycosylphosphatidylinositol-anchored decay accelerating factor (GPI-DAF) and transmembrane DAF gene expression in wild-type and GPI-DAF gene knockout mice using polyclonal and monoclonal antibodies with dual or single specificity. Immunology 104, 207-214 (2001). doi.org:10.1046/j.1365-2567.2001.01280.x.
57. Haeryfar, S. M. & Hoskin, D. W. Thy-1: more than a mouse pan-T cell marker. J Immunol 173, 3581-3588 (2004). doi.org:10.4049/jimmunol.173.6.3581.
58. Wandel, E., Saalbach, A., Sittig, D., Gebhardt, C. & Aust, G. Thy-1 (CD90) is an interacting partner for CD97 on activated endothelial cells. J Immunol 188, 1442-1450 (2012). doi.org:10.4049/jimmunol.1003944.
59. Niu, M. et al. Structural basis for CD97 recognition of the decay-accelerating factor CD55 suggests mechanosensitive activation of adhesion GPCRs. J Biol Chem 296, 100776 (2021). doi.org:10.1016/j.jbc.2021.100776.
60. Doronina, S. O. et al. Development of potent monoclonal antibody auristatin conjugates for cancer therapy. Nat Biotechnol 21, 778-784 (2003). doi.org:10.1038/nbt832.
61. Razpotnik, R., Novak, N., Curin Serbec, V. & Rajcevic, U. Targeting Malignant Brain Tumors with Antibodies. Front Immunol 8, 1181 (2017). doi.org:10.3389/fimmu.2017.01181.
62. Eichler, W., Hamann, J. & Aust, G. Expression characteristics of the human CD97 antigen. Tissue Antigens 50, 429-438 (1997). doi.org:10.1111/j.1399-0039.1997.tb02897.x.
63. Liang, X. et al. Glioblastoma glycolytic signature predicts unfavorable prognosis, immunological heterogeneity, and ENO1 promotes microglia M2 polarization and cancer cell malignancy. Cancer Gene Ther (2022). doi.org:10.1038/s41417-022-00569-9.
64. Lunt, S. Y. & Vander Heiden, M. G. Aerobic glycolysis: meeting the metabolic requirements of cell proliferation. Annu Rev Cell Dev Biol 27, 441-464 (2011). doi.org:10.1146/annurev-cellbio-092910−154237.
65. Lavoie, H., Gagnon, J. & Therrien, M. ERK signalling: a master regulator of cell behaviour, life and fate. Nat Rev Mol Cell Biol 21, 607-632 (2020). doi.org:10.1038/s41580-020-0255-7.
66. Bando, H. et al. Phosphorylation of the 6-phosphofructo-2-kinase/fructose 2,6-bisphosphatase/PFKFB3 family of glycolytic regulators in human cancer. Clin Cancer Res 11, 5784-5792 (2005). doi.org:10.1158/1078-0432.Ccr-05-0149.
67. Houles, T. et al. RSK Regulates PFK-2 Activity to Promote Metabolic Rewiring in Melanoma. Cancer Res 78, 2191-2204 (2018). doi.org:10.1158/0008-5472.Can-17-2215.
68. Hall, A. et al. Dysfunctional oxidative phosphorylation makes malignant melanoma cells addicted to glycolysis driven by the (V600E)BRAF oncogene. Oncotarget 4, 584-599 (2013). doi.org:10.18632/oncotarget.965.
69. Soto-Heredero, G., G6mez de Las Heras, M. M., Gaband6-Rodriguez, E., Oller, J. & Mittelbrunn, M. Glycolysis—a key player in the inflammatory response. Febs j 287, 3350-3369 (2020). doi.org:10.1111/febs.15327.
70. Baillet, A. et al. Unexpected function of the phagocyte NADPH oxidase in supporting hyperglycolysis in stimulated neutrophils: key role of 6-phosphofructo-2-kinase. Faseb j 31, 663-673 (2017). doi.org:10.1096/fj.201600720R.
71. Yu, Q. et al. Regulations of Glycolytic Activities on Macrophages Functions in Tumor and Infectious Inflammation. Front Cell Infect Microbiol 10, 287 (2020). doi.org:10.3389/fcimb.2020.00287.
72. Chang, C. H. et al. Posttranscriptional control of T cell effector function by aerobic glycolysis. Cell 153, 1239-1251 (2013). doi.org:10.1016/j.cell.2013.05.016
73. Capasso, M. et al. Costimulation via CD55 on human CD4+ T cells mediated by CD97. J Immunol 177, 1070-1077 (2006). doi.org:10.4049/jimmunol.177.2.1070.
74. Kishore, M. et al. Regulatory T Cell Migration Is Dependent on Glucokinase-Mediated Glycolysis. Immunity 47, 875-889.e810 (2017). doi.org:10.1016/j.immuni.2017.10.017.
75. Guak, H. et al. Glycolytic metabolism is essential for CCR7 oligomerization and dendritic cell migration. Nat Commun 9, 2463 (2018). doi.org:10.1038/s41467-018-04804-6.
76. Wu, D. et al. Single-cell metabolic imaging reveals a SLC2A3-dependent glycolytic burst in motile endothelial cells. Nat Metab 3, 714-727 (2021). doi.org:10.1038/s42255-021-00390-y.
77. Shiraishi, T. et al. Glycolysis is the primary bioenergetic pathway for cell motility and cytoskeletal remodeling in human prostate and breast cancer cells. Oncotarget 6, 130-143 (2015). doi.org:10.18632/oncotarget.2766.
78. Eichler, W. et al. The role of ADGRE5/CD97 in human retinal pigment epithelial cell growth and survival. Ann N Y Acad Sci 1456, 64-79 (2019). doi.org:10.1111/nyas.14210.
79. Yin, Y. et al. CD97 Promotes Tumor Aggressiveness Through the Traditional G Protein-Coupled Receptor-Mediated Signaling in Hepatocellular Carcinoma. Hepatology 68, 1865-1878 (2018). doi.org:10.1002/hep.30068.
80. Sauzay, C., Voutetakis, K., Chatziioannou, A., Chevet, E. & Avril, T. CD90/Thy-1, a Cancer-Associated Cell Surface Signaling Molecule. Front Cell Dev Biol 7, 66 (2019). doi.org:10.3389/fcell.2019.00066.
81. Avril, T. et al. CD90 Expression Controls Migration and Predicts Dasatinib Response in Glioblastoma. Clin Cancer Res 23, 7360-7374 (2017). doi.org:10.1158/1078-0432.Ccr-17-1549.
82. Leyton, L. et al. Thy-1/CD90 a Bidirectional and Lateral Signaling Scaffold. Front Cell Dev Biol 7, 132 (2019). doi.org:10.3389/fcell.2019.00132.
83. Chesnelong, C. et al. Lactate dehydrogenase A silencing in IDH mutant gliomas. Neuro Oncol 16, 686-695 (2014). doi.org:10.1093/neuonc/not243.
84. Grassian, A. R. et al. IDH1 mutations alter citric acid cycle metabolism and increase dependence on oxidative mitochondrial metabolism. Cancer Res 74, 3317-3331 (2014). doi.org:10.1158/0008-5472.Can-14-0772-t.
85. Frenster, J. D. & Placantonakis, D. G. Establishing Primary Human Glioblastoma Tumorsphere Cultures from Operative Specimens. Methods Mol Biol 1741, 63-69 (2018). doi.org:10.1007/978-1-4939-7659-1_4.
86. Frenster, J. D., Inocencio, J. & Placantonakis, D. G. Lentiviral Transduction of Primary Human Glioblastoma Cultures. Methods Mol Biol 1741, 81-89 (2018). doi.org:10.1007/978-1-4939-7659-1_6.
87. Xu, Z., Kader, M., Sen, R. & Placantonakis, D. G. Orthotopic Patient-Derived Glioblastoma Xenografts in Mice. Methods Mol Biol 1741, 183-190 (2018). doi.org:10.1007/978-1-4939-7659-1_14.
88. Frenster, J. D. & Placantonakis, D. G. Bioluminescent In Vivo Imaging of Orthotopic Glioblastoma Xenografts in Mice. Methods Mol Biol 1741, 191-198 (2018). doi.org:10.1007/978-1-4939-7659-1_15.
89. Fellouse, F. A. et al. High-throughput generation of synthetic antibodies from highly functional minimalist phage-displayed libraries. J Mol Biol 373, 924-940 (2007). doi.org:10.1016/j.jmb.2007.08.005.
90. Paduch, M. et al. Generating conformation-specific synthetic antibodies to trap proteins in selected functional states. Methods 60, 3-14 (2013). doi.org:10.1016/j.ymeth.2012.12.010.
91. Miller, K. R. et al. T cell receptor-like recognition of tumor in vivo by synthetic antibody fragment. PLoS One 7, e43746 (2012). doi.org:10.1371/journal.pone.0043746.
92. Oury, J. et al. Mechanism of disease and therapeutic rescue of Dok7 congenital myasthenia. Nature 595, 404-408 (2021). doi.org:10.1038/s41586-021-03672-3.
93. Lo, M. et al. Effector-attenuating Substitutions That Maintain Antibody Stability and Reduce Toxicity in Mice. J Biol Chem 292, 3900-3908 (2017). doi.org:10.1074/jbc.M116.767749.
94. Doronina, S. O. et al. Enhanced activity of monomethylauristatin F through monoclonal antibody delivery: effects of linker technology on efficacy and toxicity. Bioconjug Chem 17, 114-124 (2006). doi.org:10.1021/bc0502917.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An antibody, or antigen-binding fragment thereof, that binds to at least one isoform of cluster of differentiation 97 (CD97).

2. The antibody or antigen-binding fragment of claim 1, comprising
(a) a heavy chain complementarity determining region 1 (CDR1), a heavy chain CDR2, a heavy chain CDR3 of a heavy chain variable region (VH) comprising an amino acid sequence of SEQ ID NO: 2, 10, 16, 22, 26, 32, 36, 42, 46, 50, 54, 56, 60, 64, 70, 74, 78, 81, 31, or 73; or a heavy chain CDR1 and a heavy chain CDR2 of a VH comprising an amino acid sequence of SEQ ID NO: 10 and a heavy chain CDR3 comprising N at position 100b, W at position 100e, and/or Y at position 100f, wherein the position numbering is according to Kabat, and/or
(b) a light chain CDR1, a light chain CDR2, and a light chain CDR3 of a light chain variable region (VL) comprising an amino acid sequence of SEQ ID NO: 1, 9, 15, 21, 25, 35, 41, 49, 59, 63, 69, 77, or 53.

3. (canceled)

4. The antibody or antigen-binding fragment of claim 1, comprising
a) a heavy chain CDR1, a heavy chain CDR2, a heavy chain CDR3 of a VH comprising an amino acid sequence of SEQ ID NO: 2, and a light chain CDR1, a light chain CDR2, and a light chain CDR3 of a VL comprising an amino acid sequence of SEQ ID NO: 1;
b) a heavy chain CDR1, a heavy chain CDR2, a heavy chain CDR3 of a VH comprising an amino acid sequence of SEQ ID NO: 10, and a light chain CDR1, a light chain CDR2, and a light chain CDR3 of a VL comprising an amino acid sequence of SEQ ID NO: 9;
c) a heavy chain CDR1, a heavy chain CDR2, a heavy chain CDR3 of a VH comprising an amino acid sequence of SEQ ID NO: 16, and a light chain CDR1, a light chain CDR2, and a light chain CDR3 of a VL comprising an amino acid sequence of SEQ ID NO: 15;
d) a heavy chain CDR1, a heavy chain CDR2, a heavy chain CDR3 of a VH comprising an amino acid sequence of SEQ ID NO: 22, and a light chain CDR1, a light chain CDR2, and a light chain CDR3 of a VL comprising an amino acid sequence of SEQ ID NO: 21;
e) a heavy chain CDR1, a heavy chain CDR2, a heavy chain CDR3 of a VH comprising an amino acid sequence of SEQ ID NO: 26, and a light chain CDR1, a light chain CDR2, and a light chain CDR3 of a VL comprising an amino acid sequence of SEQ ID NO: 25;
f) a heavy chain CDR1, a heavy chain CDR2, a heavy chain CDR3 of a VH comprising an amino acid sequence of SEQ ID NO: 32, and a light chain CDR1, a light chain CDR2, and a light chain CDR3 of a VL comprising an amino acid sequence of SEQ ID NO: 21;
g) a heavy chain CDR1, a heavy chain CDR2, a heavy chain CDR3 of a VH comprising an amino acid sequence of SEQ ID NO: 36, and a light chain CDR1, a light chain CDR2, and a light chain CDR3 of a VL comprising an amino acid sequence of SEQ ID NO: 35;
h) a heavy chain CDR1, a heavy chain CDR2, a heavy chain CDR3 of a VH comprising an amino acid sequence of SEQ ID NO: 42, and a light chain CDR1, a light chain CDR2, and a light chain CDR3 of a VL comprising an amino acid sequence of SEQ ID NO: 41;
i) a heavy chain CDR1, a heavy chain CDR2, a heavy chain CDR3 of a VH comprising an amino acid sequence of SEQ ID NO: 46, and a light chain CDR1, a light chain CDR2, and a light chain CDR3 of a VL comprising an amino acid sequence of SEQ ID NO: 21;
j) a heavy chain CDR1, a heavy chain CDR2, a heavy chain CDR3 of a VH comprising an amino acid sequence of SEQ ID NO: 50, and a light chain CDR1, a light chain CDR2, and a light chain CDR3 of a VL comprising an amino acid sequence of SEQ ID NO: 49;
k) a heavy chain CDR1, a heavy chain CDR2, a heavy chain CDR3 of a VH comprising an amino acid sequence of SEQ ID NO: 54, and a light chain CDR1, a light chain CDR2, and a light chain CDR3 of a VL comprising an amino acid sequence of SEQ ID NO: 21;
1) a heavy chain CDR1, a heavy chain CDR2, a heavy chain CDR3 of a VH comprising an amino acid sequence of SEQ ID NO: 56, and a light chain CDR1, a light chain CDR2, and a light chain CDR3 of a VL comprising an amino acid sequence of SEQ ID NO: 21;
m) a heavy chain CDR1, a heavy chain CDR2, a heavy chain CDR3 of a VH comprising an amino acid sequence of SEQ ID NO: 60, and a light chain CDR1, a light chain CDR2, and a light chain CDR3 of a VL comprising an amino acid sequence of SEQ ID NO: 59;
n) a heavy chain CDR1, a heavy chain CDR2, a heavy chain CDR3 of a VH comprising an amino acid sequence of SEQ ID NO: 64, and a light chain CDR1, a light chain CDR2, and a light chain CDR3 of a VL comprising an amino acid sequence of SEQ ID NO: 63;
o) a heavy chain CDR1, a heavy chain CDR2, a heavy chain CDR3 of a VH comprising an amino acid sequence of SEQ ID NO: 70, and a light chain CDR1, a light chain CDR2, and a light chain CDR3 of a VL comprising an amino acid sequence of SEQ ID NO: 69;
p) a heavy chain CDR1, a heavy chain CDR2, a heavy chain CDR3 of a VH comprising an amino acid sequence of SEQ ID NO: 74, and a light chain CDR1, a light chain CDR2, and a light chain CDR3 of a VL comprising an amino acid sequence of SEQ ID NO: 21;
q) a heavy chain CDR1, a heavy chain CDR2, a heavy chain CDR3 of a VH comprising an amino acid sequence of SEQ ID NO: 78, and a light chain CDR1, a light chain CDR2, and a light chain CDR3 of a VL comprising an amino acid sequence of SEQ ID NO: 77;
r) a heavy chain CDR1, a heavy chain CDR2, a heavy chain CDR3 of a VH comprising an amino acid sequence of SEQ ID NO: 81, and a light chain CDR1, a light chain CDR2, and a light chain CDR3 of a VL comprising an amino acid sequence of SEQ ID NO: 21;
s) a heavy chain CDR1, a heavy chain CDR2, a heavy chain CDR3 of a VH comprising an amino acid sequence of SEQ ID NO: 31, and a light chain CDR1, a light chain CDR2, and a light chain CDR3 of a VL comprising an amino acid sequence of SEQ ID NO: 21; or
t) a heavy chain CDR1, a heavy chain CDR2, a heavy chain CDR3 of a VH comprising an amino acid sequence of SEQ ID NO: 73, and a light chain CDR1, a light chain CDR2, and a light chain CDR3 of a VL comprising an amino acid sequence of SEQ ID NO: 53.

5.-6. (canceled)

7. The antibody or antigen-binding fragment of claim 1, comprising
a) a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 5;
b) a heavy chain CDR1 comprising the amino acid sequence of SEQ ID NO: 12, a heavy chain CDR2 comprising the amino acid sequence of SEQ ID NO: 13, a heavy chain CDR3 comprising the amino acid sequence of SEQ ID NO: 14, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 11;
c) a heavy chain CDR1 comprising the amino acid sequence of SEQ ID NO: 6, a heavy chain CDR2 comprising the amino acid sequence of SEQ ID NO: 18, a heavy chain CDR3 comprising the amino acid sequence of SEQ ID NO: 19, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 17;
d) a heavy chain CDR1 comprising the amino acid sequence of SEQ ID NO: 6, a heavy chain CDR2 comprising the amino acid sequence of SEQ ID NO: 23, a heavy chain CDR3 comprising the amino acid sequence of SEQ ID NO: 24, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
e) a heavy chain CDR1 comprising the amino acid sequence of SEQ ID NO: 6, a heavy chain CDR2 comprising the amino acid sequence of SEQ ID NO: 28, a heavy chain CDR3 comprising the amino acid sequence of SEQ ID NO: 29, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 27;
f) a heavy chain CDR1 comprising the amino acid sequence of SEQ ID NO: 6, a heavy chain CDR2 comprising the amino acid sequence of SEQ ID NO: 30, a heavy chain CDR3 comprising the amino acid sequence of SEQ ID NO: 33, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
g) a heavy chain CDR1 comprising the amino acid sequence of SEQ ID NO: 37, a heavy chain CDR2 comprising the amino acid sequence of SEQ ID NO: 38, a heavy chain CDR3 comprising the amino acid sequence of SEQ ID NO: 39, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 34;
h) a heavy chain CDR1 comprising the amino acid sequence of SEQ ID NO: 6, a heavy chain CDR2 comprising the amino acid sequence of SEQ ID NO: 43, a heavy chain CDR3 comprising the amino acid sequence of SEQ ID NO: 44, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 40;
i) a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 47, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
j) a heavy chain CDR1 comprising the amino acid sequence of SEQ ID NO: 6, a heavy chain CDR2 comprising the amino acid sequence of SEQ ID NO: 51, a heavy chain CDR3 comprising the amino acid sequence of SEQ ID NO: 52, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 48;
k) a heavy chain CDR1 comprising the amino acid sequence of SEQ ID NO: 6, a heavy chain CDR2 comprising the amino acid sequence of SEQ ID NO: 23, a heavy chain CDR3 comprising the amino acid sequence of SEQ ID NO: 24, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
1) a heavy chain CDR1 comprising the amino acid sequence of SEQ ID NO: 6, a heavy chain CDR2 comprising the amino acid sequence of SEQ ID NO: 13, a heavy chain CDR3 comprising the amino acid sequence of SEQ ID NO: 57, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
m) a heavy chain CDR1 comprising the amino acid sequence of SEQ ID NO: 6, a heavy chain CDR2 comprising the amino acid sequence of SEQ ID NO: 61, a heavy chain CDR3 comprising the amino acid sequence of SEQ ID NO: 62, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 58;
n) a heavy chain CDR1 comprising the amino acid sequence of SEQ ID NO: 6, a heavy chain CDR2 comprising the amino acid sequence of SEQ ID NO: 66, a heavy chain CDR3 comprising the amino acid sequence of SEQ ID NO: 67, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 65;
o) a heavy chain CDR1 comprising the amino acid sequence of SEQ ID NO: 6, a heavy chain CDR2 comprising the amino acid sequence of SEQ ID NO: 71, a heavy chain CDR3 comprising the amino acid sequence of SEQ ID NO: 72, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 68;
p) a heavy chain CDR1 comprising the amino acid sequence of SEQ ID NO: 6, a heavy chain CDR2 comprising the amino acid sequence of SEQ ID NO: 23, a heavy chain CDR3 comprising the amino acid sequence of SEQ ID NO: 75, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
q) a heavy chain CDR1 comprising the amino acid sequence of SEQ ID NO: 6, a heavy chain CDR2 comprising the amino acid sequence of SEQ ID NO: 79, a heavy chain CDR3 comprising the amino acid sequence of SEQ ID NO: 80, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 76;
r) a heavy chain CDR1 comprising the amino acid sequence of SEQ ID NO: 6, a heavy chain CDR2 comprising the amino acid sequence of SEQ ID NO: 23, a heavy chain CDR3 comprising the amino acid sequence of SEQ ID NO: 82, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20;
s) a heavy chain CDR1 comprising the amino acid sequence of SEQ ID NO: 6, a heavy chain CDR2 comprising the amino acid sequence of SEQ ID NO: 23, a heavy chain CDR3 comprising the amino acid sequence of SEQ ID NO: 45, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 20; or
t) a heavy chain CDR1 comprising the amino acid sequence of SEQ ID NO: 37, a heavy chain CDR2 comprising the amino acid sequence of SEQ ID NO: 83, a heavy chain CDR3 comprising the amino acid sequence of SEQ ID NO: 84, a light chain CDR1 comprising the amino acid sequence of SEQ ID NO: 3, a light chain CDR2 comprising the amino acid sequence of SEQ ID NO: 4, and a light chain CDR3 comprising the amino acid sequence of SEQ ID NO: 55.

8. (canceled)

9. The antibody or antigen-binding fragment of claim 1, comprising
a) a VH comprising an amino acid sequence of SEQ ID NO: 2, and a VL comprising an amino acid sequence of SEQ ID NO: 1;
b) a VH comprising an amino acid sequence of SEQ ID NO: 10, and a VL comprising an amino acid sequence of SEQ ID NO: 9;
c) a VH comprising an amino acid sequence of SEQ ID NO: 16, and a VL comprising an amino acid sequence of SEQ ID NO: 15;
d) a VH comprising an amino acid sequence of SEQ ID NO: 22, and a VL comprising an amino acid sequence of SEQ ID NO: 21;
e) a VH comprising an amino acid sequence of SEQ ID NO: 26, and a VL comprising an amino acid sequence of SEQ ID NO: 25;
f) a VH comprising an amino acid sequence of SEQ ID NO: 32, and a VL comprising an amino acid sequence of SEQ ID NO: 21;
g) a VH comprising an amino acid sequence of SEQ ID NO: 36, and a VL comprising an amino acid sequence of SEQ ID NO: 35;
h) a VH comprising an amino acid sequence of SEQ ID NO: 42, and a VL comprising an amino acid sequence of SEQ ID NO: 41;
i) a VH comprising an amino acid sequence of SEQ ID NO: 46, and a VL comprising an amino acid sequence of SEQ ID NO: 21;
j) a VH comprising an amino acid sequence of SEQ ID NO: 50, and a VL comprising an amino acid sequence of SEQ ID NO: 49;
k) a VH comprising an amino acid sequence of SEQ ID NO: 54, and a VL comprising an amino acid sequence of SEQ ID NO: 21;
1) a VH comprising an amino acid sequence of SEQ ID NO: 56, and a VL comprising an amino acid sequence of SEQ ID NO: 21;
m) a VH comprising an amino acid sequence of SEQ ID NO: 60, and a VL comprising an amino acid sequence of SEQ ID NO: 59;
n) a VH comprising an amino acid sequence of SEQ ID NO: 64, and a VL comprising an amino acid sequence of SEQ ID NO: 63;
o) a VH comprising an amino acid sequence of SEQ ID NO: 70, and a VL comprising an amino acid sequence of SEQ ID NO: 69;
p) a VH comprising an amino acid sequence of SEQ ID NO: 74, and a VL comprising an amino acid sequence of SEQ ID NO: 21;
q) a VH comprising an amino acid sequence of SEQ ID NO: 78, and a VL comprising an amino acid sequence of SEQ ID NO: 77;
r) a VH comprising an amino acid sequence of SEQ ID NO: 81, and a VL comprising an amino acid sequence of SEQ ID NO: 21;
s) a VH comprising an amino acid sequence of SEQ ID NO: 31, and a VL comprising an amino acid sequence of SEQ ID NO: 21; or
t) a VH comprising an amino acid sequence of SEQ ID NO: 73, and a VL comprising an amino acid sequence of SEQ ID NO: 53.

10.-16. (canceled)

17. The antibody or antigen-binding fragment of claim 1, wherein the antibody or antigen-binding fragment has one or more characteristics selected from:
a) binds to isoform 1, isoform 2, and/or isoform 3 of CD97;
b) binds to one or more amino acid residues of isoform 1 of CD97 selected from T351, Q376, R380, K382, K519, and T524:
c) binds to the G protein-coupled receptor (GPCR) autoproteolysis-inducing (GAIN) domain in CD97;
d) binds to the first epidermal growth factor (EGF)-like domain in CD97;
e) binds to the third EGF-like domain in CD97;
f) binds to isoform 1 of CD97 at a dissociation constant (K_{D}) about 1×10⁻⁷ M or less;
g) binds to isoform 2 of CD97 at a K_{D} about 1×10⁻⁷ M or less;
h) does not bind to EGF-like module-containing mucin-like hormone receptor-like 2 (EMR2); and
i) does not bind to either isoform 1 or isoform 4 of EMR2.

18.-30. (canceled)

31. The antibody or antigen-binding fragment of claim 2, wherein the antibody or antigen-binding fragment is a human antibody, a humanized antibody, a chimeric antibody, a murine antibody, a monoclonal antibody, a single chain antibody, a bispecific antibody or antigen-binding fragment thereof, a Fab, a Fab′, a F(ab′)2, a Fv, a scFv, or a nanobody.

32.-34. (canceled)

35. The antibody or antigen-binding fragment of claim 2, wherein the antibody or antigen-binding fragment is of IgG1, IgG2, IgG3, or IgG4 subclass.

36. The antibody or antigen-binding fragment of claim 35, wherein the antibody or antigen-binding fragment is of IgG1 subclass.

37. The antibody or antigen-binding fragment of claim 36, wherein the antibody or antigen-binding fragment comprises L234A, L235A, S239C, and/or P329G mutations in the IgG1 Fc region.

38. (canceled)

39. An antibody-drug conjugate comprising the antibody or antigen-binding fragment of claim 2, conjugated to a heterologous moiety.

40-46. (canceled)

47. A method for making the antibody-drug conjugate of claim 39 comprising
(a) contacting the antibody or antigen-binding fragment with the heterologous moiety under the conditions favorable for conjugation of the antibody or antigen-binding fragment to the heterologous moiety; and
(b) optionally, isolating the antibody-drug conjugate produced in step (a).

48. A polynucleotide encoding the antibody or antigen-binding fragment of claim 2.

49. A vector comprising the polynucleotide of claim 48.

50. A host cell comprising the polynucleotide of claim 48.

51.-52. (canceled)

53. A method of producing an antibody or antigen-binding fragment, wherein said method comprises culturing the host cell of claim 50, and isolating said antibody or antigen-binding fragment.

54. A pharmaceutical composition comprising the antibody or antigen-binding fragment of claim 2 and a pharmaceutically acceptable carrier or diluent.

55. A kit comprising (i) the antibody or antigen-binding fragment of claim 2 and (ii) packaging for the same.

56. A method of killing a cell expressing at least an isoform of CD97, comprising contacting the cell with an effective amount of the antibody or antigen-binding fragment of claim 2.

57. (canceled)

58. A method of treating or preventing a cancer associated with at least an isoform of CD97 in a subject in need thereof, comprising administering to the subject an effective amount of the antibody or antigen-binding fragment of claim 2.

59.-75. (canceled)
